SEQUENCE LISTING (602 sequences)

SEQ ID NO: 1 - protein, 10 aa, synthetic construct
YLGSGIHSGA

SEQ ID NO: 2 - protein, 33 aa, synthetic construct
PDRKAAVSHWQQQSYLGSGIHSGATTTAPSLSG

SEQ ID NO: 3 - protein, 27 aa, synthetic construct
KAAVSHWQQQSYLGSGIHSGATTTAPS

SEQ ID NO: 4 - protein, 25 aa, synthetic construct
AAVSHWQQQSYLGSGIHSGATTTAP

SEQ ID NO: 5 - protein, 33 aa, synthetic construct
RKAAVSHWQQQSYLGSGIHSGATTTAPSLSGKG

SEQ ID NO: 6 - protein, 27 aa, synthetic construct
AVSHWQQQSYLGSGIHSGATTTAPSLS

SEQ ID NO: 7 - protein, 25 aa, synthetic construct
VSHWQQQSYLGSGIHSGATTTAPSL

SEQ ID NO: 8 - protein, 39 aa, synthetic construct
MATQADLMELDMAMEPDRKAAVSHWQQQSYLGSGIHSGA

SEQ ID NO: 9 - protein, 78 aa, synthetic construct
MATQADLMELDMAMEPDRKAAVSHWQQQSYLGSGIHSGATTTAPSLSGKGNPEEEDVDTSQVLYEWEQGFSQSFTQEQ

SEQ ID NO: 10 - protein, 67 aa, synthetic construct
MATQADLMELDMAMEPDRKAAVSHWQQQSYLGSGIHSGATTTAPSLSGKGNPEEEDVDTSQVLYEWE

SEQ ID NO: 11 - protein, 57 aa, synthetic construct
DLMELDMAMEPDRKAAVSHWQQQSYLGSGIHSGATTTAPSLSGKGNPEEEDVDTSQV

SEQ ID NO: 12 - protein, 47 aa, synthetic construct
DMAMEPDRKAAVSHWQQQSYLGSGIHSGATTTAPSLSGKGNPEEEDV

SEQ ID NO: 13 - protein, 37 aa, synthetic construct
PDRKAAVSHWQQQSYLGSGIHSGATTTAPSLSGKGNP

SEQ ID NO: 14 - protein, 18 aa, synthetic construct
QQQSYLGSGIHSGATTTA

SEQ ID NO: 15 - protein, 781 aa, synthetic construct
MATQADLMELDMAMEPDRKAAVSHWQQQSYLGSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 16 - protein, 9 aa, synthetic construct
TTAPPLSGK

SEQ ID NO: 17 - protein, 780 aa, synthetic construct
MATQADLMELDMAMEPDRKAAVSHWQQQSYLDSGIHSGATTAPPLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 18 - protein, 49 aa, synthetic construct
MATQADLMELDMAMEPDRKAAVSHWQQQSYLDSGIHSGATTTAPPLSGK

SEQ ID NO: 19 - protein, 78 aa, synthetic construct
MATQADLMELDMAMEPDRKAAVSHWQQQSYLDSGIHSGATTTAPPLSGKGNPEEEDVDTSQVLYEWEQGFSQSFTQEQ

SEQ ID NO: 20 - protein, 68 aa, synthetic construct
MATQADLMELDMAMEPDRKAAVSHWQQQSYLDSGIHSGATTTAPPLSGKGNPEEEDVDTSQVLYEWEQ

SEQ ID NO: 21 - protein, 58 aa, synthetic construct
DMAMEPDRKAAVSHWQQQSYLDSGIHSGATTTAPPLSGKGNPEEEDVDTSQVLYEWEQ

SEQ ID NO: 22 - protein, 48 aa, synthetic construct
AVSHWQQQSYLDSGIHSGATTTAPPLSGKGNPEEEDVDTSQVLYEWEQ

SEQ ID NO: 23 - protein, 38 aa, synthetic construct
QSYLDSGIHSGATTTAPPLSGKGNPEEEDVDTSQVLYE

SEQ ID NO: 24 - protein, 33 aa, synthetic construct
SYLDSGIHSGATTTAPPLSGKGNPEEEDVDTSQ

SEQ ID NO: 25 - protein, 28 aa, synthetic construct
DSGGIHSGATTTAPPLSGKGNPEEEDVD

SEQ ID NO: 26 - protein, 25 aa, synthetic construct
SGIHSGATTTAPPLSGKGNPEEEDV

SEQ ID NO: 27 - protein, 34 aa, synthetic construct
IHSGATTTAPPLSGKGNPEEEDVDTSQVLYEWEQ

SEQ ID NO: 28 - protein, 412 aa, synthetic construct
DRKAAVSHWQQQSYLDSGIHSGATTTAPPLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLT

SEQ ID NO: 29 - protein, 166 aa, synthetic construct
MATQADLMELDMAMEPDRKAAVSHWQQQSYLDSGIHSGATTTAPPLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQV

SEQ ID NO: 30 - protein, 17 aa, synthetic construct
SGATTTAPPLSGKGNPE

SEQ ID NO: 31 - protein, 9 aa, synthetic construct
MLWCGTCFV

SEQ ID NO: 32 - protein, 9 aa, synthetic construct
RPRDCYRRM

SEQ ID NO: 33 - protein, 9 aa, synthetic construct
TLCAYIYEL

SEQ ID NO: 34 - protein, 8 aa, synthetic construct
YLEDFFVV

SEQ ID NO: 35 - protein, 9 aa, synthetic construct
MMKVMMTAL

SEQ ID NO: 36 - protein, 10 aa, synthetic construct
FKLLGALLLL

SEQ ID NO: 37 - protein, 9 aa, synthetic construct
ALAALVAAL

SEQ ID NO: 38 - protein, 10 aa, synthetic construct
KLNHDILAMV

SEQ ID NO: 39 - protein, 9 aa, synthetic construct
VLLEVHVPA

SEQ ID NO: 40 - protein, 9 aa, synthetic construct
RPTGKRCCI

SEQ ID NO: 41 - protein, 10 aa, synthetic construct
SPLTSRTPRI

SEQ ID NO: 42 - protein, 10 aa, synthetic construct
RLLEINMKEI

SEQ ID NO: 43 - protein, 9 aa, synthetic construct
QLLGFLQRL

SEQ ID NO: 44 - protein, 9 aa, synthetic construct
FSRQRLPAF

SEQ ID NO: 45 - protein, 9 aa, synthetic construct
GLATVRAYL

SEQ ID NO: 46 - protein, 9 aa, synthetic construct
CICNRRFAL

SEQ ID NO: 47 - protein, 10 aa, synthetic construct
IPSNSTRLSV

SEQ ID NO: 48 - protein, 10 aa, synthetic construct
GIYTHVTYYL

SEQ ID NO: 49 - protein, 9 aa, synthetic construct
RLAGFAARI

SEQ ID NO: 50 - protein, 9 aa, synthetic construct
FIMNQFKAA

SEQ ID NO: 51 - protein, 11 aa, synthetic construct
GPRMPQTAKKV

SEQ ID NO: 52 - protein, 8 aa, synthetic construct
LIRVKSNL

SEQ ID NO: 53 - protein, 8 aa, synthetic construct
FTEYIHAY

SEQ ID NO: 54 - protein, 10 aa, synthetic construct
GPGGGGSGTL

SEQ ID NO: 55 - protein, 10 aa, synthetic construct
LVRKRDVYVV

SEQ ID NO: 56 - protein, 9 aa, synthetic construct
FLGHHDLKM

SEQ ID NO: 57 - protein, 9 aa, synthetic construct
SSREFAAAL

SEQ ID NO: 58 - protein, 9 aa, synthetic construct
STGIKFNSI

SEQ ID NO: 59 - protein, 9 aa, synthetic construct
RVRGVEYAV

SEQ ID NO: 60 - protein, 10 aa, synthetic construct
LQKVKETLSM

SEQ ID NO: 61 - protein, 9 aa, synthetic construct
LKKNHFVPA

SEQ ID NO: 62 - protein, 9 aa, synthetic construct
LAANGPTAL

SEQ ID NO: 63 - protein, 9 aa, synthetic construct
QRSSSVQSM

SEQ ID NO: 64 - protein, 9 aa, synthetic construct
RPQHGKKLC

SEQ ID NO: 65 - protein, 10 aa, synthetic construct
CVLMRLQEKL

SEQ ID NO: 66 - protein, 9 aa, synthetic construct
ERILTLLEL

SEQ ID NO: 67 - protein, 10 aa, synthetic construct
VQMAPVQPRI

SEQ ID NO: 68 - protein, 10 aa, synthetic construct
YPYQAANGCA

SEQ ID NO: 69 - protein, 10 aa, synthetic construct
RLLETFFNFV

SEQ ID NO: 70 - protein, 10 aa, synthetic construct
YVPITGMPTV

SEQ ID NO: 71 - protein, 10 aa, synthetic construct
YIMDNKLSQI

SEQ ID NO: 72 - protein, 8 aa, synthetic construct
RRSHRLSK

SEQ ID NO: 73 - protein, 9 aa, synthetic construct
FSNYSSCHY

SEQ ID NO: 74 - protein, 11 aa, synthetic construct
SIIPDFDIYYV

SEQ ID NO: 75 - protein, 10 aa, synthetic construct
LLSPDVSQGL

SEQ ID NO: 76 - protein, 10 aa, synthetic construct
LESNTIIDVY

SEQ ID NO: 77 - protein, 9 aa, synthetic construct
NMYYFRIQA

SEQ ID NO: 78 - protein, 10 aa, synthetic construct
EECVKAFSQF

SEQ ID NO: 79 - protein, 9 aa, synthetic construct
STTGVSSEF

SEQ ID NO: 80 - protein, 9 aa, synthetic construct
YESLKEGRY

SEQ ID NO: 81 - protein, 10 aa, synthetic construct
RLKGIWNWRA

SEQ ID NO: 82 - protein, 10 aa, synthetic construct
SLSEEDEYSI

SEQ ID NO: 83 - protein, 9 aa, synthetic construct
VIKNAIASL

SEQ ID NO: 84 - protein, 9 aa, synthetic construct
GLSEDIQKA

SEQ ID NO: 85 - protein, 9 aa, synthetic construct
QARKYLAQS

SEQ ID NO: 86 - protein, 9 aa, synthetic construct
NTMDLPEQI

SEQ ID NO: 87 - protein, 10 aa, synthetic construct
EEPASTVCIM

SEQ ID NO: 88 - protein, 9 aa, synthetic construct
SGSVVVQTV

SEQ ID NO: 89 - protein, 9 aa, synthetic construct
ERQFKEEML

SEQ ID NO: 90 - protein, 10 aa, synthetic construct
MTAGFCYGTW

SEQ ID NO: 91 - protein, 9 aa, synthetic construct
MPNWLSVLV

SEQ ID NO: 92 - protein, 9 aa, synthetic construct
EPFRDTAVW

SEQ ID NO: 93 - protein, 9 aa, synthetic construct
AVIAEAHSW

SEQ ID NO: 94 - protein, 10 aa, synthetic construct
KSSSRPGGPY

SEQ ID NO: 95 - protein, 10 aa, synthetic construct
NAMRFFNLSW

SEQ ID NO: 96 - protein, 9 aa, synthetic construct
LTPVILALW

SEQ ID NO: 97 - protein, 10 aa, synthetic construct
HVWENNNPFR

SEQ ID NO: 98 - protein, 9 aa, synthetic construct
STQRPNGTW

SEQ ID NO: 99 - protein, 10 aa, synthetic construct
SPSLMSETEW

SEQ ID NO: 100 - protein, 9 aa, synthetic construct
MAESLRSSR

SEQ ID NO: 101 - protein, 10 aa, synthetic construct
CSVTQAGVQW

SEQ ID NO: 102 - protein, 9 aa, synthetic construct
VPFVTEALY

SEQ ID NO: 103 - protein, 9 aa, synthetic construct
VVRLLSMSR

SEQ ID NO: 104 - protein, 9 aa, synthetic construct
RSVQVSLML

SEQ ID NO: 105 - protein, 9 aa, synthetic construct
GAVAATRLY

SEQ ID NO: 106 - protein, 11 aa, synthetic construct
FSDVYHLNISW

SEQ ID NO: 107 - protein, 10 aa, synthetic construct
SPLGSLDRPW

SEQ ID NO: 108 - protein, 9 aa, synthetic construct
EGYNTKNTR

SEQ ID NO: 109 - protein, 9 aa, synthetic construct
LSLLEKDYF

SEQ ID NO: 110 - protein, 10 aa, synthetic construct
KTVPMVPPGI

SEQ ID NO: 111 - protein, 10 aa, synthetic construct
VDSSKMIFKF

SEQ ID NO: 112 - protein, 9 aa, synthetic construct
SISEEITPR

SEQ ID NO: 113 - protein, 10 aa, synthetic construct
LPRMECSGAI

SEQ ID NO: 114 - protein, 10 aa, synthetic construct
RGDIFENVNY

SEQ ID NO: 115 - protein, 9 aa, synthetic construct
MTGSSGVDR

SEQ ID NO: 116 - protein, 9 aa, synthetic construct
TTDRCIRFW

SEQ ID NO: 117 - protein, 10 aa, synthetic construct
MPESPYLVPV

SEQ ID NO: 118 - protein, 9 aa, synthetic construct
TPVRIRSNR

SEQ ID NO: 119 - protein, 9 aa, synthetic construct
RTQVDFRGR

SEQ ID NO: 120 - protein, 10 aa, synthetic construct
NFMSLQEILK

SEQ ID NO: 121 - protein, 9 aa, synthetic construct
IALPLPPGI

SEQ ID NO: 122 - protein, 11 aa, synthetic construct
HTIPVPPTTVR

SEQ ID NO: 123 - protein, 11 aa, synthetic construct
KDTQEFNLSAY

SEQ ID NO: 124 - protein, 9 aa, synthetic construct
LFGLAVKSR

SEQ ID NO: 125 - protein, 10 aa, synthetic construct
DVKKDDMSTR

SEQ ID NO: 126 - protein, 11 aa, synthetic construct
HVCNPSTLGGR

SEQ ID NO: 127 - protein, 9 aa, synthetic construct
ANRGALPAR

SEQ ID NO: 128 - protein, 9 aa, synthetic construct
RRRELTDQL

SEQ ID NO: 129 - DNA, 18 bp, synthetic construct
  source 1..18: mol_type = other DNA
ggacaaarcmttgascag

SEQ ID NO: 130 - DNA, 18 bp, synthetic construct
  source 1..18: mol_type = other DNA
aaggaccaagtgtttcag

SEQ ID NO: 131 - DNA, 18 bp, synthetic construct
  source 1..18: mol_type = other DNA
gctcagtcagtggctcag

SEQ ID NO: 132 - DNA, 18 bp, synthetic construct
  source 1..18: mol_type = other DNA
swtgctaagaccacmcag

SEQ ID NO: 133 - DNA, 18 bp, synthetic construct
  source 1..18: mol_type = other DNA
ggagagrrtgtggrgcwg

SEQ ID NO: 134 - DNA, 18 bp, synthetic construct
  source 1..18: mol_type = other DNA
agccaaaagatagaacag

SEQ ID NO: 135 - DNA, 18 bp, synthetic construct
  source 1..18: mol_type = other DNA
gaaraccaggtgrmgcas

SEQ ID NO: 136 - DNA, 18 bp, synthetic construct
  source 1..18: mol_type = other DNA
rcccagtcdgtgasccag

SEQ ID NO: 137 - DNA, 18 bp, synthetic construct
  source 1..18: mol_type = other DNA
ggarattcagtgryccag

SEQ ID NO: 138 - DNA, 18 bp, synthetic construct
  source 1..18: mol_type = other DNA
aaaaaysaagtggagcag

SEQ ID NO: 139 - DNA, 18 bp, synthetic construct
  source 1..18: mol_type = other DNA
ctmcataywctggagyag

SEQ ID NO: 140 - DNA, 18 bp, synthetic construct
  source 1..18: mol_type = other DNA
crgaaggaggtggagcag

SEQ ID NO: 141 - DNA, 18 bp, synthetic construct
  source 1..18: mol_type = other DNA
gcycagaarrtaacycaa

SEQ ID NO: 142 - DNA, 18 bp, synthetic construct
  source 1..18: mol_type = other DNA
gcycagasagtcactcag

SEQ ID NO: 143 - DNA, 18 bp, synthetic construct
  source 1..18: mol_type = other DNA
agtcaacagggagaagag

SEQ ID NO: 144 - DNA, 18 bp, synthetic construct
  source 1..18: mol_type = other DNA
ggagactcggttacccag

SEQ ID NO: 145 - DNA, 18 bp, synthetic construct
  source 1..18: mol_type = other DNA
aaacaggaggtgacrcag

SEQ ID NO: 146 - DNA, 18 bp, synthetic construct
  source 1..18: mol_type = other DNA
ggaatacaagtggagcag

SEQ ID NO: 147 - DNA, 12 bp, synthetic construct
  source 1..12: mol_type = other DNA
caggtgaaacaa

SEQ ID NO: 148 - DNA, 18 bp, synthetic construct
  source 1..18: mol_type = other DNA
rwrctgaamgtggaacaa

SEQ ID NO: 149 - DNA, 18 bp, synthetic construct
  source 1..18: mol_type = other DNA
ggacaacaggtaatgcaa

SEQ ID NO: 150 - DNA, 15 bp, synthetic construct
  source 1..15: mol_type = other DNA
acccagctggagcag

SEQ ID NO: 151 - DNA, 18 bp, synthetic construct
  source 1..18: mol_type = other DNA
aaagtggagcagagtcct

SEQ ID NO: 152 - DNA, 18 bp, synthetic construct
  source 1..18: mol_type = other DNA
gaccagcaagttaagcaa

SEQ ID NO: 153 - DNA, 15 bp, synthetic construct
  source 1..15: mol_type = other DNA
caaccagtgcagagt

SEQ ID NO: 154 - DNA, 18 bp, synthetic construct
  source 1..18: mol_type = other DNA
cagagggtcattcaatcc

SEQ ID NO: 155 - DNA, 18 bp, synthetic construct
  source 1..18: mol_type = other DNA
aaggatgtgatacagagt

SEQ ID NO: 156 - DNA, 18 bp, synthetic construct
  source 1..18: mol_type = other DNA
agccaagaactggagcag

SEQ ID NO: 157 - DNA, 18 bp, synthetic construct
  source 1..18: mol_type = other DNA
ggtcaacagctgaatcag

SEQ ID NO: 158 - DNA, 18 bp, synthetic construct
  source 1..18: mol_type = other DNA
gaagacaaggtggtacaa

SEQ ID NO: 159 - DNA, 18 bp, synthetic construct
  source 1..18: mol_type = other DNA
caactgccagtggaacag

SEQ ID NO: 160 - DNA, 18 bp, synthetic construct
  source 1..18: mol_type = other DNA
agcaattcagtcaagcag

SEQ ID NO: 161 - protein, 15 aa, synthetic construct
CAVGGSGGGADGLTF

SEQ ID NO: 162 - protein, 11 aa, synthetic construct
CAVGINARLMF

SEQ ID NO: 163 - protein, 14 aa, synthetic construct
CAGPMKTSYDKVIF

SEQ ID NO: 164 - protein, 16 aa, synthetic construct
CAVRDLGAGGGNKLTF

SEQ ID NO: 165 - protein, 12 aa, synthetic construct
CAASKPGNQFYF

SEQ ID NO: 166 - protein, 17 aa, synthetic construct
CVVNRLHSYNYGQNFVF

SEQ ID NO: 167 - protein, 14 aa, synthetic construct
CAVRGTDSWGKLQF

SEQ ID NO: 168 - protein, 12 aa, synthetic construct
CASSAVGNTIYF

SEQ ID NO: 169 - protein, 15 aa, synthetic construct
CVVSEGDSGGFKTIF

SEQ ID NO: 170 - protein, 16 aa, synthetic construct
CASSPLAGGISDTQYF

SEQ ID NO: 171 - protein, 12 aa, synthetic construct
CASSSANYGYTF

SEQ ID NO: 172 - protein, 13 aa, synthetic construct
CASSTLVRETQYF

SEQ ID NO: 173 - protein, 15 aa, synthetic construct
CSAREYQGVKSPLHF

SEQ ID NO: 174 - protein, 13 aa, synthetic construct
CASSFKMNTEAFF

SEQ ID NO: 175 - DNA, 6699 bp, synthetic construct
  source 1..6699: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagaccctgcaggttaattaagccaccaagtcttctttttttgaagacttcagaaccctgatcctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaacttccagaacctgagcgtgatcggcttccggatcctgctgctgaaggtggccggcttcaacctgctgatgaccctgagactgtggtccagccgggccaagagatctggcagcggcgccaccaatttcagcctgctgaaacaggccggcgacgtggaagagaaccctaagtcttctttttgaagacttgatctgaacaaggtgttccccccagaggtggccgtgttcgagccttctgaggccgagatcagccacacccagaaagccaccctcgtgtgcctggccaccggctttttccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgtgtcctatcagcagggcgtgctgagcgccaccatcctgtacgagatcctgctgggcaaggccaccctgtacgccgtgctggtgtctgccctggtgctgatggccatggtcaagcggaaggacttctgaatttgtagatcgacggatccctcgagctcaagcttcgaattctgcagtcgacggtaccgcgggcccgggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgatttaaagacaggatatcagtggtccaggctctagttttgactcaacaatatcaccagctgaagcctatagagtacgagccatagataaaataaaagattttatttagtctccagaaaaagggggg

SEQ ID NO: 176 - DNA, 5192 bp, synthetic construct
  source 1..5192: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcagatcttacgtagctagcggatcccaattgctcgagcctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 177 - protein, 369 aa, Homo sapiens
MPRAPKRQRCMPEEDLQSQSETQGLEGAQAPLAVEEDASSSTSTSSSFPSSFPSSSSSSSSSCYPLIPSTPEEVSADDETPNPPQSAQIACSSPSVVASLPLDQSDEGSSSQKEESPSTLQVLPDSESLPRSEIDEKVTDLVQFLLFKYQMKEPITKAEILESVIRNYEDHFPLLFSEASECMLLVFGIDVKEVDPTGHSFVLVTSLGLTYDGMLSDVQSMPKTGILILILSIVFIEGYCTPEEVIWEALNMMGLYDGMEHLIYGEPRKLLTQDWVQENYLEYRQVPGSDPARYEFLWGPRAHAEIRKMSLLKFLAKVNGSDPRSFPLWYEEALKDEEERAQDRIATTDDTTAMASASSSATGSFSYPE

SEQ ID NO: 178 - protein, 31 aa, Homo sapiens
EEVIWEALNMMGLYDGMEHLIYGEPRKLLTQ

SEQ ID NO: 179 - protein, 9 aa, Homo sapiens
GLYDGMEHL

SEQ ID NO: 180 - protein, 15 aa, Homo sapiens
EEVIWEALNMMGLYD

SEQ ID NO: 181 - protein, 15 aa, Homo sapiens
ALNMMGLYDGMEHLI

SEQ ID NO: 182 - protein, 15 aa, Homo sapiens
LYDGMEHLIYGEPRK

SEQ ID NO: 183 - protein, 31 aa, Homo sapiens
GPRAHAEIRKMSLLKFLAKVNGSDPRSFPLW

SEQ ID NO: 184 - protein, 9 aa, Homo sapiens
SLLKFLAKV

SEQ ID NO: 185 - protein, 15 aa, Homo sapiens
GPRAHAEIRKMSLLK

SEQ ID NO: 186 - protein, 15 aa, Homo sapiens
EIRKMSLLKFLAKVN

SEQ ID NO: 187 - protein, 15 aa, Homo sapiens
LLKFLAKVNGSDPRS

SEQ ID NO: 188 - protein, 31 aa, Homo sapiens
SESLPRSEIDEKVTDLVQFLLFKYQMKEPIT

SEQ ID NO: 189 - protein, 9 aa, Homo sapiens
KVTDLVQFL

SEQ ID NO: 190 - protein, 15 aa, Homo sapiens
SESLPRSEIDEKVTD

SEQ ID NO: 191 - protein, 15 aa, Homo sapiens
SEIDEKVTDLVQFLL

SEQ ID NO: 192 - protein, 15 aa, Homo sapiens
VTDLVQFLLFKYQMK

SEQ ID NO: 193 - protein, 31 aa, Homo sapiens
QVPGSDPARYEFLWGPRAHAEIRKMSLLKFL

SEQ ID NO: 194 - protein, 9 aa, Homo sapiens
FLWGPRAHA

SEQ ID NO: 195 - protein, 15 aa, Homo sapiens
QVPGSDPARYEFLWG

SEQ ID NO: 196 - protein, 15 aa, Homo sapiens
PARYEFLWGPRAHAE

SEQ ID NO: 197 - protein, 15 aa, Homo sapiens
LWGPRAHAEIRKMSL

SEQ ID NO: 198 - protein, 31 aa, Homo sapiens
VLVTSLGLTYDGMLSDVQSMPKTGILILILS

SEQ ID NO: 199 - protein, 9 aa, Homo sapiens
GMLSDVQSM

SEQ ID NO: 200 - protein, 15 aa, Homo sapiens
VLVTSLGLTYDGMLS

SEQ ID NO: 201 - protein, 15 aa, Homo sapiens
GLTYDGMLSDVQSMP

SEQ ID NO: 202 - protein, 15 aa, Homo sapiens
MLSDVQSMPKTGILI

SEQ ID NO: 203 - protein, 31 aa, Homo sapiens
VTDLVQFLLFKYQMKEPITKAEILESVIRNY

SEQ ID NO: 204 - protein, 10 aa, Homo sapiens
YQMKEPITKA

SEQ ID NO: 205 - protein, 15 aa, Homo sapiens
VTDLVQFLLFKYQMK

SEQ ID NO: 206 - protein, 15 aa, Homo sapiens
FLLFKYQMKEPITKA

SEQ ID NO: 207 - protein, 15 aa, Homo sapiens
QMKEPITKAEILESV

SEQ ID NO: 208 - protein, 31 aa, Homo sapiens
CMLLVFGIDVKEVDPTGHSFVLVTSLGLTYD

SEQ ID NO: 209 - protein, 9 aa, Homo sapiens
EVDPTGHSF

SEQ ID NO: 210 - protein, 15 aa, Homo sapiens
CMLLVFGIDVKEVDP

SEQ ID NO: 211 - protein, 15 aa, Homo sapiens
GIDVKEVDPTGHSFV

SEQ ID NO: 212 - protein, 15 aa, Homo sapiens
VDPTGHSFVLVTSLG

SEQ ID NO: 213 - protein, 31 aa, Homo sapiens
KYQMKEPITKAEILESVIRNYEDHFPLLFSE

SEQ ID NO: 214 - protein, 10 aa, Homo sapiens
EILESVIRNY

SEQ ID NO: 215 - protein, 15 aa, Homo sapiens
KYQMKEPITKAEILE

SEQ ID NO: 216 - protein, 15 aa, Homo sapiens
PITKAEILESVIRNY

SEQ ID NO: 217 - protein, 15 aa, Homo sapiens
ILESVIRNYEDHFPL

SEQ ID NO: 218 - protein, 13 aa, Homo sapiens
RNYEDHFPLLFSE

SEQ ID NO: 219 - protein, 31 aa, Homo sapiens
IVFIEGYCTPEEVIWEALNMMGLYDGMEHLI

SEQ ID NO: 220 - protein, 9 aa, Homo sapiens
EVIWEALNM

SEQ ID NO: 221 - protein, 15 aa, Homo sapiens
IVFIEGYCTPEEVIW

SEQ ID NO: 222 - protein, 15 aa, Homo sapiens
YCTPEEVIWEALNMM

SEQ ID NO: 223 - protein, 15 aa, Homo sapiens
VIWEALNMMGLYDGM

SEQ ID NO: 224 - protein, 31 aa, Homo sapiens
YGEPRKLLTQDWVQENYLEYRQVPGSDPARY

SEQ ID NO: 225 - protein, 9 aa, Homo sapiens
WVQENYLEY

SEQ ID NO: 226 - protein, 15 aa, Homo sapiens
YGEPRKLLTQDWVQE

SEQ ID NO: 227 - protein, 15 aa, Homo sapiens
LLTQDWVQENYLEYR

SEQ ID NO: 228 - protein, 15 aa, Homo sapiens
VQENYLEYRQVPGSD

SEQ ID NO: 229 - protein, 31 aa, Homo sapiens
RNYEDHFPLLFSEASECMLLVFGIDVKEVDP

SEQ ID NO: 230 - protein, 9 aa, Homo sapiens
SEASECMLL

SEQ ID NO: 231 - protein, 15 aa, Homo sapiens
RNYEDHFPLLFSEAS

SEQ ID NO: 232 - protein, 15 aa, Homo sapiens
FPLLFSEASECMLLV

SEQ ID NO: 233 - protein, 15 aa, Homo sapiens
EASECMLLVFGIDVK

SEQ ID NO: 234 - protein, 31 aa, Homo sapiens
ESPSTLQVLPDSESLPRSEIDEKVTDLVQFL

SEQ ID NO: 235 - protein, 9 aa, Homo sapiens
SESLPRSEI

SEQ ID NO: 236 - protein, 15 aa, Homo sapiens
ESPSTLQVLPDSESL

SEQ ID NO: 237 - protein, 15 aa, Homo sapiens
QVLPDSESLPRSEID

SEQ ID NO: 238 - protein, 15 aa, Homo sapiens
ESLPRSEIDEKVTDL

SEQ ID NO: 239 - protein, 31 aa, Homo sapiens
DQSDEGSSSQKEESPSTLQVLPDSESLPRSE

SEQ ID NO: 240 - protein, 10 aa, Homo sapiens
EESPSTLQVL

SEQ ID NO: 241 - protein, 15 aa, Homo sapiens
DQSDEGSSSQKEESP

SEQ ID NO: 242 - protein, 15 aa, Homo sapiens
SSSQKEESPSTLQVL

SEQ ID NO: 243 - protein, 15 aa, Homo sapiens
ESPSTLQVLPDSESL

SEQ ID NO: 244 - protein, 31 aa, Homo sapiens
PNPPQSAQIACSSPSVVASLPLDQSDEGSSS

SEQ ID NO: 245 - protein, 9 aa, Homo sapiens
SSPSVVASL

SEQ ID NO: 246 - protein, 15 aa, Homo sapiens
PNPPQSAQIACSSPS

SEQ ID NO: 247 - protein, 15 aa, Homo sapiens
AQIACSSPSVVASLP

SEQ ID NO: 248 - protein, 15 aa, Homo sapiens
SPSVVASLPLDQSDE

SEQ ID NO: 249 - protein, 31 aa, Homo sapiens
AVEEDASSSTSTSSSFPSSFPSSSSSSSSSC

SEQ ID NO: 250 - protein, 9 aa, Homo sapiens
TSSSFPSSF

SEQ ID NO: 251 - protein, 15 aa, Homo sapiens
AVEEDASSSTSTSSS

SEQ ID NO: 252 - protein, 15 aa, Homo sapiens
SSSTSTSSSFPSSFP

SEQ ID NO: 253 - protein, 15 aa, Homo sapiens
SSSFPSSFPSSSSSS

SEQ ID NO: 254 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 255 - protein, 31 aa, Homo sapiens
LISRALVVTHTYLEPGPVTAQVVLQAAIPLT

SEQ ID NO: 256 - protein, 9 aa, Homo sapiens
YLEPGPVTA

SEQ ID NO: 257 - protein, 15 aa, Homo sapiens
LISRALVVTHTYLEP

SEQ ID NO: 258 - protein, 15 aa, Homo sapiens
VVTHTYLEPGPVTAQ

SEQ ID NO: 259 - protein, 15 aa, Homo sapiens
LEPGPVTAQVVLQAA

SEQ ID NO: 260 - protein, 31 aa, Homo sapiens
AVVLASLIYRRRLMKQDFSVPQLPHSSSHWL

SEQ ID NO: 261 - protein, 9 aa, Homo sapiens
RLMKQDFSV

SEQ ID NO: 262 - protein, 15 aa, Homo sapiens
AVVLASLIYRRRLMK

SEQ ID NO: 263 - protein, 15 aa, Homo sapiens
LIYRRRLMKQDFSVP

SEQ ID NO: 264 - protein, 15 aa, Homo sapiens
LMKQDFSVPQLPHSS

SEQ ID NO: 265 - protein, 31 aa, Homo sapiens
TLIGANASFSIALNFPGSQKVLPDGQVIWVN

SEQ ID NO: 266 - protein, 10 aa, Homo sapiens
ALNFPGSQKV

SEQ ID NO: 267 - protein, 15 aa, Homo sapiens
TLIGANASFSIALNF

SEQ ID NO: 268 - protein, 15 aa, Homo sapiens
ASFSIALNFPGSQKV

SEQ ID NO: 269 - protein, 15 aa, Homo sapiens
LNFPGSQKVLPDGQV

SEQ ID NO: 270 - protein, 31 aa, Homo sapiens
VSGLSIGTGRAMLGTHTMEVTVYHRRGSRSY

SEQ ID NO: 271 - protein, 9 aa, Homo sapiens
MLGTHTMEV

SEQ ID NO: 272 - protein, 15 aa, Homo sapiens
VSGLSIGTGRAMLGT

SEQ ID NO: 273 - protein, 15 aa, Homo sapiens
GTGRAMLGTHTMEVT

SEQ ID NO: 274 - protein, 15 aa, Homo sapiens
LGTHTMEVTVYHRRG

SEQ ID NO: 275 - protein, 31 aa, Homo sapiens
WSQKRSFVYVWKTWGQYWQVLGGPVSGLSIG

SEQ ID NO: 276 - protein, 9 aa, Homo sapiens
KTWGQYWQV

SEQ ID NO: 277 - protein, 15 aa, Homo sapiens
WSQKRSFVYVWKTWG

SEQ ID NO: 278 - protein, 15 aa, Homo sapiens
FVYVWKTWGQYWQVL

SEQ ID NO: 279 - protein, 15 aa, Homo sapiens
TWGQYWQVLGGPVSG

SEQ ID NO: 280 - protein, 31 aa, Homo sapiens
IMSTESITGSLGPLLDGTATLRLVKRQVPLD

SEQ ID NO: 281 - protein, 10 aa, Homo sapiens
GPLLDGTATL

SEQ ID NO: 282 - protein, 15 aa, Homo sapiens
IMSTESITGSLGPLL

SEQ ID NO: 283 - protein, 15 aa, Homo sapiens
ITGSLGPLLDGTATL

SEQ ID NO: 284 - protein, 15 aa, Homo sapiens
PLLDGTATLRLVKRQ

SEQ ID NO: 285 - protein, 31 aa, Homo sapiens
DLVLKRCLLHLAVIGALLAVGATKVPRNQDW

SEQ ID NO: 286 - protein, 9 aa, Homo sapiens
AVIGALLAV

SEQ ID NO: 287 - protein, 15 aa, Homo sapiens
DLVLKRCLLHLAVIG

SEQ ID NO: 288 - protein, 15 aa, Homo sapiens
CLLHLAVIGALLAVG

SEQ ID NO: 289 - protein, 15 aa, Homo sapiens
VIGALLAVGATKVPR

SEQ ID NO: 290 - protein, 31 aa, Homo sapiens
YCLNVSLADTNSLAVVSTQLIMPGQEAGLGQ

SEQ ID NO: 291 - protein, 9 aa, Homo sapiens
SLAVVSTQL

SEQ ID NO: 292 - protein, 15 aa, Homo sapiens
YCLNVSLADTNSLAV

SEQ ID NO: 293 - protein, 15 aa, Homo sapiens
LADTNSLAVVSTQLI

SEQ ID NO: 294 - protein, 15 aa, Homo sapiens
LAVVSTQLIMPGQEA

SEQ ID NO: 295 - protein, 31 aa, Homo sapiens
VPLAHSSSAFTITDQVPFSVSVSQLRALDGG

SEQ ID NO: 296 - protein, 9 aa, Homo sapiens
ITDQVPFSV

SEQ ID NO: 297 - protein, 15 aa, Homo sapiens
VPLAHSSSAFTITDQ

SEQ ID NO: 298 - protein, 15 aa, Homo sapiens
SSAFTITDQVPFSVS

SEQ ID NO: 299 - protein, 15 aa, Homo sapiens
TDQVPFSVSVSQLRA

SEQ ID NO: 300 - protein, 31 aa, Homo sapiens
SRQLRTKAWNRQLYPEWTEAQRLDCWRGGQV

SEQ ID NO: 301 - protein, 9 aa, Homo sapiens
QLYPEWTEA

SEQ ID NO: 302 - protein, 15 aa, Homo sapiens
SRQLRTKAWNRQLYP

SEQ ID NO: 303 - protein, 15 aa, Homo sapiens
KAWNRQLYPEWTEAQ

SEQ ID NO: 304 - protein, 15 aa, Homo sapiens
LYPEWTEAQRLDCWR

SEQ ID NO: 305 - protein, 31 aa, Homo sapiens
VPFSVSVSQLRALDGGNKHFLRNQPLTFALQ

SEQ ID NO: 306 - protein, 10 aa, Homo sapiens
ALDGGNKHFL

SEQ ID NO: 307 - protein, 15 aa, Homo sapiens
VPFSVSVSQLRALDG

SEQ ID NO: 308 - protein, 15 aa, Homo sapiens
VSQLRALDGGNKHFL

SEQ ID NO: 309 - protein, 15 aa, Homo sapiens
LDGGNKHFLRNQPLT

SEQ ID NO: 310 - protein, 31 aa, Homo sapiens
STQLIMPGQEAGLGQVPLIVGILLVLMAVVL

SEQ ID NO: 311 - protein, 9 aa, Homo sapiens
GLGQVPLIV

SEQ ID NO: 312 - protein, 15 aa, Homo sapiens
STQLIMPGQEAGLGQ

SEQ ID NO: 313 - protein, 15 aa, Homo sapiens
PGQEAGLGQVPLIVG

SEQ ID NO: 314 - protein, 15 aa, Homo sapiens
LGQVPLIVGILLVLM

SEQ ID NO: 315 - protein, 31 aa, Homo sapiens
TGMTPEKVPVSEVMGTTLAEMSTPEATGMTP

SEQ ID NO: 316 - protein, 10 aa, Homo sapiens
EVMGTTLAEM

SEQ ID NO: 317 - protein, 15 aa, Homo sapiens
TGMTPEKVPVSEVMG

SEQ ID NO: 318 - protein, 15 aa, Homo sapiens
KVPVSEVMGTTLAEM

SEQ ID NO: 319 - protein, 15 aa, Homo sapiens
VMGTTLAEMSTPEAT

SEQ ID NO: 320 - protein, 31 aa, Homo sapiens
SGTTSVQVPTTEVISTAPVQMPTAESTGMTP

SEQ ID NO: 321 - protein, 10 aa, Homo sapiens
EVISTAPVQM

SEQ ID NO: 322 - protein, 15 aa, Homo sapiens
SGTTSVQVPTTEVIS

SEQ ID NO: 323 - protein, 15 aa, Homo sapiens
QVPTTEVISTAPVQM

SEQ ID NO: 324 - protein, 15 aa, Homo sapiens
VISTAPVQMPTAEST

SEQ ID NO: 325 - protein, 31 aa, Homo sapiens
IWVNNTIINGSQVWGGQPVYPQETDDACIFP

SEQ ID NO: 326 - protein, 9 aa, Homo sapiens
QVWGGQPVY

SEQ ID NO: 327 - protein, 15 aa, Homo sapiens
IWVNNTIINGSQVWG

SEQ ID NO: 328 - protein, 15 aa, Homo sapiens
IINGSQVWGGQPVYP

SEQ ID NO: 329 - protein, 15 aa, Homo sapiens
VWGGQPVYPQETDDA

SEQ ID NO: 330 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 331 - protein, 31 aa, Homo sapiens
AFLSVKKQFEELTLGEFLKLDRERAKNKIAK

SEQ ID NO: 332 - protein, 9 aa, Homo sapiens
LTLGEFLKL

SEQ ID NO: 333 - protein, 15 aa, Homo sapiens
AFLSVKKQFEELTLG

SEQ ID NO: 334 - protein, 15 aa, Homo sapiens
KQFEELTLGEFLKLD

SEQ ID NO: 335 - protein, 15 aa, Homo sapiens
TLGEFLKLDRERAKN

SEQ ID NO: 336 - protein, 30 aa, Homo sapiens
MGAPTLPPAWQPFLKDHRISTFKNWPFLEG

SEQ ID NO: 337 - protein, 10 aa, Homo sapiens
TLPPAWQPFL

SEQ ID NO: 338 - protein, 15 aa, Homo sapiens
MGAPTLPPAWQPFLK

SEQ ID NO: 339 - protein, 15 aa, Homo sapiens
PPAWQPFLKDHRIST

SEQ ID NO: 340 - protein, 15 aa, Homo sapiens
FLKDHRISTFKNWPF

SEQ ID NO: 341 - protein, 31 aa, Homo sapiens
GAPTLPPAWQPFLKDHRISTFKNWPFLEGCA

SEQ ID NO: 342 - protein, 9 aa, Homo sapiens
FLKDHRIST

SEQ ID NO: 343 - protein, 15 aa, Homo sapiens
GAPTLPPAWQPFLKD

SEQ ID NO: 344 - protein, 15 aa, Homo sapiens
PAWQPFLKDHRISTF

SEQ ID NO: 345 - protein, 15 aa, Homo sapiens
LKDHRISTFKNWPFL

SEQ ID NO: 346 - protein, 31 aa, Homo sapiens
AWQPFLKDHRISTFKNWPFLEGCACTPERMA

SEQ ID NO: 347 - protein, 9 aa, Homo sapiens
STFKNWPFL

SEQ ID NO: 348 - protein, 15 aa, Homo sapiens
AWQPFLKDHRISTFK

SEQ ID NO: 349 - protein, 15 aa, Homo sapiens
KDHRISTFKNWPFLE

SEQ ID NO: 350 - protein, 15 aa, Homo sapiens
TFKNWPFLEGCACTP

SEQ ID NO: 351 - protein, 31 aa, Homo sapiens
FLEGCACTPERMAEAGFIHCPTENEPDLAQC

SEQ ID NO: 352 - protein, 10 aa, Homo sapiens
RMAEAGFIHC

SEQ ID NO: 353 - protein, 15 aa, Homo sapiens
FLEGCACTPERMAEA

SEQ ID NO: 354 - protein, 15 aa, Homo sapiens
CTPERMAEAGFIHCP

SEQ ID NO: 355 - protein, 15 aa, Homo sapiens
AEAGFIHCPTENEPD

SEQ ID NO: 356 - DNA, 2998 bp, synthetic construct
  source 1..2998: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 357 - DNA, 695 bp, synthetic construct
  source 1..695: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 358 - DNA, 2187 bp, synthetic construct
  source 1..2187: mol_type = other DNA
ccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 359 - DNA, 116 bp, synthetic construct
  source 1..116: mol_type = other DNA
gctagcgtttaaacttaagcttggtaccgagctcggatccactagtccagtgtggtggaattctgcagatatccagcacagtggcggccgctcgagtctagagggcccgtttaaac

SEQ ID NO: 360 - DNA, 6259 bp, synthetic construct
  source 1..6259: mol_type = other DNA
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgataattcggcttatttaaattccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctcggcgcgcctctcgagttactaactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggctcgagtgggcccaagtttaaacgctcctccgacgtccccaggcagaatggcggttccctaaacgagcattgcttatatagacctcccattaggcacgcctaccgcccatttacgtcaatggaacgcccatttgcgtcattgcccctccccattgacgtcaatggggatgtacttggcagccatcgcgggccatttaccgccattgacgtcaatgggagtactgccaatgtaccctggcgtacttccaatagtaatgtacttgccaagttactattaatagatattgatgtactgccaagtgggccatttaccgtcattgacgtcaatagggggcgtgagaacggatatgaatgggcaatgagccatcccattgacgtcaatggtgggtggtcctattgacgtcaatgggcattgagccaggcgggccatttaccgtaattgacgtcaatgggggaggcgccatatacgtcaataggaccgcccatatgacgtcaataggaaagaccatgctaagccgaattatcgcggctatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaatagacgcgtaaaaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 361 - DNA, 2999 bp, synthetic construct
  source 1..2999: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 362 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 363 - protein, 781 aa, synthetic construct; identical to SEQ ID NO: 15

SEQ ID NO: 364 - protein, 781 aa, synthetic construct
MATQADLMELDMAMEPDRKAAVSHWQQQSYLDSGIHSGATTTAPPLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 365 - DNA, 26 bp, synthetic construct
  source 1..26: mol_type = other DNA
ccgctcgagacagggttctggatatt

SEQ ID NO: 366 - DNA, 36 bp, synthetic construct
  source 1..36: mol_type = other DNA
ttttccttttgcggccgcgaacacgtttttcaggtc

SEQ ID NO: 367 - protein, 15 aa, synthetic construct
CAVGGSGGGADGLTF

SEQ ID NO: 368 - protein, 275 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGGSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 369 - protein, 11 aa, synthetic construct
CAVGINARLMF

SEQ ID NO: 370 - protein, 272 aa, Homo sapiens
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVGINARLMFGDGTQLVVKPNIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 371 - protein, 14 aa, synthetic construct
CAGPMKTSYDKVIF

SEQ ID NO: 372 - protein, 271 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGPMKTSYDKVIFGPGTSLSVIPNIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 373 - protein, 16 aa, synthetic construct
CAVRDLGAGGGNKLTF

SEQ ID NO: 374 - protein, 276 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDLGAGGGNKLTFGTGTQLKVELNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 375 - protein, 12 aa, synthetic construct
CAASKPGNQFYF

SEQ ID NO: 376 - protein, 271 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASKPGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 377 - protein, 17 aa, synthetic construct
CVVNRLHSYNYGQNFVF

SEQ ID NO: 378 - protein, 277 aa, Homo sapiens
MMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNRLHSYNYGQNFVFGPGTRLSVLPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 379 - protein, 14 aa, synthetic construct
CAVRGTDSWGKLQF

SEQ ID NO: 380 - protein, 273 aa, Homo sapiens
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVRGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 381 - protein, 12 aa, synthetic construct
CASSAVGNTIYF

SEQ ID NO: 382 - protein, 307 aa, Homo sapiens
MTIRLLCYVGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSAVGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 383 - protein, 15 aa, synthetic construct
CVVSEGDSGGFKTIF

SEQ ID NO: 384 - protein, 313 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPLAGGISDTQYFGPGTRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 385 - protein, 16 aa, synthetic construct
CASSPLAGGISDTQYF

SEQ ID NO: 386 - protein, 313 aa, Homo sapiens; identical to SEQ ID NO: 384

SEQ ID NO: 387 - protein, 12 aa, synthetic construct
CASSSANYGYTF

SEQ ID NO: 388 - protein, 308 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSANYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 389 - protein, 13 aa, synthetic construct
CASSTLVRETQYF

SEQ ID NO: 390 - protein, 310 aa, Homo sapiens
MGPGLLCWALLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSTLVRETQYFGPGTRLLVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 391 - protein, 15 aa, synthetic construct
CSAREYQGVKSPLHF

SEQ ID NO: 392 - protein, 327 aa, Homo sapiens
MEAVVTTLPREGGVRPSRKMLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSAREYQGVKSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 393 - protein, 13 aa, synthetic construct
CASSFKMNTEAFF

SEQ ID NO: 394 - protein, 308 aa, Homo sapiens
MGPRLLFWALLCLLGIGPVEAGVTQSPTHLIKTRGQQVTLRCSPISGHTSVYWYQQALGLGLQFLLWYDEGEERNRGNFPPRFSGRQFPNYSSELNVNALELEDSALYLCASSFKMNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 395-396 - skipped (2 entries)

SEQ ID NO: 397 - protein, 22 aa, synthetic construct
MLWCGTCFVTNNMKGSEVSLEK

SEQ ID NO: 398 - protein, 33 aa, synthetic construct
SGMPIAPRSSVDRPRDCYRRMFQQIHRKMPDLQ

SEQ ID NO: 399 - protein, 33 aa, synthetic construct
EILQKILDDLFLHTLCAYIYELATAFTEFYDSC

SEQ ID NO: 400 - protein, 33 aa, synthetic construct
DPWIGKLLYLEDFFVVSDYRGFGIGSEILKNLS

SEQ ID NO: 401 - protein, 33 aa, synthetic construct
MQTDIDTEVPSEPVMMKVMMTALKFKRSMRQLS

SEQ ID NO: 402 - protein, 33 aa, synthetic construct
QGFYCGVGLFTRFKLLGALLLLALALFLGFLQL

SEQ ID NO: 403 - protein, 33 aa, synthetic construct
TKVDDCVTALAALVAALHVFRIECPRRLSQTFN

SEQ ID NO: 404 - protein, 33 aa, synthetic construct
LILHVECRARRRQRSAKLNHDILAMVSVFLVSS

SEQ ID NO: 405 - protein, 33 aa, synthetic construct
AQVVLSFQAYPTARCVLLEVHVPAALVQFGQSV

SEQ ID NO: 406 - protein, 33 aa, synthetic construct
LGHIFDDGPRPTGKRCCINSAALSFTPADSSGT

SEQ ID NO: 407 - protein, 33 aa, synthetic construct
AEEIVMYMNNMSSPLTSRTPRIDLQRACDDKLN

SEQ ID NO: 408 - protein, 33 aa, synthetic construct
FQMLRRDNEKIEASLRLLEINMKEIKETLRQYL

SEQ ID NO: 409 - protein, 33 aa, synthetic construct
LAVKDWPWWQLLGFLQRLLSATIGTEQLRAKEE

SEQ ID NO: 410 - protein, 33 aa, synthetic construct
MLGGCEFSRQRLPAFGLNPKRLHFRQLGRQRRK

SEQ ID NO: 411 - protein, 33 aa, synthetic construct
FQPNSGAQGEYAGLATVRAYLNQKGEGHRTVCL

SEQ ID NO: 412 - protein, 33 aa, synthetic construct
IMEVHKEKGYGCCICNRRFALKATYHAHMVIHR

SEQ ID NO: 413 - protein, 33 aa, synthetic construct
TYSMQMQMIPSNSTRLSVAYQGNQGLNQSFSEQ

SEQ ID NO: 414 - protein, 33 aa, synthetic construct
SWGEGCAQPNRPGIYTHVTYYLDWIHHYVPKKP

SEQ ID NO: 415 - protein, 33 aa, synthetic construct
GRIVLAGREMTRLAGFAARITELMQVLKDLNHG

SEQ ID NO: 416 - protein, 33 aa, synthetic construct
RESRKDLVLIDSLFIMNQFKAAERMNIGKPNAK

SEQ ID NO: 417 - protein, 33 aa, synthetic construct
KKKLKILESKEKNTQGPRMPQTAKKVQRTVLEK

SEQ ID NO: 418 - protein, 33 aa, synthetic construct
DRVRELFSIDPKTGLIRVKSNLDYEENGMLEID

SEQ ID NO: 419 - protein, 33 aa, synthetic construct
LGSQDALPIATAFTEYIHAYFRGHSPSCLARVT

SEQ ID NO: 420 - protein, 33 aa, synthetic construct
RREGPGAAPAFAGPGGGGSGTLEAEGRAGARAG

SEQ ID NO: 421 - protein, 33 aa, synthetic construct
FLRNSTIPHLVRKRDVYVVEFHRQSPAKILNCT

SEQ ID NO: 422 - protein, 33 aa, synthetic construct
QWMNDTDCFLGHHDLKMCIIGLSILLELQNRPP

SEQ ID NO: 423 - protein, 33 aa, synthetic construct
KPKSYEAYYARARAKRSSREFAAALEDLNEAIK

SEQ ID NO: 424 - protein, 33 aa, synthetic construct
ISIWDRSTGIKFNSIQQDLGCGASLGVISDNLL

SEQ ID NO: 425 - protein, 33 aa, synthetic construct
LRAKVLTLDGMNPRVRGVEYAVRGPIVQRALEL

SEQ ID NO: 426 - protein, 33 aa, synthetic construct
KKQEQEVKTEAEKVILQKVKETLSMEKIPPMVT

SEQ ID NO: 427 - protein, 33 aa, synthetic construct
KWKEIQVGDVIRLKKNHFVPADILLLSSSEPNS

SEQ ID NO: 428 - protein, 33 aa, synthetic construct
ADPKVAFGSLAANGPTALVDKVSNPKTNGDSQQ

SEQ ID NO: 429 - protein, 33 aa, synthetic construct
LLSPINQNTLQRSSSVQSMVSSATYGGSDDYIG

SEQ ID NO: 430 - protein, 33 aa, synthetic construct
QIHERPQHGKKLCDCKECASFSSLENLQRHMAA

SEQ ID NO: 431 - protein, 33 aa, synthetic construct
KRNLSDIDQSFNKVAECVLMRLQEKLKGVEEGT

SEQ ID NO: 432 - protein, 33 aa, synthetic construct
IWQLLQAGQPPDLERILTLLELEKAERHGSRAR

SEQ ID NO: 433 - protein, 33 aa, synthetic construct
FGFNPLEFENFSWRGYVQMAPVQPRIVARLNLT

SEQ ID NO: 434 - protein, 33 aa, synthetic construct
IYGGYPYQAANGCAYNANQQPYPASAALGADGE

SEQ ID NO: 435 - protein, 33 aa, synthetic construct
THFKLPKYRRLLETFFNFVMLVDYIFQELIRQL

SEQ ID NO: 436 - protein, 33 aa, synthetic construct
TVYQQGVGYVPITGMPTVCSPGMVPVALPPAAV

SEQ ID NO: 437 - protein, 33 aa, synthetic construct
LKKELAGALAYIMDNKLSQIEGVVPFGANNAFT

SEQ ID NO: 438 - protein, 33 aa, synthetic construct
QDSAPQPALPQVQIPFPRRSHRLSKEGILPPSA

SEQ ID NO: 439 - protein, 33 aa, synthetic construct
AFTDSLITRNFSNYSSCHYRSCLLPQLHPQPDS

SEQ ID NO: 440 - protein, 33 aa, synthetic construct
AYVFHDEFVASMIKIPSDTFSIIPDFDIYYVYG

SEQ ID NO: 441 - protein, 33 aa, synthetic construct
EAVYTMSPTAGPNHHLLSPDVSQGLVLAVSSDG

SEQ ID NO: 442 - protein, 33 aa, synthetic construct
CMQARLVGEALLESNTIIDVYCSPSLRCVQTAH

SEQ ID NO: 443 - protein, 33 aa, synthetic construct
ISGDRLTHQIMDLNLDNMYYFRIQARNSKGVGP

SEQ ID NO: 444 - protein, 33 aa, synthetic construct
LHSGEKPYKCEECVKAFSQFGHLTTHRIIHTGE

SEQ ID NO: 445 - protein, 33 aa, synthetic construct
GLVSAYGPGLEGSTTGVSSEFIVNTLNAGSGAL

SEQ ID NO: 446 - protein, 33 aa, synthetic construct
EEYEDIRQDHYESLKEGRYLPLSQARKSGFQMD

SEQ ID NO: 447 - protein, 33 aa, synthetic construct
LDELTHEFLQILEKTPNRLKGIWNWRACEAAKK

SEQ ID NO: 448 - protein, 33 aa, synthetic construct
MFLVLLPYLKVKLEKLVSSLSEEDEYSIHPPSS

SEQ ID NO: 449 - protein, 33 aa, synthetic construct
CRVTATKDSTTSDVIKNAIASLRLDGTKCYVLV

SEQ ID NO: 450 - protein, 33 aa, synthetic construct
DLLAVLRGLSEDIQKAEDASKFELCQLLPLFLP

SEQ ID NO: 451 - protein, 33 aa, synthetic construct
IKFSEADYFGNVLQARKYLAQSDFFWLRKAVPK

SEQ ID NO: 452 - protein, 33 aa, synthetic construct
ARLVVLAIANTMDLPEQIMMNRVSSRLGLTRMC

SEQ ID NO: 453 - protein, 33 aa, synthetic construct
ASDCALSLRPEEPASTVCIMQGPGPTKAPSGRP

SEQ ID NO: 454 - protein, 33 aa, synthetic construct
RKKVVHVLEGESGSVVVQTVPGQVVSHRGGTIV

SEQ ID NO: 455 - protein, 33 aa, synthetic construct
SMLRNERQFKEEMLAEQLKQAEELRQYKVLVHS

SEQ ID NO: 456 - protein, 33 aa, synthetic construct
ATSAVSMPLNSPTMEASGWSQEEMTAGFCYGTW

SEQ ID NO: 457 - protein, 33 aa, synthetic construct
TLCEEAFFRFAVPTKFMPNWLSVLVDNLPGTKV

SEQ ID NO: 458 - protein, 33 aa, synthetic construct
KPPDFPVSCQDEPFRDTAVWPPPVPAEHRAPPQ

SEQ ID NO: 459 - protein, 33 aa, synthetic construct
VEVFDLNAGCSAAVIAEAHSWPVHQICQNKGSS

SEQ ID NO: 460 - protein, 33 aa, synthetic construct
FTNSKLLMMPSVNKSSSRPGGPYLQPSHVNLLS

SEQ ID NO: 461 - protein, 33 aa, synthetic construct
TAVPDLYFENAMRFFNLSWRVTADQLRKAPNRD

SEQ ID NO: 462 - protein, 33 aa, synthetic construct
KDQPSAYSKVQWLTPVILALWEAEAGGSLEGFL

SEQ ID NO: 463 - protein, 33 aa, synthetic construct
KKTRIISHVWENNNPFRIVLVKGNKLNTEETVK

SEQ ID NO: 464 - protein, 33 aa, synthetic construct
PAATETGKYIASTQRPNGTWRKQRRVKEGYVPQ

SEQ ID NO: 465 - protein, 33 aa, synthetic construct
IVLHDRIVSVISSPSLMSETEWVGYPFRLFDFT

SEQ ID NO: 466 - protein, 26 aa, synthetic construct
MAESLRSSRRSLYKLVGSPPWKEAFR

SEQ ID NO: 467 - protein, 33 aa, synthetic construct
PIAEKLTGSCSVTQAGVQWCSHSSLQPQLPWLN

SEQ ID NO: 468 - protein, 33 aa, synthetic construct
NSFTFAPNGVPFVTEALYGPKYTFFNNVLMFSP

SEQ ID NO: 469 - protein, 28 aa, synthetic construct
MAAAAQSRVVRLLSMSRSAITAIATSVC

SEQ ID NO: 470 - protein, 33 aa, synthetic construct
AMKPLPVVSGLRSVQVSLMLNMLQESVAPALSC

SEQ ID NO: 471 - protein, 33 aa, synthetic construct
YEAGEELTGPGAVAATRLYGLEPAHPLLYSRLA

SEQ ID NO: 472 - protein, 33 aa, synthetic construct
HCSLATLIGLCLRVNFSDVYHLNISWKSTFLKE

SEQ ID NO: 473 - protein, 33 aa, synthetic construct
LIQYCHSKGITVTAYSPLGSLDRPWAKPEDPSL

SEQ ID NO: 474 - protein, 33 aa, synthetic construct
GNTTRRCLLEGYNTKNTRQDNSILKVLISMQLM

SEQ ID NO: 475 - protein, 33 aa, synthetic construct
KRSRGQVLFDKVCEHLSLLEKDYFGLTYRDAEN

SEQ ID NO: 476 - protein, 33 aa, synthetic construct
PSAMYCDELKLKTVPMVPPGIKYLYLRNNQIDH

SEQ ID NO: 477 - protein, 33 aa, synthetic construct
PSAATNWTAGLLVDSSKMIFKFDGRQGAKVPDG

SEQ ID NO: 478 - protein, 33 aa, synthetic construct
HPLLQDALRISQNFLSSISEEITPRRQSMTVKK

SEQ ID NO: 479 - protein, 33 aa, synthetic construct
KAKVIQPDGVLLLLPRMECSGAISAHHNLPLPG

SEQ ID NO: 480 - protein, 33 aa, synthetic construct
WDQPVRYHRGDIFENVNYVQFGEDSSTSSMMSV

SEQ ID NO: 481 - protein, 33 aa, synthetic construct
GGYDKDGRGPMTGSSGVDRGGFKNFGGHRDYGP

SEQ ID NO: 482 - protein, 33 aa, synthetic construct
HQHGLLASGGGTTDRCIRFWNTLTGQPLQCIDT

SEQ ID NO: 483 - protein, 33 aa, synthetic construct
GNYEVSIKFNDEHMPESPYLVPVIAPSDDARRL

SEQ ID NO: 484 - protein, 33 aa, synthetic construct
MGLADTKIPRSGTPVRIRSNRSACVLRIPSQNQ

SEQ ID NO: 485 - protein, 33 aa, synthetic construct
DFRNRDVSDLDFRDKDRTQVDFRGRGSGTTDLD

SEQ ID NO: 486 - protein, 33 aa, synthetic construct
KDMLYKMLSENFMSLQEILKTPDHAPSRTFYLY

SEQ ID NO: 487 - protein, 33 aa, synthetic construct
NLPPSGPPAVVNIALPLPPGIAPPPPPGFGPHM

SEQ ID NO: 488 - protein, 33 aa, synthetic construct
PPAVSPREALMEAMHTIPVPPTTVRTDSLGKDA

SEQ ID NO: 489 - protein, 33 aa, synthetic construct
ANFYKDSGITYLGIKAKDTQEFNLSAYFERAAD

SEQ ID NO: 490 - protein, 33 aa, synthetic construct
LRTYLLTRDDHGLFGLAVKSRGDGTKFPELVIQ

SEQ ID NO: 491 - protein, 33 aa, synthetic construct
RKLPDVKKDDMSTRRTSHGEPKSAVPFNQYLPN

SEQ ID NO: 492 - protein, 21 aa, synthetic construct
LGRPGTVTHVCNPSTLGGRGG

SEQ ID NO: 493 - protein, 33 aa, synthetic construct
GGGPGEPRGQQPANRGALPARALDPHPSPLITL

SEQ ID NO: 494 - protein, 33 aa, synthetic construct
RERNKLAAAKCRNRRRELTDQLQAETDQLEEEK

SEQ ID NO: 495 - protein, 275 aa, Homo sapiens; identical to SEQ ID NO: 368

SEQ ID NO: 496 - protein, 272 aa, Homo sapiens; identical to SEQ ID NO: 370

SEQ ID NO: 497 - protein, 271 aa, Homo sapiens; identical to SEQ ID NO: 372

SEQ ID NO: 498 - protein, 276 aa, Homo sapiens; identical to SEQ ID NO: 374

SEQ ID NO: 499 - protein, 271 aa, Homo sapiens; identical to SEQ ID NO: 376

SEQ ID NO: 500 - protein, 277 aa, Homo sapiens; identical to SEQ ID NO: 378

SEQ ID NO: 501 - protein, 273 aa, Homo sapiens; identical to SEQ ID NO: 380

SEQ ID NO: 502 - protein, 307 aa, Homo sapiens; identical to SEQ ID NO: 382

SEQ ID NO: 503 - protein, 313 aa, Homo sapiens; identical to SEQ ID NO: 384

SEQ ID NO: 504 - protein, 313 aa, Homo sapiens; identical to SEQ ID NO: 384

SEQ ID NO: 505 - protein, 308 aa, Homo sapiens; identical to SEQ ID NO: 388

SEQ ID NO: 506 - protein, 310 aa, Homo sapiens; identical to SEQ ID NO: 390

SEQ ID NO: 507 - protein, 327 aa, Homo sapiens; identical to SEQ ID NO: 392

SEQ ID NO: 508 - protein, 308 aa, Homo sapiens; identical to SEQ ID NO: 394

SEQ ID NO: 509 - protein, 14 aa, Homo sapiens
CAVRDKGSARQLTF

SEQ ID NO: 510 - protein, 14 aa, Homo sapiens
CSARDLYRNTEAFF

SEQ ID NO: 511 - protein, 14 aa, Homo sapiens
CAVSAGNNNARLMF

SEQ ID NO: 512 - protein, 14 aa, Homo sapiens
CSARDLYRNTEAFF

SEQ ID NO: 513 - protein, 12 aa, Homo sapiens
CALDSSASKIIF

SEQ ID NO: 514 - protein, 12 aa, Homo sapiens
CASSRQGQEQYF

SEQ ID NO: 515 - protein, 13 aa, Homo sapiens
CAEKGAYGQNFVF

SEQ ID NO: 516 - protein, 18 aa, Homo sapiens
CASSSDALAGGIHRKLFF

SEQ ID NO: 517 - protein, 13 aa, Homo sapiens
CAFNAGNNRKLIW

SEQ ID NO: 518 - protein, 14 aa, Homo sapiens
CASSSFRYNQPQHF

SEQ ID NO: 519 - protein, 11 aa, Homo sapiens
CAGVFEARLMF

SEQ ID NO: 520 - protein, 13 aa, Homo sapiens
CASSLLQGTTAFF

SEQ ID NO: 521 - protein, 12 aa, Homo sapiens
CAVEDQGGKLIF

SEQ ID NO: 522 - protein, 14 aa, Homo sapiens
CASSQDFAGSEQYF

SEQ ID NO: 523 - protein, 16 aa, Homo sapiens
CAVNDPPVDSSYKLIF

SEQ ID NO: 524 - protein, 11 aa, Homo sapiens
CASSFADTQYF

SEQ ID NO: 525 - protein, 16 aa, Homo sapiens
CAVNDPPVDSSYKLIF

SEQ ID NO: 526 - protein, 11 aa, Homo sapiens
CASSFADTQYF

SEQ ID NO: 527 - protein, 12 aa, Homo sapiens
CALAGGSYIPTF

SEQ ID NO: 528 - protein, 11 aa, Homo sapiens
CASADSYEQYF

SEQ ID NO: 529 - protein, 12 aa, Homo sapiens
CAFGDSWGKFQF

SEQ ID NO: 530 - protein, 15 aa, Homo sapiens
CASSIDQTGITEAFF

SEQ ID NO: 531 - protein, 14 aa, Homo sapiens
CAAIRSGSARQLTF

SEQ ID NO: 532 - protein, 16 aa, Homo sapiens
CASSQDLGVAYNEQFF

SEQ ID NO: 533 - protein, 12 aa, Homo sapiens
CAYSGSARQLTF

SEQ ID NO: 534 - protein, 14 aa, Homo sapiens
CASSFGGNTETQYF

SEQ ID NO: 535 - protein, 14 aa, Homo sapiens
CGTEAIGGTYKYIF

SEQ ID NO: 536 - protein, 14 aa, Homo sapiens
CASSFGGNTETQYF

SEQ ID NO: 537 - protein, 13 aa, Homo sapiens
CIDSGGSNYKLTF

SEQ ID NO: 538 - protein, 12 aa, Homo sapiens
CASSPAGPAQYF

SEQ ID NO: 539 - protein, 15 aa, Homo sapiens
CAGRLSSGGSYIPTF

SEQ ID NO: 540 - protein, 15 aa, Homo sapiens
CASSAIGTPSGEQFF

SEQ ID NO: 541 - protein, 14 aa, Homo sapiens
CAVGSDGAGNMLTF

SEQ ID NO: 542 - protein, 15 aa, Homo sapiens
CASTRDESYNSPLHF

SEQ ID NO: 543 - protein, 19 aa, Homo sapiens
CAVGGRSNAGGTSYGKLTF

SEQ ID NO: 544 - protein, 15 aa, Homo sapiens
CASTRDESYNSPLHF

SEQ ID NO: 545 - protein, 15 aa, Homo sapiens
CAASVGGAGANNLFF

SEQ ID NO: 546 - protein, 13 aa, Homo sapiens
CASSVDIYNEQFF

SEQ ID NO: 547 - protein, 14 aa, Homo sapiens
CAMREPYNFNKFYF

SEQ ID NO: 548 - protein, 14 aa, Homo sapiens
CATHEGWDTGELFF

SEQ ID NO: 549 - protein, 13 aa, Homo sapiens
CAAGGTSYGKLTF

SEQ ID NO: 550 - protein, 14 aa, Homo sapiens
CATTSGSPAGELFF

SEQ ID NO: 551 - protein, 14 aa, Homo sapiens
CAAETRNNNARLMF

SEQ ID NO: 552 - protein, 14 aa, Homo sapiens
CAGRLAGASGELFF

SEQ ID NO: 553 - protein, 13 aa, Homo sapiens
CAFLLSDGQKLLF

SEQ ID NO: 554 - protein, 14 aa, Homo sapiens
CAGRLAGASGELFF

SEQ ID NO: 555 - protein, 14 aa, Homo sapiens
CAAAGYSSASKIIF

SEQ ID NO: 556 - protein, 15 aa, Homo sapiens
CASSLGPPGQTEAFF

SEQ ID NO: 557 - protein, 15 aa, Homo sapiens
CAMSAPWDTGNQFYF

SEQ ID NO: 558 - protein, 15 aa, Homo sapiens
CASSHGQGKDGELFF

SEQ ID NO: 559 - protein, 12 aa, Homo sapiens
CAAEGNTDKLIF

SEQ ID NO: 560 - protein, 16 aa, Homo sapiens
CASSQDPAGRWTEAFF

SEQ ID NO: 561 - protein, 11 aa, Homo sapiens
CTTSGTYKYIF

SEQ ID NO: 562 - protein, 14 aa, Homo sapiens
CASSPRQGPGELFF

SEQ ID NO: 563 - protein, 15 aa, Homo sapiens
CAVEEISGGYQKVTF

SEQ ID NO: 564 - protein, 15 aa, Homo sapiens
CASSENRGRADTQYF

SEQ ID NO: 565 - protein, 15 aa, Homo sapiens
CAVEEISGGYQKVTF

SEQ ID NO: 566 - protein, 15 aa, Homo sapiens
CASSENRGRADTQYF

SEQ ID NO: 567 - protein, 15 aa, Homo sapiens
CAVEEISGGYQKVTF

SEQ ID NO: 568 - protein, 15 aa, Homo sapiens
CASSENRGRADTQYF

SEQ ID NO: 569 - protein, 12 aa, Homo sapiens
CAESIGDYKLSF

SEQ ID NO: 570 - protein, 17 aa, Homo sapiens
CASSQELVGPNTGELFF

SEQ ID NO: 571 - protein, 14 aa, Homo sapiens
CAATSMDSNYQLIW

SEQ ID NO: 572 - protein, 13 aa, Homo sapiens
CASSIEQGVAQYF

SEQ ID NO: 573 - protein, 15 aa, Homo sapiens
CAFTYSGGGADGLTF

SEQ ID NO: 574 - protein, 16 aa, Homo sapiens
CATSREGATFYNEQFF

SEQ ID NO: 575 - protein, 20 aa, Homo sapiens
CIVRVAGFLYSGGGADGLTF

SEQ ID NO: 576 - protein, 16 aa, Homo sapiens
CASSVGEFTGTDTQYF

SEQ ID NO: 577 - protein, 12 aa, Homo sapiens
CVVSRGDYKLSF

SEQ ID NO: 578 - protein, 13 aa, Homo sapiens
CAINPDSNQPQHF

SEQ ID NO: 579 - protein, 10 aa, Homo sapiens
CAVGNARLMF

SEQ ID NO: 580 - protein, 13 aa, Homo sapiens
CAINPDSNQPQHF

SEQ ID NO: 581 - protein, 14 aa, Homo sapiens
CAVSPMTGFQKLVF

SEQ ID NO: 582 - protein, 17 aa, Homo sapiens
CASSELEGDGSGNTIYF

SEQ ID NO: 583 - protein, 13 aa, Homo sapiens
CAASGTSYGKLTF

SEQ ID NO: 584 - protein, 17 aa, Homo sapiens
CASSQGGSGTLFHEQFF

SEQ ID NO: 585 - protein, 13 aa, Homo sapiens
CAVPHGATNKLIF

SEQ ID NO: 586 - protein, 12 aa, Homo sapiens
CASKGVVYEQYF

SEQ ID NO: 587 - protein, 15 aa, Homo sapiens
CAEIPFSGGYNKLIF

SEQ ID NO: 588 - protein, 15 aa, Homo sapiens
CASSGRTGGGYEQYF

SEQ ID NO: 589 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 590 - DNA, 660 bp, Homo sapiens
  source 1..660: mol_type = genomic DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 591 - DNA, 658 bp, synthetic construct
  source 1..658: mol_type = other DNA
atgtgccccgctcggtccctgctgctggtcgctaccctggtcctgctggatcacctgtcactggctcgaaatctgcctgtcgctacccccgatccatgaatgttcccctgcctgcaccatacccagaacctgctgcgggccgtgtccaatatgctgcagaaagctagacagacactggagttttacccttgtacttctgacgaaatcgatcacgacgatattactaacgacaaaacctccacagtcgaagcctgcctgccactggagctgaccaagaacgaatcatgtctgaatagcagggagacttccttcatcaccaacgggtcttgcctggctagtcgcaagaccagcttcatgatggcactgtgcctgagctccatctacgaggatcgaagatgtatcaggtggaattcaaaaccatgaacgctaagctcctgatggaccctaaacgacagatctttctggatcagaatatgctggcagtgattgacgagctgatgcaggccctgaacttcaatagcgaaaccgtcccacacaaatcctcccttgaagaaccggatttttataaaactaaaatcaagctctgcatacttcttcatgctttcagaattcgggcagtgactattgatcgctcatgtcatacctgaacgcttcctaa

SEQ ID NO: 592 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 593 - DNA, 986 bp, Homo sapiens
  source 1..986: mol_type = genomic DNA
ccctgatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgc

SEQ ID NO: 594 - DNA, 985 bp, synthetic construct
  source 1..985: mol_type = other DNA
atgtgccatcagcagctggtcatctcttggtttagtctggtgtttctggcttctccactggtcgctatctgggaactgaaaaaggatgtgtacgtggtcgagctggactggtatccagatgcacccggagaaatggtggtcctgacctgcgacacacccgaggaagatggcatcacttggaccctggaccagagctccgaggtgctgggatctggcaagacactgactattcaggtcaaagaattcggggatgccggacagtacacatgtcacaagggcggggaggtgctgagtcactcactgctgctgctgcataagaaagaagacggcatctggtctactgacattctgaaggatcagaaagagcctaagaacaaaaccttcctgagatgcgaagctaagaattatagtgggaggtttacctgttggtggctgaccacaatctcaactgacctgacctttagcgtgaaatctagtagggggtcaagcgatccacagggagtgacctgcggagctgctacactgagcgccgagcgggtgagaggagacaacaaggagtacgaatatagcgtcgagtgccaggaagattcagcctgtcccgcagccgaggaatccctgcctatcgaagtgatggtggacgctgtgcacaagctgaaatacgaaaactacacatcctctttctttattcgcgacatcattaagccagatccccctaaaaacctgcagctgaagcccctgaaaaattcccgacaggtggaggtctctctgggaataccctgatacatggagcactccacattcttatttcagtctgactttctgcgtgcaggtccagggcaagagcaaaagggagaagaaagaccgcgtgttcaccgataagacatccgctactgtcatctgtcgaaaaaacgcaagcatttccgtgcgggcacaggataggtattattccagcagttggtctgagtgggcttccgtcccttgtagt

SEQ ID NO: 595 - DNA, 66 bp, synthetic construct
  source 1..66: mol_type = other DNA
ggaagcggagctactaacttcagcctgctgaagcaggctggagacgtggaggagaaccctggacct

SEQ ID NO: 596 - protein, 22 aa, synthetic construct
GSGATNFSLLKQAGDVEENPGP

SEQ ID NO: 597 - DNA, 63 bp, synthetic construct
  source 1..63: mol_type = other DNA
ggaagcggagagggcagaggaagtctgctaacatgcggtgacgtcgaggagaatcctggacct

SEQ ID NO: 598 - protein, 21 aa, synthetic construct
GSGEGRGSLLTCGDVEENPGP

SEQ ID NO: 599 - DNA, 69 bp, synthetic construct
  source 1..69: mol_type = other DNA
ggaagcggacagtgtactaattatgctctcttgaaattggctggagatgttgagagcaaccctggacct

SEQ ID NO: 600 - protein, 23 aa, synthetic construct
GSGQCTNYALLKLAGDVESNPGP

SEQ ID NO: 601 - DNA, 75 bp, synthetic construct
  source 1..75: mol_type = other DNA
ggaagcggagtgaaacagactttgaattttgaccttctcaagttggcgggagacgtggagtccaaccctggacct

SEQ ID NO: 602 - protein, 25 aa, synthetic construct
GSGVKQTLNFDLLKLAGDVESNPGP

(19) United States
(12) Patent Application Publication
(10) Pub. No.: US 2025/0302955 A1
(43) Pub. Date: Oct. 2, 2025

(54) INDIVIDUALIZED CANCER EPITOPES AND METHODS OF USING THE SAME

(71) Applicant: GENEOS THERAPEUTICS, INC., Philadelphia, PA (US)

(72) Inventors: Alfredo PERALES-PUCHALT, Valencia (ES); Niranjan SARDESAI, Blue Bell, PA (US)

(73) Assignee: GENEOS THERAPEUTICS, INC., Philadelphia, PA (US)

(21) Appl. No.: 18/864,854

(22) PCT Filed: May 10, 2023
(86) PCT No.: PCT/US2023/066854

§ 371 Date: Nov. 11, 2024

Related U.S. Application Data
(60) Provisional application No. 63/340,058, filed on May 10, 2022.
(60) Provisional application No. 63/342,608, filed on May 16, 2022.

(51) Int. Cl. A61K 40/11; A61K 31/711; A61K 35/17; A61K 40/32; A61K 40/42; A61K 45/06; A61P 35/04; C12N 5/0783; C12Q 1/6869

(52) U.S. Cl. CPC A61K 40/11; A61K 31/711; A61K 35/17; A61K 40/32; A61K 40/4201; A61K 45/06; A61P 35/04; C12N 5/0636; C12Q 1/6869; A61K 2239/53

(57) ABSTRACT

The present disclosure relates to methods of treating cancer or preventing metastases of a cancer in a subject in need thereof. The disclosure further relates to compositions comprising a heterogeneous population of T cells with reactivity to individualized cancer epitopes, or neoantigens, that are useful for adoptive immunotherapy and methods for making such T cell compositions.

### CROSS-REFERENCE TO RELATED APPLICATIONS

This application claims the benefit of U.S. Application No. 63/342,608, filed on May 16, 2022, and U.S. Application No. 63/340,058, filed on May 10, 2022, the contents of which are hereby incorporated by reference in their entireties.

### REFERENCE TO SEQUENCE LISTING

The Sequence Listing submitted May 10, 2023 as an XML file named “GENE-001-PCT_SL” created on May 10, 2023 and having a size of 569,374 bytes, is hereby incorporated by reference pursuant to 37 C.F.R. § 1.52 (e) (5).

### FIELD

The present disclosure relates to methods of treating cancer or methods of preventing metastases of a cancer in a subject in need of therapy or prevention. The disclosure further relates to compositions comprising a heterogeneous populations of T cells with reactivity to individualized cancer epitopes, or neoantigens, that are useful for adoptive immunotherapy and methods for making such T cell compositions.

### BACKGROUND

After a person's immune system first sees an antigen, a population of T cells that recognize the antigen is generated over a period of days and these T cells determine the nature of the response to that antigen thereafter. Antigen recognition and specificity by a T cell is conferred by the structural characteristic of the T cell receptor (TCR) expressed on the T cell surface. Antigen specificity of a T cell is therefore characterized by the presence and function of the specific TCR exhibited by the T cell. A single T cell has TCRs capable of binding to a single antigen presented in combination with a specific Major Histocompatibility Complex molecule, or MHC.

Adoptive transfer of ex vivo expanded antigen-specific T cells was shown to confer immunity against CMV and EBV as early as in the 1990s. See Riddell et al., Science, 1992, 257:238; Rooney et al., Blood, 1998, 92:1549-1555. Adoptive cell therapy using tumor infiltrating lymphocytes (TIL) or cells that have been genetically engineered to express an anti-cancer antigen TCR was also shown to produce positive clinical responses in some cancer patients. However, over the course of tumor progression, the immune response to the tumor became focused on a small number of “dominant” antigens, which were ineffective in promoting tumor regression. In past attempts of using ex vivo expanded T cells for immunotherapy, tumor associated dominant antigen-responsive T cells were inadvertently expanded, leading to inconsistencies in the outcome. Furthermore, obstacles to the successful use of adoptive cell therapy for the widespread treatment of cancer and other diseases remain. For example, T cells and TCRs that specifically recognize cancer antigens may be difficult to identify and/or isolate from a patient.

### SUMMARY OF THE DISCLOSURE

The disclosure relates to methods of treating cancer or methods of preventing metastases of a cancer in a subject in need of therapy or prevention.

In one aspect, the disclosure relates to a method of treating cancer comprising one or a plurality of neoantigens in a subject in need thereof, the method comprising: (a) administering to the subject in need thereof one or a plurality of nucleic acid sequences encoding the one or plurality of neoantigens; (b) allowing a time period sufficient for clonal T cells primed against the one or plurality of neoantigens in the subject to expand to a biologically significant number; (c) isolating the clonal T cells from the subject; (d) identifying one or a plurality of nucleotide sequences encoding a subset of T cell receptors (TCRs) that are highly immunogenic in response to the one or plurality of neoantigens in the subject; and (e) administering a therapeutically effective amount of T cells comprising a nucleic acid molecule encoding one or a plurality of the subset of TCRs to the subject in need thereof.

In some embodiments, the clonal T cells are isolated by drawing a blood sample from the subject and sorting the peripheral blood mononuclear cells (PBMCs) from the sample according to receptor expression on the PBMC surface.

In some embodiments, step (d) comprises performing an assay measuring one or a combination of: (i) the avidity or affinity of cells expressing the TCRs to bind cells in vitro; and (ii) the percentage of CD8+ and/or CD4+ on cells expressing the TCRs.

In some embodiments, the method further comprises sequencing the one or plurality of nucleotide sequences encoding the subset of TCRs that are highly immunogenic from the T cells expressing the TCRs.

In some embodiments, the method further comprises identifying the one or plurality of neoantigens from a tissue sample removed from the subject.

In some embodiments, the tissue sample comprises a tissue from a brushing, biopsy, or surgical resection of the subject.

In some embodiments, the method is free of an in vitro expansion of PBMC and/or tumor infiltrating lymphocytes.

In some embodiments, a total number of the clonal T cells primed against the one or plurality of neoantigens in the subject comprise from about 0.01% to about 10% CD8+ reactivity to the one or plurality of neoantigens.

In some embodiments, step (a) comprises administering a nucleic acid molecule comprising the one or plurality of nucleotide sequences encoding the one or plurality of neoantigens. In some embodiments, the nucleic acid molecule encodes from about 10 to about 55 neoantigens. In some embodiments, each neoantigen encoded by the nucleic acid molecule is separated from another by one or a plurality of linkers. In some embodiments, the one or plurality of linkers comprise a furin protease cleavage site or a porcine teschovirus-1 2A (P2A) cleavage site.

In some embodiments, the nucleic acid molecule is a plasmid. In some embodiments, an expressible nucleic acid sequence is positioned within a multiple cloning site of (i) a plasmid chosen from pVAX1, pcDNA3.1 (+), pCI mammalian expression vector, pSI vector, pZeoSV2 (+), phCMV1, pTCP and pIRES; or (ii) a plasmid comprising at least 70% sequence identity to a plasmid chosed from pVAX1, pcDNA3.1 (+), pCI mammalian expression vector, pSI vector, pZeoSV2 (+), phCMV1, pTCP and pIRES.

In some embodiments, the nucleic acid molecule is GNOS-PV02.

In some embodiments, the cancer is selected from the group consisting of: non-small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

In another aspect, the disclosure relates to a method of treating cancer expressing one or a plurality of neoantigens in a subject in need thereof, the method comprising: (a) administering one or a plurality of nucleic acid sequences encoding the one or plurality of neoantigens to the subject in need thereof; and (b) administering a therapeutically effective amount of T cells comprising one or a plurality of nucleic acid sequences encoding one or a plurality of T cell receptors (TCRs) or functional fragments thereof from the subject that are highly immunogenic in response to the one or plurality of neoantigens to the subject.

In some embodiments, the method is free of an in vitro expansion of PBMC and/or tumor infiltrating lymphocytes.

In some embodiments, the method further comprises allowing the subject to elicit an immune response against the one or plurality of neoantigens.

In some embodiments, the method further comprises sequencing the one or plurality of nucleic acid sequences encoding the one or plurality of TCRs or functional fragments thereof from T cells isolated from the subject after step (a) but prior to step (b).

In some embodiments, the method comprises, after step (a), allowing a time period sufficient for the subject to expand a clonal T cell population primed against the one or plurality of neoantigens, wherein the clonal T cell population comprises from about 25% to about 50% CD8+ reactivity to the one or plurality of neoantigens.

In some embodiments, method comprise a step comprising administering a nucleic acid molecule comprising the one or plurality of nucleic acid sequences encoding the one or plurality of neoantigens.

In some embodiments, the expressible nucleic acid sequence encodes from about 10 to about 55 neoantigens. In some embodiments, each neoantigen encoded by the nucleic acid molecule is separated from another by one or a plurality of linkers. In some embodiments, the one or plurality of linkers comprise a furin protease cleavage site or a porcine teschovirus-1 2A (P2A) cleavage site.

In some embodiments, the nucleic acid molecule is a plasmid. In some embodiments, the nucleic acid molecule is GNOS-PV02.

In some embodiments, an expressible nucleic acid sequence is positioned within a multiple cloning site of (i) a plasmid chosed from pVAX1, pcDNA3.1 (+), pCI mammalian expression vector, pSI vector, pZeoSV2 (+), phCMV1, pTCP and pIRES, GNOS-PV02; or (ii) a plasmid comprising at least 70% sequence identity to a plasmid chosed from pVAX1, pcDNA3.1 (+), pCI mammalian expression vector, pSI vector, pZeoSV2 (+), GNOS-PV02, phCMV1, pTCP and pIRES.

In some embodiments, the cancer is selected from the group consisting of: non-small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

In yet another aspect, the disclosure relates to a method of manufacturing a population of T cells expressing one or a plurality of TCRs, or functional fragments thereof, that recognize one or a plurality of neoantigens, the method comprising: (a) administering one or a plurality of nucleic acid sequences encoding the one or plurality of neoantigens to a subject comprising one or a plurality of cells expressing the one or plurality of neoantigens; and (b) isolating clonally derived T cells expressing the one or plurality of TCRs or functional fragments thereof from the subject.

In some embodiments, the method is free of an in vitro expansion of PBMC and/or tumor infiltrating lymphocytes. In some embodiments, the method further comprises sequencing one or plurality of nucleic acid sequences encoding the one or plurality of TCRs or functional fragments thereof after step (b).

In some embodiments, the method further comprises, after step (a), a step of allowing a time period sufficient for the subject to expand a clonal T cell population primed against the one or plurality of neoantigens.

In some embodiments, the clonal T cell population comprises from about 25% to about 50% CD8+ reactivity to the one or plurality of neoantigens.

In some embodiments, the method further comprises transferring one or plurality of nucleic acid sequences encoding the one or plurality of TCRs or functional fragments thereof into T cells obtained from the subject.

In yet another aspect, the disclosure relates to a method of preventing metastases of a cancer comprising one or a plurality of neoantigens in a subject, the method comprising: (a) administering to the subject one or a plurality of nucleic acid sequences encoding the one or plurality of neoantigens; (b) allowing a time period sufficient for clonal T cells primed against the one or plurality of neoantigens in the subject to expand to a biologically significant number; (c) isolating the clonal T cells from the subject; (d) identifying one or a plurality of nucleotide sequences encoding a subset of TCRs that are highly immunogenic in response to the one or plurality of neoantigens in the subject; and (e) administering a therapeutically effective amount of T cells comprising a nucleic acid molecule encoding one or a plurality of the subset of TCRs to the subject in need thereof.

In some embodiments, the clonal T cells are isolated by drawing a blood sample from the subject and sorting the peripheral blood mononuclear cells (PBMCs) from the sample according to receptor expression on the PBMC surface.

In some embodiments, step (d) comprises performing an assay measuring one or a combination of: (i) the avidity or affinity of cells expressing the TCRs to bind cells in vitro; and (ii) the percentage of CD8+ and/or CD4+ on cells expressing the TCRs.

In some embodiments, the method further comprises sequencing the one or plurality of nucleotide sequences encoding the subset of TCRs that are highly immunogenic from the T cells expressing the TCRs.

In some embodiments, the method further comprises identifying the one or plurality of neoantigens from a tissue sample removed from the subject.

In some embodiments, the tissue sample comprises a tissue from a brushing, biopsy, or surgical resection of the subject.

In some embodiments, the method is free of an in vitro expansion of PBMC and/or tumor infiltrating lymphocytes.

In some embodiments, a total number of the clonal T cells primed against the one or plurality of neoantigens in the subject comprise from about 25% to about 50% CD8+ reactivity to the one or plurality of neoantigens.

In some embodiments, step (a) comprises administering a nucleic acid molecule comprising the one or plurality of nucleotide sequences encoding the one or plurality of neoantigens.

In some embodiments, the nucleic acid molecule encodes from about 10 to about 55 neoantigens. In some embodiments, the disclosure relates to a composition comprising a nucleic acid molecule that encodes from about 10 to about 55 neoantigens. In some embodiments, the disclosure relates to a composition comprising a nucleic acid molecule that encodes from about 19 to about 60 neoantigens. In some embodiments, the disclosure relates to a composition comprising a nucleic acid molecule that encodes from about 20 to about 60 neoantigens. In some embodiments, the disclosure relates to a composition comprising a nucleic acid molecule that encodes from about 20 to about 65 neoantigens.

In some embodiments, each neoantigen encoded by the nucleic acid molecule is separated from another by one or a plurality of linkers.

In some embodiments, the one or plurality of linkers comprise a furin protease cleavage site or a porcine teschovirus-1 2A (P2A) cleavage site.

In some embodiments, the nucleic acid molecule is a plasmid.

In some embodiments, the nucleic acid molecule is positioned within a multiple cloning site of (i) a plasmid chosed from pVAX1, pcDNA3.1 (+), pCI mammalian expression vector, pSI vector, pZeoSV2 (+), phCMV1, pTCP and pIRES; or (ii) a plasmid comprising at least 70% sequence identity to a plasmid chosed from pVAX1, pcDNA3.1 (+), pCI mammalian expression vector, pSI vector, pZeoSV2 (+), phCMV1, pTCP and pIRES.

In some embodiments, the nucleic acid molecule is GNOS-PV02.

In some embodiments, the cancer is selected from the group consisting of: non-small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

### BRIEF DESCRIPTION OF THE DRAWINGS

FIG. 1 shows a schematic of a method of the disclosure for developing a personalized T cell vaccine based on tumor-specific neoantigens. Because the majority of neoantigens are unique to an individual patient's cancer, the “mutanome” of each patient's tumor is determined, allowing for the identification of candidate neoantigens to be targeted by the vaccine. Neoantigen-specific T cells are activated and expanded in vivo by administering one or more DNA or RNA vaccines encoding one or more patient-specific neoantigens. The T cells are then isolated from the patient and assayed to identify T cell receptors (TCRs) that are highly immunogenic in response to the one or more patient-specific neoantigens. Once identified, engineered T cell expressing such TCRs can be generated and administered to the patient.

FIG. 2 shows the predicted MHC class I/II binding affinity (nM) of pool compositions for immune analysis with the GEN-PV-001 vaccine.

FIG. 3 shows the use of 33 amino acid long sequences for each neoantigen in the vaccine allow encoding for potential CD8 and CD4 epitopes encompassing the neoantigen, which permits a more effective adaptive immune response. CD8 epitopes are more commonly 9 amino acids in length but can range from 8 to 14 amino acids, and CD4 epitopes are generally 15 amino acids in length but can range from 9 to 25 amino acids. See Chong et al., Mol. Cell Proteomics, 2018, 17 (3): 533-548. This means that a 33 amino acid sequence should encompass the whole predicted epitopes in neoantigens with overlapping CD8 and CD4 epitope.

FIG. 4A-4B show the immune responses to the tested neoantigen. FIG. 4A: Immune responses (IFNγ ELISpot) to the tested neoantigen (marked in the schematic with a star) is not significantly different when it is located in position 1, 10, 20, 30 or 40 of a 40-epitope neoantigen DNA vaccine (4806 nucleotide insert). FIG. 4B: Immune responses (IFNγ ELISpot) to a vaccine containing 30 epitopes (27 neoantigens and 3 tumor associated antigens) divided into 4 pools: Pool 1 contains vaccine epitopes 1-8; Pool 2 contains vaccine epitopes 9-16; Pool 3 contains vaccine epitopes 17-23; and Pool 4 contains vaccine epitopes 24-30. Following vaccination of a patient with anaplastic astrocytoma, responses were found in all 4 pools, showing that DNA neoantigen vaccines express as protein antigens present through out the whole length of the DNA sequence.

FIG. 5 shows that T cell responses (IFNγ ELISpot) were detected to 17 neoantigens out of 30 encoded by the GEN-PV-001 vaccine.

FIG. 6 shows the heat maps of intracellular cytokine staining (ICS) analysis. Both CD8+ and CD4+ T cell responses were detected to the epitopes encoded by the GEN-PV-001 vaccine and the responses were to multiple markers. Heat map ranges are in % and represent the % of CD4+ or CD8+ T cells that express the indicated marker in a peptide-specific manner (peptide stimulated-vehicle control).

FIG. 7A-7B show neoantigen DNA vaccines generated CD8 and CD4 responses to multiple epitopes in cancer patients. FIG. 7A: Heat map ranges are in % and represent the % of CD4+ or CD8+ T cells that express the indicated marker in a peptide-specific manner (peptide stimulated-vehicle control). FIG. 7B: Representative flow plots showing the presence of CD8+, CD4+ T cells that produce IFNγ, TNFα, or both in a peptide specific manner.

FIG. 8A is a non-limiting example of a manufacturing process for personalized DNA vaccines. Needle-to-needle has been achieved in as low as 6 weeks and can be regularly achieved in 6-8 weeks. FIG. 8B is a non-limiting example of a clinical trial.

FIG. 9A shows a spider plot showing the first 12 patients of a clinical trial at the time of the data cut. FIG. 9B is a waterfall plot showing the best overall response achieved by the first 12 subjects of the clinical trial at the time of the data cut. Best overall response shows 25% partial response rate and 67% Disease Control Rate. FIG. 9C is tumor imaging scans (day 0 vs week 27 post-treatment) of patients categorized as PR. Red arrows point at the tumors.

FIG. 10A shows a bar graph that all patients analyzed to date (n=10) have newly detected and expanded T cell clones after treatment with GNOS-PV02. FIG. 10B shows a cumulative frequency of expanded clones in peripheral blood (PBMC, left) and in the tumor tissue (right) pre-vs post-vaccination (week 9) per patient. FIG. 10C shows expansion of pre-vaccination clones (dots along the X axis) and detection of multiple new T cell clones (dots along Y axis) post-vaccination in blood and tumor tissue from subject Pt 7. Arrows highlight infiltration of high frequency clones from blood into the tumor 9 weeks post-vaccination (only top 6 clones shown for clarity). Most abundant clones show an active phenotype (CD8+CD69+) as assessed by TCRβ and RNA sequencing. Approx. 75% of new TIL clones were undetectable in blood prior to vaccination.

FIG. 11A shows patient-specific clonal TCR sequences were gene optimized using GOAL algorithm and inserted into the pMXs-IRES-GFP retroviral plasmid vector containing viral packaging signal, transcriptional and processing elements, and GFP reporter gene. FIG. 11B shows an example of anti-tumor specific T cell reactivity post-vaccination evaluated by ELISpot (subject Pt 8). PBMCs were stimulated with a pool of, or individual peptides encoded in the personalized GNOS-PV02 treatment. FIG. 11C shows representative images of activated, GFP positive, CD8 and CD4 TCR-engineered T cells (subject Pt 8), stimulated with ATP1A1-ALB (10 ug/mL). TNTC, Too Numerous To Count; EOT, End of Treatment.

FIG. 12 shows that PTCVs drive neoantigen-specific responses that are detected in blood. FIG. 12A shows results of ELISpot assays from PBMC samples for the presence of vaccine-induced neoantigen-specific responses prior to and post-personalized GNOS-PV02 vaccination without cytokine stimulation. The post-vaccination response is the ‘best’ (highest magnitude) response for each patient across time points. FIG. 12B shows Positive neoantigens pre- and post-vaccination (black and red bars, respectively) relative to the total number (grey bars) included in each patient's PTCV as defined by IFNγ ELISpot assay. FIG. 12C shows percentage of positive responding epitopes by clinical response group and pre-versus on-treatment timepoint. FIG. 12D shows Spearman correlation between positive epitopes versus the total number of neoantigens included in each patient's PTCV. FIG. 12 E shows Neoantigen-specific T cell activation evaluated by stimulating patient-derived PBMCs (week 9 or 12) with DMSO or patient-specific epitope pools ex vivo by intracellular cytokine staining. FIGS. 12F and G show polyfunctionality assessment via Boolean gating of CD4+ or CD8+cytokine+ populations. T cell activation (CD69 and CD107a) and proliferation (Ki67) were assessed together with the double positive expression of granzyme A (GrzA) and perforin (Prf) to evaluate the cytolytic potential of neoantigen-reactive T cells. Results are represented as % positive cell populations (frequency of parent). Filled circles represent individual patients, the box extends from the 25th to the 75th percentile, the line inside the box is the median, and the whiskers extend from the minimum to maximum values. Four patients (1CR, 3PR) were analyzed.

FIG. 13 shows that GNOS-PV02 results in the expansion of new T cell clones that traffic to the tumor. FIG. 13A shows that in 14 out of 14 subjects T cell clones expanded in the periphery and the new or expanded clones were enriched in the matched tumor sample for each patient. Total in PBMC and tumor-associated T cell expansion is calculated by comparing post-treatment over pre-treatment PBMC or tumor samples respectively (differential abundance statistical analysis). FIG. 13B shows cumulative frequencies of peripherally expanded TCR rearrangements tracked in pre- and post-treatment tumor biopsies. FIG. 13C shows significantly expanded clone numbers found in pre- and post-treatment tumor biopsies. Circles represent individual patients, the box extends from the 25th to the 75th percentile, the line inside the box is the median, and the whiskers extend from the minimum to maximum values. FIGS. 13D and E show TCR clonality and TCR repertoire richness in tumor biopsies of 14 evaluated patients (bar-line and stacked bar plots). Error bars correspond to the upper SE of each group. Simpson clonality reports the distribution of TCR rearrangements in a sample, where 0 indicates an even distribution of frequencies and 1 indicates an asymmetric distribution. Lower numbers indicate focused TCR diversity.

FIG. 14 shows that post-vaccination expanded TCR clones identified in the tumor are reactive to PTCV encoded antigens. FIG. 14A shows the most frequent TCRs identified by TCRβ and RNA sequencing in a patient. Pre-vaccination versus week 9 post-vaccination (Pair-wise scatter plot). Blue asterisks show selected high-frequency new T cell clones detected in the PBMC post-vaccination, and their abundance in the tumor. Orange, green, and grey circles represent expanded, contracted, and not significantly changed T cell clones, respectively. FIG. 14B shows evaluated TCRs selected for cloning based on their occurrence in high frequency in the PBMC and trafficking to the tumor post-vaccination. FIG. 14C shows results of patient-specific clonal TCR sequences gene optimized and inserted into the pMXs-IRES-GFP retroviral plasmid vector containing viral packaging signal, transcriptional and processing elements, and GFP reporter gene. FIGS. 14D and 14E show TCR-engineered T cells (GFP positive) from unvaccinated PBMC were stimulated for 6 hours with increasing concentrations of epitope pools (2, 10, 25 μg/mL), and the expression of CD69 was evaluated by flow cytometry. Peptide pools 1 and 2 comprise the first or second half of the neoantigens included in the PTCV.

### DETAILED DESCRIPTION

The disclosed method and compositions may be understood more readily by reference to the following detailed description of particular embodiments and the examples included therein and to the figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present disclosure which will be limited only by the appended claims.

### Definitions

It must be noted that as used herein and in the appended claims, the singular forms “a”, “an,” and “the” include plural reference unless the context clearly dictates otherwise. Thus, for example, reference to “a nucleic acid sequence” includes a plurality of nucleotides that are formed, reference to “the nucleic acid sequence” is a reference to one or more nucleic acid sequences and equivalents thereof known to those skilled in the art, and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of +20%, +10%, +5%, +1%, +0.9%, +0.8%, +0.7%, +0.6%, +0.5%, +0.4%, +0.3%, +0.2% or +0.1% from the specified value as such variations are appropriate to perform the disclosed methods. When “about” is present before a series of numbers or a range, it is understood that “about” can modify each of the numbers in the series or range.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by binding of a cell surface moiety. For example, in the context of receptors like a TCR, such stimulation entails the binding of a receptor and a subsequent signal transduction event. In some embodiments, activate means that TCR associates with a tumor-specific epitope or antigen, and the cell comprising the TCR responds by releasing immunostimulatory agents in response to the association. Further, the stimulation event may activate a cell and upregulate or downregulate expression or secretion of a molecule. Thus, binding of cell surface moieties, even in the absence of a direct signal transduction event, may result in the reorganization of cytoskeletal structures, or in the coalescing of cell surface moieties, each of which could serve to enhance, modify, or alter subsequent cellular responses. In some embodiments, an epitope is activated or highly immunogenic if, upon association with the neoantigens or tumor-specific epitope, the cell releases IFNgamma, TNFalpha, or IL-2. In some embodiments, a neoantigen or TCR is highly immunogenic if a cell comprising the TCR is clonally expanded, exposed to the neoantigens and a population greater or equal to about 20%, 25%, 30%, 35% or about 40% or secrete IFNgamma, TNFalpha, or IL-2. In some embodiments, a neoantigen or TCR is highly immunogenic to or with a neoantigens if a cell comprising the TCR is clonally expanded, exposed to the neoantigens and a cell population greater or equal to about 20%, 25%, 30%, 35% or about 40% or express one or more immunostimulatory agents is in response to the presence of or association with the neoantigen. In some embodiments, highly immunogenic TCRs are those TCRs, in respect to binding or associating with a neoantigen,

As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

Activation can be defined as: >50 IFNγ spots/1×106 PBMC as evaluated by ELISpot; >0.05% IFNγ positive T cells by FACS, >100 μg/mL by ELISA, and >2-fold IFNγ mRNA expression. Moreover, activation can be defined by CD137 and/or CD69 expression, which can be measured by RNA sequencing, Flow cytometry, or ELISA. Alternatively, the cells can be selected sorted out utilizing bead-bound or column-bound antibodies against CD137, CD69, CD25 and/or CD38.

As used herein, the term “adjuvant” is meant to refer to any molecule added to the DNA plasmid vaccines described herein to enhance the immunogenicity of the antigens encoded by the DNA plasmids and the encoding nucleic acid sequences described hereinafter. 00072 The term “allogeneic” as used herein refers to medical therapy in which the donor and recipient are different individuals of the same species.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiment, to B without A (optionally including elements other than A); in yet another embodiment, to both A and B (optionally including other elements).

As used herein, an “antigen” or “Ag” refers to a molecule that elicits an immune response and this immune response may involve antibody production, activation of specific immunologically-competent cells (e.g., T cells), or both. An antigen may be, for example, a peptide, glycopeptide, polypeptide, glycopolypeptide, polynucleotide, polysaccharide, lipid or the like. An antigen can be synthesized, produced recombinantly, or derived from a biological sample using methods known in the art. For example, novel antigens can be generated using methods known in the art such as chromosome rearrangement or breakage. Exemplary biological samples that can contain one or more antigens include tissue samples, tumor samples, cells, biological fluids, or combinations thereof. Antigens can be produced by cells that have been modified or genetically engineered to express an antigen. The term “antigen” includes antigenic determinants, such as peptides with lengths of 6, 7, 8, 9, 10, 11, 12, 13, 14, 15, 16, 17, 18, 19, 20, 21, 22 or more amino acid residues that bind to MHC molecules, form parts of MHC Class I or II complexes, or that are recognized when complexed with such molecules.

The term “antigen presenting cell (APC)” as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

As used herein, the term “anti-tumor response” refers to an immune system response including but not limited to activating T-cells to attack an antigen or an antigen presenting cell.

The term “autologous” as used herein refers to medical therapy in which the donor and recipient are the same person.

The term “cancer” as used herein is meant to refer to any disease that is caused by, or results in, inappropriately high levels of cell division, inappropriately low levels of apoptosis, or both. Examples of cancer include, but are not limited to, non-small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

The term “checkpoint inhibitor” as used herein is meant to refer to any small molecule chemical compound, antibody, nucleic acid molecule, or polypeptide, or fragments thereof, that inhibits the inhibitory pathways, allowing more extensive immune activity. In certain embodiments, the checkpoint inhibitor is an inhibitor of the programmed death-1 (PD-1) pathway, for example an anti-PD1 antibody, such as, but not limited to Nivolumab. In other embodiments, the checkpoint inhibitor is an anti-cytotoxic T-lymphocyte-associated antigen (CTLA-4) antibody. In further additional embodiments, the checkpoint inhibitor is targeted at a member of the TNF superfamily such as CD40, OX40, CD 137, GITR, CD27 or TIM-3. In some cases targeting a checkpoint inhibitor is accomplished with an inhibitory antibody or similar molecule. In other cases, it is accomplished with an agonist for the target; examples of this class include the stimulatory targets OX40 and GITR.

The term “combination therapy” as used herein is meant to refer to administration of one or more therapeutic agents in a sequential manner, that is, wherein each therapeutic agent is administered at a different time, as well as administration of these therapeutic agents, or at least two of the therapeutic agents, in a substantially simultaneous manner. Sequential administration, for example, includes administering separately (i) a pharmaceutical composition comprising an effective amount of a pooled sample of tumor specific neoantigens, or DNA/RNA encoding the same and a pharmaceutically acceptable salt, carrier or diluent, (ii) a pharmaceutical composition comprising an effective amount of IL-12 or DNA/RNA encoding the same and (iii) an effective amount of a checkpoint inhibitor and a pharmaceutically acceptable salt, carrier or diluent. Substantially simultaneous administration can be accomplished, for example, by administering to the subject a single capsule having a fixed ratio of each therapeutic agent or in multiple, single capsules for each of the therapeutic agents. For example, one combination therapy of the present disclosure may comprise a pooled sample of tumor specific neoantigens, or DNA/RNA encoding the same, IL-12 or DNA/RNA encoding the same, and a checkpoint inhibitor, and a pharmaceutically acceptable salt, carrier or diluent administered at the same or different time. In some embodiments, the pharmaceutical composition of the disclosure can be formulated as a single, co-formulated pharmaceutical composition comprising the two or three compounds. As another example, in some embodiments, a combination of the present disclosure (e.g., DNA neoantigen vaccines, IL-12 and a checkpoint inhibitor) is formulated as separate pharmaceutical compositions that can be administered at the same or different time. As used herein, the term “simultaneously” is meant to refer to administration of one or more agents at the same time. For example, in certain embodiments, a cancer vaccine or immunogenic composition and a checkpoint inhibitor are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, subcutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, in some embodiments, one component of a particular combination is administered by intravenous injection while the other component(s) of the combination is administered orally. The components may be administered in any therapeutically effective sequence. A “combination” embraces groups of compounds or non-drug therapies useful as part of a combination therapy.

The term “cytotoxic T-cell” or “cytotoxic T lymphocyte” as used herein is a type of immune cell that comprises a CD receptor capable of recognizing one or a plurality of antigens and that can kill certain cells, including foreign cells, tumor cells, and cells expressing that antigen. Cytotoxic T cells can be separated from other blood cells, grown ex vivo, and then given to a patient to kill tumor or viral cells expressing antigens recognizable to the cell. A cytotoxic T cell is a subset of white blood cells and a type of lymphocyte.

The term “dendritic cell” or “DC” as used herein describes a diverse population of morphologically similar cell types found in a variety of lymphoid and non-lymphoid tissues, see Steinman, Ann. Rev. Immunol. 9:271-296 (1991).

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other.

As used herein, the term “endogenous” or “native” refers to a gene, protein, or activity that is present in an in vivo host cell. A gene, protein, or activity that is mutated, overexpressed, shuffled, duplicated, or otherwise altered as compared to a normal, unmutated gene, protein, or activity is still considered to be endogenous or native to that particular in vivo host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000 or more nucleotides or amino acids.

The term “functional fragment” refers to any portion of a polypeptide that is of a sufficient length to retain at least partial biological function similar to or substantially similar to the biological function of the wild-type polypeptide upon which the fragment is based. A functional fragment of a TCR disclosed herein is a fragment of the TCs disclosed herein and maintains at least a partial binding affinity to its target. In some embodiments, a functional fragment has a length of at least about 10, about 20, about 30, about 40, about 50, about 60, about 70, about 80, about 90, or about 100 contiguous amino acids. In some embodiments, the functional fragment has a length of 25 amino acids, 26 amino acids, 27 amino acids, 28 amino acids, 29 amino acids, 30 amino acids, 31 amino acids, 32 amino acids, 33 amino acids, 34 amino acids, or 35 amino acids. In some embodiments, the functional fragment has a length of between about 25 amino acids and about 35 amino acids. In some embodiments, the functional fragment has a length of between about 27 amino acids and about 35 amino acids. In some embodiments, the functional fragment has a length of between about 29 amino acids and about 35 amino acids. In some embodiments, the functional fragment has a length of between about 31 amino acids and about 35 amino acids. In some embodiments, the functional fragment is a fragment of the TCRs disclosed herein and has a length of at least about 25, 50, 75, 100, 150, 200, 250, 300, 350, 400, 450, or 500 amino acids.

As used herein, the term “genetic construct” is meant to refer to a DNA or RNA molecule that comprises a nucleotide or ribonucleotide sequence encoding one or plurality of amino acid sequences. In some embodiments, the amino acid sequence is a protein, fragment of protein or antigen. In some embodiments, the genetic construct comprises one or a plurality of coding sequences, and one or a plurality of regulatory sequences. In some embodiments, the coding sequence includes an initiation sequence and a termination sequence operably linked to regulatory elements. In some embodiments, regulatory elements comprise a promoter and polyadenylation signal capable of directing expression in the cells of an individual to whom the DNA or RNA molecule is administered.

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In some embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” or “exogenous” refers to a non-native enzyme, protein, or other activity encoded by an exogenous nucleic acid molecule introduced into the host cell, even if the host cell encodes a homologous protein or activity.

The term “host cell” as used herein refers to a cell that can be used to express a genetic construct, such as nucleic acids of the disclosure. It can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. The phrase “recombinant host cell” can be used to denote a host cell that has been transformed or transfected with a nucleic acid to be expressed. A host cell also can be a cell that comprises the claimed nucleic acid sequences but does not express it at a level sufficient to elicit an immunogenic response unless a regulatory sequence is introduced into the host cell such that it becomes operably linked with the nucleic acid. It is understood that, in some embodiments, the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “hybridize” as used herein is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

As used herein, an “immune cell” or “immune system cell” means any cell of the immune system that originates from a hematopoietic stem cell in the bone marrow, which gives rise to two major lineages, a myeloid progenitor cell (which gives rise to myeloid cells such as monocytes, macrophages, dendritic cells, megakaryocytes, mast cells, thrombocytes, erythrocytes, and granulocytes) and a lymphoid progenitor cell (which gives rise to lymphoid cells, or “lymphocytes”). As used herein, the term “lymphocyte” refers to a subtype of white blood cell of a vertebrate immune system that is characterized by its predominant presence in lymph and, generally, by a large nucleus. Lymphocytes include, for example, T cells (CD4+ T cells, CD8+ T cells, CD4-CD8-double-negative T cells, γδ T cells, regulatory T cells), B cells, and natural killer (NK) cells. Other exemplary immune system cells include macrophages and dendritic cells, as well as other myeloid cells as described herein. Macrophages and dendritic cells may be referred to as “professional antigen presenting cells” (or “professional APCs”), which are specialized cells that can activate T cells when a major histocompatibility complex (MHC) receptor on the surface of the APC interacts with a TCR on the surface of a T cell. Alternatively, any hematopoietic stem cell or immune system cell can be converted into an APC by introducing a nucleic acid molecule that expresses an antigen recognized by a TCR or by another antigen binding protein (e.g., chimeric antigen receptor or antibody). Immune cells or lymphocytes used in vaccine compositions or methods of treatment of this disclosure may be autologous, allogeneic, or syngeneic to a subject to receive the composition or the method of treatment.

The term “immune checkpoint” as used herein is meant to refer to inhibitory pathways that slow down or stop immune reactions and prevent excessive tissue damage from uncontrolled activity of immune cells.

The term “immune response” is used herein is meant to refer to the activation of a host's immune system, e.g., that of a mammal, in response to the introduction of nucleic acid molecules comprising a nucleotide sequence encoding neoantigens as described herein.

As used herein, an “immunogenicity enhancer” comprises a molecule encoded by a polynucleotide contained in a host cell, such as a T cell, that enhances immunogenicity of an exogenous neoantigen encoded by a polynucleotide contained in the cell. An immunogenicity enhancer encoded by a host cell can provide localized and concentrated adjuvant activity that improves an immune response against a neoantigen. Exemplary immunogenicity enhancers include IL-12 (such as a membrane-tethered IL-12), a GM-CSF, an inducible cell death factor, a bacterial flagellin, a CD80, a CD137L, a CD40L, a secreted IL-2, a secreted IL-2 that binds T cells independent of CD25, a secreted IL-15, a secreted IL-15-IL-15Ra complex, a secreted IFNβ, a secreted IFN-α1, a secreted IL-7, or any combination thereof. In some embodiments, an immunogenicity enhancer is endogenously expressed by the host cell (e.g., the host cell endogenously expresses, for example, GM-CSF), in which case the host cell is engineered to increase the expression of the immunogenicity enhancer, or the immunogenicity enhancer is exogenous to the host cell.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof. In some embodiments, the subject in need thereof is a human seeking prevention of cancer. In some embodiments, the subject in need thereof is a human diagnosed with cancer. In some embodiments, the subject in need thereof is a human seeking treatment for cancer. In some embodiments, the subject in need thereof is a human undergoing treatment for cancer. In some embodiments, the subject in need thereof is a healthy subject.

The term “introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid molecule may be incorporated into the genome of a cell (e.g., chromosome, plasmid, plastid, or mitochondrial DNA), converted into an autonomous replicon, comprised in an episomal expression vector (see, e.g., Van Caenenbroeck et al., Eur. J. Biochem. 267:5665 (2000)), or transiently expressed (e.g., transfected mRNA).

The term “isolated” as used herein means that the polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptide of the invention.

The term “ligand” as used herein is meant to refer to a molecule which has a structure complementary to that of a receptor and is capable of forming a complex with this receptor. According to embodiments of the disclosure, a ligand is to be understood as meaning in particular a peptide or peptide fragment which has a suitable length and suitable binding motifs in its amino acid sequence, so that the peptide or peptide fragment is capable of forming a complex with proteins of MHC class I or MHC class II.

The terms “MHC molecules,” “MHC proteins” or “HLA proteins” as used herein are meant to refer to proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles. Structurally, MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The heavy chain of the MHC molecules of class I is preferably an HLA-A, HLA-B or HLA-C monomer, and the light chain is β-2-microglobulin. MHC molecules of class II consist of an α-chain and a β-chain and are capable of binding a peptide of about 15 to 24 amino acids if this peptide has suitable binding motifs, and presenting it to T-helper cells. The α-chain and the β-chain are in particular HLA-DR, HLA-DQ and HLA-DP monomers.

As used herein, “mutation” refers to a change in the sequence of a nucleic acid molecule or polypeptide molecule as compared to a reference, wild-type, or endogenous nucleic acid sequence or polypeptide sequence, respectively. A mutation can result in several different types of change in sequence, including substitution, insertion or deletion of nucleotide(s) or amino acid(s). In some embodiments, a mutation is a substitution of one or more codons or amino acids. In some embodiments, a mutation is an insertion of one or more codons or amino acids. In some embodiments, a mutation is a deletion of one or more codons or amino acids. In some embodiments, a mutation is a combination of any of a substitution of one or more codons or amino acids, an insertion of one or more codons or amino acids, and a deletion of one or more codons or amino acids.

As used herein, a “neoantigen” refers to a host cellular product containing a structural change, alteration or mutation that creates a new antigen or antigenic epitope that has not previously been observed in the subject's genome (i.e., in a sample of healthy tissue from the subject) or been “seen” or recognized by the host's immune system. Neoantigens may originate, for example, from coding polynucleotides having alterations (substitution, addition, deletion) that result in an altered or mutated product, or from the insertion of an exogenous nucleic acid molecule or protein into a cell, or from exposure to environmental factors (e.g., chemical, radiological) resulting in a genetic change. Neoantigens may arise separately from a tumor antigen, or may arise from or be associated with a tumor antigen. “Tumor neoantigen” (or “tumor-specific neoantigen”) refers to a protein comprising a neoantigenic determinant associated with, arising from, or arising within a tumor cell or plurality of cells within a tumor. Tumor neoantigenic determinants are found on, for example, antigenic tumor proteins or peptides that contain one or more somatic mutations encoded by the DNA of tumor cells, as well as proteins or peptides from viral open reading frames associated with virus-associated tumors (e.g., cervical cancers, some head and neck cancers). For example, tumor neoantigens may arise within or from any of the exemplary tumor or other antigens, as well as from “driver” cancer antigens (e.g., G12D neoantigen from KRAS described in Tran et al., N. Eng. J. Med. 375:2255-2262 (2016)), as well as in mutated B-Raf, SF31, MYD88, DDX3X, MAPK1, GNB1, and others).

The term “neoantigen mutation” as used herein refers to a mutation that is predicted to encode a neoantigenic peptide. Such neoantigenic peptides may be expressed on the surface of a cancer cell in a subject.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of neoantigens. In some embodiments, the disclosure relates to a pharmaceutical composition comprising a first, second, third or more nucleic acid molecule, each of which independently or cocommitantly (e.g. in trans) encode one or a plurality of neoantigens and at least one of each plasmid comprising one or more of the formulae disclosed herein.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06, incorporated by reference herein.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

A “peripheral blood mononuclear cell” or “PBMC” as used herein is any peripheral blood cell having a round nucleus. These cells consist of lymphocytes (T cells, B cells, NK cells) and monocytes. In humans, lymphocytes make up the majority of the PBMC population, followed by monocytes, and a small percentage of dendritic cells.

The term “pharmaceutically acceptable” as used herein refers to approved or approvable by a regulatory agency of the Federal or a state government or listed in the U.S. Pharmacopeia or other generally recognized pharmacopeia for use in animals, including humans.

The term “pharmaceutically acceptable excipient, carrier or diluent” as used herein is meant to refer to an excipient, carrier or diluent that can be administered to a subject, together with an agent, and which does not destroy the pharmacological activity thereof and is nontoxic when administered in doses sufficient to deliver a therapeutic amount of the agent.

The term “pharmaceutically acceptable salt” of tumor specific neoantigens as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH2) n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The term “pharmaceutical composition” includes, without limitation, (i) a pharmaceutical composition comprising an effective amount of a pooled sample of tumor specific neoantigens, or DNA/RNA encoding the same and a pharmaceutically acceptable salt, carrier or diluent, (ii) a pharmaceutical composition comprising an effective amount of IL-12 or DNA/RNA encoding the same and (iii) an effective amount of a checkpoint inhibitor and a pharmaceutically acceptable salt, carrier or diluent. It further includes any composition comprising two or three of these ingredients and a pharmaceutically acceptable salt, carrier or diluent.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH2, NHR, N2 or CN, wherein R is C1-C6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in US20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

The terms “polypeptide,” “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the invention. A purified polypeptide of the present disclosure, for instance, is a polypeptide that is at least from about 70% to about 100% pure, i.e., the polypeptide is present in a composition wherein the polypeptide constitutes from about 70% to about 100% by weight of the total composition. In some embodiments, the purified polypeptide of the present disclosure is from about 75% to about 99% by weight pure, from about 80% to about 99% by weight pure, from about 90 to about 99% by weight pure, or from about 95% to about 99% by weight pure. Likewise, a purified polynucleotide of the present disclosure is a polynucleotide that is at least from about 70% to about 100% pure, i.e., the polynucleotide is present in a composition wherein the polynucleotide constitutes from about 70% to about 100% by weight of the total composition. In some embodiments, the purified polynucleotide of the present disclosure is from about 75% to about 99% by weight pure, from about 80% to about 99% by weight pure, from about 90 to about 99% by weight pure, or from about 95% to about 99% by weight pure.

The term “receptor” as used herein, is meant to refer to a biological molecule or a molecule grouping capable of binding a ligand. A receptor may serve, to transmit information in a cell, a cell formation or an organism. The receptor comprises at least one receptor unit and preferably two receptor units, where each receptor unit may consist of a protein molecule, in particular a glycoprotein molecule. The receptor has a structure which complements that of a ligand and may complex the ligand as a binding partner. The information is transmitted in particular by conformational changes of the receptor following complexation of the ligand on the surface of a cell. According to embodiments of the disclosure, a receptor is to be understood as meaning in particular proteins of MHC classes I and II capable of forming a receptor/ligand complex with a ligand, in particular a peptide or peptide fragment of suitable length.

As used herein, the term “sample” refers generally to a limited quantity of something which is intended to be similar to and represent a larger amount of that something. In the present disclosure, a sample is a collection, swab, brushing, scraping, biopsy, removed tissue, or surgical resection that is to be testing for the absence, presence or grading of a hyperproliferative tissue, which, in some cases is cancerous tissue or one or a plurality of cells. In some embodiments, samples are taken from a patient or subject that is believed to have a cancer, hyperplasia, pre-cancerous or comprise one or more tumor cells. In some embodiments, a sample believed to contain one or more hyperproliferative cells is compared to a “control sample” that is known not to contain one or more hyperproliferative cells. This disclosure contemplates using any one or a plurality of disclosed samples herein to identify, detect, sequence and/or quantify the amount of neoantigens (highly or minimally immunogenic) within a particular sample. In some embodiments, the methods relate to the step of exposing a swab, brushing or other sample from an environment to a set of reagents sufficient to isolate and/or sequence the DNA and RNA of one or a plurality of cells in the sample.

As used herein, the term “small molecule” refers to a low molecular weight (<900 daltons) organic compound that may help regulate a biological process, with a size on the order of 1 nm. Most drugs are small molecules.

As used herein, “specifically binds” or “specific for” refers to an association or union of a binding protein (e.g., a receptor, an antibody, CAR, or TCR) or a binding component (or fusion protein thereof) to a target molecule with an affinity or Ka (i.e., an equilibrium association constant of a particular binding interaction with units of 1/M) equal to or greater than 105 M-1 (which equals the ratio of the on-rate [kon] to the off-rate [koff] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Ka of at least about 107 M-1, at least about 108 M-1, at least about 109 M-1, at least about 1010 M-1, at least about 1011 M-1, at least about 1012 M-1, or at least about 1013 M-1. “Low affinity” binding proteins or binding domains refer to those binding proteins or binding domains having a Ka of up to about 107 M-1, up to about 106 M-1, or up to about 105 M-1. In some embodiments, affinity is defined as an equilibrium dissociation constant (Kd) of a particular binding interaction with units of M (e.g., 10-5 M to 10-13 M). A variety of assays are known for identifying binding domains that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173; 5,468,614, or the equivalent, all incorporated herein by reference). In some embodiments, one or a plurality of TCRs bind with high affinity to one or a plurality of respective neoantigens. Binding with high affinity means that, in some embodiments, the TCRs binds to the neoantigens with a Ka or Kd of about 500 nM or less.

The phrase “stringent hybridization conditions” or “stringent conditions” as used herein is meant to refer to conditions under which a nucleic acid molecule will hybridize another nucleic acid molecule, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g. 10 to 50 nucleotides) and at least about 60° C. for longer probes, primers or oligonucleotides. Stringent conditions may also be achieved with the addition of destabilizing agents, such as formamide.

As used herein, the terms “subject,” “individual,” “host,” and “patient” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human. In some embodiments, the subject is a dog, horse, pig, sheep, cat, cow, donkey, llama, emu, or goat.

By “substantially identical” is meant nucleic acid molecule or polypeptide exhibiting at least about 50% sequence identity to a reference nucleic acid sequence (for example, any one of the nucleic acid sequences described herein) or amino acid sequence (for example, any one of the amino acid sequences described herein). In some embodiments, such a sequence is at least about 60% sequence identity to the reference sequence used for comparison. In some embodiments, such a sequence is at least about 70% sequence identity to the reference sequence used for comparison. In some embodiments, such a sequence is at least about 80% sequence identity to the reference sequence used for comparison. In some embodiments, such a sequence is at least about 85% sequence identity to the reference sequence used for comparison. In some embodiments, such a sequence is at least about 90% sequence identity to the reference sequence used for comparison. In some embodiments, such a sequence is at least about 95% sequence identity to the reference sequence used for comparison. In some embodiments, such a sequence is at least about 99% sequence identity to the reference sequence used for comparison.

A “T cell” (or “T lymphocyte”) is an immune system cell that matures in the thymus and produces T cell receptors (TCRs), which can be obtained (enriched or isolated) from, for example, peripheral blood mononuclear cells (PBMCs) and are referred to herein as “bulk” T cells. After isolation of T cells, both cytotoxic (CD8+) and helper (CD4+) T cells can be sorted into naïve, memory, and effector T cell subpopulations, either before or after expansion. T cells can be naïve (not exposed to antigen; increased expression of CD62L, CCR7, CD28, CD3, CD127, and CD45RA, and decreased expression of CD45RO as compared to central memory T cell (TCM)), memory T cells (TM) (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or TCM). Effector T cells (TE) refers to antigen-experienced CD8+ cytotoxic T lymphocytes that has decreased expression of CD62L, CCR7, CD28, and are positive for granzyme and perforin as compared to TCM. Helper T cells (Th) are CD4+ cells that influence the activity of other immune cells by releasing cytokines. CD4+ T cells can activate and suppress an adaptive immune response, and which action is induced will depend on presence of other cells and signals. T cells can be collected in accordance with known techniques, and the various subpopulations or combinations thereof can be enriched or depleted by known techniques, such as by affinity binding to antibodies, flow cytometry, or immunomagnetic selection.

The term “T-cell epitope” as used herein is meant to refer to a peptide sequence which can be bound by the MHC molecules of class I or II in the form of a peptide-presenting MHC molecule or MHC complex and then, in this form, be recognized and bound by cytotoxic T-lymphocytes or T-helper cells, respectively.

A “T-cell population” can include thymocytes, immature T lymphocytes, mature T lymphocytes, resting T lymphocytes and activated T-lymphocytes. The T-cell population can include αβ T-cells, including CD4+ T-cells, CD8+ T cells, γδ T-cells, Natural Killer T-cells, or any other subset of T-cells.

The term “T cell receptor” (TCR), as used herein, refers to an immunoglobulin superfamily member (having a variable binding domain, a constant domain, a transmembrane region, and a short cytoplasmic tail; see, e.g., Janeway et al., Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains, a variable domain (e.g., α-chain variable domain or Va, β-chain variable domain or Vβ; typically amino acids 1 to 116 based on Kabat numbering, Kabat et al., “Sequences of Proteins of Immunological Interest,” US Dept. Health and Human Services, Public Health Service National Institutes of Health, 1991, 5th ed.) at the N-terminus, and one constant domain (e.g., α-chain constant domain or Ca, typically amino acids 117 to 259 based on Kabat, β-chain constant domain or Cp, typically amino acids 117 to 295 based on Kabat) adjacent to the cell membrane. Also like immunoglobulins, the variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, a TCR is found on the surface of T cells (or “T lymphocytes”) and associates with the CD3 complex. The source of a TCR as used in the disclosure may be from various animal species, such as a human, mouse, rat, rabbit or other mammal. In some embodiments, the source of a TCR as used in the disclosure is from the subject from which the neoantigen used for in vivo T cells activation and expansion is identified.

As used herein, the term “therapeutic” means an agent utilized to treat, combat, ameliorate, prevent or improve an unwanted condition or disease of a patient.

The term “therapeutic effect” as used herein is meant to refer to some extent of relief of one or more of the symptoms of a disorder (e.g., a neoplasia or tumor) or its associated pathology. A “therapeutically effective amount” as used herein is meant to refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the disclosure employed in a pharmaceutical composition at levels lower than that required in order to achieve the desired therapeutic effect and gradually increase the dosage until the desired effect is achieved. In some embodiments, the therapeutically effective amount is an amount effective to shrink a solid tumor by about 2% in total mass as compared to its mass or estimated mass before treatment, by about 4% in total mass, by about 6% in total mass, by about 8% in total mass, by about 10% in total mass, by about 15% in total mass, by about 20% in total mass, by about 25% in total mass, by about 30% in total mass, by about 35% in total mass, by about 40% in total mass, by about 45% in total mass, or by about 50% in total mass as compared to the total mass of the solid tumor before the treatment. In some embodiments, for any therapeutic agent described herein the therapeutically effective amount is initially determined from preliminary in vitro studies and/or animal models. In some embodiments, a therapeutically effective dose is determined from human data. In some embodiments, the applied dose is adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference.

The terms “treat,” “treated,” “treating,” “treatment,” and the like as used herein are meant to refer to reducing or ameliorating a disorder and/or symptoms associated therewith (e.g., a cancer or tumor). “Treating” may refer to administration of the neoantigen vaccines described herein to a subject after the onset, or suspected onset, of a cancer. “Treating” may also refer to administration of the engineered TCR transgenic T cells described herein to the subject after the onset, or suspected onset, of a cancer. “Treating” includes the concepts of “alleviating,” which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. Thus, as used herein, the term “treating cancer” is not intended to be an absolute term. In some aspects, the compositions and methods of the disclosure seek to reduce the size of a tumor or number of cancer cells, cause a cancer to go into remission, or prevent growth in size or cell number of cancer cells. In some circumstances, treatment with the compositions and methods of the disclosure leads to an improved prognosis.

As used herein, a “tumor antigen” or “tumor-associated antigen” or “TAA” refers to a mutated protein found in an oncogenic or tumor cell that elicits a humoral immune response, a cellular immune response, or both, which may be found only in tumor cells or may be found in tumor cells and other normal cells. In some embodiments, a TAA is a product of a mutated oncogene (e.g., p53, raf, ras, myc, EGFR). In some embodiments, a TAA is a mutated tumor suppressor gene (e.g., pRb, TP53, PTEN, CD95). In some embodiments, a TAA is a mutated gene that overexpresses or aberrantly expresses a cellular protein, or the like.

The term “vaccine” as used herein is meant to refer to a composition for generating immunity for the prophylaxis and/or treatment of diseases (e.g., cancer). Accordingly, vaccines are medicaments which comprise antigens and are intended to be used in humans or animals for generating specific defense and protective substance by vaccination. A “vaccine composition” or a “neoantigen vaccine composition” can include a pharmaceutically acceptable excipient, earner or diluent.

“Tumor infiltrating lymphocyte” or “TIL,” as used herein, refers to a type of immune cell that has moved from the blood into a tumor. Tumor-infiltrating lymphocytes are capable of recognizing and killing cancer cells. In some embodiments for cancer therapy, tumor-infiltrating lymphocytes are removed from a patient's tumor, grown in large numbers in vitro, and then administered back to the patient to help the immune system treat the subject and kill one or a plurality of cancer cells.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein.

The vector can comprise heterologous nucleic acid encoding a neoantigen and can further comprise an initiation codon, which can be upstream of the neoantigen coding sequence, and a stop codon, which can be downstream of the neoantigen coding sequence. The initiation and termination codon can be in frame with the neoantigen coding sequence. The vector can also comprise a promoter that is operably linked to the neoantigen coding sequence. The promoter operably linked to the neoantigen coding sequence can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the contents of which are incorporated herein in its entirety.

The vector can also comprise a polyadenylation signal, which can be downstream of the HA coding sequence. The polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal can be a polyadenylation signal from a pCEP4 vector (Invitrogen, San Diego, Calif.).

The vector can also comprise an enhancer upstream of the neoantigen coding. The enhancer can be necessary for DNA expression. The enhancer can be human actin, human myosin, human hemoglobin, human muscle creatine or a viral enhancer such as one from CMV, HA, RSV or EBV. Polynucleotide function enhances are described in U.S. Pat. Nos. 5,593,972, 5,962,428, and WO94/016737, the contents of each are fully incorporated by reference. The vector can also comprise a mammalian origin of replication in order to maintain the vector extrachromosomally and produce multiple copies of the vector in a cell.

### The Disclosure Relates to Pahramceitic

In some embodiments, the nucleic acid molecule or the pharmaceutical composition comprises a DNA backbone that comprises all of the lowercase basepairs from any of the above-identified plasmids, wherein a first lowercase backbone sequence and a second lowercase backbone sequence flank the expressible nucleic acid sequence encoding the plurality of tumor-specific antigen sequences, such as Formula I, Formula I (a), Formula II or Formula III(a).

In some embodiments, a nucleic acid molecule comprises a nucleic acid sequence comprising Formula I ([(AEDn)-(linker)]n-[AEDn+1]), wherein the each linker is independently selectable from about 0 to about 25 natural or non-natural nucleic acids in length. In some embodiments, a nucleic acid molecule comprises a nucleic acid sequence comprising Formula I ([(AEDn)-(linker)]n-[AEDn+1]), wherein the each linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25. In some embodiments, each linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is about 21 natural or non-natural nucleic acids in length. In certain embodiments, two linkers can be used together, in a fusion. Accordingly, in some embodiments, the first linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the second linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the first linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, the second linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length.

In some embodiments, the at least one linker comprises from about 15 to about 300 nucleotides and encodes a n amino acid cleavage site. In some embodiments, each linker positioned between each AED is the same nucleotide sequence comprising from about 15 to about 120 nucleotides and encodes an amino acid cleavage site

In some embodiments, the formula (e.g. [(AEDn)-(linker)]n-[AEDn+1]) comprises at least 1, 2, 3, 4, 5, 6, 7, 8, 9, 10 or more linkers.

In some embodiments, the vector can be LLC, TC1, ID8, pGX0001, pGX4501, pGX4503, pGX4504, pGX4505, pGX4506 and/or pGX6001 or any one or more regulatory or non-coding sequences of LLC, TC1, ID8, pGX0001, pGX4501, pGX4503, pGX4504, pGX4505, pGX4506 and/or pGX6001. In some embodiments, the vector comprises the sequence that is pVAX1. The backbone of the vector can be pAV0242. The vector can be a replication defective adenovirus type 5 (Ad5) vector.

In some embodiments, the vector comprises a regulatory sequence, which can be well suited for gene expression in a mammalian or human cell into which the vector is administered. The neoantigen coding sequence can comprise a codon, which can allow more efficient transcription of the coding sequence in the host cell.

In some embodiments, the vector is pSE420 (Invitrogen, San Diego, Calif.), which can be used for protein production in Escherichia coli (E. coli) further comprising an expressible nucleic acid. The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which can be used for protein production in mammalian cells such as Chinese hamster ovary (CHO) cells. The vector can be expression vectors or systems to produce protein by routine techniques and readily available starting materials including Sambrook et al., Molecular Cloning: A Laboratory Manual, Second Ed., Cold Spring Harbor (1989), which is incorporated fully by reference.

Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.). Vectors of the disclosure are those polynucleotide sequences, such as plasmids, that comprise nucleic acid sequences encoding one or a plurality of neoantigens or TCR sequences that highly associate with the one or plurality of neoantigens.

In some embodiment in which the nucleic acid molecules comprising an expressible nucleic acid sequence, the expressible nucleic acid sequence of Formula I is positioned within the multiple cloning site of a plasmid selected from the group consisting of LLC, TC1, ID8, pGX0001, pGX4501, pGX4503, pGX4504, pGX4505, pGX4506 and/or pGX6001. In some embodiments, the nucleic acid sequence of Formula I is positioned with the multiple cloning site of LLC. In some embodiments, the nucleic acid sequence of Formula I is positioned with the multiple cloning site of TC1. In some embodiments, the nucleic acid sequence of Formula I is positioned with the multiple cloning site of ID8. In some embodiments, the nucleic acid sequence of Formula I is positioned with the multiple cloning site of pGX0001. In some embodiments, the nucleic acid sequence of Formula I is positioned with the multiple cloning site of pGX4501. In some embodiments, the nucleic acid sequence of Formula I is positioned with the multiple cloning site of pGX4503. In some embodiments, the nucleic acid sequence of Formula I is positioned with the multiple cloning site of pGX4504. In some embodiments, the nucleic acid sequence of Formula I is positioned within the multiple cloning site of pGX4505. In some embodiments, the nucleic acid sequence of Formula I is positioned with the multiple cloning site of pGX4506. In some embodiments, the plasmid is pGX4505 or a sequence that is 70%, 80%, 90%, 91%, 92%, 93%, 94%, 95%, 96%, 97%, 98%, 99% homologous to each of the above-identified nucleotide sequences. In some embodiments, the plasmid is pGX0001 or a sequence that comprises about 70%, 80%, 90%, 91%, 92%, 93%, 94%, 95%, 96%, 97%, 98% or 99% sequence identity to SEQ ID NO:356. In some embodiments, the plasmid is pGX6001 or a sequence that comprises about 70%, 80%, 90%, 91%, 92%, 93%, 94%, 95%, 96%, 97%, 98% or 99% sequence identity to SEQ ID NO: 360.

TABLE W
Plasmid Backbone Sequences

| | |
|---|---|
| SEQ ID NO: 356 | gctgcttcgcgatgtacgggccagatatacgcgttgacattgattattgactagttattaatagtaatcaattacggggtcattagttc |
| pGX0001 full | atagcccatatatggagttccgcgttacataacttacggtaaatggcccgcctggctgaccgcccaacgacccccgcccattga |
| plasmid DNA | cgtcaataatgacgtatgttcccatagtaacgccaatagggactttccattgacgtcaatgggtggagtatttacggtaaactgccc |
| sequence: | acttggcagtacatcaagtgtatcatatgccaagtacgccccctattgacgtcaatgacggtaaatggcccgcctggcattatgcc |
| | cagtacatgaccttatgggactttcctacttggcagtacatctacgtattagtcatcgctattaccatggtgatgcggttttggcagta |
| | catcaatgggcgtggatagcggtttgactcacggggatttccaagtctccaccccattgacgtcaatgggagtttgttttggcacc |
| | aaaatcaacgggactttccaaaatgtcgtaacaactccgccccattgacgcaaatgggcggtaggcgtgtacggtgggaggtct |
| | atataagcagagctctctggctaactagagaacccactgcttactggcttatcgaaattaatacgactcactatagggagacccaa |
| | gctggctagcgtttaaacttaagcttggtaccgagctcggatccactagtccagtgtggtggaattctgcagatatccagcacagt |
| | ggggccgctcgagtctagagggcccgtttaaacccgctgatcagcctcgactgtgccttctagttgccagccatctgttgtttgc |
| | ccctcccccgtgccttccttgaccctggaaggtgccactcccactgtcctttcctaataaaatgaggaaattgcatcgcattgtctg |
| | agtaggtgtcattctattctggggggtgggggtgggcaggacagcaagggggaggattgggaagacaatagcaggcatgctg |
| | gggatgcggtgggctctatggcttctactgggcggttttatggacagcaagcgaaccggaattgccagctggggcgccctctg |
| | gtaaggttgggaagccctgcaaagtaaactggatggctttettgccgccaaggatctgatggcgcaggggatcaagctctgatc |
| | aagagacaggatgaggatcgtttcgcatgattgaacaagatggattgcacgcaggttctccggccgcttgggtggagaggctat |
| | tcggctatgactgggcacaacagacaatcggctgctctgatgccgccgtgttccggctgtcagcgcaggggcgcccggttcttt |
| | ttgtcaagaccgacctgtccggtgccctgaatgaactgcaagacgaggcagcgcggctatcgtggctggccacgacgggcgt |
| | tccttgcgcagctgtgctcgacgttgtcactgaagcgggaagggactggctgctattgggcgaagtgccggggcaggatetcc |
| | tgtcatctcaccttgctcctgccgagaaagtatccatcatggctgatgcaatgcggcggctgcatacgcttgatccggctacctgc |
| | ccattcgaccaccaagcgaaacatcgcatcgagcgagcacgtactoggatggaagccggtcttgtcgatcaggatgatctgga |
| | cgaagagcatcaggggctcgcgccagccgaactgttcgccaggctcaaggcgagcatgcccgacggcgaggatctcgtcgt |
| | gacccatggcgatgcctgcttgccgaatatcatggtggaaaatggccgcttttctggattcatcgactgtggccggctgggtgtg |
| | gcggaccgctatcaggacatagcgttggctacccgtgatattgctgaagagcttggcggcgaatgggctgaccgcttcctcgtg |
| | ctttacggtatcgccgctcccgattcgcagcgcatcgccttctategccttcttgacgagttcttctgaattattaacgcttacaatttc |
| | ctgatgcggtattttctccttacgcatctgtgcggtatttcacaccgcatcaggtggcacttttcggggaaatgtgcgeggaacccc |
| | tatttgtttatttttctaaatacattcaaatatgtatccgctcatgagacaataaccctgataaatgcttcaataatagcacgtgctaaaa |
| | cttcatttttaatttaaaaggatctaggtgaagatcctttttgataatctcatgaccaaaatcccttaacgtgagttttcgttccactgag |
| | cgtcagaccccgtagaaaagatcaaaggatcttcttgagatcctttttttctgcgcgtaatctgctgcttgcaaacaaaaaaaccac |
| | cgctaccagcggtggtttgtttgccggatcaagagctaccaactctttttccgaaggtaactggcttcagcagagegcagatacc |
| | aaatactgttcttctagtgtagccgtagttaggccaccacttcaagaactctgtagcaccgcctacatacctcgctctgctaatcctg |
| | ttaccagtggctgctgccagtggcgataagtcgtgtcttaccgggttggactcaagacgatagttaccggataaggcgcagcgg |
| | tcgggctgaacggggggttcgtgcacacagcccagcttggagcgaacgacctacaccgaactgagatacctacagcgtgag |
| | ctatgagaaagcgccacgcttcccgaagggagaaaggcggacaggtatccggtaagcggcagggtcggaacaggagagc |
| | gcacgagggagcttccagggggaaacgcctggtatctttatagtcctgtcgggtttcgccacctctgacttgagcgtcgatttttgt |
| | gatgctcgtcaggggggcggagcctatggaaaaacgccagcaacgcggcctttttacggttcctggccttttgctggccttttgc |
| | tcacatgttctt |
| SEQ ID NO: 357 | Gctgcttcgcgatgtacgggccagatatacgcgttgacattgattattgactagttattaatagtaatcaattacggggtcattagtt |
| pGX0001 | catagcccatatatggagttccgcgttacataacttacggtaaatggcccgcctggctgaccgcccaacgacccccgcccattg |
| first DNA | acgtcaataatgacgtatgttcccatagtaacgccaatagggactttccattgacgtcaatgggtggagtatttacggtaaactgcc |
| backbone | cacttggcagtacatcaagtgtatcatatgccaagtacgccccctattgacgtcaatgacggtaaatggcccgcctggcattatgc |
| | ccagtacatgaccttatgggactttcctacttggcagtacatctacgtattagtcatcgctattaccatggtgatgcggttttggcagt |
| | acatcaatgggcgtggatagcggtttgactcacggggatttccaagtctccaccccattgacgtcaatgggagtttgttttggcac |
| | caaaatcaacgggactttccaaaatgtcgtaacaactccgccccattgacgcaaatgggcggtaggcgtgtacggtgggaggt |
| | ctatataagcagagctctctggctaactagagaacccactgcttactggcttatcgaaattaatacgactcactatagggagaccc |
| | aagctg |
| SEQ ID NO: 358 | ccgctgatcagcctcgactgtgccttctagttgccagccatctgttgtttgcccctcccccgtgccttccttgaccctggaaggtgc |
| pGX0001 | cactcccactgtcctttcctaataaaatgaggaaattgcatcgcattgtctgagtaggtgtcattctattctggggggggggggg |
| Second DNA | gcaggacagcaagggggaggattgggaagacaatagcaggcatgctggggatgcggtgggctctatggcttctactgggcg |
| backbone | gttttatggacagcaagcgaaccggaattgccagctggggcgccctctggtaaggttgggaagccctgcaaagtaaactggat |
| | ggctttcttgccgccaaggatctgatggcgcaggggatcaagctctgatcaagagacaggatgaggatcgtttcgcatgattga |
| | acaagatggattgcacgcaggttctccggccgcttgggtggagaggctattcggctatgactgggcacaacagacaatcggct |
| | gctctgatgccgccgtgttccggctgtcagcgcaggggcgcccggttctttttgtcaagaccgacctgtccggtgccctgaatga |
| | actgcaagacgaggcagcgcggctatcgtggctggccacgacgggcgttccttgcgcagctgtgctcgacgttgtcactgaag |
| | cgggaagggactggctgctattgggcgaagtgccggggcaggatctcctgtcatctcaccttgctcctgccgagaaagtatcca |
| | tcatggctgatgcaatgcggcggctgcatacgcttgatccggctacctgcccattcgaccaccaagegaaacategcatcgagc |
| | gagcacgtactcggatggaagccggtcttgtcgatcaggatgatctggacgaagagcatcaggggctegcgccagccgaact |
| | gttcgccaggctcaaggcgagcatgcccgacggcgaggatctcgtcgtgacccatggcgatgcctgcttgccgaatatcatgg |
| | tggaaaatggccgcttttctggattcatcgactgtggccggctgggtgtggcggaccgctatcaggacatagcgttggctaccc |
| | gtgatattgctgaagagcttggcggcgaatgggctgaccgcttcctcgtgctttacggtatcgccgctcccgattcgcagcgcat |
| | cgccttctatcgccttcttgacgagttcttctgaattattaacgcttacaatttcctgatgcggtattttctccttacgcatctgtgcggta |
| | tttcacaccgcatcaggtggcacttttcggggaaatgtgcgcggaacccctatttgtttatttttctaaatacattcaaatatgtatccg |
| | ctcatgagacaataaccctgataaatgcttcaataatagcacgtgctaaaacttcatttttaatttaaaaggatctaggtgaagatcct |
| | ttttgataatctcatgaccaaaatcccttaacgtgagttttcgttccactgagcgtcagaccccgtagaaaagatcaaaggatcttct |
| | tgagatcctttttttctgcgcgtaatctgctgcttgcaaacaaaaaaaccaccgctaccagcggtggtttgtttgccggatcaagag |
| | ctaccaactctttttccgaaggtaactggcttcagcagagcgcagataccaaatactgttcttctagtgtagccgtagttaggccac |
| | cacttcaagaactctgtagcaccgcctacatacctcgctctgctaatcctgttaccagtggctgctgccagtggcgataagtcgtg |
| | tcttaccgggttggactcaagacgatagttaccggataaggcgcagcggtcgggctgaacggggggttcgtgcacacagccc |
| | agcttggagcgaacgacctacaccgaactgagatacctacagcgtgagctatgagaaagcgccacgcttcccgaagggaga |
| | aaggcggacaggtatccggtaagcggcagggtcggaacaggagagcgcacgagggagcttccagggggaaacgcctggt |
| | atctttatagtcctgtcgggtttcgccacctctgacttgagcgtcgatttttgtgatgctcgtcaggggggcggagcctatggaaaa |
| | acgccagcaacgcggcctttttacggttcctggccttttgctggccttttgctcacatgttctt |
| SEQ ID NO: 359 | Gctagcgtttaaacttaagcttggtaccgagctcggatccactagtccagtgtggtggaattctgcagatatccagcacagtggc |
| Multiple | ggccgctcgagtctagagggcccgtttaaac |
| cloning site | |
| SEQ ID NO: 360 | aaatgggggcgctgaggtctgcctcgtgaagaaggtgttgctgactcataccaggcctgaatcgccccatcatccagccagaa |
| pGX6001 full | agtgagggagccacggttgatgagagctttgttgtaggtggaccagttggtgattttgaacttttgctttgccacggaacggtctgc |
| plasmid | gttgtcgggaagatgcgtgatctgatccttcaactcagcaaaagttcgatttattcaacaaagccgccgtcccgtcaagtcagcgt |
| sequence | aatgctctgccagtgttacaaccaattaaccaattctgcgttcaaaatggtatgcgttttgacacatccactatatatccgtgtcgttct |
| | gtccactcctgaatcccattccagaaattctctagcgattccagaagtttctcagagtcggaaagttgaccagacattacgaactg |
| | gcacagatggtcataacctgaaggaagatctgattgcttaactgcttcagttaagaccgacgcgctcgtcgtataacagatgcga |
| | tgatgcagaccaatcaacatggcacctgccattgctacctgtacagtcaaggatggtagaaatgttgtcggtccttgcacacgaat |
| | attacgccatttgcctgcatattcaaacagctcttctacgataagggcacaaatcgcatcgtggaacgtttgggcttctaccgattta |
| | gcagtttgatacactttctctaagtatccacctgaatcataaatcggcaaaatagagaaaaattgaccatgtgtaagcggccaatct |
| | gattccacctgagatgcataatctagtagaatctcttcgctatcaaaattcacttccaccttccactcaccggttgtccattcatggct |
| | gaactctgcttcctctgttgacatgacacacatcatctcaatatccgaatacggaccatcagtctgacgaccaagagagccataaa |
| | caccaatagccttaacatcatccccatatttatccaatattcgttccttaatttcatgaacaatcttcattctttcttctctagtcattattatt |
| | ggtccgttcataacaccccttgtattactgtttatgtaagcagacagttttattgttcatgatgatatatttttatcttgtgcaatgtaacat |
| | cagagattttgagacacaacgtggctttccccggcccatgaccaaaatcccttaacgtgagttttcgttccactgagcgtcagacc |
| | ccgtagaaaagatcaaaggatcttcttgagatcctttttttctgcgcgtaatctgctgcttgcaaacaaaaaaaccaccgctaccag |
| | cggtggtttgtttgccggatcaagagctaccaactctttttccgaaggtaactggcttcagcagagcgcagataccaaatactgttc |
| | ttctagtgtagccgtagttaggccaccacttcaagaactctgtagcaccgcctacatacctcgctctgctaatcctgttaccagtgg |
| | ctgctgccagtggcgataagtcgtgtcttaccgggttggactcaagacgatagttaccggataaggcgcagcggtcgggctga |
| | acggggggttcgtgcacacagcccagcttggagcgaacgacctacaccgaactgagatacctacagcgtgagctatgagaaa |
| | gcgccacgcttcccgaagggagaaaggcggacaggtatccggtaagcggcagggtcggaacaggagagcgcacgaggg |
| | agcttccagggggaaacgcctggtatctttatagtcctgtcgggtttcgccacctctgacttgagcgtcgatttttgtgatgctcgtc |
| | aggggggcggagcctatggaaaaacgccagcaacgeggcctttttacggttcctggccttttgetggccttttgctcacatgttct |
| | ttcctgcgttatcccctgattctgtggataaccgtattaccgcctttgagtgagctgataccgctcgccgcagccgaacgaccgag |
| | cgcagcgagtcagtgagcgaggaagcggaagagcgcctgatgcggtattttctccttacgcatctgtgcggtatttcacaccgc |
| | atatggtgcactctcagtacaatctgctctgatgccgcatagttaagccagtatctgctccctgcttgtgtgttggaggtcgctgagt |
| | agtgcgcgagcaaaatttaagctacaacaaggcaaggcttgaccgacaattgcatgaagaatctgcttagggttaggcgttttgc |
| | gctgcttcgcgatgtacgggccagatatagccgcggcatcgatgataattcggcttatttaaattccccagcatgcctgctattgtc |
| | ttcccaatcctcccccttgctgtcctgccccaccccaccccccagaatagaatgacacctactcagacaatgcgatgcaatttcct |
| | cattttattaggaaaggacagtgggagtggcaccttccagggtcaaggaaggcacgggggaggggcaaacaacagatggct |
| | ggcaactagaaggcacagtcgaggctgatcagcgagctcggcgcgcctctcgagttactaactgcagggcacagatgcccat |
| | tcgctccaagatgagctatagtageggtcctgggcccgcacgctaatgetggcatttttgcggcagatgaccgtggetgaggtct |
| | tgtccgtgaagactctatctttcttttctctcttgctcttgccctggacctgaacgcagaatgtcagggagaagtaggaatgtggagt |
| | actccaggtgtcagggtactcccagctgacctccacctgccgagaattctttaatggcttcagctgcaagttcttgggtgggtcag |
| | gtttgatgatgtccctgatgaagaagctgctggtgtagttttcatacttgagcttgtgaacggcatccaccatgacctcaatgggca |
| | gactctcctcagcagctgggcaggcactgtcctcctggcactccactgagtactcatactccttgttgtcccctctgactctctctg |
| | cagagagtgtagcagctccgcacgtcaccccttgggggtcagaagagcctctgctgcttttgacactgaatgtcaaatcagtact |
| | gattgtcgtcagccaccagcaggtgaaacgtccagaataattcttggcctcgcatcttagaaaggtcttatttttgggttctttctggt |
| | cctttaaaatatcagtggaccaaattccatcttcctttttgtgaagcagcaggagcgaatggcttagaacctcgcctcctttgtgaca |
| | ggtgtactggccagcatctccaaactctttgacttggatggtcagggttttgccagagcctaagacctcactgctctggtccaagg |
| | tccaggtgataccatcttcttcaggggtgtcacaggtgaggaccaccatttctccaggggcatccggataccaatccaattctacg |
| | acataaacatctttcttcagttcccatatggccacgaggggagatgccagaaaaaccagggaaaaccaagagatgaccaactg |
| | ctggtgacacatggtggctcgagtgggcccaagtttaaacgctcctccgacgtccccaggcagaatggcggttccctaaacga |
| | gcattgcttatatagacctcccattaggcacgcctaccgcccatttacgtcaatggaacgcccatttgegtcattgcccctccccat |
| | tgacgtcaatggggatgtacttggcagccatcgcgggccatttaccgccattgacgtcaatgggagtactgccaatgtaccctgg |
| | cgtacttccaatagtaatgtacttgccaagttactattaatagatattgatgtactgccaagtgggccatttaccgtcattgacgtcaa |
| | tagggggcgtgagaacggatatgaatgggcaatgagccatcccattgacgtcaatggtgggtggtcctattgacgtcaatgggc |
| | attgagccaggcgggccatttaccgtaattgacgtcaatgggggaggcgccatatacgtcaataggaccgcccatatgacgtca |
| | ataggaaagaccatgctaagccgaattatcgcggctatctgaggggactagggtgtgtttaggcgaaaagcggggcttcggttg |
| | tacgcggttaggagtcccctcaggatatagtagtttcgcttttgcatagggagggggaaatgtagtcttatgcaatactcttgtagtc |
| | ttgcaacatggtaacgatgagttagcaacatgccttacaaggagagaaaaagcaccgtgcatgccgattggtggaagtaaggt |
| | ggtacgatcgtgccttattaggaaggcaacagacgggtctgacatggattggacgaaccactgaattccgcattgcagagatatt |
| | gtatttaagtgcctagctcgatacaataaacgccatttgaccattcaccacattggtgtgcacctccaagcttcgaccaattctcatg |
| | tttgacagcttatcatcgcagatccgggcaacgttgttgccattgctgcaggcgcagaactggtaggtatggaagatctatacatt |
| | gaatcaatattggcaattagccatattagtcattggttatatagcataaatcaatattggctattggccattgcatacgttgtatctatat |
| | cataatatgtacatttatattggctcatgtccaatatgaccgccatgttgacattgattattgactagttattaatagtaatcaattacgg |
| | ggtcattagttcatagcccatatatggagttccgcgttacataacttacggtaaatggcccgcctggctgaccgcccaacgaccc |
| | ccgcccattgacgtcaataatgacgtatgttcccatagtaacgccaatagggactttccattgacgtcaatgggtggagtatttacg |
| | gtaaactgcccacttggcagtacatcaagtgtatcatatgccaagtcegccccctattgacgtcaatgacggtaaatggccegcc |
| | tggcattatgcccagtacatgaccttacgggactttcctacttggcagtacatctacgtattagtcatcgctattaccatggtgatgc |
| | ggttttggcagtacatcaatgggcgtggatagcggtttgactcacggggatttccaagtctccaccccattgacgtcaatgggagt |
| | ttgttttggcaccaaaatcaacgggactttccaaaatgtcgtaacaactccgccccattgacgcaaatgggcggtaggcgtgtac |
| | ggtgggaggtctatataagcagagctcgtttagtgaaccgtcagatcgcctggagacgccatccacgctgttttgacctccatag |
| | aagacaccgggaccgatccagcctccgcgggcgcgcgtcgaccaccatgtgtccagcgcgcagcctcctccttgtggctacc |
| | ctggtcctcctggaccacctcagtttggccagaaacctccccgtggccactccagacccaggaatgttcccatgccttcaccact |
| | cccaaaacctgctgagggccgtcagcaacatgctccagaaggccagacaaactctagaattttacccttgcacttctgaagaga |
| | ttgatcatgaagatatcacaaaagataaaaccagcacagtggaggcctgtttaccattggaattaaccaagaatgagagttgcct |
| | aaattccagagagacctctttcataactaatgggagttgcctggcctccagaaagacctcttttatgatggccctgtgccttagtagt |
| | atttatgaagacttgaagatgtaccaggtggagttcaagaccatgaatgcaaagcttctgatggatcctaagaggcagatctttcta |
| | gatcaaaacatgctggcagttattgatgagctgatgcaggccctgaatttcaacagtgagactgtgccacaaaaatcctcccttga |
| | agaaccggatttttataaaactaaaatcaagctctgcatacttcttcatgctttcagaattcgggcagtgactattgatagagtgatga |
| | gctatctgaatgcttcctaatagacgcgtaaaaagatccagacatgataagatacattgatgagtttggacaaaccacaactagaa |
| | tgcagtgaaaaaaatgctttatttgtgaaatttgtgatgctattgctttatttgtaaccattataagctgcaataaacaagttaacaaca |
| | acaattgcattcattttatgtttcaggttcagggggaggtgtgggaggtttttt |

The disclosure also relates to a nucleic acid molecule comprising a first nucleic acid sequence and a second nucleic acid sequence, wherein the first nucleic acid sequence is a DNA backbone domain of the nucleic acid molecule and the second nucleic acid sequence is an expressible nucleic acid sequence; wherein the expressible nucleic acid sequence comprises a plurality of antigen expression domains, in 5′ to 3′ orientation. In some embodiments, the expressible nucleic acid sequence comprises a nucleic acid sequence encoding a linker at the 5′ end of the first antigen expression domain. In some embodiments, the expressible nucleic acid sequence encodes a linker between each of the antigen expression domains. In some embodiments, the expressible nucleic acid sequence encodes a leader sequence, a plurality of antigen expression domains, each antigen expression domain separated by a linker sequence. In some embodiments, there are at least 20 antigen expression domains. In some embodiments, there are at least 20, 25, 30, 35, 40, 45, 50, 55, 60, 65 or 70 or more antigen expression domains. In some embodiments, the nucleic acid molecule comprises Formula I, Ia, II, IIa, or IIIa. In some embodiments, the nucleic acid molecule comprises one or a plurality of regulatory sequences operably linked to the expressible nucleic acid sequence. In some embodiments, the first DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:356 or a functional fragment that comprises at least about 70%, 75%, 80%, 85%, 90%, 91%, 92%, 93%, 94%, 95%, 96%, 97%, 98%, 99% sequence identity to SEQ ID NO:356. In some embodiments, the first DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:360 or a functional fragment that comprises at least about 70%, 75%, 80%, 85%, 90%, 91%, 92%, 93%, 94%, 95%, 96%, 97%, 98%, 99% sequence identity to SEQ ID NO:360.

The disclosure also relates to a nucleic acid molecule comprising a first, second and third nucleic acid sequence, wherein the first nucleic acid sequence is a first DNA backbone domain of the nucleic acid molecule, the second nucleic acid sequence is the second DNA backbone domain of the nucleic acid molecule and the third nucleic acid sequence is an expressible nucleic acid sequence; wherein the expressible nucleic acid sequence comprises a plurality of antigen expression domains, in 5′ to 3′ orientation. In some embodiments, the expressible nucleic acid sequence comprises a nucleic acid sequence encoding a linker at the 5′ end of the first antigen expression domain. In some embodiments, the expressible nucleic acid sequence encodes a linker between each of the antigen expression domains. In some embodiments, the expressible nucleic acid sequence encodes a leader sequence, a plurality of antigen expression domains, each antigen expression domain separated by a linker sequence. In some embodiments, there are at least 20 antigen expression domains. In some embodiments, there are at least 20, 25, 30, 35, 40, 45, 50, 55, 60, 65 or 70 or more antigen expression domains. In some embodiments, the nucleic acid molecule comprises Formula I, Ia, II, IIa, or IIIa. In some embodiments, the nucleic acid molecule comprises one or a plurality of regulatory sequences operably linked to the expressible nucleic acid sequence.

In some embodiments, the first DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:357 or a functional fragment that comprises at least about 70%, 75%, 80%, 85%, 90%, 91%, 92%, 93%, 94%, 95%, 96%, 97%, 98%, 99% sequence identity to SEQ ID NO:357. In some embodiments, the second DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:358 or a functional fragment that comprises at least about 70%, 75%, 80%, 85%, 90%, 91%, 92%, 93%, 94%, 95%, 96%, 97%, 98%, 99% sequence identity to SEQ ID NO:358.

In some embodiments, compositions or pharmaceutical compositions administered to a subject to elicit an antigen-specific immune response again one or a plurality of neoantigens comprise a nucleic acid molecule comprising: (i) a first DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:357 or a functional fragment that comprises at least about 70%, 75%, 80%, 85%, 90%, 91%, 92%, 93%, 94%, 95%, 96%, 97%, 98%, 99% sequence identity to SEQ ID NO:357; (ii) a second DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:358 or a functional fragment that comprises at least about 70%, 75%, 80%, 85%, 90%, 91%, 92%, 93%, 94%, 95%, 96%, 97%, 98%, 99% sequence identity to SEQ ID NO:358; and (iii) a third nucleic acid positioned between each of the first and second backbone sequences, wherein the third nucleic acid sequence is an expressible nucleic acid sequence encoding a plurality of neoantigens. In some embodiments the third nucleic acid sequence comprises an expressible nucleic acid sequence encoding at least about 20, 25, 30, 35, 40, 45, 50 55, or 60 or more neoantigens. In some embodiments, the expressible nucleic acid sequence comprises a nucleic acid sequence encoding, in 5′ to 3′ orientation, an Ig leader sequence and a plurality of antigen expression domains, wherein the antigen expression domains are separated by a linker. In some embodiments, the antigen expression domain consist of a single antigen sequence that encodes a tumor-specific neoantigens of the subject. In some embodiments, each antigen expression domain is from about 25 to about 40 nucleotides in length. In some embodiments, a nucleic acid molecule comprises a nucleic acid sequence comprising Formula I ([(AEDn)-(linker)]n-[AEDn+1]), wherein the each linker is independently selectable from about 0 to about 25 natural or non-natural nucleic acids in length and wherein n is a positive integer from about 10 to about 40.

In some embodiments, the nucleic acid sequence comprises at least 2, 3, 4, 5, 6, 7, 8, 9, or 10 or more linker domains and the nucleic acid sequence comprises Formula IV:

$$[ ( AEDn ) - ( linker ) ] n - ( AEDn + 1 ) - ( linker ) n + 1 ] n - AEED ( 3 ’ terminal )$$

wherein each AED is independently selectable from any one or plurality of tumor associated antigens from a subject and wherein n is any positive integer from about 1 to about 50 and wherein each “linker” is a nucleic acid sequence encoding one or a plurality of amino acid cleavage sites. Each linker may be the same or independently selectable to comprise one or a plurality of the linkers disclosed herein. In some embodiments, the linker is a furin cleavage site from about 9 to about 105 nucleotides in length and encodes an amino acid sequence that is an amino acid cleavage site. In some embodiments, the nucleic acid sequence is a component of a nucleic acid molecule. In some compositions contemplated herein, the composition comprises 1, 2, 3, 4, 5, or more nucleic acid molecules each of which expressing any of the patterns or formulae of AEDs disclosed herein.

In some embodiments, the experssible nucleic acid sequence comprises at least 2, 3, 4, 5, 6, 7, 8, 9, or 10 or more linker domains and the nucleic acid sequence comprises Formula III(a):

$$Leader Sequence - [ ( AEDn ) - ( linker ) ] n - ( AEDn + 1 ) - linker n + 1 - ( AEDn + 2 ) ] n$$

wherein each AED is independently selectable from any one or plurality of tumor associated antigens from a subject and wherein n is any positive integer from about 20 to about 50 and wherein each “linker” is a nucleic acid sequence encoding one or a plurality of amino acid cleavage sites. Each linker may be the same or independently selectable to comprise one or a plurality of the linkers disclosed herein; and wherein each “-” represents a bond between each subunit. In some embodiments, the linker is a furin cleavage site from about 9 to about 105 nucleotides in length and encodes an amino acid sequence that is an amino acid cleavage site. In some embodiments, the nucleic acid sequence is a component of a nucleic acid molecule. In some embodiments, the Formula III(a) comprises a third linker bonded to the 3′ end of third AED sequence. In some embodiments, the last AED sequence in 5′ to 3′ orientation free of a bond to a linker on its 3′ terminal end.

The disclosures also relates to a nucleic acid sequence comprising a coding region and a non-coding region, the coding region consisting of the Formula I (b):

$$[ ( AED 1 ) - ( linker ) - ( AED 2 ) - ( linker ) ] n - [ ( AED 3 ) ] n + 1 ,$$

wherein n is a positive integer from about 1 to about 30, wherein each “linker” encodes one or a plurality of amino acid cleavages sequences, and wherein the non-coding region comprises at least one regulatory sequence operably linked to one or more AEDs; and wherein, in the 5′ to 3′ orientation, AED3 is the terminal antigen expression domain in a sequence of AEDs. In some embodiments, n is 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more, and wherein AED1 and AED2 are each an antigen expression domain that is independently selectable as an antigen sequence. In some embodiments, the regulatory sequence is any of the regulatory sequences depicted in the Figures or a functional fragment that comprises at least 70%, 75%, 80%, 85%, 90%, 91%, 92%, 93%, 94%, 95%, 96% 97%, 98% or 99% sequence identity to the regulatory sequence depicted in the Figures.

The disclosures also relates to a nucleic acid sequence comprising a coding region and a non-coding region, the coding region consisting of the Formula I (b):

$$Ig Leader Sequence - [ ( AED ) - ( linker ) - ( AED ) - ( linker ) ] n - [ ( AED ) ] n + 1 ,$$

wherein n is a positive integer from about 1 to about 30, wherein each “linker” encodes one or a plurality of amino acid cleavages sequences, and wherein the non-coding region comprises at least one regulatory sequence operably linked to one or more AEDs; and wherein, in the 5′ to 3′ orientation, AED3 is the terminal antigen expression domain in a sequence of AEDs. In some embodiments, n is 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more, and wherein AED1 and AED2 are each an antigen expression domain that is independently selectable as an antigen sequence. In some embodiments, the regulatory sequence is any of the regulatory sequences depicted in the Figures or a functional fragment that comprises at least 70%, 75%, 80%, 85%, 90%, 91%, 92%, 93%, 94%, 95%, 96% 97%, 98% or 99% sequence identity to the regulatory sequence depicted in the Figures.

In some embodiments, compositions of the disclosure relate to any nucleic acid molecule comprising (i) at least 70%, 80% or 90% sequence identity to any plasmid backbone identified herein; and (ii) an expressible nucleic acid sequence comprising one or a plurality of antigen expression domains separated by nucleic acid sequences that encode protein cleavage sites. In some embodiments, the expressible nucleic acid sequence encodes from about 19 to about 60 different amino acid sequences that are antigens. In some embodiments, the disclosure relates to cells comprising nucleic acid molecule comprising (i) at least 70%, 80% or 90% sequence identity to any plasmid backbone identified herein; and (ii) an expressible nucleic acid sequence comprising one or a plurality of antigen expression domains separated by nucleic acid sequences that encode protein cleavage sites. In some embodiments, the disclosure relates to cells comprising one or a plurality of amino acid sequences encoded by the one or plurality of antigen expression domains, wherein the amino acid sequences are cleaved upon expression and exposure to intracellular protease activity.

### Identification of Neoantigens

The disclosure relates to methods of treating a cancer comprising one or a plurality of neoantigens in a subject in need thereof. The disclosure further relates to methods of preventing metastases of a cancer comprising one or a plurality of neoantigens in a subject in need thereof. Because tumor neoantigens often arise from somatic “passenger” mutations in the DNA of tumor cells, many tumor neoantigens are unique to an individual patient's cancer. Therefore, for the disclosed methods to proceed, one or a plurality of cancer-specific neoantigens it is necessary to determine the “mutanome” of the subject's tumor and identify candidate tumor neoantigens. In some embodiments therefore, the methods of the disclosure comprises identifying one or a plurality of neoantigens that are unique to the subject. In some embodiments, a neoantigen comprises an antigenic peptide or epitope from a protein encoded by a nucleic acid molecule having a missense mutation, nonstop mutation, splice variant, gene fusion, frameshift mutation (e.g., addition or deletion), or combinations thereof, as compared to the wild-type nucleic acid molecule.

Neoantigens may be identified using any of several well-known techniques (see, e.g., Rajasagi et al., Blood 124:453, 2014). For example, in some embodiments, a population of tumor-specific neoantigens is identified by sequencing the tumor DNA (or RNA) and DNA (or RNA) from normal tissue of each patient to identify tumor-specific mutations and determining the patient's HLA allotype. In some embodiments, the population of tumor specific neoantigens and their cognate native antigens are subject to bioinformatic analysis using validated algorithms to predict which tumor-specific mutations create epitopes that could bind to the patient's HLA allotype, and in particular which tumor-specific mutations create epitopes that could bind to the patient's HLA allotype more effectively than the cognate native antigen. Based on this analysis, identified nucleotide sequences corresponding to these mutations are designed for each patient, and used together as a cancer vaccine to immunize the subject in some embodiments.

In some embodiments, the methods of the disclosure comprises a step of identifying one or more subject-specific neoantigen mutations in the subject, wherein the subject has been diagnosed with, suspected of having, or comprises one or more hyperproliferative cells (e.g. such as a tumor, the step comprising (a) sequencing a nucleic acid sample from the tumor of the subject and a nucleic acid sample from a non-tumor sample of the subject; (b) analyzing the sequences to determine coding and non-coding regions; (c) identifying sequences comprising tumor-specific non-synonymous or non-silent mutations not present in the non-tumor sample; (d) identifying single nucleotide variations and single nucleotide insertions and deletions; (e) producing subject-specific peptides encoded by the sequences comprising tumor-specific non-synonymous or non-silent mutations not present in the non-tumor sample; and (f) measuring the binding characteristics of the subject-specific peptides, wherein each subject-specific peptide is an expression product of subject-specific DNA neoantigen not present in the non-tumor sample, thereby identifying one or more subject-specific DNA neoantigens in a subject.

Any cell type or tissue may be utilized to obtain nucleic acid samples for use in the sequencing methods described herein. In some embodiments, the DNA or RNA sample is obtained from a neoplasia, a tumor or a bodily fluid, e.g., blood, obtained by known techniques (e.g. venipuncture) or saliva. In other embodiments, nucleic acid tests can be performed on dry samples (e.g. hair or skin).

The subject-specific neoantigen mutations may be any mutation in any gene which encodes a mutated amino acid sequence (also referred to as a “non-silent mutation”) and which is expressed in a cancer cell but not in a normal, noncancerous cell. Non-limiting examples of cancer-specific mutations that may be identified in the disclosed methods include missense, nonsense, insertion, deletion, duplication, frameshift, and repeat expansion mutations. In some embodiments, the disclosed method comprises identifying at least one gene containing a cancer-specific mutation which encodes a mutated amino acid sequence. However, the number of genes containing such a cancer-specific mutation that may be identified in the disclosed methods is not limited and may include more than one gene (for example, about 2, about 3, about 4, about 5, about 10, about 11, about 12, about 13, about 14, about 15, about 20, about 25, about 30, about 40, about 50, about 60, about 70, about 80, about 90, about 100, about 150, about 200, about 400, about 600, about 800, about 1000, about 1500, about 2000 or more, or a range defined by any two of the foregoing values). Likewise, in some embodiments, the disclosed methods comprise identifying at least one cancer-specific mutation which encodes a mutated amino acid sequence. However, the number of such cancer-specific mutations that may be identified in the disclosed methods is not limited and may include more than one cancer-specific mutation (for example, about 2, about 3, about 4, about 5, about 10, about 11, about 12, about 13, about 14, about 15, about 20, about 25, about 30, about 40, about 50, about 60, about 70, about 80, about 90, about 100, about 150, about 200, about 400, about 600, about 800, about 1000, about 1500, about 2000 or more, or a range defined by any two of the foregoing values). In some embodiments in which more than one cancer-specific mutation is identified, the cancer-specific mutations are located in the same gene. In other embodiments in which more than one cancer-specific mutation is identified, the cancer-specific mutations are located in different genes.

In some embodiments, identifying one or more cancer-specific mutations in the nucleic acid of a cancer cell comprises sequencing a complete or substantially complete exome, a whole genome, or whole transcriptome of the cancer cell. Sequencing may be carried out in any suitable manner known in the art. Examples of sequencing techniques that can be useful in the disclosed methods include, but not limited to, Next Generation Sequencing (NGS) (also referred to as “massively parallel sequencing technology”) or Third Generation Sequencing. NGS refers to non-Sanger-based high-throughput DNA sequencing technologies. With NGS, millions or billions of DNA strands may be sequenced in parallel, yielding substantially more throughput and minimizing the need for the fragment-cloning methods that are often used in Sanger sequencing of genomes. In NGS, nucleic acid templates may be randomly read in parallel along the entire genome by breaking the entire genome into small pieces. NGS may, advantageously, provide nucleic acid sequence information of a whole genome, exome, or transcriptome in very short time periods, e.g., within about 1 to about 2 weeks, within about 1 to about 7 days, or within less than about 24 hours. Multiple NGS platforms which are commercially available or which are described in the literature can be used in the context of the disclosed methods, e.g., those described in Zhang et al., J. Genet. Genomics, 38 (3): 95-109 (2011) and Voelkerding et al., Clinical Chemistry, 55:641-658 (2009).

Non-limiting examples of NGS technologies and platforms include sequencing-by-synthesis (also known as “pyrosequencing”) (as implemented, e.g., using the GS-FLX 454 Genome Sequencer, 454 Life Sciences (Branford, Conn.), ILLUMINA SOLEXA Genome Analyzer (Illumina Inc., San Diego, Calif.), or the ILLUMINA HISEQ 2000 Genome Analyzer (Illumina), or as described in, e.g., Ronaghi et al., Science, 281 (5375): 363-365 (1998)), sequencing-by-ligation (as implemented, e.g., using the SOLID platform (Life Technologies Corporation, Carlsbad, Calif.) or the POLONATOR G.007 platform (Dover Systems, Salem, N.H.)), single-molecule sequencing (as implemented, e.g., using the PACBIO RS system (Pacific Biosciences (Menlo Park, Calif.) or the HELISCOPE platform (Helicos Biosciences (Cambridge, Mass.)), nano-technology for single-molecule sequencing (as implemented, e.g., using the GRIDON platform of Oxford Nanopore Technologies (Oxford, UK), the hybridization-assisted nano-pore sequencing (HANS) platforms developed by Nabsys (Providence, R.I.), and the ligase-based DNA sequencing platform with DNA nanoball (DNB) technology referred to as probe-anchor ligation (cPAL)), electron microscopy-based technology for single-molecule sequencing, and ion semiconductor sequencing.

In some embodiments therefore, a population of cancer-specific neoantigens is identified by sequencing the cancer/tumor and normal DNA of a patient to identify cancer-specific mutations, and determining the patient's HLA allotype. In some embodiments, the population of cancer-specific neoantigens and their cognate native antigens is subject to bioinformatic analysis using validated algorithms to predict which cancer-specific mutations create epitopes that could bind to the patient's HLA allotype, and in particular which cancer-specific mutations create epitopes that could bind to the patient's HLA allotype more effectively than the cognate native antigen. Based on this analysis, in some embodiments, identified nucleotide sequences corresponding to these mutations are designed for each patient, and used together as a personalized cancer vaccine to immunize the patient.

In some embodiments, the methods of the disclosure comprises a step of identifying one or more neoantigen mutations in a subject, wherein the subject has been diagnosed with, suspected of having, or comprises one or more hyperproliferative cells (e.g. such as a tumor). In some embodiments, the methods of the disclosure comprises a step of identifying one or more subject-specific neoantigen mutations in a subject, wherein the subject has been diagnosed with, suspected of having, or comprises one or more hyperproliferative cells (e.g. such as a tumor). In some embodiments, the methods of the disclosure comprises a step of identifying one or more subject-specific neoantigen mutations in a subject, wherein the subject has been diagnosed with, suspected of having, or comprises one or more hyperproliferative cells (e.g. such as a tumor) characterized by the presence or quantity of a plurality of neoantigen mutations, the method comprising sequencing a nucleic acid sample from a tumor of the subject and of a non-tumor sample of the subject; analyzing the sequence to determine coding and non-coding regions; identifying sequences comprising tumor-specific non-synonymous or non-silent mutations not present in the non-tumor sample; identifying single nucleotide variations and single nucleotide insertions and deletions; producing subject-specific peptides encoded by the sequences comprising tumor-specific non-synonymous or non-silent mutations not present in the non-tumor sample; and measuring the binding characteristics of the of the subject-specific peptides, wherein each subject-specific peptide is an expression product of subject-specific DNA neoantigen not present in the non-tumor sample, thereby identifying one or more subject-specific DNA neoantigens in a subject. Measuring the binding characteristics of the subject-specific peptides can be carried out by one or more of measuring the binding of the subject-specific peptides to T-cell receptor; measuring the binding of the subject-specific peptides to a HLA protein of the subject; or measuring the binding of the subject-specific peptides to transporter associated with antigen processing (TAP).

Efficiently choosing which particular mutations to utilize as immunogen requires identification of the patient HLA type and the ability to predict which mutated peptides would efficiently bind to the patient's HLA alleles. Therefore, in some embodiments, measuring the binding of the subject-specific peptides to T-cell receptor comprises measuring the binding of the subject-specific peptides to a HLA protein of the subject.

In some embodiments, the subject-specific peptides bind to HLA proteins of the subject with an IC50 of from about 10 nM to about 550 nM. In some embodiments, the subject-specific peptides bind to HLA proteins of the subject with an IC50 of from about 20 nM to about 500 nM. In some embodiments, the subject-specific peptides bind to HLA proteins of the subject with an IC50 of from about 30 nM to about 450 nM. In some embodiments, the subject-specific peptides bind to HLA proteins of the subject with an IC50 of from about 40 nM to about 400 nM. In some embodiments, the subject-specific peptides bind to HLA proteins of the subject with an IC50 of from about 50 nM to about 350 nM. In some embodiments, the subject-specific peptides bind to HLA proteins of the subject with an IC50 of from about 60 nM to about 300 nM. In some embodiments, the subject-specific peptides bind to HLA proteins of the subject with an IC50 of from about 70 nM to about 250 nM. In some embodiments, the subject-specific peptides bind to HLA proteins of the subject with an IC50 of from about 80 nM to about 200 nM. In some embodiments, the subject-specific peptides bind to HLA proteins of the subject with an IC50 of from about 90 nM to about 200 nM. In some embodiments, the subject-specific peptides bind to HLA proteins of the subject with an IC50 of from about 100 nM to about 150 nM.

In some embodiments, the subject-specific peptides bind to HLA proteins of the subject with an IC50 of from about 10 nM to about 100 nM. In some embodiments, the subject-specific peptides bind to HLA proteins of the subject with an IC50 of from about 20 nM to about 150 nM. In some embodiments, the subject-specific peptides bind to HLA proteins of the subject with an IC50 of from about 30 nM to about 175 nM. In some embodiments, the subject-specific peptides bind to HLA proteins of the subject with an IC50 of from about 40 nM to about 200 nM. In some embodiments, the subject-specific peptides bind to HLA proteins of the subject with an IC50 of from about 50 nM to about 225 nM. In some embodiments, the subject-specific peptides bind to HLA proteins of the subject with an IC50 of from about 60 nM to about 250 nM. In some embodiments, the subject-specific peptides bind to HLA proteins of the subject with an IC50 of from about 70 nM to about 275 nM. In some embodiments, the subject-specific peptides bind to HLA proteins of the subject with an IC50 of from about 80 nM to about 300 nM. In some embodiments, the subject-specific peptides bind to HLA proteins of the subject with an IC50 of from about 90 nM to about 325 nM. In some embodiments, the subject-specific peptides bind to HLA proteins of the subject with an IC50 of from about 100 nM to about 350 nM. In some embodiments, the subject-specific peptides bind to HLA proteins of the subject with an IC50 of from about 110 nM to about 375 nM. In some embodiments, the subject-specific peptides bind to HLA proteins of the subject with an IC50 of from about 120 nM to about 400 nM. In some embodiments, the subject-specific peptides bind to HLA proteins of the subject with an IC50 of from about 130 nM to about 425 nM. In some embodiments, the subject-specific peptides bind to HLA proteins of the subject with an IC50 of from about 140 nM to about 450 nM. In some embodiments, the subject-specific peptides bind to HLA proteins of the subject with an IC50 of from about 150 nM to about 475 nM. In some embodiments, the subject-specific peptides bind to HLA proteins of the subject with an IC50 of from about 175 nM to about 500 nM.

In some embodiments, the subject-specific peptides bind to HLA proteins of the subject with an IC50 of less than about 550 nM. In some embodiments, the subject-specific peptides bind to HLA proteins of the subject with an IC50 of less than about 500 nM. In some embodiments, the subject-specific peptides bind to HLA proteins of the subject with an IC50 of less than about 450 nM. In some embodiments, the subject-specific peptides bind to HLA proteins of the subject with an IC50 of less than about 400 nM. In some embodiments, the subject-specific peptides bind to HLA proteins of the subject with an IC50 of less than about 350 nM. In some embodiments, the subject-specific peptides bind to HLA proteins of the subject with an IC50 of less than about 300 nM. In some embodiments, the subject-specific peptides bind to HLA proteins of the subject with an IC50 of less than about 250 nM. In some embodiments, the subject-specific peptides bind to HLA proteins of the subject with an IC50 of less than about 200 nM. In some embodiments, the subject-specific peptides bind to HLA proteins of the subject with an IC50 of less than about 150 nM. In some embodiments, the subject-specific peptides bind to HLA proteins of the subject with an IC50 of less than about 100 nM. In some embodiments, the subject-specific peptides bind to HLA proteins of the subject with an IC50 of less than about 90 nM. In some embodiments, the subject-specific peptides bind to HLA proteins of the subject with an IC50 of less than about 80 nM. In some embodiments, the subject-specific peptides bind to HLA proteins of the subject with an IC50 of less than about 70 nM. In some embodiments, the subject-specific peptides bind to HLA proteins of the subject with an IC50 of less than about 60 nM. In some embodiments, the subject-specific peptides bind to HLA proteins of the subject with an IC50 of less than about 50 nM.

In some embodiments, the disclosed method further comprises a step of ranking the subject-specific peptides based on the binding characteristics. In some embodiments, the disclosed method further comprises a step of measuring the CD8+ T cell immune response generated by the subject-specific peptides. Methods of measuring the CD8+ T cell response are known in the art and described herein.

In some embodiments, the disclosed method further comprises formulating the subject-specific neoantigens into an immunogenic composition for administration to the subject. In some embodiments, the subject-specific neoantigens are formulated into an immunogenic composition in form of DNAs. In some embodiments, the subject-specific neoantigens are formulated into an immunogenic composition in form of RNAs. In some embodiments, the subject-specific neoantigens are formulated into an immunogenic composition in form of proteins. In the embodiments where the subject-specific neoantigens are formulated into an immunogenic composition in form of DNAs, the DNA neoantigens can be subcloned into one or a plurality of vectors, which in some embodiments, are one or a plurality of plasmids. Methods of administering DNA vaccines, RNA vaccines or protein vaccines are known in the art.

In some embodiments, the top 200 ranked neoantigen mutations are included in the immunogenic composition for administration to the subject. In some embodiments, the top 150 ranked neoantigen mutations are included in the immunogenic composition for administration to the subject. In some embodiments, the top 100 ranked neoantigen mutations are included in the immunogenic composition for administration to the subject. In some embodiments, the top 50 ranked neoantigen mutations are included in the immunogenic composition for administration to the subject. In some embodiments, the top 25 ranked neoantigen mutations are included in the immunogenic composition for administration to the subject. In some embodiments, the top 10 ranked neoantigen mutations are included in the immunogenic composition for administration to the subject. In some embodiments, the top 5 ranked neoantigen mutations are included in the immunogenic composition for administration to the subject. In some embodiments, about 5 to about 20 top-ranked neoantigen mutations (by frequency) are included in the immunogenic composition for administration to the subject. In some embodiments, about 10 to about 20 top-ranked neoantigen mutations are included in the immunogenic composition for administration to the subject. In some embodiments, the top about 25 to about 100 top-ranked neoantigen mutations are included in the immunogenic composition for administration to the subject. In some embodiments, about 50 to about 100 top-ranked neoantigen mutations are included in the immunogenic composition for administration to the subject. In some embodiments, about 100 to about 200 top-ranked ranked neoantigen mutations are included in the immunogenic composition for administration to the subject. In some embodiments, ranking is determined by ordering the neoantigens on a list from of lowest IC₅₀ value to highest IC₅₀ value.

In some embodiments therefore, the methods of the disclosure comprise a step of identifying or selecting one or a plurality of cancer-specific neoantigens from a subject, the step comprising: (a) sequencing the DNA and/or RNA from a cancer/tumor sample of the subject; (b) measuring the binding of the subject-specific neoantigens to a T-cell receptor; and measuring the binding of the subject-specific neoantigens to a HLA protein of the subject; (c) selecting one or a plurality of neoantigens from the sample if the subject-specific neoantigens binds to HLA proteins of the subject with an IC₅₀ of less than about 500 nM, 400 nM, 300 nM, 200 nM, or 100 nM; and, optionally (d) ordering the neoantigens in order of lowest IC50 value to highest IC50 value.

Administration of Neoantigen(s) into Subject

The disclosure relates to a pharmaceutical composition comprising a therapeutically effective amount of a nucleic acid molecule disclosed herein; and a pharmaceutically acceptable carrier. In some embodiments, the disclosure relates to a composition comprising a first pharmaceutical composition comprising a therapeutically effective amount of a nucleic acid molecule disclosed herein; and a pharmaceutically acceptable carrier; and a second pharmaceutical composition comprising a nucleic acid molecule encoding a therapeutically effective amount of IL-12 or a functional fragment thereof; and a pharmaceutically acceptable carrier. In some embodiments, the nucleic acid molecule disclosed herein comprises at least one expressible nucleic acid sequence that encodes from about 19 to about 50 neoantigens from a subject or a functional fragments thereof. In some embodiments, the neoantigens, or nucleic acid sequences encoding the same, are selected by administering a vaccine to a subject, isolating PBMC or antigen presenting cells of the subject, exposing the PBMC or antigen presenting cells of the subject to a cell comprising one or a plurality of nucleic acid molecule encoding one or a plurality of TCRs, and selecting one or a plurality of TCRs that bind to the neoantigens based on quantified strength of binding between the TCR and the neoantigen.

To activate the subject's immunity to the cancer-specific neoantigens, the methods of disclosure further comprise administering the cancer- and subject-specific neoantigens into the subject. Administration results in a subject-specific immune response becoming elicited in response to the neoantigens. Immune cells from the subject can be isolated, modified and then re-administered back into the subject as a treatment. The cancer- and subject-specific neoantigens can be administered into the subject in form of peptides, DNAs and/or RNAs. In some embodiments, the cancer- and subject-specific neoantigens are administered into the subject in form of peptides in a composition comprising one or more pharmaceutically acceptable carriers. In some embodiments, the cancer- and subject-specific neoantigens are administered into the subject in form of DNAs in a composition comprising one or more pharmaceutically acceptable carriers. In some embodiments, the cancer- and subject-specific neoantigens are administered into the subject in form of RNAs in a composition comprising one or more pharmaceutically acceptable carriers. In some embodiments, the cancer- and subject-specific neoantigens are administered into the subject in form of a mixture of DNAs and RNAs in a composition comprising one or more pharmaceutically acceptable carriers.

In the embodiments where the cancer- and subject-specific neoantigens are administered into the subject in form of DNAs, the DNA neoantigens can be subcloned into one or a plurality of vectors, exemplified by those vectors disclosed herein, which, in some embodiments, are one or a plurality of plasmids. In some embodiments therefore, the disclosed methods further comprise a step of generating a DNA vaccine, or manufacturing a pharmaceutical composition comprising such DNA vaccines, the step comprising performing any one or more of the aforementioned steps and further comprising subcloning a nucleic acid sequence encoding the one or plurality of neoantigens into one or more nucleic acid molecules; and optionally, suspending the resultant nucleic acid molecules in one or more pharmaceutically acceptable carriers.

Any method of generating DNA vaccines that express neoantigens or short peptides can be used in the disclosed methods. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. As an example, DNA vaccines suitable for the methods of the disclosure can be generated using the methods disclosed in International Application Publication No. WO 2019/227106, incorporated herein by reference. Briefly, one or a plurality of nucleotide sequences encoding one or a plurality of cancer-/tumor-specific neoantigens can be subcloned into a nucleic acid molecule. In some embodiments, only one nucleotide sequence encoding one single cancer-/tumor-specific neoantigen is subcloned into the nucleic acid molecule. In some embodiments, two or more nucleotide sequences encoding the same cancer-/tumor-specific neoantigen are subcloned into the nucleic acid molecule. In some embodiments, two or more nucleotide sequences encoding two or more cancer-/tumor-specific neoantigens are subcloned into the nucleic acid molecule.

In some embodiments therefore, the nucleic acid molecule, or the resultant DNA vaccine, encodes from about 1 to about 100 neoantigens. In some embodiments, the nucleic acid molecule, or the resultant DNA vaccine, encodes from about 2 to about 95 neoantigens. In some embodiments, the nucleic acid molecule, or the resultant DNA vaccine, encodes from about 3 to about 90 neoantigens. In some embodiments, the nucleic acid molecule, or the resultant DNA vaccine, encodes from about 4 to about 85 neoantigens. In some embodiments, the nucleic acid molecule, or the resultant DNA vaccine, encodes from about 5 to about 80 neoantigens. In some embodiments, the nucleic acid molecule, or the resultant DNA vaccine, encodes from about 6 to about 75 neoantigens. In some embodiments, the nucleic acid molecule, or the resultant DNA vaccine, encodes from about 7 to about 70 neoantigens. In some embodiments, the nucleic acid molecule, or the resultant DNA vaccine, encodes from about 8 to about 65 neoantigens. In some embodiments, the nucleic acid molecule, or the resultant DNA vaccine, encodes from about 9 to about 60 neoantigens. In some embodiments, the nucleic acid molecule, or the resultant DNA vaccine, encodes from about 10 to about 55 neoantigens. In some embodiments, the nucleic acid molecule, or the resultant DNA vaccine, encodes from about 20 to about 65 neoantigens. In some embodiments, the nucleic acid molecule, or the resultant DNA vaccine, encodes from at or greater than 20 neoantigens. In some embodiments, all the neoantigens encoded by the nucleic acid molecule, or the resultant DNA vaccine, are the same neoantigen. In some embodiments, at least about two or more of the neoantigens encoded by the nucleic acid molecule, or the resultant DNA vaccine, are different from each other.

In some embodiments, the nucleic acid molecule, or the resultant DNA vaccine, encodes one single neoantigen. In some embodiments, the nucleic acid molecule, or the resultant DNA vaccine, encodes about 2 different neoantigens. In some embodiments, the nucleic acid molecule, or the resultant DNA vaccine, encodes about 3 different neoantigens. In some embodiments, the nucleic acid molecule, or the resultant DNA vaccine, encodes about 4 different neoantigens. In some embodiments, the nucleic acid molecule, or the resultant DNA vaccine, encodes about 5 different neoantigens. In some embodiments, the nucleic acid molecule, or the resultant DNA vaccine, encodes about 6 different neoantigens. In some embodiments, the nucleic acid molecule, or the resultant DNA vaccine, encodes about 7 different neoantigens. In some embodiments, the nucleic acid molecule, or the resultant DNA vaccine, encodes about 8 different neoantigens. In some embodiments, the nucleic acid molecule, or the resultant DNA vaccine, encodes about 9 different neoantigens. In some embodiments, the nucleic acid molecule, or the resultant DNA vaccine, encodes about 10 different neoantigens. In some embodiments, the nucleic acid molecule, or the resultant DNA vaccine, encodes about 20 different neoantigens. In some embodiments, the nucleic acid molecule, or the resultant DNA vaccine, encodes at or greater than about 20 different neoantigens. In some embodiments, the nucleic acid molecule, or the resultant DNA vaccine, encodes about 30 different neoantigens. In some embodiments, the nucleic acid molecule, or the resultant DNA vaccine, encodes about 40 different neoantigens. In some embodiments, the nucleic acid molecule, or the resultant DNA vaccine, encodes about 50 different neoantigens. In some embodiments, the nucleic acid molecule, or the resultant DNA vaccine, encodes about 55 different neoantigens. In some embodiments, the nucleic acid molecule, or the resultant DNA vaccine, encodes about 60 different neoantigens. In some embodiments, the nucleic acid molecule, or the resultant DNA vaccine, encodes about 65 different neoantigens. In some embodiments, the nucleic acid molecule, or the resultant DNA vaccine, encodes about 70 different neoantigens. In some embodiments, the nucleic acid molecule, or the resultant DNA vaccine, encodes about 75 different neoantigens. In some embodiments, the nucleic acid molecule, or the resultant DNA vaccine, encodes about 80 different neoantigens. In some embodiments, the nucleic acid molecule, or the resultant DNA vaccine, encodes about 85 different neoantigens. In some embodiments, the nucleic acid molecule, or the resultant DNA vaccine, encodes about 90 different neoantigens. In some embodiments, the nucleic acid molecule, or the resultant DNA vaccine, encodes about 95 different neoantigens. In some embodiments, the nucleic acid molecule, or the resultant DNA vaccine, encodes about 100 different neoantigens. In some embodiments, the nucleic acid molecule, or the resultant DNA vaccine, encodes more than about 100 different neoantigens.

In the embodiments where the nucleic acid molecule, or the resultant DNA vaccine, encodes more than one neoantigen, each neoantigen encoded by the nucleic acid molecule can be separated from another by one or a plurality of linkers. In some embodiments therefore, the nucleic acid molecule, or the resultant DNA vaccine, of the disclosure further comprise one or a plurality of nucleotide sequences encoding one or plurality of linkers. In some embodiments, each linker is independently selectable from about 0 to about 30, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, or about 24 to about 25 amino acids in length. In some embodiments, each linker is independently selectable from about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acids in length.

In the embodiments where more than one linker is encoded by the nucleic acid molecule, or the resultant DNA vaccine, of the disclosure, the length of each linker can be the same or different. In some embodiments, the length of each linker is the same. In some embodiments, the length of each linker is different. For example, in some embodiments, the length of a first linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acids in length, and a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acids in length, where the length of the first linker is different from the length of the second linker. Various configurations can be envisioned by the present disclosure, where the nucleic acid molecule or the DNA vaccine of the disclosure comprises 1, 2, 3, 4, 5, 6, 7, 8, 9, 10 or more linkers, wherein the linkers are of similar or different lengths. In some embodiments, the linkers are all of the same lengths and the are at least about 19 linkers.

In some embodiments, two linkers can be used together as a fusion peptide encoded by one single nucleotide sequence. Accordingly, in some embodiments, the first linker is independently selectable from about 0 to about 30, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, or about 24 to about 25 amino acids in length. In some embodiments, the second linker is independently selectable from about 0 to about 30, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, or about 24 to about 25 amino acids in length. In some embodiments, the first linker is independently selectable from about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acids in length. In some embodiments, the second linker is independently selectable from about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 amino acids in length.

The disclosure relates to a nucleic acid molecule comprising at least one expressible nucleic acid sequence that comprises any of the Formula included herein and that encode any of the antigens identified in Tables M, N, and/or O or functional fragments of those sequences that are at least about 70%, 80%, 85%, 86%, 87%, 88%, 89%, 90%, 91%, 92%, 93%, 94%, 95%, 96%, 97%, 98%, or 99% sequence identity to those antigen identified in Tables M, N, and/or O. In some embodiments, the nucleic acid molecules comprise a nucleic acid sequence encoding at least about 19, 20, 21, 22, 23, 24, 25, 26, 27, 28, 29, 30, 31, 32, 33, 34, 35, 36, 37, 38, 39, 40, 41, 42, 43, 44, or 45 or more different antigen domains, wherein at least one of the antigen domains is chosen from the antigens identified in Tables M, N, and/or O or functional fragments of those sequences that are at least about 70%, 80%, 85%, 86%, 87%, 88%, 89%, 90%, 91%, 92%, 93%, 94%, 95%, 96%, 97%, 98%, or 99% sequence identity to those antigen identified in Tables M, N, and/or O. The disclosure relates to a cell comprising a nucleic acid molecule comprising at least one expressible nucleic acid sequence that comprises any of the Formula included herein and that encode any of the antigens identified in Tables M, N, and/or O or functional fragments of those sequences that are at least about 70%, 80%, 85%, 86%, 87%, 88%, 89%, 90%, 91%, 92%, 93%, 94%, 95%, 96%, 97%, 98%, or 99% sequence identity to those antigen identified in Tables M, N, and/or O. In some embodiments, the composition of the disclosure comprise a cell comprising a nucleic acid molecule comprising at least one expressible nucleic acid sequence that comprises any of the Formula included herein and that encode any of the antigens identified in Tables M, N, and/or O or functional fragments of those sequences that are at least about 70%, 80%, 85%, 86%, 87%, 88%, 89%, 90%, 91%, 92%, 93%, 94%, 95%, 96%, 97%, 98%, or 99% sequence identity to those antigen identified in Tables M, N, and/or O and at least one cell comprising a nucleic acid molecule comprising a nucleic acid sequence encoding a TCR sequence. In some embodiments, the cell comprises a plasmid encoding one or a plurality of TCR sequences chosen from any one or more from Tables Q, R, X, Y or Z, or functional fragment thereof that are at least about 70%, 80%, 85%, 86%, 87%, 88%, 89%, 90%, 91%, 92%, 93%, 94%, 95%, 96%, 97%, 98%, or 99% sequence identity to those TCR sequences in Tables Q, R, X, Y, and Z.

In some embodiments, one or a plurality of the linkers encoded by the nucleic acid molecule or the DNA vaccine of the disclosure comprise an amino acid cleavage site. Any amino acid cleavage site may be used. One non-limiting example is a furin protease cleavage site, which is recognized by a protease known as furin that resides in the trans-Golgi network of eukaryotic cells. Furin functions to cleave proteins at a step just prior to their delivery to their final cellular destination. Furin recognizes a consensus amino acid sequence, RXRR, RXRK or KXKR (where X is any amino acid, Moehring et al., 1993, incorporated by reference in its entirety herein) and cuts proteins which contain these sequences when they reach the trans-Golgi network. Furin is a Ca2+-dependent serine endoprotease that cleaves protein precursors with a high specificity after the multiple basic motifs shown in Table 1 below.

TABLE 1
Basic motifs of furin cleavage site.

| | |
|---|---|
| Canonic | —R^{P4}—X^{P3}—(K/R)^{P2}—R^{P1}↓X^{P1′}—X^{P2′}—X^{P3′}—X^{P4′} |
| Alternative | —R^{P6}—X^{P5}—X^{P4}—X^{P3}—(K/R)^{P2}—R^{P1}↓X^{P1′}—X^{P2′}—X^{P3′}—X^{P4′} |
| Minimal | —R^{P4}—X^{P3}—X^{P2}—R^{P1}↓X^{P1′}—X^{P2′}—X^{P3′}—X^{P4′} |

Another non-limiting example of the amino acid cleavage site is a cleavage site recognized by a 2A peptide, which is a “self-cleaving” small peptide. The average length of 2A peptides is 18-22 amino acids. The designation “2A” refers to a specific region of picornavirus polyproteins. Of the 2A peptides identified to date, four are widely used in research: FMDV 2A (abbreviated herein as F2A); equine rhinitis A virus (ERAV) 2A (E2A); porcine teschovirus-1 2A (P2A) and Thoseaasigna virus 2A (T2A). The former three viruses belong to picornaviruses and the latter is an insect virus. DNA and corresponding amino acid sequences of various 2A peptides are shown below in Table 2. Underlined sequences encode amino acids GSG, which were added to improve cleavage efficiency. P2A indicates porcine teschovirus-1 2A; T2A, Thoseaasigna virus 2A; E2A, equine rhinitis A virus (ERAV) 2A; F2A, FMDV 2A.

TABLE 2
DNA and corresponding amino acid sequences of various 2A peptides.
Table discloses SEQ ID NOS 595-602, respectively, in order of appearance.

| | |
|---|---|
| P2A | GGA AGC GGA GCT ACT AAC TTC AGC CTG CTG AAG CAG GCT GGA |
| | G S G A T N F S L L K Q A G |
| | GAC GTG GAG GAG AAC CCT GGA CCT |
| | D V E E N P G P |
| T2A | GGA AGC GGA GAG GGC AGA GGA AGT CTG CTA ACA TGC GGT GAC |
| | G S G E G R G S L L T C G D |
| | GTC GAG GAG AAT CCT GGA CCT |
| | V E E N P G P |
| E2A | GGA AGC GGA CAG TGT ACT AAT TAT GCT CTC TTG AAA TTG GCT |
| | G S G Q C T N Y A L L K L A |
| | GGA GAT GTT GAG AGC AAC CCT GGA CCT |
| | G D V E S N P G P |
| F2A | GGA AGC GGA GTG AAA CAG ACT TTG AAT TTT GAC CTT CTC AAG |
| | G S G V K Q T L N F D L L K |
| | TTG GCG GGA GAC GTG GAG TCC AAC CCT GGA CCT |
| | L A G D V E S N P G P |

In some embodiments, the one or plurality of the linkers encoded by the nucleic acid molecule or the DNA vaccine of the disclosure comprise a furin protease cleavage site. In some embodiments, the one or plurality of the linkers encoded by the nucleic acid molecule or the DNA vaccine of the disclosure comprise a furin-sensitive cleavage site comprising the sequence R-X-[R/K]-R, where R denotes arginine, X is any amino acid, and K is lysine. The “R/K” indicates that this amino acid is either arginine or lysine.

In some embodiments, the one or plurality of the linkers encoded by the nucleic acid molecule or the DNA vaccine of the disclosure comprise a 2A cleavage site. In some embodiments, the one or plurality of the linkers encoded by the nucleic acid molecule or the DNA vaccine of the disclosure comprise a porcine teschovirus-1 2A (P2A) cleavage site. In some embodiments, the one or plurality of the linkers encoded by the nucleic acid molecule or the DNA vaccine of the disclosure comprise a Thoseaasigna virus 2A (T2A) cleavage site. In some embodiments, the one or plurality of the linkers encoded by the nucleic acid molecule or the DNA vaccine of the disclosure comprise an equine rhinitis A virus (ERAV) 2A 9 (E2A) cleavage site. In some embodiments, the one or plurality of the linkers encoded by the nucleic acid molecule or the DNA vaccine of the disclosure comprise a FMDV 2A (F2A) cleavage site.

In some embodiments, the nucleic acid molecule or the DNA vaccine of the disclosure further comprises a nucleotide sequence encoding a leader sequence. A “leader sequence,” or sometimes referred to as a “signal peptide,” is an amino acid sequence that typically directs localization of a protein. Leader sequences used herein preferably facilitate secretion of the protein from the cell in which it is produced. Leader sequences are often cleaved from the remainder of the protein, often referred to as the mature protein, upon secretion from the cell. Leader sequences, when present, are linked at the N terminus of the protein. A non-limiting example of the leader sequences is an IgE leader sequence described in U.S. Application Publication No. 2016/0175427, which is incorporated by reference in its entirety herein.

In some embodiments, the nucleic acid molecule or the DNA vaccine of the disclosure may also comprise one or a plurality of regulatory sequences. When present, such one or plurality of regulatory sequences are operably linked to the nucleotide sequence encoding the neoantigens, linkers, and/or leader sequences. Examples of regulatory sequences include, but not limited to, promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

In some embodiments, the nucleic acid molecule or the DNA vaccine of the disclosure is comprised in a vector or a plasmid. In some embodiments, the vector is a plasmid. The plasmid may be useful for transfecting cells with the nucleic acid molecule or the DNA vaccine of the disclosure, which the transformed cells express the neoantigens to elicit an immune response. In some embodiments, the composition of the disclosure relate to a nucleic acid molecule comprising a nucleic acid sequence encoding a plurality of neoantigens. In some embodiments, the plasmid further comprises an initiation codon, which may be upstream or downstream of the neoantigen-coding sequence, and a stop codon. In some embodiments, the initiation codon and stop codon are in frame with the neoantigen-coding sequence.

In some embodiments, the plasmid also comprises a promoter that is operably linked to the coding sequence. In some embodiments, the promoter operably linked to the coding sequence is a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. In some embodiments, the promoter is a promoter from a human gene such as human actin, human myosin, human hemoglobin, human muscle creatine, or human metalothionein. In some embodiments, the promoter is a tissue specific promoter, such as a muscle or skin specific promoter, natural or synthetic. Examples of such promoters are described in US patent application publication no. US20040175727, the contents of which are incorporated herein in its entirety. In some embodiments, the plasmid also comprises a polyadenylation signal, which may be downstream of the coding sequence. In some embodiments, the polyadenylation signal is a SV40 polyadenylation signal, LTR polyadenylation signal, bovine growth hormone (bGH) polyadenylation signal, human growth hormone (hGH) polyadenylation signal, or human β-globin polyadenylation signal. In some embodiments, the SV40 polyadenylation signal is a polyadenylation signal from a pCEP4 plasmid (Invitrogen, San Diego, CA).

In some embodiments, the plasmid also comprises an enhancer upstream of the coding sequence. In some embodiments, the enhancer is human actin, human myosin, human hemoglobin, human muscle creatine or a viral enhancer such as one from CMV, FMDV, RSV or EBV. Polynucleotide function enhancers are described in, for instance, U.S. Pat. Nos. 5,593,972, 5,962,428, and WO94/016737, the contents of each are fully incorporated by reference. In some embodiments, the plasmid also comprises a mammalian origin of replication in order to maintain the plasmid extrachromosomally and produce multiple copies of the plasmid in a cell. IN some embodiments, the plasmid is pVAX1, pCEP4 or pREP4 from ThermoFisher Scientific (San Diego, CA), which comprises the Epstein Barr virus origin of replication and nuclear antigen EBNA-1 coding region, which produces high copy episomal replication without integration.

In some embodiments, the vector is pVAX1 or a pVax1 variant with changes such as the variant plasmid described herein. The variant pVax1 plasmid is a 2998 basepair variant of the backbone vector plasmid pVAX1 (Invitrogen, Carlsbad CA). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993. The nucleic acid sequence for the pVAX1 (SEQ ID NO: 361) backbone sequence is as follows:

gactcttcgcgatgtacgggccagatatacgcgttgacattgattattgactagttattaatagtaatcaattacggggtcattagttcatagcc
catatatggagttccgcgttacataacttacggtaaatggcccgcctggctgaccgcccaacgacccccgcccattgacgtcaataatgacgt
atgttcccatagtaacgccaatagggactttccattgacgtcaatgggtggactatttacggtaaactgcccacttggcagtacatcaagtgtat
catatgccaagtacgccccctattgacgtcaatgacggtaaatggcccgcctggcattatgcccagtacatgaccttatgggactttcctactt
ggcagtacatctacgtattagtcatcgctattaccatggtgatgcggttttggcagtacatcaatgggcgtggatagcggtttgactcacgggg
atttccaagtctccaccccattgacgtcaatgggagtttgttttggcaccaaaatcaacgggactttccaaaatgtcgtaacaactccgccccat
tgacgcaaatgggcggtaggcgtgtacggtgggaggtctatataagcagagctctctggctaactagagaacccactgcttactggcttatc
gaaattaatacgactcactatagggagacccaagctggctagcgtttaaacttaagcttggtaccgagctcggatccactagtccagtgtggt
ggaattctgcagatatccagcacagtggcggccgctcgagtctagagggcccgtttaaacccgctgatcagcctcgactgtgccttctagtt
gccagccatctgttgtttgcccctcccccgtgccttccttgaccctggaaggtgccactcccactgtcctttcctaataaaatgaggaaattgca
tcgcattgtctgagtaggtgtcattctattctggggggtggggtggggcaggacagcaagggggaggattgggaagacaatagcaggcat
gctggggatgcggtgggctctatggcttctactgggcggttttatggacagcaagcgaaccggaattgccagctggggcgccctctggtaa
ggttgggaagccctgcaaagtaaactggatggctttctcgccgccaaggatctgatggcgcaggggatcaagctctgatcaagagacagg
atgaggatcgtttcgcatgattgaacaagatggattgcacgcaggttctccggccgcttgggtggagaggctattcggctatgactgggcac
aacagacaatcggctgctctgatgccgccgtgttccggctgtcagcgcaggggcgcccggttctttttgtcaagaccgacctgtccggtgcc
ctgaatgaactgcaagacgaggcagcgcggctatcgtggctggccacgacgggcgttccttgcgcagctgtgctcgacgttgtcactgaa
gcgggaagggactggctgctattgggcgaagtgccggggcaggatctcctgtcatctcaccttgctcctgccgagaaagtatccatcatgg
ctgatgcaatgcggcggctgcatacgcttgatccggctacctgcccattcgaccaccaagcgaaacatcgcatcgagcgagcacgtactc
ggatggaagccggtcttgtcgatcaggatgatctggacgaagagcatcaggggctcgcgccagccgaactgttcgccaggctcaaggcg
agcatgcccgacggcgaggatctcgtcgtgacccatggcgatgcctgcttgccgaatatcatggtggaaaatggccgcttttctggattcatc
gactgtggccggctgggtgtggcggaccgctatcaggacatagcgttggctacccgtgatattgctgaagagcttggcggcgaatgggct
gaccgcttcctcgtgctttacggtatcgccgctcccgattcgcagcgcatcgccttctatcgccttcttgacgagttcttctgaattattaacgc
ttacaatttcctgatgcggtattttctccttacgcatctgtgcggtatttcacaccgcatacaggtggcacttttcggggaaatgtgcgcggaac
ccctatttgtttatttttctaaatacattcaaatatgtatccgctcatgagacaataaccctgataaatgcttcaataatagcacgtgctaaaac
ttcatttttaatttaaaaggatctaggtgaagatcctttttgataatctcatgaccaaaatcccttaacgtgagttttcgttccactgagcgtca
gaccccgtagaaaagatcaaaggatcttcttgagatcctttttttctgcgcgtaatctgctgcttgcaaacaaaaaaaccaccgctaccagcggt
ggtttgtttgccggatcaagagctaccaactctttttccgaaggtaactggcttcagcagagcgcagataccaaatactgtccttctagtgtagc
cgtagttaggccaccacttcaagaactctgtagcaccgcctacatacctcgctctgctaatcctgttaccagtggctgctgccagtggcgataag
tcgtgtcttaccgggttggactcaagacgatagttaccggataaggcgcagcggtcgggctgaacggggggttcgtgcacacagcccagcttgga
gcgaacgacctacaccgaactgagatacctacagcgtgagctatgagaaagcgccacgcttcccgaagggagaaaggcggacaggtatcc
ggtaagcggcagggtcggaacaggagagcgcacgagggagcttccagggggaaacgcctggtatctttatagtcctgtcgggtttcgcc
acctctgacttgagcgtcgatttttgtgatgctcgtcaggggggcggagcctatggaaaaacgccagcaacgcggcctttttacggttcctgg
gcttttgctggccttttgctcacatgttctt

In some embodiments, the nucleic acid molecule comprises at least about 70%, 80%, 90%, 95%, 96%, 97%, 98%, 99% or 100% sequence identity to pVAX1 backbone or a functional fragment thereof and the nucleic acid sequence further comprises an expressible nucleic acid sequence within the multiple cloning site.

Other vectors or plasmids that can be used herein to produce the vaccine of the present disclosure include, but not limited to, pcDNA3.1 (+), pCI mammalian expression vector, pSI vector, pZeoSV2 (+), phCMV1, pTCP and pIRES.

In some embodiments, the nucleic acid molecule or the DNA vaccine of the disclosure is administered into the subject in an amount sufficient to elicit a cellular immune response. A “cellular immune response” is meant to include a cellular response directed to cells characterized by presentation of an antigen with class I or class II MHC. The cellular response relates to cells called T cells or T-lymphocytes which act as either “helpers” or “killers.” The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells. In some embodiments, the present disclosure involves the stimulation of an anti-cancer CTL response against cancer cells expressing one or more cancer-specific neoantigens and preferably presenting such cancer-specific neoantigens with class I MHC.

In some embodiments, the nucleic acid molecule or the DNA vaccine of the disclosure is administered into the subject in an amount sufficient to elicit a CD8+ T cell response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a CD8+T and/or CD4+ T cell response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a CD4+ T cell response against any one or plurality of neoantigens encoded by the nucleic acid molecule or the DNA vaccine.

In some embodiments, the nucleic acid molecule or the DNA vaccine of the disclosure is administered into the subject in an amount sufficient to elicit a subpopulation of T cells that comprise greater than at least about 25% CD4+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 30% CD4+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 40% CD4+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 50% CD4+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 60% CD4+ T cells in response against any one or plurality of neoantigens encoded by the nucleic acid molecule or the DNA vaccine.

In some embodiments, the nucleic acid molecule or the DNA vaccine of the disclosure is administered into the subject in an amount sufficient to elicit a subpopulation of T cells that comprise greater than at least about 25% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 30% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 40% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 50% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 60% CD8+ T cells in response against any one or plurality of neoantigens encoded by the nucleic acid molecule or the DNA vaccine.

In some embodiments, the nucleic acid molecule or the DNA vaccine of the disclosure is administered into the subject in an amount sufficient to elicit a subpopulation of T cells that comprise greater than at least about 25% CD4+ T cells and greater than at least about 25% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 30% CD4+ T cells and greater than at least about 30% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 40% CD4+T cells and greater than at least about 40% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 50% CD4+ T cells and greater than at least about 50% CD8+ T cells in response against any one or plurality of neoantigens encoded by the nucleic acid molecule or the DNA vaccine. In some embodiments, the nucleic acid molecule or the DNA vaccine of the disclosure is administered into the subject in an amount sufficient to elicit a subpopulation of T cells that comprise greater than at least about 60% CD4+ T cells and greater than at least about 60% CD8+ T cells in response against any one or plurality of neoantigens encoded by the nucleic acid molecule or the DNA vaccine.

In some embodiments, the nucleic acid molecule or the DNA vaccine of the disclosure is administered into the subject at a quantity of from about 1 nanogram to 100 milligrams; from about 1 microgram to about 10 milligrams; from about 0.1 microgram to about 10 milligrams; or from about 1 milligram to about 2 milligram. In some embodiments, a pharmaceutical composition comprising the nucleic acid molecule or the DNA vaccine of the disclosure in an amount of from about 1 nanogram to about 1000 micrograms of DNA is used for administration.

The nucleic acid molecule or the DNA vaccine of the disclosure, or a vector or plasmids comprising the same, can be administered, or delivered, to the subject by several well-known technologies, including but not limited to DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. In some embodiments, the neoantigens are delivered via DNA injection and along with in vivo electroporation.

The nucleic acid molecule or the DNA vaccine of the disclosure, or a vector or plasmids comprising the same, can be administered by electroporation. This can be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation can be accomplished using an in vivo electroporation device, for example CELLECTRA® EP system (Inovio Pharmaceuticals, Inc., Blue Bell, PA) or Elgen electroporator (Inovio Pharmaceuticals, Inc.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. In some embodiments, the feedback mechanism is performed by an analog closed-loop circuit. The feedback occurs every 50 μs, 20 μs, 10 us or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present disclosure, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119 (e) to U.S. Provisional Application Ser. Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference in its entirety.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference in its entirety. The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes. The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to a method of injecting DNA are contemplated herein. The above-patents are incorporated by reference in their entireties.

In some embodiments, the cancer- and subject-specific neoantigens are administered into the subject in form of RNAs. In such embodiments, the neoantigen-coding RNAs can be delivered as a transgene in an RNA vaccine (such as Moderna's mRNA platform). RNA vaccines are non-infectious, non-integrating and are naturally degrading. RNA vaccines have been shown to stimulate strong B cell responses to encoded transgenes. Naked RNA can be formulated with lipid nanoparticles (LNPs) to encapsulate RNA protecting the transgene from degradation.

Compositions comprising RNA nucleic acid sequences can be delivered via lipid-containing nanoparticles. In some embodiments, the composition includes at least one RNA polynucleotide having an open reading frame encoding one or a plurality of cancer-specific neoantigens having at least one modification, at least one 5′ terminal cap and is formulated within a lipid nanoparticle. In some embodiments, at least one chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. In some embodiments, the 5′-capping of polynucleotides is completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G (5′)ppp(5′) A; G (5′)ppp(5′) G; m7G(5′)ppp(5′) A; m7G(5′)ppp(5′) G (New England BioLabs, Ipswich, Mass.). In other embodiments, the 5′-capping of modified RNA is completed post-transcriptionally using a Vaccinia Virus Capping Enzyme to generate the “Cap 0” structure: m7G(5′)ppp(5′) G (New England BioLabs, Ipswich, Mass.). Cap 1 structure may be generated using both Vaccinia Virus Capping Enzyme and a 2′-O methyl-transferase to generate m7G(5′)ppp(5′) G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source. When transfected into mammalian cells, the modified mRNAs have a stability of from about 12 to about 18 hours or more than about 18 hours, e.g., 24, 36, 48, 60, 72, or greater than about 72 hours.

In some embodiments, a lipid nanoparticle comprises a cationic lipid, a PEG-modified lipid, a sterol, and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino) butanoyl)oxy) heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530).

In some embodiments, the neoantigen-coding RNAs of the disclosure are formulated in a lipid nanoparticle. In some embodiments, the neoantigen-coding RNAs of the disclosure are formulated in a lipid-polycation complex, referred to as a cationic lipid nanoparticle. The formation of the lipid nanoparticle may be accomplished by methods known in the art and/or as described in U.S. Publication No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Publication No. WO2012013326 or U.S. Publication No. US20130142818; each of which is herein incorporated by reference in its entirety. In some embodiments, the neoantigen-coding RNAs of the disclosure are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components, and biophysical parameters such as size. In one example by Semple et al. (Nature Biotech. 2010 28:172-176; herein incorporated by reference in its entirety), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid was shown to more effectively deliver siRNA to various antigen presenting cells (Basha et al. Mol Ther. 2011 19:2186-2200; herein incorporated by reference in its entirety).

In some embodiments, lipid nanoparticle formulations may comprise 35% to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles is 5:1 to 20:1, 10:1 to 25:1, 15:1 to 30:1, and/or at least 30:1.

In some embodiments, the ratio of PEG in the lipid nanoparticle formulations is increased or decreased and/or the carbon chain length of the PEG lipid is modified from C14 to C18 to alter the pharmacokinetics and/or biodistribution of the lipid nanoparticle formulations. As a non-limiting example, in some embodiments, lipid nanoparticle formulations contains from about 0.5% to about 3.0%, from about 1.0% to about 3.5%, from about 1.5% to about 4.0%, from about 2.0% to about 4.5%, from about 2.5% to about 5.0%, and/or from about 3.0% to about 6.0% of the lipid molar ratio of PEG-c-DOMG (R-3-[(co-methoxy-poly(ethyleneglycol) 2000) carbamoyl)]-1,2-dimyristyloxypropyl-3-amine) (also referred to herein as PEG-DOMG) as compared to the cationic lipid, DSPC, and cholesterol. In some embodiments, the PEG-c-DOMG is replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). In some embodiments, the cationic lipid is selected from any lipid known in the art such as, but not limited to, DLin-MC3-DMA, DLin-DMA, C12-200, and DLin-KC2-DMA.

In some embodiments, the neoantigen-coding RNAs of the disclosure is formulated as a nanoparticle that comprises at least one lipid. In some embodiments, the lipid is selected from, but is not limited to, DLin-DMA, DLin-K-DMA, 98N12-5, C12-200, DLin-MC3-DMA, DLin-KC2-DMA, DODMA, PLGA, PEG, PEG-DMG, (12Z,15Z)—N,N-dimethyl-2-nonylhenicosa-12,15-dien-1-amine (L608), N,N-dimethyl-1-[(1S,2R)-2-octylcyclopropyl]heptadecan-8-amine (L530), PEGylated lipids, and amino alcohol lipids.

In some embodiments, a lipid nanoparticle formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), e.g., from about 35% to about 65%, from about 45% to about 65%, about 60%, about 57.5%, about 50% or about 40% on a molar basis. In some embodiments, a lipid nanoparticle formulation includes from about 0.5% to about 15% on a molar basis of the neutral lipid, e.g., from about 3% to about 12%, from about 5% to about 10%, or about 15%, about 10%, or about 7.5% on a molar basis. Examples of neutral lipids include, without limitation, DSPC, POPC, DPPC, DOPE, and SM. In some embodiments, the formulation includes from about 5% to about 50% on a molar basis of the sterol (e.g., from about 15% to about 45%, from about 20% to about 40%, about 40%, about 38.5%, about 35%, or about 31% on a molar basis. A non-limiting example of a sterol is cholesterol. In some embodiments, a lipid nanoparticle formulation includes from about 0.5% to about 20% on a molar basis of the PEG or PEG-modified lipid (e.g., from about 0.5% to about 10%, from about 0.5% to about 5%, about 0.5%, about 1.0%, about 1.5%, about 3.5%, or about 5% on a molar basis. In some embodiments, a PEG or PEG modified lipid comprises a PEG molecule of an average molecular weight of about 2,000 Da. In some embodiments, a PEG or PEG modified lipid comprises a PEG molecule of an average molecular weight of less than about 2,000, for example about 1,500 Da, about 1,000 Da, or about 500 Da. Non-limiting examples of PEG-modified lipids include PEG-distearoyl glycerol (PEG-DMG) (also referred herein as PEG-C14 or C14-PEG), and PEG-cDMA (further discussed in Reyes et al. J. Controlled Release, 107, 276-287 (2005) the content of which is herein incorporated by reference in its entirety).

In some embodiments, lipid nanoparticle formulations include about 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), about 0.5-15% of the neutral lipid, about 5-50% of the sterol, and about 0.5-20% of the PEG or PEG-modified lipid on a molar basis. In some embodiments, lipid nanoparticle formulations include about 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), about 3-12% of the neutral lipid, about 15-45% of the sterol, and about 0.5-10% of the PEG or PEG-modified lipid on a molar basis. In some embodiments, lipid nanoparticle formulations include about 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), about 5-10% of the neutral lipid, about 25-40% of the sterol, and about 0.5-10% of the PEG or PEG-modified lipid on a molar basis. In some embodiments, lipid nanoparticle formulations include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino) butanoyl)oxy) heptadecanedioate (L319), about 7.5% of the neutral lipid, about 31% of the sterol, and about 1.5% of the PEG or PEG-modified lipid on a molar basis.

In some embodiments, the neoantigen-coding RNAs of the disclosure include at least one ribonucleic acid (RNA) polynucleotide having an open reading frame encoding one or a plurality of cancer-specific neoantigens, wherein at least about 80% of the uracil in the open reading frame have a chemical modification, optionally wherein the neoantigen-coding RNAs are formulated in a lipid nanoparticle. In some embodiments, the neoantigen-coding RNAs are formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5 (12) 1708-1713); herein incorporated by reference in its entirety) and hyaluronan-coated liposomes (Quiet Therapeutics, Israel). In some embodiments, the neoantigen-coding RNAs may be formulated in a lyophilized gel-phase liposomal composition as described in U.S. Publication No. US2012060293, herein incorporated by reference in its entirety.

In some embodiments, the nanoparticle formulations comprise a phosphate conjugate. The phosphate conjugate may increase in vivo circulation times and/or increase the targeted delivery of the nanoparticle. In some embodiments, the conjugates for use with the present disclosure is made by the methods described in International Publication No. WO2013033438 or U.S. Publication No. US20130196948, the content of each of which is herein incorporated by reference in its entirety. As a non-limiting example, the phosphate conjugates may include a compound of any one of the formulas described in International Publication No. WO2013033438, herein incorporated by reference in its entirety. In particular, the present disclosure relates to a pharmaceutical composition comprising nanoparticles which comprise RNA encoding one or a plurality of cancer-specific neoantigen, wherein:
(i) the number of positive charges in the nanoparticles does not exceed the number of negative charges in the nanoparticles; and/or
(ii) the nanoparticles have a neutral or net negative charge; and/or
(iii) the charge ratio of positive charges to negative charges in the nanoparticles is 1.4:1 or less; and/or
(iv) the zeta potential of the nanoparticles is about 0 or less.

In some embodiments, the nanoparticles described herein are colloidally stable for at least about 2 hours in the sense that no aggregation, precipitation or increase of size and polydispersity index by more than about 30% as measured by dynamic light scattering takes place. In some embodiments, the charge ratio of positive charges to negative charges in the nanoparticles is from about 1.4:1 to about 1:8, from about 1.2:1 to about 1:4, from about 1:1 to about 1:3, from about 1:1.2 to about 1:2, from about 1:1.2 to about 1:1.8, from about 1:1.3 to about 1:1.7, from about 1:1.4 to about 1:1.6, or about 1:1.5. In some embodiments, the zeta potential of the nanoparticles is about −5 or less, about −10 or less, about −15 or less, about −20 or less, or about −25 or less. In various embodiments, the zeta potential of the nanoparticles is about −35 or higher, about −30 or higher, or about −25 or higher. In some embodiments, the nanoparticles have a zeta potential from about 0 mV to about −50 mV, from about 0 mV to about −40 mV or from about −10 mV to about −30 mV.

In some embodiments, the pharmaceutical compositions of the disclosure comprise a nanoparticle or a liposome that encapsulates a DNA, RNA or DNA/RNA hybrid comprising at least one expressible nucleic acid sequence. Liposomes are microscopic lipidic vesicles often having one or more bilayers of a vesicle-forming lipid, such as a phospholipid, and are capable of encapsulating a drug. Different types of liposomes may be employed in the context of the present disclosure, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation and the selection of the type of vesicles to be used will depend on the preferred mode of administration. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. In some embodiments, one or a plurality of the above-described phases are present in the nanoparticulate RNA formulations of the present disclosure.

For formation of RNA lipoplexes from RNA and liposomes, any suitable method of forming liposomes can be used so long as it provides the envisaged RNA lipoplexes. In some embodiments, liposomes are formed using standard methods such as the reverse evaporation method (REV), the ethanol injection method, the dehydration-rehydration method (DRV), sonication or other suitable methods.

After liposome formation, the liposomes can be sized to obtain a population of liposomes having a substantially homogeneous size range.

Bilayer-forming lipids have typically two hydrocarbon chains, particularly acyl chains, and a head group, either polar or nonpolar. Bilayer-forming lipids are either composed of naturally occurring lipids or of synthetic origin, including the phospholipids, such as phosphatidylcholine, phosphatidylethanolamine, phosphatide acid, phosphatidylinositol, and sphingomyelin, where the two hydrocarbon chains are typically between about 14-22 carbon atoms in length, and have varying degrees of unsaturation. Other suitable lipids for use in the composition of the present invention include glycolipids and sterols such as cholesterol and its various analogs which can also be used in the liposomes.

Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2 (spermine carboxamide)ethyl]-N,N-dimethyl-1-propanamium trifluoroacetate (DOSPA). In some embodiments, the cationic lipids are DOTMA, DOTAP, DODAC, and DOSPA. In some embodiments, the cationic lipid is DOTMA.

In addition, the nanoparticles described herein preferably further include a neutral lipid in view of structural stability and the like. The neutral lipid can be appropriately selected in view of the delivery efficiency of the RNA-lipid complex. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, sterol, and cerebroside. In some embodiments, the neutral lipid is DOPE and/or DOPC. In some embodiments, the neutral lipid is DOPE. In the case where a cationic liposome includes both a cationic lipid and a neutral lipid, the molar ratio of the cationic lipid to the neutral lipid can be appropriately determined in view of stability of the liposome and the like.

In some embodiments, the nanoparticles described herein comprise phospholipids. In some embodiments, the phospholipids are glycerophospholipids. Examples of glycerophospholipid include, without being limited thereto, three types of lipids: (i) zwitterionic phospholipids, which include, for example, phosphatidylcholine (PC), egg yolk phosphatidylcholine, soybean-derived PC in natural, partially hydrogenated or fully hydrogenated form, dimyristoyl phosphatidylcholine (DMPC) sphingomyelin (SM); (ii) negatively charged phospholipids: which include, for example, phosphatidylserine (PS), phosphatidylinositol (PI), phosphatidic acid (PA), phosphatidylglycerol (PG) dipalmipoyl PG, dimyristoyl phosphatidylglycerol (DMPG); synthetic derivatives in which the conjugate renders a zwitterionic phospholipid negatively charged such is the case of methoxy-polyethylene,glycol-distearoyl phosphatidylethanolamine (mPEG-DSPE); and (iii) cationic phospholipids, which include, for example, phosphatidylcholine or sphingomyelin of which the phosphomonoester was O-methylated to form the cationic lipids.

Association of RNA to the lipid carrier can occur, for example, by the RNA filling interstitial spaces of the carrier, such that the carrier physically entraps the RNA, or by covalent, ionic, or hydrogen bonding, or by means of adsorption by non-specific bonds. Whatever the mode of association, the RNA must retain its therapeutic, i.e. antigen-encoding, properties.

In some embodiments, the nanoparticles comprise at least one lipid. In some embodiments, the nanoparticles comprise at least one cationic lipid. The cationic lipid can be monocationic or polycationic. Any cationic amphiphilic molecule, eg, a molecule which comprises at least one hydrophilic and lipophilic moiety is a cationic lipid within the meaning of the present invention. In some embodiments, the positive charges are contributed by the at least one cationic lipid and the negative charges are contributed by the RNA. In some embodiments, the nanoparticles comprise at least one helper lipid. The helper lipid may be a neutral or an anionic lipid. In some embodiments, the helper lipid is a natural lipid, such as a phospholipid or an analogue of a natural lipid. In some embodiments, the helper lipid is a fully synthetic lipid, or lipid-like molecule, with no similarities with natural lipids. In some embodiments, the cationic lipid and/or the helper lipid is a bilayer forming lipid.

In some embodiments, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof. In some embodiments, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof. In some embodiments, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In some embodiments, in this ratio, the molar amount of the cationic lipid results from the molar amount of the cationic lipid multiplied by the number of positive charges in the cationic lipid. In various embodiments, the lipids are not functionalized such as functionalized by mannose, histidine and/or imidazole, the nanoparticles do not comprise a targeting ligand such as mannose functionalized lipids and/or the nanoparticles do not comprise one or more of the following: pH dependent compounds, cationic polymers such as polymers containing histidine and/or polylysine, wherein the polymers may optionally be PEGylated and/or histidylated, or divalent ions such as Ca 2+.

In various embodiments, the RNA nanoparticles may comprise peptides, preferentially with a molecular weight of up to 2500 Da.

In the nanoparticles described herein, the lipid may form a complex with and/or may encapsulate the RNA. In some embodiments, the nanoparticles comprise a lipoplex or liposome. In some embodiments, the lipid is comprised in a vesicle encapsulating said RNA. In some embodiments, the vesicle is a multilamellar vesicle, an unilamellar vesicle, or a mixture thereof. In some embodiments, the vesicle is a liposome. In some embodiments, the nanoparticles are lipoplexes comprising DOTMA and DOPE in a molar ratio of from about 10:0 to about 1:9, from about 8:2 to about 3:7, or from about 7:3 to about 5:5 and wherein the charge ratio of positive charges in DOTMA to negative charges in the RNA is from about 1.8:2 to about 0.8:2, from about 1.6:2 to about 1:2, from about 1.4:2 to about 1.1:2, or about 1.2:2.

In some embodiments, the nanoparticles are lipoplexes comprising DOTMA and Cholesterol in a molar ratio of from about 10:0 to about 1:9, from about 8:2 to about 3:7, or from about 7:3 to about 5:5 and wherein the charge ratio of positive charges in DOTMA to negative charges in the RNA is from about 1.8:2 to about 0.8:2, from about 1.6:2 to about 1:2, from about 1.4:2 to about 1.1:2, or about 1.2:2. In some embodiments, the nanoparticles are lipoplexes comprising DOTAP and DOPE in a molar ratio of from about 10:0 to about 1:9, from about 8:2 to about 3:7, or from about 7:3 to about 5:5 and wherein the charge ratio of positive charges in DOTMA to negative charges in the RNA is from about 1.8:2 to about 0.8:2, from about 1.6:2 to about 1:2, from about 1.4:2 to about 1.1:2, or about 1.2:2. In some embodiments, the nanoparticles are lipoplexes comprising DOTMA and DOPE in a molar ratio of from about 2:1 to about 1:2, or from about 2:1 to about 1:1, and wherein the charge ratio of positive charges in DOTMA to negative charges in the RNA is about 1.4:1 or less. In some embodiments, the nanoparticles are lipoplexes comprising DOTMA and cholesterol in a molar ratio of from about 2:1 to about 1:2, or from about 2:1 to about 1:1, and wherein the charge ratio of positive charges in DOTMA to negative charges in the RNA is about 1.4:1 or less. In some embodiments, the nanoparticles are lipoplexes comprising DOTAP and DOPE in a molar ratio of from about 2:1 to about 1:2, or from about 2:1 to about 1:1, and wherein the charge ratio of positive charges in DOTAP to negative charges in the RNA is about 1.4:1 or less. In some embodiments, the nanoparticles have an average diameter in the range of from about 50 nm to about 1000 nm, from about 50 nm to about 400 nm, about 100 nm to about 300 nm, or from about 150 nm to about 200 nm. In some embodiments, the nanoparticles have a diameter in the range of from about 200 to about 700 nm, from about 200 to about 600 nm, from about 250 to about 550 nm, from about 300 to about 500 nm, or from about 200 to about 400 nm.

In some embodiments, the polydispersity index of the nanoparticles described herein as measured by dynamic light scattering is about 0.5 or less, about 0.4 or less, or about 0.3 or less. In some embodiments, the nanoparticles described herein are obtainable by one or more of the following: (i) incubation of liposomes in an aqueous phase with the RNA in an aqueous phase, (ii) incubation of the lipid dissolved in an organic, water miscible solvent, such as ethanol, with the RNA in aqueous solution, (iii) reverse phase evaporation technique, (iv) freezing and thawing of the product, (v) dehydration and rehydration of the product, (vi) lyophilization and rehydration of the of the product, or (vii) spray drying and rehydration of the product.

In some embodiments, the nanoparticle formulation comprises a polymer conjugate. In some embodiments, the polymer conjugate is a water-soluble conjugate. In some embodiments, the polymer conjugate has a structure as described in U.S. Publication No. 20130059360, the content of which is herein incorporated by reference in its entirety. In some embodiments, polymer conjugates with the polynucleotides of the present disclosure are made using the methods and/or segmented polymeric reagents described in U.S. Publication No. 20130072709, herein incorporated by reference in its entirety. In other embodiments, the polymer conjugate has pendant side groups comprising ring moieties such as, but not limited to, the polymer conjugates described in U.S. Publication No. US20130196948, the contents of which is herein incorporated by reference in its entirety.

In some embodiments, the nanoparticle formulations comprise a conjugate to enhance the delivery of nanoparticles of the present invention in a subject. Further, the conjugate may inhibit phagocytic clearance of the nanoparticles in a subject. In some embodiments, the conjugate is a “self” peptide designed from the human membrane protein CD47 (e.g., the “self” particles described by Rodriguez et al. (Science 2013, 339, 971-975), herein incorporated by reference in its entirety). As shown by Rodriguez et al., the self peptides delayed macrophage-mediated clearance of nanoparticles which enhanced delivery of the nanoparticles. In other embodiments, the conjugate is the membrane protein CD47 (e.g., see Rodriguez et al. Science 2013, 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, about 100% of the uracil in the open reading frame have a chemical modification. In some embodiments, a chemical modification is in the 5′-position of the uracil. In some embodiments, a chemical modification is a N1-methyl pseudouridine. In some embodiments, about 100% of the uracil in the open reading frame have a N1-methyl pseudouridine in the 5′-position of the uracil.

In some embodiments, the neoantigen-coding RNA of the disclosure is administered into the subject in form of a RNA vaccine. In some embodiments, efficacy of the RNA vaccines of the disclosure can be significantly enhanced when combined with a flagellin adjuvant, in particular, when one or more RNA vaccines are combined with an mRNA encoding flagellin.

RNA vaccines combined with the flagellin adjuvant (e.g., mRNA-encoded flagellin adjuvant) have superior properties in that they may produce much larger antibody titers and produce responses earlier than commercially available vaccine formulations. While not wishing to be bound by theory, it is believed that the RNA vaccines, for example, as mRNA polynucleotides, are better designed to produce the appropriate protein conformation upon translation, for both the antigen and the adjuvant, as the RNA vaccines co-opt natural cellular machinery. Unlike traditional vaccines, which are manufactured ex vivo and may trigger unwanted cellular responses, RNA vaccines are presented to the cellular system in a more native fashion.

In some embodiments, the RNA vaccines of the disclosure comprise at least one RNA (e.g., mRNA) polynucleotide having an open reading frame encoding one or a plurality of cancer-specific and subject-specific neoantigens and at least one RNA (e.g., mRNA polynucleotide) having an open reading frame encoding a flagellin adjuvant. In some embodiments, at least one flagellin polypeptide (e.g., encoded flagellin polypeptide) is a flagellin protein. In some embodiments, at least one flagellin polypeptide (e.g., encoded flagellin polypeptide) is an immunogenic flagellin fragment. In some embodiments, at least one flagellin polypeptide and at least one antigenic polypeptide are encoded by a single RNA (e.g., mRNA) polynucleotide. In other embodiments, at least one flagellin polypeptide and at least one antigenic polypeptide are each encoded by a different RNA polynucleotide.

In some embodiments, the disclosed method further comprises formulating the cancer-specific and subject-specific neoantigens into an immunogenic composition for administration to the subject. In some embodiments, the cancer-specific and subject-specific neoantigens are formulated into an immunogenic composition in form of DNAs. In some embodiments, the cancer-specific and subject-specific neoantigens are formulated into an immunogenic composition in form of RNAs. In some embodiments, the cancer-specific and subject-specific neoantigens are formulated into an immunogenic composition in form of proteins. In the embodiments where the cancer-specific and subject-specific neoantigens are formulated into an immunogenic composition in form of DNAs and/or RNAs, the DNA/RNA neoantigens can be subcloned into one or a plurality of vectors, which in some embodiments, are one or a plurality of plasmids. Methods of administering DNA vaccines, RNA vaccines or protein vaccines are known in the art. One of skill in the art can determine which therapeutic regimen is appropriate on a subject by subject basis, depending, for example, on their cancer and their immune status (e.g., T-cell, B cell or NK cell activity and/or numbers).

Routes of administration include, but are not limited to, intramuscular, intranasally, intradermally, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. In some embodiments, genetic constructs are administered by means including, but not limited to, traditional syringes, needleless injection devices, “microprojectile bombardment gone guns,” or other physical methods such as electroporation (“EP”), “hydrodynamic method,” or ultrasound.

For therapeutic or immunization purposes, nucleic acid molecules encoding the one or plurality of neoantigens can also be administered to the subject. A number of methods are conveniently used to deliver the nucleic acids to the subject. For instance, the nucleic acid can be delivered directly, as “naked DNA.” This approach is described, for instance, in Wolff et al., Science 247:1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO1996/18372; WO 1993/24640; Mannino & Gould-Fogerite, BioTechniques 6 (7): 682-691 (1988); U.S. Pat. No. 5,279,833; WO 1991/06309; and Feigner et al., Proc. Natl. Acad. Sci. USA 84:7413-7414 (1987).

RNA encoding the neoantigen can also be used for delivery (see, e.g., Kiken et al, 2011; Su et al, 2011).

A pharmaceutically acceptable carrier or excipient can include such functional molecules as vehicles, adjuvants, carriers or diluents, which are known and readily available to the public. In some embodiments, the pharmaceutically acceptable carrier is an adjuvant. In some embodiments, the pharmaceutically acceptable excipient is a transfection facilitating agent. In some embodiments, the transfection facilitating agent is a polyanion, polycation, or lipid, and more preferably poly-L-glutamate. In some embodiments, the nucleic acid molecule, or DNA plasmid, is delivered to the cells in conjunction with administration of a polynucleotide function enhancer or a genetic vaccine facilitator agent (or transfection facilitating agent). Polynucleotide function enhancers are described in U.S. Pat. Nos. 5,593,972, 5,962,428, and International Patent Application No. PCT/US94/00899 filed Jan. 26, 1994, which are each incorporated herein by reference in their entireties. Genetic vaccine facilitator agents are described in U.S. Patent Application Ser. No. 021,579 filed Apr. 1, 1994, which is incorporated herein by reference in its entirety. The transfection facilitating agent can be administered in conjunction with nucleic acid molecules as a mixture with the nucleic acid molecule or administered separately simultaneously, before or after administration of nucleic acid molecules. Examples of transfection facilitating agents includes surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

In some embodiments, the DNA plasmids are delivered with genes for proteins which further enhance the immune response. Examples of such genes are those which encode other cytokines and lymphokines such as alpha-interferon, gamma-interferon, platelet derived growth factor (PDGF), TNF α, TNFβ, GM-CSF, epidermal growth factor (EGF), IL-1, IL-2, IL-4, IL-5, IL-6, IL-10, IL-12, IL-18, MHC, CD80, CD86 and IL-15 including IL-15 having the signal sequence deleted and optionally including the signal peptide from IgE. Other genes which may be useful include those encoding: MCP-1, MIP-1α, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the disclosure can be varied so as to obtain an amount of the active ingredient which is effective to achieve the desired therapeutic response for a particular patient, composition, and mode of administration, without being toxic to the patient. Generally, agents or pharmaceutical compositions of the disclosure are administered in an amount sufficient to induce immunogenic response in the subject.

The one or a plurality of nucleic acid molecules encoding cancer-specific and subject-specific neoantigens described herein, or compositions comprising the same, for administration to the subject may comprise DNA quantities of from about 1 nanogram to 10 milligrams; from about 1 microgram to about 10 milligrams; from about 0.1 microgram to about 10 milligrams; or from about 100 microgram to about 1 milligram. In some embodiments, the one or a plurality of nucleic acid molecules encoding cancer-specific and subject-specific neoantigens described herein, or compositions comprising the same, for administration to the subject comprise from about 5 nanograms to about 1000 micrograms of DNA. In some embodiments, the one or a plurality of nucleic acid molecules encoding cancer-specific and subject-specific neoantigens described herein, or compositions comprising the same, for administration to the subject comprise from about 10 nanograms to about 800 micrograms of DNA. In some embodiments, the one or a plurality of nucleic acid molecules encoding cancer-specific and subject-specific neoantigens described herein, or compositions comprising the same, for administration to the subject comprise from about 0.1 to about 500 micrograms of DNA. In some embodiments, the one or a plurality of nucleic acid molecules encoding cancer-specific and subject-specific neoantigens described herein, or compositions comprising the same, for administration to the subject comprise from about 1 to about 350 micrograms of DNA. In some embodiments, the one or a plurality of nucleic acid molecules encoding cancer-specific and subject-specific neoantigens described herein, or compositions comprising the same, for administration to the subject comprise from about 25 to about 250 micrograms of DNA. In some embodiments, the one or a plurality of nucleic acid molecules encoding cancer-specific and subject-specific neoantigens described herein, or compositions comprising the same, for administration to the subject comprise from about 100 microgram to about 1 milligram DNA.

The one or a plurality of nucleic acid molecules encoding cancer-specific and subject-specific neoantigens described herein, or compositions comprising the same, for administration to the subject according to the disclosure are formulated according to the mode of administration to be used. In cases where they are injectable pharmaceutical compositions, they are sterile, pyrogen free and particulate free. An isotonic formulation can be used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

In some embodiments, the DNA formulations for use with a muscle or skin EP device have high DNA concentrations, such as concentrations that include microgram to tens of milligram quantities, or milligram quantities, of DNA in small volumes that are optimal for delivery to the skin, such as small injection volume, ideally 25-200 microliters (μL). In some embodiments, the DNA formulations have high DNA concentrations, such as 1 mg/mL or greater (mg DNA/volume of formulation). In some embodiments, the DNA formulation has a DNA concentration that provides for gram quantities of DNA in 200 μL of formula. In some embodiments, the DNA formulation has a DNA concentration that provides for gram quantities of DNA in 100 μL of formula.

The DNA plasmids of the disclosure for use with the electroporation devices can be formulated or manufactured using a combination of known devices and techniques, such as being manufactured using an optimized plasmid manufacturing technique that is described in U.S. Patent Application Publication No. 20090004716, incorporated by reference in its entirety herein. In some embodiments, the DNA plasmids used can be formulated at concentrations greater than or equal to 10 mg/mL. The manufacturing techniques also include or incorporate various devices and protocols that are commonly known to those of ordinary skill in the art, in addition to those described in U.S. Patent Application Publication No. 20090004716 and those described in U.S. Pat. No. 7,238,522, incorporated by reference in their entireties herein. The high concentrations of plasmids used with the skin electroporation devices and delivery techniques described herein allow for administration of plasmids into the ID/SC space in a reasonably low volume and aids in enhancing expression and immunization effects.

The amounts and dosage regimens administered to a subject will depend on a number of factors, such as the mode of administration, the nature of the condition being treated, the body weight of the subject being treated and the judgment of the prescribing physician. The quantity of nucleic acid molecules (DNA or RNA) included within therapeutically active formulations according to the disclosure is an effective amount for inducing immunogenic response to one or a plurality of neoantigens in the subject. Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., inducing immunogenic response) is observed in the treated subject, with minimal or acceptable toxic side effects. Applicable methods for determining an appropriate dose and dosing schedule for administration of a pharmaceutical composition of the present disclosure are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

### Activation, Expansion and Isolation of Neoantigen-Specific T Cells

Once administered into the subject, the cancer-specific and subject-specific neoantigens activate the subject's immune response to produce T cells that are specific to the cancer-specific and subject-specific neoantigens, hereinafter called “clonal T cells.” Such clonal T cells primed against one or a plurality of the cancer-specific and subject-specific neoantigens can then be expanded in vivo in the subject after certain time period. A primed T cell is one in which the T cells respond to an epitope such that the T cells expand in number. This generally takes about 3-5 days and peaks around 7-10 days.

In some embodiments, the subject is diagnosed of having cancer. In some embodiments, the subject is suspected of having cancer. In some embodiments, the subject has previously been treated, and not responded to checkpoint inhibitor therapy.

In some embodiments, the nucleic acid molecule is administered to the subject by electroporation. In some embodiments, the method is free of a step using electroporation for nucleic acid sequence administration.

In some embodiments, the cancer-specific and subject-specific neoantigens activate the CD8+ T cell immune response in the subject. In some embodiments, activating the CD8+ T cell immune response comprises activating from about 0.01% to about 50% CD8+T cells with reactivity to the one or plurality of neoantigens. In some embodiments, activating the CD8+ T cell immune response comprises activating from about 0.05% to about 50% CD8+ T cells with reactivity to the one or plurality of neoantigens. In some embodiments, activating the CD8+ T cell immune response comprises activating from about 0.1% to about 50% CD8+ T cells with reactivity to the one or plurality of neoantigens. In some embodiments, activating the CD8+ T cell immune response comprises activating from about 0.2% to about 50% CD8+ T cells with reactivity to the one or plurality of neoantigens. In some embodiments, activating the CD8+ T cell immune response comprises activating from about 0.3% to about 50% CD8+ T cells with reactivity to the one or plurality of neoantigens. In some embodiments, activating the CD8+ T cell immune response comprises activating from about 0.4% to about 50% CD8+ T cells with reactivity to the one or plurality of neoantigens. In some embodiments, activating the CD8+ T cell immune response comprises activating from 0.5% to about 50% CD8+ T cells with reactivity to the one or plurality of neoantigens. In some embodiments, activating the CD8+ T cell immune response comprises activating from about 0.6% to about 50% CD8+ T cells with reactivity to the one or plurality of neoantigens. In some embodiments, activating the CD8+ T cell immune response comprises activating from about 0.7% to about 50% CD8+ T cells with reactivity to the one or plurality of neoantigens. In some embodiments, activating the CD8+ T cell immune response comprises activating from about 0.8% to about 50% CD8+ T cells with reactivity to the one or plurality of neoantigens. In some embodiments, activating the CD8+ T cell immune response comprises activating from about 0.9% to about 50% CD8+ T cells with reactivity to the one or plurality of neoantigens. In some embodiments, activating the CD8+ T cell immune response comprises activating from about 1% to about 50% CD8+ T cells with reactivity to the one or plurality of neoantigens. In some embodiments, activating the CD8+ T cell immune response comprises activating from about 2% to about 50% CD8+ T cells with reactivity to the one or plurality of neoantigens. In some embodiments, activating the CD8+ T cell immune response comprises activating from about 3% to about 50% CD8+ T cells with reactivity to the one or plurality of neoantigens. In some embodiments, activating the CD8+ T cell immune response comprises activating from about 4% to about 50% CD8+ T cells with reactivity to the one or plurality of neoantigens. In some embodiments, activating the CD8+ T cell immune response comprises activating from about 5% to about 50% CD8+ T cells with reactivity to the one or plurality of neoantigens. In some embodiments, activating the CD8+ T cell immune response comprises activating from about 6% to about 50% CD8+ T cells with reactivity to the one or plurality of neoantigens. In some embodiments, activating the CD8+ T cell immune response comprises activating from about 7% to about 50% CD8+ T cells with reactivity to the one or plurality of neoantigens. In some embodiments, activating the CD8+ T cell immune response comprises activating from about 8% to about 50% CD8+ T cells with reactivity to the one or plurality of neoantigens. In some embodiments, activating the CD8+ T cell immune response comprises activating from about 9% to about 50% CD8+ T cells with reactivity to the one or plurality of neoantigens. In some embodiments, activating the CD8+ T cell immune response comprises activating from about 10% to about 50% CD8+ T cells with reactivity to the one or plurality of neoantigens. In some embodiments, activating the CD8+ T cell immune response comprises activating from about 15% to about 50% CD8+ T cells with reactivity to the one or plurality of neoantigens. In some embodiments, activating the CD8+ T cell immune response comprises activating from about 20% to about 50% CD8+ T cells with reactivity to the one or plurality of neoantigens. In some embodiments, activating the CD8+ T cell immune response comprises activating from about 25% to about 50% CD8+ T cells with reactivity to the one or plurality of neoantigens.

In some embodiments, activating the CD8+ T cell immune response comprises activating from about 0.01% to about 50% CD8+ T cells with reactivity to the one or plurality of neoantigens that are IFN-γ positive. In some embodiments, activating the CD8+ T cell immune response comprises activating from about 0.05% to about 50% CD8+ T cells with reactivity to the one or plurality of neoantigens that are IFN-γ positive. In some embodiments, activating the CD8+ T cell immune response comprises activating from about 0.1% to about 50% CD8+ T cells with reactivity to the one or plurality of neoantigens that are IFN-γ positive. In some embodiments, activating the CD8+ T cell immune response comprises activating from about 0.2% to about 50% CD8+ T cells with reactivity to the one or plurality of neoantigens that are IFN-γ positive. In some embodiments, activating the CD8+ T cell immune response comprises activating from about 0.3% to about 50% CD8+ T cells with reactivity to the one or plurality of neoantigens that are IFN-positive. In some embodiments, activating the CD8+ T cell immune response comprises activating from about 0.4% to about 50% CD8+ T cells with reactivity to the one or plurality of neoantigens that are IFN-γ positive. In some embodiments, activating the CD8+ T cell immune response comprises activating from 0.5% to about 50% CD8+ T cells with reactivity to the one or plurality of neoantigens that are IFN-γ positive. In some embodiments, activating the CD8+ T cell immune response comprises activating from about 0.6% to about 50% CD8+ T cells with reactivity to the one or plurality of neoantigens that are IFN-γ positive. In some embodiments, activating the CD8+ T cell immune response comprises activating from about 0.7% to about 50% CD8+ T cells with reactivity to the one or plurality of neoantigens that are IFN-γ positive. In some embodiments, activating the CD8+ T cell immune response comprises activating from about 0.8% to about 50% CD8+ T cells with reactivity to the one or plurality of neoantigens that are IFN-γ positive. In some embodiments, activating the CD8+ T cell immune response comprises activating from about 0.9% to about 50% CD8+ T cells with reactivity to the one or plurality of neoantigens that are IFN-γ positive. In some embodiments, activating the CD8+ T cell immune response comprises activating from about 1% to about 50% CD8+ T cells with reactivity to the one or plurality of neoantigens that are IFN-γ positive. In some embodiments, activating the CD8+ T cell immune response comprises activating from about 2% to about 50% CD8+ T cells with reactivity to the one or plurality of neoantigens that are IFN-γ positive. In some embodiments, activating the CD8+ T cell immune response comprises activating from about 3% to about 50% CD8+ T cells with reactivity to the one or plurality of neoantigens that are IFN-γ positive. In some embodiments, activating the CD8+ T cell immune response comprises activating from about 4% to about 50% CD8+ T cells with reactivity to the one or plurality of neoantigens that are IFN-γ positive. In some embodiments, activating the CD8+ T cell immune response comprises activating from about 5% to about 50% CD8+ T cells with reactivity to the one or plurality of neoantigens that are IFN-γ positive. In some embodiments, activating the CD8+ T cell immune response comprises activating from about 6% to about 50% CD8+ T cells with reactivity to the one or plurality of neoantigens that are IFN-γ positive. In some embodiments, activating the CD8+ T cell immune response comprises activating from about 7% to about 50% CD8+ T cells with reactivity to the one or plurality of neoantigens that are IFN-γ positive. In some embodiments, activating the CD8+ T cell immune response comprises activating from about 8% to about 50% CD8+ T cells with reactivity to the one or plurality of neoantigens that are IFN-γ positive. In some embodiments, activating the CD8+ T cell immune response comprises activating from about 9% to about 50% CD8+ T cells with reactivity to the one or plurality of neoantigens that are IFN-γ positive. In some embodiments, activating the CD8+ T cell immune response comprises activating from about 10% to about 50% CD8+ T cells with reactivity to the one or plurality of neoantigens that are IFN-γ positive. In some embodiments, activating the CD8+ T cell immune response comprises activating from about 15% to about 50% CD8+ T cells with reactivity to the one or plurality of neoantigens that are IFN-γ positive. In some embodiments, activating the CD8+ T cell immune response comprises activating from about 20% to about 50% CD8+ T cells with reactivity to the one or plurality of neoantigens that are IFN-γ positive. In some embodiments, activating the CD8+ T cell immune response comprises activating from about 25% to about 50% CD8+ T cells with reactivity to the one or plurality of neoantigens that are IFN-γ positive.

In some embodiments, the activation of CD8+ T cells is accomplished after no more than about 1 hour of contact with antigen presenting cells expressing or comprising the nucleic acid sequences encoding the cancer-specific and subject-specific neoantigens in the subject. In some embodiments, the activation of CD8+ T cells is accomplished after no more than about 2 hours of contact with antigen presenting cells expressing or comprising the nucleotic acid sequences encoding the cancer-specific and subject-specific neoantigens in the subject. In some embodiments, the activation of CD8+ T cells is accomplished after no more than about 3 hours of contact with antigen presenting cells expressing or comprising the nucleotic acid sequences encoding the cancer-specific and subject-specific neoantigens in the subject. In some embodiments, the activation of CD8+ T cells is accomplished after no more than about 4 hours of contact with antigen presenting cells expressing or comprising the nucleotic acid sequences encoding the cancer-specific and subject-specific neoantigens in the subject. In some embodiments, the activation of CD8+ T cells is accomplished after no more than about 5 hours of contact with antigen presenting cells expressing or comprising the nucleotic acid sequences encoding the cancer-specific and subject-specific neoantigens in the subject. In some embodiments, the activation of CD8+ T cells is accomplished after no more than about 6 hours of contact with antigen presenting cells expressing or comprising the nucleotic acid sequences encoding the cancer-specific and subject-specific neoantigens in the subject. In some embodiments, the activation of CD8+ T cells is accomplished after no more than about 7 hours of contact with antigen presenting cells expressing or comprising the nucleotic acid sequences encoding the cancer-specific and subject-specific neoantigens in the subject. In some embodiments, the activation of CD8+ T cells is accomplished after no more than about 8 hours of contact with antigen presenting cells expressing or comprising the nucleotic acid sequences encoding the cancer-specific and subject-specific neoantigens in the subject. In some embodiments, the activation of CD8+ T cells is accomplished after no more than about 9 hours of contact with antigen presenting cells expressing or comprising the nucleotic acid sequences encoding the cancer-specific and subject-specific neoantigens in the subject. In some embodiments, the activation of CD8+ T cells is accomplished after no more than about 10 hours of contact with antigen presenting cells expressing or comprising the nucleotic acid sequences encoding the cancer-specific and subject-specific neoantigens in the subject. In some embodiments, the activation of CD8+ T cells is accomplished after more than about 10 hours of contact with antigen presenting cells expressing or comprising the nucleotic acid sequences encoding the cancer-specific and subject-specific neoantigens in the subject.

In some embodiments, PBMCs from the subject are taken as a sample and then that sample is run through an activation assay in order to identity T cell populations that are activated in the presence of neoantigens.

In some embodiments, the activation of CD8+ T cell immune response comprises expanding CD8+ T cells in culture that are specific to the cancer-specific and subject-specific neoantigens in the subject to a biologically significant number or concentration in fluid. In some embodiments, the biologically significant number of the neoantigen-specific CD8+ T cells is from about 100 cells per mL of blood in a subject to about 200 cells per mL of blood in a subject. If the cell are clonally isolated and expanded in some embodiments, the biologically significant number of the neoantigen-specific CD8+ T cells is from about 100 cells per mL of pharmaceutically acceptable carrier to about 200 cells per mL of pharmaceutically acceptable carrier. If the cell are clonally isolated and expanded in some embodiments, the biologically significant number of the neoantigen-specific CD8+ T cells is from about 75 cells per mL of pharmaceutically acceptable carrier to about 150 cells per mL of pharmaceutically acceptable carrier. In some embodiments, the CD8+ T cells are suspended in sterile buffered saline. In some embodiments, the CD8+ cells are in a pharmaceutically acceptable carrier such as sterile saline for enabling administration back to the subject.

Activation of T cells can be detected and measured in a variety of ways. Methods for detecting specific T cell activation include detecting the proliferation of T cells, the production of cytokines (e.g., lymphokines), or the generation of cytolytic activity. Methods for measuring T cell activities include, but not limited to, the induction of proliferation of T cells, the induction of signaling in T cells, the induction of expression of activation markers in T cells, such as interferon-gamma (IFN-γ), the induction of cytokine secretion by T cells, and the cytotoxic activity of T cells. For example, in some embodiments, CD8+ T cell activation is measured by a proliferation assay. In other embodiments, the activation is measured after stimulation of cells or cell sample by the encoded nucleic acid sequences after cells have been isolated from the subject.

In some embodiments, the activation of CD8+ T-cells is assessed or measured by determining secretion of cytokines, such as gamma interferon (IFN-γ), tumor necrosis factor alpha (TNFa), interleukin-12 (IL-12) or interleukin 2 (IL-2). In some embodiments, ELISA is used to determine cytokine secretion, for example secretion of gamma interferon (IFN-γ), tumor necrosis factor alpha (TNFa), interleukin-12 (IL-12) or interleukin 2 (IL-2). In some embodiments, the ELISPOT (enzyme-linked immunospot) technique is used to detect T cells that secrete a given cytokine (e.g., gamma interferon (IFN-γ)) in response to stimulation with the cancer-specific and subject-specific neoantigens, or any compositions comprising the same. T cells are placed in wells which have been coated with anti-IFN-γ antibodies. The secreted IFN-γ is captured by the coated antibody and then revealed with a second antibody coupled to a chromogenic substrate. Thus, locally secreted cytokine molecules form spots, with each spot corresponding to one IFN-γ-secreting cell. The number of spots allows one to determine the frequency of IFN-γ-secreting cells in the analyzed sample. The ELISPOT assay has also been described for the detection of tumor necrosis factor alpha, interleukin-4 (IL-4), IL-5, IL-6, IL-10, IL-12, granulocyte-macrophage colony-stimulating factor, and granzyme B-secreting lymphocytes (Klinman D, Nutman T. Current protocols in immunology. New York, N.Y: John Wiley & Sons, Inc.; 1994. pp. 6.19.1-6.19.8, incorporated by reference in its entirety herein).

Flow cytometric analyses of intracellular cytokines may also be used to measure the cytokine content, but provides no information on the number of T cells that actually secrete the cytokine. When T cells are treated with inhibitors of secretion such as monensin or brefeldin A, they accumulate cytokines within their cytoplasm upon activation (e.g. with the nucleic acid molecules of the present invention). After fixation and permeabilization of the lymphocytes, intracellular cytokines can be quantified by cytometry. This technique allows the determination of the cytokines produced, the type of cells that produce these cytokines, and the quantity of cytokine produced per cell.

In some embodiments, the activation of CD8+ T-cells is determined by assaying the cytotoxic activity of the CD8+ T-cells. The cytotoxic activity of T cells may be assessed by any suitable technique known to those of skill in the art. For example, a sample comprising T cells that have been exposed to one or a plurality of the neoantigens according to the disclosure can be assayed for cytotoxic activity in a standard cytotoxic assay. Such assays may include, but are not limited to, the chromium release CTL assay and the Alamar Blue™ fluorescence assay known in the art.

In some embodiments, activation and expansion of neoantigen-specific T cells as disclosed herein enhances expression of certain key molecules in T cells that protect again apoptosis or otherwise prolong survival in vivo or in vitro. Apoptosis usually results from induction of a specific signal in the T cell. Thus, the neoantigens may provide for protecting a T cell from cell death resulting from stimulation of the T cell. Therefore, also included in the disclosed methods is the enhanced T cell growth by protection from premature death or from absence or depletion of recognized T cell growth markers, such as Bcl-xL, growth factors, cytokines, or lymphokines normally necessary for T cell survival, as well as from Fas or Tumor Necrosis Factor Receptor (TNFR) cross-linking or by exposure to certain hormones or stress.

The neoantigen-specific T cells produced by the subject may be isolated in a variety of ways. In some embodiments, the neoantigen-specific T cells are isolated by drawing a blood sample from the subject and sorting the peripheral blood mononuclear cell (PBMC) from the sample according to receptor expression on the PBMC surface. In some embodiments, the isolation of the neoantigen-specific T cells further comprises removing a human tissue sample from the subject. In some embodiments, the human tissue sample removed for isolation of the neoantigen-specific T cells comprises a tissue from a brushing, biopsy, or surgical resection of the subject. In some embodiments, the neoantigen-specific T cells are isolated based on expression of T cell activation markers by cell sorting or other appropriate techniques known in the art. In some embodiments, the disclosed methods further comprise determining which neoantigens are immunologically recognized by the T cells (e.g., by process of elimination).

In some embodiments, selecting the neoantigen-specific T cells comprises selecting the T cells (i) that secrete a greater amount of one or more cytokines as compared to the amount of the one or more cytokines secreted by a negative control or (ii) in which at least twice as many of the numbers of T cells secrete one or more cytokines as compared to the numbers of negative control T cells that secrete the one or more cytokines. The one or more cytokines may comprise any cytokine the secretion of which by a T cell is characteristic of T cell activation (e.g., a TCR expressed by the T cells specifically binding to and immunologically recognizing the neoantigen). Non-limiting examples of cytokines, the secretion of which is characteristic of T cell activation, include IFN-γ, IL-2, and tumor necrosis factor alpha (TNF-α), granulocyte/monocyte colony stimulating factor (GM-CSF), IL-4, IL-5, IL-9, IL-10, IL-17, and IL-22.

In some embodiments, the T cells are considered to have antigenic specificity for the neoantigen, and thus neoantigen-specific T cells, if the T cells secrete at least twice as much IFN-γ as compared to the amount of IFN-γ secreted by a negative control. In some embodiments, the negative control is, for example, autologous T cells (e.g., derived from PBMCs). IFN-γ secretion may be measured by methods known in the art such as, for example, enzyme-linked immunosorbent assay (ELISA).

In some embodiments, the disclosed methods further comprise isolating a nucleotide sequence that encodes the T cell receptor (TCR), or the antigen-binding portion thereof, from the selected neoantigen-specific T cells, wherein the TCR, or the antigen-binding portion thereof, has antigenic specificity for the neoantigen. In some embodiments, the disclosed methods further comprise identifying one or a plurality of nucleotide sequences encoding a subset of TCRs, or the antigen-binding portions thereof, that are highly immunogenic in response to the one or plurality of neoantigens in the subject. In some embodiments, the one or plurality of nucleotide sequences encoding the subset of TCRs that are highly immunogenic are identified by performing an assay measuring the avidity or affinity of cells expressing the TCRs to bind cells in vitro. In some embodiments, the one or plurality of nucleotide sequences encoding the subset of TCRs that are highly immunogenic are identified by performing an assay measuring the percentage of CD8+ and/or CD4+ on cells expressing the TCRs, or the antigen-binding portions thereof. In some embodiments, the methods may further comprise expanding cells expressing the TCRs, or the antigen-binding portions thereof, in culture prior to identification of one or a plurality of nucleotide sequences encoding a subset of TCRs, or the antigen-binding portions thereof, that are highly immunogenic in response to the one or plurality of neoantigens in the subject. In some embodiments, the methods may further comprise sequencing the nucleotide sequence encoding the one or plurality of TCRs, or the antigen-binding portions thereof, that are highly immunogenic from the cells expressing the TCRs, or the antigen-binding portions thereof.

The term “highly immunogenic,” as used herein, means that a T cell, TCR, or the antigen-binding portion thereof, expressed by the T cell, can specifically bind to and immunologically recognize the cancer-specific and subject-specific neoantigens of a degree that stimulates a biological response, such as secretion of cytokines after exposure to the neoantigens.

The “antigen-binding portion” of the TCR, as used herein, refers to any portion comprising contiguous amino acids of the TCR of which it is a part, provided that the antigen-binding portion specifically binds to the cancer-specific and subject-specific neoantigen. The term “antigen-binding portion” refers to any part or fragment of a TCR, which part or fragment retains the biological activity of the TCR of which it is a part (the parent TCR). Antigen-binding portions encompass, for example, those parts of a TCR that retain the ability to specifically bind to the cancer-specific and subject-specific neoantigen, or detect, treat, or prevent cancer, to a similar extent, the same extent, or to a higher extent, as compared to the parent TCR. In reference to the parent TCR, the functional portion can comprise, for instance, about 10%, 25%, 30%, 50%, 68%, 80%, 90%, 95%, or more in length, of the parent TCR.

The antigen-binding portion can comprise an antigen-binding portion of either or both of the α and β chains of the TCR, such as a portion comprising one or more of the complementarity determining region (CDR) 1, CDR2, and CDR3 of the variable region(s) of the α chain and/or β chain of the TCR. In some embodiments, the antigen-binding portion can comprise the amino acid sequence of the CDR 1 of the α chain (CDR1α), the CDR2 of the α chain (CDR2α), the CDR3 of the α chain (CDR3α), the CDR1 of the β chain (CDR1β), the CDR2 of the β chain (CDR2β), the CDR3 of the β chain (CDR3β), or any combination thereof. In some embodiments, the antigen-binding portion comprises the amino acid sequences of CDR1a, CDR2α, and CDR3α; the amino acid sequences of CDR1β, CDR2β, and CDR3β; or the amino acid sequences of all of CDR1α, CDR2α, CDR3α, CDR1β, CDR2β, and CDR3β of the TCR.

In some embodiments, the antigen-binding portion may comprise a combination of a variable region and a constant region. In some embodiments, the antigen-binding portion can comprise the entire length of the α or β chain, or both of the α and β chains, of the TCR.

Isolating the nucleotide sequence that encodes the TCR, or the antigen-binding portion thereof, from the selected T cells may be carried out in any suitable manner known in the art. For example, the method may comprise isolating RNA from the selected T cells and sequencing the TCR, or the antigen-binding portion thereof, using established molecular cloning techniques and reagents such as, for example, 5′ Rapid Amplification of cDNA Ends (RACE) polymerase chain reaction (PCR) using TCR-α and TCR-β chain constant primers.

The disclosure relates to a nucleic acid molecule comprising a first, second and third nucleic acid sequence, wherein the first nucleic acid sequence is a first DNA backbone domain of the nucleic acid molecule, the second nucleic acid sequence is the second DNA backbone domain of the nucleic acid molecule and the third nucleic acid sequence is an expressible nucleic acid sequence; wherein the expressible nucleic acid sequence comprises a plurality of antigen expression domains, in 5′ to 3′ orientation; wherein each antigen expression domain comprises a nucleotide sequence comprising at least about 70%, 75%, 80%, 85%, 90%, 91%, 92%, 93%, 94%, 95%, 96%, 97%, 98%, 99% or 100% sequence identity to SEQ ID NO:1-128 or a functional fragment that comprises at least about 70%, 75%, 80%, 85%, 90%, 91%, 92%, 93%, 94%, 95%, 96%, 97%, 98%, 99% sequence identity to any of SEQ ID NO:1-128.

The disclosure relates to a nucleic acid molecule comprising a first, second and third nucleic acid sequence, wherein the first nucleic acid sequence is a first DNA backbone domain of the nucleic acid molecule, the second nucleic acid sequence is the second DNA backbone domain of the nucleic acid molecule and the third nucleic acid sequence is an expressible nucleic acid sequence; wherein the expressible nucleic acid sequence comprises a plurality of antigen expression domains, in 5′ to 3′ orientation; wherein each antigen expression domain comprises a nucleotide sequence comprising at least about 70%, 75%, 80%, 85%, 90%, 91%, 92%, 93%, 94%, 95%, 96%, 97%, 98%, 99% or 100% sequence identity to SEQ ID NO: 397-494 or a functional fragment that comprises at least about 70%, 75%, 80%, 85%, 90%, 91%, 92%, 93%, 94%, 95%, 96%, 97%, 98%, 99% sequence identity to any of SEQ ID NO:397-494.

The disclosure relates to a cell comprising a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO: 161-167 or a functional fragment that comprises at least about 70%, 75%, 80%, 85%, 90%, 91%, 92%, 93%, 94%, 95%, 96%, 97%, 98%, 99% sequence identity to any of SEQ ID NO: 161-167.

The disclosure relates to a cell comprising a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO:367-394 or a functional fragment that comprises at least about 70%, 75%, 80%, 85%, 90%, 91%, 92%, 93%, 94%, 95%, 96%, 97%, 98%, 99% sequence identity to any of SEQ ID NO:367-394.

The disclosure relates to a cell comprising a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO:495-501 or a functional fragment that comprises at least about 70%, 75%, 80%, 85%, 90%, 91%, 92%, 93%, 94%, 95%, 96%, 97%, 98%, 99% sequence identity to any of SEQ ID NO:495-501.

The disclosure relates to a cell comprising a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO: 168-174 or a functional fragment that comprises at least about 70%, 75%, 80%, 85%, 90%, 91%, 92%, 93%, 94%, 95%, 96%, 97%, 98%, 99% sequence identity to any of SEQ ID NO:168-174.

The disclosure relates to a cell comprising a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO:331-355 or a functional fragment that comprises at least about 70%, 75%, 80%, 85%, 90%, 91%, 92%, 93%, 94%, 95%, 96%, 97%, 98%, 99% sequence identity to any of SEQ ID NO:331-355.

The disclosure relates to a cell comprising a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO:255-329 or a functional fragment that comprises at least about 70%, 75%, 80%, 85%, 90%, 91%, 92%, 93%, 94%, 95%, 96%, 97%, 98%, 99% sequence identity to any of SEQ ID NO:255-329.

The disclosure relates to a cell comprising a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO: 179-253 or a functional fragment that comprises at least about 70%, 75%, 80%, 85%, 90%, 91%, 92%, 93%, 94%, 95%, 96%, 97%, 98%, 99% sequence identity to any of SEQ ID NO:179-253.

The disclosure relates to a nucleic acid molecule that encodes a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO:179-253 or a functional fragment that comprises at least about 70%, 75%, 80%, 85%, 90%, 91%, 92%, 93%, 94%, 95%, 96%, 97%, 98%, 99% sequence identity to any of SEQ ID NO:179-253.

The disclosure relates to a nucleic acid molecule that encodes a TCR or antigen binding fragment thereof comprising at least about 70%, 75%, 80%, 85%, 90%, 91%, 92%, 93%, 94%, 95%, 96%, 97%, 98%, 99% or 100% sequence identity to SEQ ID NO:509-588 or a functional fragment that comprises at least about 70%, 75%, 80%, 85%, 90%, 91%, 92%, 93%, 94%, 95%, 96%, 97%, 98%, 99% sequence identity to any of SEQ ID NO:509-588.

The disclosure relates to a nucleic acid molecule that encodes a TCR or antigen binding fragment thereof comprising at least about 70%, 75%, 80%, 85%, 90%, 91%, 92%, 93%, 94%, 95%, 96%, 97%, 98%, 99% or 100% sequence identity to any of sequence identifier of Tables S, T and/or U, or a functional fragment that comprises at least about 70%, 75%, 80%, 85%, 90%, 91%, 92%, 93%, 94%, 95%, 96%, 97%, 98%, 99% sequence identity to any of sequence identifier of Tables S, T and/or U.

The disclosure relates to a nucleic acid molecule that encodes a TCR or antigen binding fragment thereof comprising at least about 70%, 75%, 80%, 85%, 90%, 91%, 92%, 93%, 94%, 95%, 96%, 97%, 98%, 99% or 100% sequence identity to any of sequence identifier of Tables Z, or a functional fragment that comprises at least about 70%, 75%, 80%, 85%, 90%, 91%, 92%, 93%, 94%, 95%, 96%, 97%, 98%, 99% sequence identity to any of sequence identifier of Table Z.

### TCR Expressing T Cells

Disclosed are cells comprising a TCR comprising one alpha and one beta subunit, wherein the alpha and beta subunits are those disclosed in Table Z. In some aspects, the TCR comprising one alpha and one beta subunit comprise one alpha and one beta subunit having at least 70%, 75%, 80%, 85%, 90%, 91%, 92%, 93%, 94%, 95%, 96%, 97%, 98%, 99% or 100% sequence identity to the alpha and beta subunits disclosed in Table Z.

Disclosed are cells comprising a TCR comprising one alpha and one beta subunit, wherein an alpha subunit is chosen from one or a combination of amino acid sequences that comprise at least about 70%, 75%, 80%, 85%, 90%, 91%, 92%, 93%, 94%, 95%, 96%, 97%, 98%, 99% or 100% sequence identity to SEQ ID NOs: 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, or 587; and wherein a beta subunit is chosen from one or a combination of amino acid sequences that comprise at least about 70%, 75%, 80%, 85%, 90%, 91%, 92%, 93%, 94%, 95%, 96%, 97%, 98%, 99% or 100% sequence identity to SEQ ID NOs: 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588.

### Methods Relating to TCR Expressing T Cells

In some embodiments, the method may comprise cloning the nucleotide sequence that encodes the TCR, or the antigen-binding portion thereof, from the clonally expanded T cells that are active against one or a plurality to neoantigens from the subject, into a recombinant expression vector using established molecular cloning techniques as described in, e.g., Green et al. (Eds.), Molecular Cloning: A Laboratory Manual, Cold Spring Harbor Laboratory Press; 4th Ed. (2012). For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the disclosure are not naturally-occurring as a whole. However, parts of the vectors can be naturally-occurring. The recombinant expression vectors can comprise any type of nucleotides, including, but not limited to DNA (e.g., complementary DCA (cDNA)) and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. In some embodiments, the non-naturally occurring or altered nucleotides or internucleotide linkages does not hinder the transcription or replication of the vector.

The recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of transposon/transposase, the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). In some embodiments, the recombinant expression vector is a viral vector, e.g., a retroviral vector.

The TCR, or the antigen-binding portion thereof, isolated by the disclosed methods may be useful for preparing cells for adoptive cell therapies. In some embodiments, the disclosure provides a method of preparing a population of cells that express a TCR, or an antigen-binding portion thereof, having highly immunogenic specificity for the cancer-specific and subject-specific neoantigens identified by the disclosed methods, the method comprising isolating a TCR, or an antigen-binding portion thereof, as described herein, and introducing the nucleotide sequence encoding the isolated TCR, or the antigen-binding portion thereof, into one or a plurality of host cells to obtain cells that express the TCR, or the antigen-binding portion thereof.

Introducing the nucleotide sequence (e.g., a recombinant expression vector) encoding the isolated TCR, or the antigen-binding portion thereof, into host cells may be carried out in any of a variety of different ways known in the art as described in, e.g., Green et al. supra. Non-limiting examples of techniques that are useful for introducing a nucleotide sequence into host cells include transformation, transduction, transfection, and electroporation.

The host cell into which the nucleotide sequence encoding the TCR, or antigen binding portion thereof, is introduced may be any type of cell that can contain the recombinant expression vector disclosed herein. In some embodiments, the host cell is a eukaryotic cell, e.g., plant, animal, fungi, or algae. In some embodiments, the host cell is a prokaryotic cell, e.g., bacteria or protozoa. In some embodiments, the host cell is a cultured cell. In other embodiments, the host cell is a primary cell, i.e., isolated directly from an organism, e.g., a human. In some embodiments, the host cell is an adherent cell. In some embodiments, the host cell is a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell is a prokaryotic cell, e.g., a DH5a cell, in some embodiments. For purposes of producing the TCR, or antigen binding portion thereof, the host cell is a mammalian cell in some embodiment. In some embodiments, the host cell is a human cell. While the host cell can be of any cell type, can originate from any type of tissue, and can be of any developmental stage, the host cell preferably is a PBL or a PBMC in some embodiments. In some embodiments, the host cell is a T cell.

In some embodiments, the PBMC include T cells. The T cells may be any type of T cell. Without being bound to a particular theory or mechanism, it is believed that less differentiated, “younger” T cells may be associated with any one or more of greater in vive persistence, proliferation, and antitumor activity as compared to more differentiated, “older” T cells. Accordingly, the disclosed methods may, advantageously, identify and isolate a TCR, or an antigen-binding portion thereof, that is highly immunogenic in response to the one or plurality of cancer-specific and subject-specific neoantigens and introduce the TCR, or an antigen-binding portion thereof, into “younger” T cells that may provide any one or more of greater in vive persistence, proliferation, and antitumor activity as compared to “older” T cells (e.g., effector cells in a patient's tumor).

In some embodiments, the host cells are autologous to the subject. In some embodiments, the TCRs, or the antigen-binding portions thereof, identified and isolated by the disclosed methods are personalized to each subject or patient. In some embodiments, the disclosed methods identify and isolate TCRs, or the antigen-binding portions thereof, that are highly immunogenic in response to the one or plurality of cancer-specific and subject-specific neoantigens that are encoded by a recurrent (also referred to as “hot-spot”) cancer-specific mutation. In some embodiments, the method comprises introducing the nucleotide sequence encoding the isolated TCR, or the antigen-binding portion thereof, into host cells that are allogeneic to the patient. For example, in some embodiments, the method comprises introducing the nucleotide sequence encoding the isolated TCR, or the antigen-binding portion thereof, into the host cells from another patient whose tumors express the same mutation in the context of the same MHC molecule.

In some embodiments, the disclosed methods further comprise expanding the numbers of host cells, such as T cells, that express the TCR, or the antigen-binding portion thereof. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990), the content of each is incorporated by reference herein. In some embodiments, expansion of the numbers of T cells is carried out by culturing the T cells with OKT3 antibody, IL-2, and feeder PBMC (e.g., irradiated allogeneic PBMC). In some embodiments, the disclosed methods generate a large number of T cells that are highly immunogenic in response to the one or plurality of cancer-specific and subject-specific neoantigens.

The T cells, that express the TCR, or the antigen-binding portion thereof, can be formulated into a composition, such as a pharmaceutical composition. In some embodiments therefore, the disclosure provides a pharmaceutical composition comprising any of the T cells that comprise a nucleic acid molecule encoding one or a plurality of TCRs, or the antigen-binding portions thereof, and a pharmaceutically acceptable carrier. In some embodiments, such pharmaceutical compositions further comprise another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

In some embodiments, the carrier in such pharmaceutical compositions is a pharmaceutically acceptable carrier. Such pharmaceutically acceptable carriers are well-known to those skilled in the art and are readily available to the public. In some embodiments, the pharmaceutically acceptable carrier is one which has no detrimental side effects or toxicity under the conditions of use.

In some embodiments, the T cells that comprise a nucleic acid molecule encoding one or a plurality of TCRs, or the antigen-binding portions thereof, or a pharmaceutical composition comprising the same, are administered back to the subject by injection, e.g., intravenously. When such T cells are administered, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In some embodiments, the pharmaceutically acceptable carrier is supplemented with human serum albumin.

It is contemplated that the T cells that comprise a nucleic acid molecule encoding one or a plurality of TCRs, or the antigen-binding portions thereof, disclosed herein, or a pharmaceutical composition comprising the same, can be used in methods of treating or preventing cancer in the subject. Without being bound to a particular theory or mechanism, the TCRs or the antigen-binding portions thereof expressed by such T cells are believed to bind specifically to a neoantigen encoded by a cancer-specific mutation found in the subject, such that the TCR, or the antigen-binding portion thereof, when expressed by a cell in the subject, is able to mediate an immune response against a target cell expressing the neoantigen. In some embodiments therefore, the disclosure provides a method of treating or preventing cancer in a patient, comprising administering to the subject any of the pharmaceutical compositions, TCRs, antigen-binding portions thereof, polypeptides, proteins, nucleic acids, recombinant expression vectors, host cells, or populations of cells described herein, in an amount effective to treat or prevent cancer in the subject.

The disclosure relates to a method of treating a subject in need thereof comprising:
(i) activating a first population of T cells from a sample;
ii) sequencing the nucleic acid expression in the T cells;
(iii) producing a vector comprising one or a plurality of nucleic acid sequences that encode a TCR from the sample;
(iv) transducing the vector into a second population of T cells;
(v) administering a therapeutically effective amount of the second population of T cells into the subject.

In some embodiments the methods of the disclosure comprise activating a first population of T cells from a sample by exposing the isolated Tcells to one or a plurality of neoantigens from the subject. Exposure can be accomplished by incubating the cells in the presence of one or a plurality of neoantigens.

In some embodiments, the step of sequencing the nucleic acid expression of the T cells comprises sequencing the nucleic acid sequences that encode one or a plurality of TCRs from the T cells, or antigen binding fragments thereof.

Methods of the disclosure can be performed by using autologous T cells from the patient for cell administration or isolated T cells cultured from other sources. In some embodiments in which autologous T cells are administered to the subject in step (v), the method further comprises a step of isolating a T cell from the sample prior to the step of sequencing. In some embodiments, the step of isolating comprises one or a combination of: flow cytometry, cell filtration, exposure to a column comprising antibodies specific for the T cells, and/or exposure to a magnetic surface comprising antibodies specific for the T cells. In some embodiments, isolated T cells from the subject are culture with a nucleic acid molecule that comprises an expressible nucleic acid sequence encoding one or a plurality of TCR sequences, or antigen binding fragments thereof. In some embodiments, the methods disclosed herein further comprise transducing the T cells with the nucleic acid molecule by transfection or lipofection. In some embodiments, isolated T cells are cultured with nucleic acid molecules that encode one or a plurality of TCRs or antigen binding fragments thereof for about 3 to about 5 days prior to administration.

For methods that involve administration of non-autologous T cell transfers, activated T cell from a sample of a subject can be used to merely identify TCR sequences through sequencing the TCR sequences or antigen binding fragments from the activated samples. RNA or DNA can be isolated from the T cells and sequenced using known sequencing techniques. Those identified nucleic acid sequences can then, in some embodiments, be synthesized de novo or sub cloned from isolated DNA to create one or a plurality of inserts. Such nucleic acid inserts can be further subcloned into one of the disclosed plasmids and resuspended in buffer sufficient for transduction of isolated non-autologous T cells. After transduction, the resultant T cells can be used for therapy.

In some embodiments, the nucleic acid molecule that is transduced in the T cells comprises an expressible nucleic acid sequence encoding one or a plurality of TCRs or antigen binding fragments thereof. In some embodiments, the nucleic acid sequence encoding one or a plurality of TCRs or antigen binding fragments thereof comprise from about 15 to about 50 nucleotides and encodes an antigen binding fragment chosen from one or a combination of those sequences provided in Tables S, T, U, or W.

The amount or dose of the T cells that comprise a nucleic acid molecule encoding one or a plurality of TCRs, or the antigen-binding portions thereof, disclosed herein, or a pharmaceutical composition comprising the same, administered (e.g., numbers of cells when the population of T cells is administered) should be sufficient to effect, e.g., a therapeutic or prophylactic response, in the subject over a reasonable time frame. For example, the dose of the T cells or pharmaceutical composition comprising the same should be sufficient to bind to the neoantigen the TCRs, or the antigen-binding portions thereof, having immunogenic to, or detect, treat or prevent cancer in a period of from about 2 hours or longer, e.g., 12 to 24 or more hours, from the time of administration. In some embodiments, the time period could be even longer. The dose will be determined by the efficacy of the particular T cells or pharmaceutical composition comprising the same administered and the condition of the patient, as well as the body weight of the patient to be treated.

Many assays for determining an administered dose are known in the art. For example, an assay, which comprises comparing the extent to which target cells are lysed or IFN-γ is secreted by T cells expressing the TCR, or the antigen-binding portion thereof, upon administration of a given dose of such T cells to a mammal among a set of mammals of which is each given a different dose of the cells, could be used to determine a starting dose to be administered to a subject. The extent to which target cells are lysed or IFN-γ is secreted upon administration of a certain dose can be assayed by methods known in the art.

The dose of the T cells that comprise a nucleic acid molecule encoding one or a plurality of TCRs, or the antigen-binding portions thereof, also will be determined by the existence, nature and extent of any adverse side effects that might accompany the administration of a particular T cell population or pharmaceutical composition comprising the same. Typically, the attending physician will decide the dosage of the T cells or pharmaceutical composition comprising the same with which to treat each individual patient, taking into consideration a variety of factors, such as age, body weight, general health, diet, sex, the T cells or pharmaceutical composition comprising the same to be administered, route of administration, and the severity of the condition being treated.

In some embodiments, the number of T cells administered per infusion may vary, for example, in the range of from one million to 200 billion cells; however, amounts below or above this exemplary range are within the scope of the disclosure. In some embodiments, the daily dose of TCR-expressed T cells administered is from about 1 million to about 200 billion cells. In some embodiments, the daily dose of TCR-expressed T cells administered is from about 10 million to about 200 billion cells. In some embodiments, the daily dose of TCR-expressed T cells administered is from about 100 million cells to about 200 billion cells. In some embodiments, the daily dose of TCR-expressed T cells administered is about 5 million cells. In some embodiments, the daily dose of TCR-expressed T cells administered is about 10 million cells. In some embodiments, the daily dose of TCR-expressed T cells administered is about 20 million cells. In some embodiments, the daily dose of TCR-expressed T cells administered is about 20 million cells. In some embodiments, the daily dose of TCR-expressed T cells administered is about 30 million cells. In some embodiments, the daily dose of TCR-expressed T cells administered is about 40 million cells. In some embodiments, the daily dose of TCR-expressed T cells administered is about 50 million cells. In some embodiments, the daily dose of TCR-expressed T cells administered is about 60 million cells. In some embodiments, the daily dose of TCR-expressed T cells administered is about 70 million cells. In some embodiments, the daily dose of TCR-expressed T cells administered is about 80 million cells. In some embodiments, the daily dose of TCR-expressed T cells administered is about 90 million cells. In some embodiments, the daily dose of TCR-expressed T cells administered is about 100 million cells. In some embodiments, the daily dose of TCR-expressed T cells administered is about 250 million cells. In some embodiments, the daily dose of TCR-expressed T cells administered is, about 350 million cells. In some embodiments, the daily dose of TCR-expressed T cells administered is about 450 million cells. In some embodiments, the daily dose of TCR-expressed T cells administered is about 500 million cells. In some embodiments, the daily dose of TCR-expressed T cells administered is about 650 million cells. In some embodiments, the daily dose of TCR-expressed T cells administered is about 800 million cells. In some embodiments, the daily dose of TCR-expressed T cells administered is about 900 million cells. In some embodiments, the daily dose of TCR-expressed T cells administered is about 1 billion cells. In some embodiments, the daily dose of TCR-expressed T cells administered is about 5 billion cells. In some embodiments, the daily dose of TCR-expressed T cells administered is about 20 billion cells. In some embodiments, the daily dose of TCR-expressed T cells administered is about 30 billion cells. In some embodiments, the daily dose of TCR-expressed T cells administered is about 40 billion cells. In some embodiments, the daily dose of TCR-expressed T cells administered is about 60 billion cells. In some embodiments, the daily dose of TCR-expressed T cells administered is about 80 billion cells. In some embodiments, the daily dose of TCR-expressed T cells administered is about 100 billion cells. In some embodiments, the daily dose of TCR-expressed T cells administered is about 120 billion cells. In some embodiments, the daily dose of TCR-expressed T cells administered is about 130 billion cells. In some embodiments, the daily dose of TCR-expressed T cells administered is about 150 billion cells. In some embodiments, the daily dose of TCR-expressed T cells administered is about 160 billion cells. In some embodiments, the daily dose of TCR-expressed T cells administered is about 170 billion cells. In some embodiments, the daily dose of TCR-expressed T cells administered is about 180 billion cells. In some embodiments, the daily dose of TCR-expressed T cells administered is about 190 billion cells. In some embodiments, the daily dose of TCR-expressed T cells administered is about 200 billion cells.

In some embodiments, when populations of T cells are administered, the cells can be cells that are allogeneic or autologous to the subject. In some embodiments, the cells are autologous to the subject. In some embodiments, the cells are allogeneic to the subject.

### Methods of Treating Cancer

The disclosure relates to methods of treating cancer or preventing metastases of a cancer in a subject in need thereof, the methods comprising first inducing, in the subject, an immune response to one or a plurality of neoantigens produced by the cancer of the subject as disclosed elsewhere herein, followed by identifying one or a plurality of nucleotide sequences encoding a subset of TCRs that are highly immunogenic in response to the one or plurality of neoantigens in the subject as disclosed elsewhere herein so that a therapeutically effective amount of T cells comprising one or a plurality of such nucleotide sequences can be administered back to the subject as disclosed elsewhere herein. The immune response induced in the subject may provide a functional T cell response to the one or more neoantigens. Neoantigens have the advantage of being found in only one or a few specific individuals, not being found in normal tissues (and, therefore, having reduced off-target immunogenicity), and not being subject to central tolerance mechanisms.

In some embodiments, the neoantigen used to induce an immune response in the subject is associated with a hyperproliferative disease or disorder (e.g., cancer), such as a tumor neoantigen or a cancer neoantigen. The identified tumor neoantigens can then be introduced into the subject to activate neoantigen-specific immune cells in the subject.

In some embodiments, treatment is determined by a clinical outcome, an increase, enhancement or prolongation of anti-tumor activity by T cells, an increase in the number of anti-tumor T cells or activated T cells as compared with the number prior to treatment, or a combination thereof. In some embodiments, clinical outcome is selected from the group consisting of tumor regression, tumor shrinkage, tumor necrosis, anti-tumor response by the immune system, tumor expansion, recurrence or spread, or a combination thereof.

In some embodiments, the disclosed methods are used to treat a patient that has been diagnosed of having cancer, or is at risk of developing cancer. In some embodiments, the subject has previously been treated, and not responded to checkpoint inhibitor therapy. In some embodiments, the subject has no detectable neoplasia but is at high risk for disease recurrence. In some embodiments, the cancer is a solid tumor. In some embodiments, the cancer has a high mutational load. In another embodiment, the cancer has a moderate mutational load. In some embodiments, the cancer has been shown to have a poor or low response to checkpoint inhibitor therapy.

In some embodiments, the cancer is selected from the group consisting of non small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

In certain embodiments, the cancer is non-small cell lung carcinoma or melanoma, both of which have been shown to have a high mutational load. In other embodiments, the cancer is ovarian cancer or glioblastoma multiforme, both of which show a moderate mutational load and have been shown to have a poor or low response to checkpoint inhibitor therapy.

In some embodiments, the disclosed methods are of a sufficient magnitude or efficacy to inhibit or retard tumor growth, induce tumor cell death, induce tumor regression, prevent or delay tumor recurrence, prevent tumor growth, prevent tumor spread and/or induce tumor elimination.

In some embodiments, the disclosed methods comprises administration of one or more additional therapeutic agents. In some embodiments, the one or more additional therapeutic agents are biologic therapeutics or a small molecules. In some embodiments, the therapeutic agent is (i) a checkpoint inhibitor or functional fragment thereof; or (ii) a nucleic acid molecule encoding a checkpoint inhibitor or a functional fragment thereof.

Checkpoint inhibitors include any agent that blocks or inhibits the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. In some embodiments, the checkpoint inhibitor targets or inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4 (belongs to the CD2 family of molecules and is expressed on all NK, γδ, and memory CD8+ (αβ) T cells), CD160 (also referred to as BY55), CGEN-15049, CHK1 and CHK2 kinases, A2aR, and various B-7 family ligands, or a combination thereof. Checkpoint inhibitors include antibodies, or antigen binding fragments thereof, other binding proteins, biologic therapeutics or small molecules, that bind to and block or inhibit the activity of one or more of CTLA-4, PDL1, PDL2, PD1, B7-H3, B7-H4, BTLA, HVEM, TIM3, GAL9, LAG3, VISTA, KIR, 2B4, CD160, CGEN-15049, CHK1, CHK2, 000342 CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-Hl; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). In some embodiments, the checkpoint inhibitors are the checkpoint inhibitors listed on Table 4.

TABLE 4
List of Checkpoint Inhibitors

| FDA Approved Checkpoint Inhibitors | |
|---|---|
| Pembrolizumab | Spartalizumab |
| Nivolumab | Camrelizumab |
| Cemiplimab | Sintilimab |
| Dostaslimab | Tislelizumab |
| Atezolizumab | Toripalimab |
| Avelumab | Tremelimumab |
| Durvalumab | |
| Ipilimumab | |
| Relatlimab | |

Checkpoint protein ligands include, but are not limited to PD-L1, PD-L2, B7-H3, B7-H4, CD28, CD86 and TIM-3.

In some embodiments, the checkpoint inhibitor is an inhibitor of the programmed death-1 (PD-1) pathway. In some embodiments, the checkpoint inhibitor is an anti-cytotoxic T-lymphocyte-associated antigen 4 (CTLA4) antibody or functional fragment thereof. In some embodiments, the checkpoint inhibitor is an anti-PDL1 antibody or functional fragment thereof. In some embodiments, the checkpoint inhibitor is an anti-PDL2 antibody or functional fragment thereof. In some embodiments, the checkpoint inhibitor is an anti-PD1 antibody or functional fragment thereof. In some embodiments, the checkpoint inhibitor is an anti-B7-H3 antibody or functional fragment thereof. In some embodiments, the checkpoint inhibitor is an anti-B7-H4 antibody or functional fragment thereof. In some embodiments, the checkpoint inhibitor is an anti-BTLA antibody or functional fragment thereof. In some embodiments, the checkpoint inhibitor is an anti-HVEM antibody or functional fragment thereof. In some embodiments, the checkpoint inhibitor is an anti-TIM3 antibody or functional fragment thereof. In some embodiments, the checkpoint inhibitor is an anti-Gal9 antibody or functional fragment thereof. In some embodiments, the checkpoint inhibitor is an anti-LAG3 antibody or functional fragment thereof. In some embodiments, the checkpoint inhibitor is an anti-VISTA antibody or functional fragment thereof. In some embodiments, the checkpoint inhibitor is an anti-KIR antibody or functional fragment thereof. In some embodiments, the checkpoint inhibitor is an anti-2B4 antibody or functional fragment thereof. In some embodiments, the checkpoint inhibitor is an anti-CD160 antibody or functional fragment thereof. In some embodiments, the checkpoint inhibitor is an anti-CHK1 antibody or functional fragment thereof. In some embodiments, the checkpoint inhibitor is an anti-CHK2 antibody or functional fragment thereof. In some embodiments, the checkpoint inhibitor is an anti-A2aR antibody or functional fragment thereof. In some embodiments, the checkpoint inhibitor is an anti-B-7 antibody or functional fragment thereof. In some embodiments, the checkpoint inhibitor is an anti-CGEN-15049 antibody or functional fragment thereof.

TABLE 3
Sequences of checkpoint proteins.

| | Representative Genbank Accession Numbers |
|---|---|
| CTLA-4 | Q53GD3.2, NP_001171515.1, NP_001171516.1, NP_079533.2, |
| | AAH14659.1, AAL75992.1 |
| PDL1 | AAP42144.1, NP_001300958.1, NP_001254635.1, NP_054862.1, |
| | XP_047279218.1, Q9NZQ7.1, AAU09634.1, AAP13470.1, 3FN3_B, |
| | 3FN3_A, 3BIS_B, 3BIS_A, 7VUX_A |
| PDL2 | AAP49001.1, AAP49000.1, NP_079515.2, XP_005251657.1, Q9BQ51.2, |
| | AAI13681.1, AAI13679.1, AAH74766.1, ABC59616.1, AAP13471.1, |
| | AAK15370.1 |
| PD1 | AAO25116.1, AJS10360.1, AJS10359.1, AJS10358.1, EAW71298.1, |
| | AAN64003.1 |
| B7-H3 | Q5ZPR3.1, CAE47548.1 |
| B7-H4 | Q7Z7D3.1, AAZ17406.1 |
| BTLA | AAI07093.1, AAI07092.1, CCQ43921.1, NP_861445.4, NP_001078826.1 |
| HVEM | AAQ89238.1, |
| TIM3 | AAL65158.1, AAL65157.1 |
| GAL9 | O00182.2 |
| LAG3 | AAH52589.1, P18627.5 |
| VISTA | NP_071436.1, Q9H7M9.3 |
| KIR | CUT93387.1, P55040.1, NP_001309097.1, NP_037421.2 |
| 2B4 | Q9BZW8.2, NP_001160135.1, NP_057466.1, NP_001160136.1, |
| | AAK00233.1, XP_011507924.1 |
| CD160 | CAG46686.1, CAG46665.1, XP_011507406.1 |
| CHK 1 | AAC51736.1, O14757.2 |
| CHK2 | BAB17231.1, O96017.1 |
| A2aR | NP_001265429.1, NP_001265428.1, NP_001265427.1 |
| B-7 | KAI2539390.1, KAI2539389.1 |

In some embodiments the therapeutic agent is a checkpoint inhibitor that is any full length amino acid sequence identified above or any fragment of the full-length amino acid above comprising about 70%, 75%, 80%, 85%, 90%, 91%, 92%, 93%, 94%, 95%, 96%, 97%, 98%, or 99% sequence identity to an amino acid sequence identified above.

In some embodiments, the therapeutic agent is an adjuvant. The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response. In some embodiments, the adjuvant can be other genes that are expressed in alternative plasmid or are delivered as proteins in combination with the plasmid above in the vaccine.

In some embodiments, the adjuvant can be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. In some embodiments, the adjuvant can be IL-12, IL-15, IL-28, CTACK, TECK, platelet derived growth factor (PDGF), TNF.alpha., TNF.beta., GM-CSF, epidermal growth factor (EGF), IL-1, IL-2, IL-4, IL-5, IL-6, IL-10, IL-12, IL-18, or a combination thereof.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

Other exemplary adjuvants include, but are not limited to, poly-ICLC (see Pharmacol Ther. 2015 February; 146:120-31, incorporated by reference in its entirety herein), 1018 ISS (see Vaccine. 2003 Jun. 2; 21 (19-20): 2461-7, incorporated by reference in its entirety herein), aluminum salts, Amplivax AS15, Bacillus Colmette-Guérin (BCG) (see Clin Immunol. 2000 January; 94 (1): 64-72, incorporated by reference in its entirety herein), CP-870,893, CpG7909 (GenBank Accession No. CS576603.1), CyaA (GenBank Accession No. KP670536.1), GM-CSF (GenBank Accession No. M11220.1), IC30 (see Expert Rev Vaccines. 2007 October; 6 (5): 741-6, incorporated by reference in its entirety herein), IC31 (see Expert Rev Vaccines. 2007 October; 6 (5): 741-6, incorporated by reference in its entirety herein), Imiquimod (see Vaccine. 2006 Mar. 10; 24 (11): 1958-6, incorporated by reference in its entirety herein), ImuFact 1MP321, IS Patch, ISS, ISCOMATRIX, Juvlmmune, LipoVac, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA micropartieles, resiquimod, S L172, Virosomes and other Virus-like particles, YF-17D, VEGF trap, R848, beta-glucan, Pam3Cys, acrylic or methacrylic polymers, copolymers of maleic anhydride and Aquila's QS21 stimulon, and a functional fragment of any thereof; or (ii) a nucleic acid molecule encoding an adjuvant selected from the group consisting of: (i) poly-ICLC, 1018 ISS, aluminum salts, Amplivax AS15, BCG, CP-870,893, CpG7909, CyaA, GM-CSF, IC30, IC31, Imiquimod, ImuFact 1MP321, IS Patch, ISS, ISCOMATRIX, JuvImmune, Lipo Vac, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, S L172, Virosomes and other Virus-like particles, YF-17D, VEGF trap, R848, beta-glucan, Pam3Cys, acrylic or methacrylic polymers, copolymers of maleic anhydride and Aquila's QS21 stimulon, or functional fragment thereof.

In some embodiments, the therapeutic agent is an immunostimulatory agent or functional fragment thereof. For example, in some embodiments, the immunostimulatory agent is an interleukin or functional fragment thereof.

In some embodiments, the therapeutic agent is a chemotherapeutic agent. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. For prostate cancer treatment, a preferred chemotherapeutic agent with which anti-CTLA-4 can be combined is paclitaxel (Taxol®).

### EXAMPLES

### Example 1

Following vaccination against 20-40 epitopes from individualized neoantigens from a sample, PBMC are collected from the patient. Then, a cocktail of cytokines (IL-2+IL-4+IL-7) are utilized in a 10-day time course to activate human T cells ex vivo (Karanikas et al. J Immunol 2003; 171:4898-4904; Roudko et al. Cell 2020; 183:1634-1649). The protocol is as follows: on day 0, PBMC are thawed and rest overnight in complete RPMI 10%-human serum media. On day 1, cells are counted and 250,000 PBMC are plated in 0.2 mL of media in 96 well plate. The antigenic peptides are added to a final concentration of 10 μg/mL. Then, the cytokines are added to the media using the following concentrations: IL-2 (20 IU/mL), IL4 (10 ng/ml), and IL-7 (10 ng/ml). Cells are left untouched in the incubator until the next time point at 37° C., 5%-CO2. On days 3 and 6, PBMC are spun down at room temperature, 1500 RPM, for 5 min. Then half of the media (0.1 mL) is carefully removed and replaced with fresh RPMI10%-human serum containing cytokines for final concentrations of: IL-2 (20 IU/mL), IL4 (10 ng/mL), and IL-7 (10 ng/ml). Cells are left untouched in the incubator until the next time point at 37° C., 5%-CO2. On day 9, cells are spun down at room temperature, 1500 RPM, for 5 min, and the media is completely replaced with fresh, cytokine-free RPMI10%-human serum media. Cells are left untouched in the incubator until the next time point at 37° C., 5%-CO2. On day 10, cells are stimulated with the corresponding peptides (10 ug/mL), and returned to the incubator at 37° C., 5%-CO2. 18 to 24 hours later, cells are stained and sorted based on CD137 and/or CD69 status.

Examples of neoantigens: Full-length and fragments 1-15.

### Neoantigens

Catenin beta-1 (CTNB1):
FASTA: >sp|P35222|CTNB1_HUMAN Catenin beta-1 OS=Homo sapiens OX=9606 GN=CTNNB1 PE=1 SV=1 (Beta catenin wild type)

Full-length protein sequence (SEQ ID NO: 362):
MATQADLMELDMAMEPDRKAAVSHWQQQSYLDSGIHSGATTTAPSLSGKGNPEEEDVDTSQVLYEWEQGF
SQSFTQEQVADIDGQYAMTRAQRVRAAMFPETLDEGMQIPSTQFDAAHPTNVQRLAEPSQMLKHAVVNLI
NYQDDAELATRAIPELTKLLNDEDQVVVNKAAVMVHQLSKKEASRHAIMRSPQMVSAIVRTMQNTNDVET
ARCTAGTLHNLSHHREGLLAIFKSGGIPALVKMLGSPVDSVLFYAITTLHNLLLHQEGAKMAVRLAGGLQ
KMVALLNKTNVKFLAITTDCLQILAYGNQESKLIILASGGPQALVNIMRTYTYEKLLWTTSRVLKVLSVC
SSNKPAIVEAGGMQALGLHLTDPSQRLVQNCLWTLRNLSDAATKQEGMEGLLGTLVQLLGSDDINVVTCA
AGILSNLTCNNYKNKMMVCQVGGIEALVRIVLRAGDREDITEPAICALRHLTSRHQEAEMAQNAVRLHYG
LPVVVKLLHPPSHWPLIKATVGLIRNLALCPANHAPLREQGAIPRLVQLLVRAHQDTQRRTSMGGTQQQF
VEGVRMEEIVEGCTGALHILARDVHNRIVIRGLNIIPLFVQLLYSPIENIQRVAAGVLCELAQDKEAAEA
IEAEGATAPLTELLHSRNEGVATYAAAVLFRMSEDKPQDYKKRLSVELTSSLFRTEPMAWNETADLGLDI
GAQGEPLGYRQDDPSYRSFHSGGYGQDALGMDPMMEHEMGGHHPGADYPVDGLPDLGHAQDLMDGLPPGD
SNQLAWFDTDL

Example of Mutated Catenin beta-1 (CTNB1_D32G) number 1 (SEQ ID NO: 363)
FASTA: >sp|P35222|CTNB1_D32G (mutated beta catenin and potential vaccine sequences)

Full-length protein sequence:
MATQADLMELDMAMEPDRKAAVSHWQQQSYLGSGIHSGATTTAPSLSGKGNPEEEDVDTSQVLY
EWEQGFSQSFTQEQVADIDGQYAMTRAQRVRAAMFPETLDEGMQIPSTQFDAAHPTNVQRLAEP
SQMLKHAVVNLINYQDDAELATRAIPELIKLLNDEDQVVVNKAAVMVHQLSKKEASRHAIMRSP
QMVSAIVRIMQNTNDVETARCTAGTLHNLSHHREGLLAIFKSGGIPALVKMLGSPVDSVLFYAI
TTLHNLLLHQEGAKMAVRLAGGLQKMVALLNKTNVKFLAITTDCLQILAYGNQESKLIILASGG
PQALVNIMRTYTYEKLLWTTSRVLKVLSVCSSNKPAIVEAGGMQALGLHLTDPSQRLVQNCLWT
LRNLSDAATKQEGMEGLLGTLVQLLGSDDINVVTCAAGILSNLTCNNYKNKMMVCQVGGIEALV
RTVLRAGDREDITEPAICALRHLTSRHQEAEMAQNAVRLHYGLPVVVKLLHPPSHWPLIKATVG
LIRNLALCPANHAPLREQGAIPRLVQLLVRAHQDTQRRTSMGGTQQQFVEGVRMEEIVEGCTGA
LHILARDVHNRIVIRGLNTIPLFVQLLYSPIENIQRVAAGVLCELAQDKEAAEAIEAEGATAPL
TELLHSRNEGVATYAAAVLFRMSEDKPQDYKKRLSVELTSSLFRTEPMAWNETADLGLDIGAQG
EPLGYRQDDPSYRSFHSGGYGQDALGMDPMMEHEMGGHHPGADYPVDGLPDLGHAQDLMDGLPP
GDSNQLAWFDTDL

TABLE N
Example of fragments from mutated Catenin beta-1 (CTNB1_D32G)
number 1 that can be used in a vaccine

| | |
|---|---|
| 1 | YLGSGIHSGA |
| 2 | PDRKAAVSHWQQQSYLGSGIHSGATTTAPSLSG |
| 3 | KAAVSHWQQQSYLGSGIHSGATTTAPS |
| 4 | AAVSHWQQQSYLGSGIHSGATTTAP |
| 5 | RKAAVSHWQQQSYLGSGIHSGATTTAPSLSGKG |
| 6 | AVSHWQQQSYLGSGIHSGATTTAPSLS |
| 7 | VSHWQQQSYLGSGIHSGATTTAPSL |
| 8 | MATQADLMELDMAMEPDRKAAVSHWQQQSYLGSGIHSGA |
| 9 | MATQADLMELDMAMEPDRKAAVSHWQQQSYLGSGIHSGATTTAPSLSGKGNPEEEDVDTSQVLY |
| | EWEQGFSQSFTQEQ |
| 10 | MATQADLMELDMAMEPDRKAAVSHWQQQSYLGSGIHSGATTTAPSLSGKGNPEEEDVDTSQVLY |
| | EWE |
| 11 | DLMELDMAMEPDRKAAVSHWQQQSYLGSGIHSGATTTAPSLSGKGNPEEEDVDTSQV |
| 12 | DMAMEPDRKAAVSHWQQQSYLGSGIHSGATTTAPSLSGKGNPEEEDV |
| 13 | PDRKAAVSHWQQQSYLGSGIHSGATTTAPSLSGKGNP |
| 14 | QQQSYLGSGIHSGATTTA |
| 15 | MATQADLMELDMAMEPDRKAAVSHWQQQSYLGSGIHSGATTTAPSLSGKGNPEEEDVDTSQVLY |
| | EWEQGFSQSFTQEQVADIDGQYAMTRAQRVRAAMFPETLDEGMQIPSTQFDAAHPINVQRLAEP |
| | SQMLKHAVVNLINYQDDAELATRAIPELIKLLNDEDQVVVNKAAVMVHQLSKKEASRHAIMRSP |
| | QMVSAIVRIMQNTNDVETARCTAGTLHNLSHHREGLLAIFKSGGIPALVKMLGSPVDSVLFYAI |
| | TTLHNLLLHQEGAKMAVRLAGGLQKMVALLNKTNVKFLAITTDCLQILAYGNQESKLIILASGG |
| | PQALVNIMRTYTYEKLLWITSRVLKVLSVCSSNKPAIVEAGGMQALGLHLTDPSQRLVQNCLWT |
| | LRNLSDAATKQEGMEGLLGTLVQLLGSDDINVVICAAGILSNLICNNYKNKMMVCQVGGIEALV |
| | RTVLRAGDREDITEPAICALRHLTSRHQEAEVAQNAVRLHYGLPVVVKLLHPPSHWPLIKATVG |
| | LIRNLALCPANHAPLREQGAIPRLVQLLVRAHQDTQRRTSMGGTQQQFVEGVRMEEIVEGCTGA |
| | LHILARDVHNRIVIRGLNTIPLFVQLLYSPIENIQRVAAGVLCELAQDKEAAEAIEAEGATAPL |
| | TELLHSRNEGVATYAAAVLFRMSEDKPQDYKKRLSVELTSSLFRTEPMAWNETADLGLDIGAQG |
| | EPLGYRQDDPSYRSFHSGGYGQDALGMDPMMEHEMGGHHPGADYPVDGLPDLGHAQDLMDGLPP |
| | GDSNQLAWFDTDL |

Example of mutated Catenin beta-1 (CTNB1_S45P) number 2
FASTA: >sp|P35222|CTNB1_S45P (mutated beta catenin and potential vaccine sequences)

Full-length protein sequence (SEQ ID NO: 364):
MATQADLMELDMAMEPDRKAAVSHWQQQSYLDSGIHSGATTTAPPLSGKGNPEEEDVDTSQVLYEWEQGF
SQSFTQEQVADIDGQYAMTRAQRVRAAMFPETLDEGMQIPSTQFDAAHPTNVQRLAEPSQMLKHAVVNLI
NYQDDAELATRAIPELTKLLNDEDQVVVNKAAVMVHQLSKKEASRHAIMRSPQMVSAIVRTMQNTNDVET
ARCTAGTLHNLSHHREGLLAIFKSGGIPALVKMLGSPVDSVLFYAITTLHNLLLHQEGAKMAVRLAGGLQ
KMVALLNKTNVKFLAITTDCLQILAYGNQESKLIILASGGPQALVNIMRTYTYEKLLWTTSRVLKVLSVC
SSNKPAIVEAGGMQALGLHLTDPSQRLVQNCLWTLRNLSDAATKQEGMEGLLGTLVQLLGSDDINVVTCA
AGILSNLICNNYKNKMMVCQVGGIEALVRTVLRAGDREDITEPAICALRHLTSRHQEAEMAQNAVRLHYG
LPVVVKLLHPPSHWPLIKATVGLIRNLALCPANHAPLREQGAIPRLVQLLVRAHQDTQRRISMGGTQQQF
VEGVRMEEIVEGCTGALHILARDVHNRIVIRGLNTIPLFVQLLYSPIENIQRVAAGVLCELAQDKEAAEA
IEAEGATAPLTELLHSRNEGVATYAAAVLFRMSEDKPQDYKKRLSVELTSSLFRTEPMAWNETADLGLDI
GAQGEPLGYRQDDPSYRSFHSGGYGQDALGMDPMMEHEMGGHHPGADYPVDGLPDLGHAQDLMDGLPPGD
SNQLAWFDTDL

TABLE M
Example of fragments from mutated Catenin beta-1 (CTNB1_S45P)
number 2 that can be used in a vaccine

| | |
|---|---|
| 16 | TTAPPLSGK |
| 17 | MATQADLMELDMAMEPDRKAAVSHWQQQSYLDSGIHSGATTAPPLSGKGNPEEEDVDTSQVLYEW |
| | EQGFSQSFTQEQVADIDGQYAMTRAQRVRAAMFPETLDEGMQIPSTQFDAAHPTNVQRLAEPSQM |
| | LKHAVVNLINYQDDAELATRAIPELTKLLNDEDQVVVNKAAVMVHQLSKKEASRHAIMRSPQMVS |
| | AIVRTMQNTNDVETARCIAGILHNLSHHREGLLAIFKSGGIPALVKMLGSPVDSVLFYAITTLHN |
| | LLLHQEGAKMAVRLAGGLQKMVALLNKTNVKFLAITTDCLQILAYGNQESKLIILASGGPQALVN |
| | IMRTYTYEKLLWITSRVLKVLSVCSSNKPAIVEAGGMQALGLHLTDPSQRLVQNCLWILRNLSDA |
| | ATKQEGMEGLLGTLVQLLGSDDINVVICAAGILSNLTCNNYKNKMMVCQVGGIEALVRTVLRAGD |
| | REDITEPAICALRHLTSRHQEAEMAQNAVRLHYGLPVVVKLLHPPSHWPLIKATVGLIRNLALCP |
| | ANHAPLREQGAIPRLVQLLVRAHQDTQRRTSMGGTQQQFVEGVRMEEIVEGCTGALHILARDVHN |
| | RIVIRGLNTIPLFVQLLYSPIENIQRVAACVLCELAQDKEAAEAIEAEGATAPLTELLHSRNEGV |
| | ATYAAAVLERMSEDKPQDYKKRLSVELISSLFRIEPMAWNETADLGLDIGAQGEPLGYRQDDPSY |
| | RSFHSGGYGQDALGMDPMMEHEMGGHHPGADYPVDGLPDLGHAQDLMDGLPPGDSNQLAWFDTDL |
| 18 | MATQADLMELDMAMEPDRKAAVSHWQQQSYLDSGIHSGATTTAPPLSGK |
| 19 | MATQADLMELDMAMEPDRKAAVSHWQQQSYLDSGIHSGATTTAPPLSGKGNPEEEDVDTSQVLYE |
| | WEQGFSQSFTQEQ |
| 20 | MATQADLMELDMAMEPDRKAAVSHWQQQSYLDSGIHSGATTTAPPLSGKGNPEEEDVDTSQVLYE |
| | WEQ |
| 21 | DMAMEPDRKAAVSHWQQQSYLDSGIHSGATTTAPPLSGKGNPEEEDVDTSQVLYEWEQ |
| 22 | AVSHWQQQSYLDSGIHSGATTTAPPLSGKGNPEEEDVDTSQVLYEWEQ |
| 23 | QSYLDSGIHSGATTTAPPLSGKGNPEEEDVDTSQVLYE |
| 24 | SYLDSGIHSGATTTAPPLSGKGNPEEEDVDTSQ |
| 25 | DSGGIHSGATTTAPPLSGKGNPEEEDVD |
| 26 | SGIHSGATTTAPPLSGKGNPEEEDV |
| 27 | IHSGATTTAPPLSGKGNPEEEDVDTSQVLYEWEQ |
| 28 | DRKAAVSHWQQQSYLDSGIHSGATTTAPPLSGKGNPEEEDVDTSQVLYEWEQGFSQSFTQEQVAD |
| | IDGQYAMTRAQRVRAAMFPETLDEGMQIPSTQFDAAHPINVQRLAEPSQMLKHAVVNLINYQDDA |
| | ELATRAIPELIKLLNDEDQVVVNKAAVMVHQLSKKEASRHAIMRSPQMVSAIVRTMQNINDVETA |
| | RCTAGTLHNLSHHREGLLAIFKSGGIPALVKMLGSPVDSVLFYAITTLHNLLLHQEGAKMAVRLA |
| | GGLQKMVALLNKTNVKFLAITTDCLQILAYGNQESKLIILASGGPQALVNIMRTYTYEKLLWITS |
| | RVLKVLSVCSSNKPAIVEAGGMQALGLHLTDPSQRLVQNCLWTLRNLSDAATKQEGMEGLLGTLV |
| | QLLGSDDINVVTCAAGILSNLT |
| 29 | MATQADLMELDMAMEPDRKAAVSHWQQQSYLDSGIHSGATTTAPPLSGKGNPEEEDVDTSQVLYE |
| | WEQGFSQSFTQEQVADIDGQYAMTRAQRVRAAMFPETLDEGMQIPSTQFDAAHPINVQRLAEPSQ |
| | MLKHAVVNLINYQDDAELATRAIPELTKLLNDEDQV |
| 30 | SGATTTAPPLSGKGNPE |

TABLE O
The following table includes tumor-associated antigens (short
fragments/epitopes) utilized to design neoantigen DNA vaccines. The first column of Table O
lists the SEQ ID NO of the peptide sequence. The fifth column of Table O lists the SEQ ID NO
of the Neoantigen Design.

| SEQ | | Gene | Peptide | SEQ | |
|---|---|---|---|---|---|
| ID | UniProt# | Symbol | sequence | ID | Neoantigen Design |
| 31 | Q9BVV6-3 | KIAA0586 | MLWCGTCFV | 397 | MLWCGTCFVTNNMKGSEVSLEK |
| 32 | O60504 | SORBS3 | RPRDCYRRM | 398 | SGMPIAPRSSVDRPRDCYRRMFQQIHRKMPDLQ |
| 33 | P54136 | RARS | TLCAYIYEL | 399 | EILQKILDDLFLHTLCAYIYELATAFTEFYDSC |
| 34 | P21673 | SAT1 | YLEDFFVV | 400 | DPWIGKLLYLEDFFVVSDYRGFGIGSEILKNLS |
| 35 | P13611/ | VCAN | MMKVMMTAL | 401 | MQTDIDTEVPSEPVMMKVMMTALKFKRSMRQLS |
| | CCDS4060.1 | | | | |
| 36 | Q6Y2X3 | DNAJC14 | FKLLGALLLL | 402 | QGFYCGVGLFTRFKLLCALLLLALALFLGFLQL |
| 37 | Q8N6K7 | SAMD3 | ALAALVAAL | 403 | TKVDDCVTALAALVAALHVFRIECPRRLSQTEN |
| 38 | Q96AM1 | MRCPRF | KLNHDILAMV | 404 | LILHVECRARRRQRSAKLNHDILAMVSVFLVSS |
| 39 | Q8NAC3 | IL17RC | VLLEVHVPA | 405 | AQVVLSFQAYPTARCVLLEVHVPAALVQFGQSV |
| 40 | Q8IXL7-2 | MSRB3 | RPTGKRCCI | 406 | LGHIFDDGPRPTGKRCCINSAALSFTPADSSGT |
| 41 | Q7Z401-2 | DENND4A | SPLTSRTPRI | 407 | AEEIVMYMNNMSSPLTSRTPRIDLQRACDDKLN |
| 42 | Q8NB16 | MLKL | RLLEINMKEI | 408 | FQMLRRDNEKIEASLRLLEINMKEIKETLRQYL |
| 43 | Q8IUG5 | MYO18B | QLLGFLQRL | 409 | LAVKDWPWWQLLGFLQRLLSATIGTEQLRAKEE |
| 44 | A0A494C108 | KRI1 | FSRQRLPAF | 410 | MLGGCEFSRQRLPAFGLNPKRLHFRQLGRQRRK |
| 45 | P23378 | GLDC | GLATVRAYL | 411 | FQPNSGAQGEYAGLATVRAYLNQKGEGHRTVCL |
| 46 | P57071 | PRDM_ 5 | CICNRRFAL | 412 | IMEVHKEKGYGCCICNRRFALKATYHAHMVIHR |
| 47 | Q9HCM1 | KIAA_551 | IPSNSTRLSV | 413 | TYSMQMQMIPSNSTRLSVAYQGNQGLNQSFSEQ |
| 48 | Q15661 | TPSAB1 | GIYTHVTYYL | 414 | SWGEGCAQPNRPGIYTHVTYYLDWIHHYVPKKP |
| 49 | P28288 | ABCD3 | RLAGFAARI | 415 | GRIVLAGREMTRLAGFAARITELMQVLKDLNHG |
| 50 | Q96L91-2 | EP400 | FIMNQFKAA | 416 | RESRKDLVLIDSLFIMNQFKAAERMNIGKPNAK |
| 51 | Q9BZE4 | GTPBP4 | GPRMPQTAKKV | 417 | KKKLKILESKEKNTQGPRMPQTAKKVQRTVLEK |
| 52 | O14917 | PCDH_7 | LIRVKSNL | 418 | DRVRELFSIDPKTGLIRVKSNLDYEENGMLEID |
| 53 | O14526 | FCHO_ | FTEYIHAY | 419 | LGSQDALPIATAFTEYIHAYFRGHSPSCLARVT |
| 54 | Q86UL8 | MAGI2 | GPGGGGSGTL | 420 | RREGPGAAPAFAGPGGGGSGTLEAEGRAGARAG |
| 55 | P53708 | ITGA8 | LVRKRDVYVV | 421 | FLRNSTIPHLVRKRDVYVVEFHRQSPAKILNCT |
| 56 | O15397 | IPO8 | FLGHHDLKM | 422 | QWMNDTDCFLGHHDLKMCIIGLSILLELQNRPP |
| 57 | Q9HCD6 | TANC2 | SSREFAAAL | 423 | KPKSYEAYYARARAKRSSREFAAALEDLNEAIK |
| 58 | Q12770 | SCAP | STGIKFNSI | 424 | ISIWDRSTGIKFNSIQQDLGCGASLGVISDNLL |
| 59 | P24298 | GPT | RVRGVEYAV | 425 | LRAKVLTLDGMNPRVRGVEYAVRGPIVQRALEL |
| 60 | Q9Y6V0 | PCLO | LQKVKETLSM | 426 | KKQEQEVKTEAEKVILQKVKETLSMEKIPPMVT |
| 61 | O43520 | ATP8B1 | LKKNHFVPA | 427 | KWKEIQVGDVIRLKKNHFVPADILLLSSSEPNS |
| 62 | Q9UKX7 | NUP50 | LAANGPTAL | 428 | ADPKVAFGSLAANGPTALVDKVSNPKINGDSQQ |
| 63 | Q6R327 | RICTOR | QRSSSVQSM | 429 | LLSPINQNTLQRSSSVQSMVSSATYGGSDDYIG |
| 64 | Q8N8Z8 | ZNF441 | RPQHGKKLC | 430 | QIHERPQHGKKLCDCKECASFSSLENLQRHMAA |
| 65 | Q13315 | ATM | CVLMRLQEKL | 431 | KRNLSDIDQSFNKVAECVLMRLQEKLKGVEEGT |
| 66 | P41229 | KDM5C | ERILTLLEL | 432 | IWQLLQAGQPPDLERILTLLELEKAERHGSRAR |
| 67 | O15230 | LAMA5 | VOMAPVQPRI | 433 | FGFNPLEFENFSWRGYVQMAPVQPRIVARLNLT |
| 68 | O43365 | HOXA3 | YPYQAANGCA | 434 | IYGGYPYQAANGCAYNANQQPYPASAALGADGE |
| 69 | Q0VDD8-2 | DNAH_4 | RLLETFFNFV | 435 | THFKLPKYRRLLETFFNFVMLVDYIFQELIRQL |
| 70 | Q9H0E2 | TOLLIP | YVPITGMPTV | 436 | TVYQQGVGYVPITGMPTVCSPGMVPVALPPAAV |
| 71 | Q14527 | HLTF | YIMDNKLSQI | 437 | LAKELAGALAYIMDNKLSQIEGVVPFGANNAFT |
| 72 | Q6PJG2 | ELMSAN1 | RRSHRLSK | 438 | QDSAPQPALPQVQIPFPRRSHRLSKEGILPPSA |
| 73 | Q92696 | RABGGTA | FSNYSSCHY | 439 | AFTDSLITRNFSNYSSCHYRSCLLPQLHPQPDS |
| 74 | Q9HCM2 | PLXNA4 | SIIPDFDIYYV | 440 | AYVFHDEFVASMIKIPSDTFSIIPDFDIYYVYG |
| 75 | Q9Y6Y1 | CAMTA1 | LLSPDVSQGL | 441 | EAVYTMSPTAGPNHHLLSPDVSQGLVLAVSSDG |
| 76 | Q8TF42/ | UBASH3B | LESNTIIDVY | 442 | CMQARLVGEALLESNTIIDVYCSPSLRCVQTAH |
| | CCDS31694.1 | | | | |
| 77 | P43146 | DCC | NMYYFRIQA | 443 | ISGDRLTHQIMDLNLDNMYYFRIQARNSKGVGP |
| 78 | A6NNF4 | ZNF726 | EECVKAFSQF | 444 | LASGEKPYKCEECVKAFSQFGHLTTHRIIHTGE |
| 79 | Q14315 | FLNC | STTGVSSEF | 445 | GLVSAYGPGLEGSTTGVSSEFIVNTLNAGSGAL |
| 80 | Q99707 | MTR | YESLKEGRY | 446 | EEYEDIRQDHYESLKEGRYLPLSQARKSGFQMD |
| 81 | O60563 | CCNT_ | RLKGIWNWRA | 447 | LDELTHEFLQILEKTPNRLKGIWNWRACEAAKK |
| 82 | O00623 | PEX12 | SLSEEDEYSI | 448 | MELVLLPYLKVKLEKLVSSLSEEDEYSIHPPSS |
| 83 | Q13459 | MY09B | VIKNAIASL | 449 | CRVTATKDSTTSDVIKNAIASLRLDGTKCYVLV |
| 84 | Q9UBB6 | NCDN | GLSEDIQKA | 450 | DLLAVLRGLSEDIQKAEDASKFELCQLLPLFLP |
| 85 | P78562 | PHEX | QARKYLAQS | 451 | IKFSEADYFGNVLQARKYLAQSDFFWLRKAVPK |
| 86 | Q13415 | ORC1 | NTMDLPEQI | 452 | ARLVVLAIANTMDLPEQIMMNRVSSRLGLTRMC |
| 87 | Q9ULL8 | SHROOM4 | EEPASTVCIM | 453 | ASDCALSLRPEEPASTVCIMQGPGPTKAPSGRP |
| 88 | Q86UW8 | HAPLN4 | SGSVVVQTV | 454 | RKKVVHVLEGESGSVVVQTVPGQVVSHRGGTIV |
| 89 | Q3BBV0 | NBPF_ | ERQFKEEML | 455 | SMLRNERQFKEEMLAEQLKQAEELRQYKVLVHS |
| 90 | CCDS32106.1 | DCAF5 | MTAGFCYGTW | 456 | ATSAVSMPLNSPTMEASGWSQEEMTAGFCYGTW |
| 91 | O00300 | TNFRSF11B | MPNWLSVLV | 457 | TLCEEAFFRFAVPTKFMPNWLSVLVDNLPGTKV |
| 92 | Q9BW62 | KATNAL1 | EPFRDTAVW | 458 | KPPDFPVSCQDEPFRDTAVWPPPVPAEHRAPPQ |
| 93 | A2RRH5-4 | WDR27 | AVIAEAHSW | 459 | VEVEDLNAGCSAAVIAEAHSWPVHQICQNKGSS |
| 94 | Q92585 | MAML1 | KSSSRPGGPY | 460 | FTNSKLLMMPSVNKSSSRPGGPYLQPSHVNLLS |
| 95 | P42892 | ECE1 | NAMRFFNLSW | 461 | TAVPDLYFENAMRFFNLSWRVTADQLRKAPNRD |
| 96 | P43897-2 | TSFM | LTPVILALW | 462 | KQQPSAYSKVQWLTPVILALWEAEAGGSLEGEL |
| 97 | P42338 | PIK3CB | HVWENNNPFR | 463 | KKTRIISHVWENNNPFRIVLVKGNKLNTEETVK |
| 98 | Q9BRP8 | WIBG | STORPNGTW | 464 | PAATETGKYIASTQRPNGTWRKQRRVKEGYVPQ |
| 99 | Q9ULK4-3 | MED23 | SPSLMSETEW | 465 | IVLHDRIVSVISSPSLMSETEWVGYPFRLFDFT |
| 100 | Q86UA6 | RPAIN | MAESLRSSR | 466 | MAESLRSSRRSLYKLVGSPPWKEAFR |
| 101 | Q9NZV5 | SEPN1 | CSVTQAGVQW | 467 | PIAEKLTGSCSVTQAGVQWCSHSSLQPQLPWLN |
| 102 | Q9UL01 | DSE | VPFVTEALY | 468 | NSFTFAPNGVPFVTEALYGPKYTFFNNVLMFSP |
| 103 | Q16795 | NDUFA9 | VVRLLSMSR | 469 | MAAAAQSRVVRLLSMSRSAITAIATSVC |
| 104 | Q8WWG9 | KCNE4 | RSVQVSLML | 470 | AMKPLPVVSGLRSVQVSLMLNMLQESVAPALSC |
| 105 | Q9HAH7 | FBRS | GAVAATRLY | 471 | YEAGEELTGPGAVAATRLYGLEPAHPLLYSRLA |
| 106 | CCDS10189.1 | FAM96A | FSDVYHLNISW | 472 | HCSLATLIGLCLRVNFSDVYHLNISWKSTFLKE |
| 107 | O60218 | AKR1B10 | SPLGSLDRPW | 473 | LIQYCHSKGITVTAYSPLGSLDRPWAKPEDPSL |
| 108 | Q5T813 | FAM102B | EGYNTKNTR | 474 | GNTTRRCLLEGYNTKNTRQDNSILKVLISMQLM |
| 109 | NM_001281535.1 | EPB4_L3 | LSLLEKDYF | 475 | KRSRGQVLFDKVCEHLSLLEKDYFGLTYRDAEN |
| 110 | P51884 | LUM | KTVPMVPPGI | 476 | PSAMYCDELKLKTVPMVPPGIKYLYLRNNQIDH |
| 111 | Q9H4D0 | CLSTN2 | VDSSKMIFKF | 477 | PSAATNWTAGLLVDSSKMIFKFDGRQGAKVPDG |
| 112 | P11274 | BCR | SISEEITPR | 478 | HPLLQDALRISQNFLSSISEEITPRRQSMTVKK |
| 113 | Q9Y2Z2 | MTO1 | LPRMECSGAI | 479 | KAKVIQPDGVLLLLPRMECSGAISAHHNLPLPG |
| 114 | Q96AE7 | TTC17 | RGDIFENVNY | 480 | WDQPVRYHRGDIFENVNYVQFGEDSSTSSMMSV |
| 115 | Q92804 | TAF15 | MTGSSGVDR | 481 | GGYDKDGRGPMTGSSGVDRGGFKNFGGHRDYGP |
| 116 | Q9UM11 | FZR1 | TTDRCIRFW | 482 | HQHGLLASGGGTTDRCIRFWNTLTGQPLQCIDT |
| 117 | NM_001164317.1 | FLNB | MPESPYLVPV | 483 | GNYEVSIKFNDEHMPESPYLVPVIAPSDDARRL |
| 118 | O75445 | USH2A | TPVRIRSNR | 484 | MGLADTKIPRSGTPVRIRSNRSACVLRIPSQNQ |
| 119 | P78332 | RBM6 | RTQVDFRGR | 485 | DERNRDVSDLDERDKDRTQVDFRGRGSGTTDLD |
| 120 | Q9BUI4 | POLR3C | NFMSLQEILK | 486 | KQMLYKMLSENFMSLQEILKTPDHAPSRTFYLY |
| 121 | Q9NW64 | RBM22 | IALPLPPGI | 487 | NLPPSGPPAVVNIALPLPPGIAPPPPPGFGPHM |
| 122 | Q68BL8 | OLFML2B | HTIPVPPTTVR | 488 | PPAVSPREALMEAMHTIPVPPTTVRTDSLGKDA |
| 123 | P51452 | DUSP3 | KDTQEFNLSAY | 489 | ANFYKDSGITYLGIKAKDTQEFNLSAYFERAAD |
| 124 | O14917 | PCDH_7 | LFGLAVKSR | 490 | LRTYLLTRDDHGLFGLAVKSRGDGTKFPELVIQ |
| 125 | Q9UPQ0 | LIMCH1 | DVKKDDMSTR | 491 | RKLPDVKKDDMSTRRTSHGEPKSAVPFNQYLPN |
| 126 | Q9P2K1-6 | CC2D2A | HVCNPSTLGGR | 492 | LGRPGTVTHVCNPSTLGGRGG |
| 127 | Q86Y38 | XYLT_ | ANRGALPAR | 493 | GGGPGEPRGQQPANRGALPARALDPHPSPLITL |
| 128 | P53539 | FOSB | RRRELTDQL | 494 | RERNKLAAAKCRNRRRELTDQLQAETDQLEEEK |

In some embodiments, the amino acid sequence or nucleic acid sequence encoding the amino acid sequence is from about 5 to about 150 amino acids in length and the amino acid sequence is a fragment from any one or plurality of full-length neoantigens from a subject. In some embodiments, the fragments comprise, consist of, or consist essentially of one or more mutations at one or plurality of positions as compared to the full-length/wild-type sequence.

After PBMC isolation from subject blood draw, 4-1BB+ and/or CD69+CD8+ and CD4+ T cells will be sorted. If a particular clone responds to an epitope and comprises a high frequency of a particular marker expression, one can isolate that clone by way of CD3+, or CD4+, or CD8+ cells flow cytometry; magnetic beads specific for CD3+ or CD4+ or CD8+ cells; or any solid state column comprising antibodies specific for CD3+ or CD4+ or CD8+ cells.

Prepare a single-cell suspension of T-Cells in DMSO or 1×PBS.

### TCR Sequencing; Methods:

The single cell (sc) RNA-seq and scTCR-seq libraries are prepared using the 10× Single Cell Immune Profiling Solution Kit. In brief, PBMC are washed once with PBS containing 0.04% bovine serum albumin (BSA) and resuspended in PBS containing 0.04% BSA to a final concentration of 100-800 cells per μl as determined by hemacytometer. Cells are captured in droplets at a targeted cell recovery of 500-7,000 cells, resulting in estimated multiplet rates of 0.4-5.4%. Following reverse transcription and cell barcoding in droplets, emulsions are broken, and cDNA purified using Dynabeads MyOne SILANE followed by PCR amplification (98° C. for 45 s; 13-18 cycles of 98° C. for 20 s, 67° C. for 30 s, 72° C. for 1 min; 72° C. for 1 min). Amplified cDNA is then used for both 5′ gene expression library construction and TCR enrichment. For gene expression library construction, 2.4-50 ng of amplified cDNA is fragmented and end-repaired, double-sided size-selected with SPRIselect beads, PCR-amplified with sample indexing primers (98° C. for 45 s; 14-16 cycles of 98° C. for 20 s, 54° C. for 30 s, 72° C. for 20 s; 72° C. for 1 min), and double-sided size-selected with SPRIselect beads. For TCR library construction, TCR transcripts are enriched from 2 μl of amplified cDNA by PCR (primer sets 1 and 2:98° C. for 45 s; 10 cycles of 98° C. for 20 s, 67° C. for 30 s, 72° C. for 1 min; 72° C. for 1 min). Following TCR enrichment, 5-50 ng of enriched PCR product is fragmented and end-repaired, size-selected with SPRIselect beads, PCR-amplified with sample-indexing primers (98° C. for 45 s; 9 cycles of 98° C. for 20 s, 54° C. for 30 s, 72° C. for 20 s; 72° C. for 1 min), and size-selected with SPRIselect beads.

Alternatively, TCR library can be performed utilizing primers for TCR V gene amplification focusing on alpha and beta families as described in Ch'ng, et al. (Eur J Immunol. 2019). Briefly, the forward primers correspond to the first amino acid-coding nucleotides of the variable mRNA (table below). The reverse primer is located at the beginning of the constant region of the TCR (a family TRAC_Rv: 5′-CC GCT CGA GAC AGG GTT CTG GAT ATT-3′ (SEQ ID NO 365), β family TRBC2_Rv: 5′-TTT TCC TTT TGC GGC CGC GAA CAC GTT TTT CAG GTC-3′ (SEQ ID NO 366).

TABLE P
Forward primers for TCR (a and B families) library
construction.

| Primer | | | SEQ ID |
|---|---|---|---|
| ID | Family | Primer sequence | |
| TRAV1 | TRAV1-1*01, TRAV1-1*02, | GGA CAA ARC MTT GAS CAG | 129 |
| | TRAV1-2*01, TRAV1-2*02 | | |
| TRAV2 | TRAV2*01, TRAV2*02 | AAG GAC CAA GTG TTT CAG | 130 |
| TRAV3 | TRAV3*01, TRAV3*02 | GCT CAG TCA GTG GCT CAG | 131 |
| TRAV4 | TRAV4*01, TRAV26-1*02, | SWT GCT AAG ACC ACM CAG | 132 |
| | TRAV26-1*01, TRAV26-1*03, | | |
| | TRAV26-2*01, TRAV26-2*02 | | |
| TRAV5 | TRAV5*01, TRAV13-2*01, | GGA GAG RRT GTG GRG CWG | 133 |
| | TRAV13-2*02, TRAV13-1*03, | | |
| | TRAV13 1*02, TRAV13 1*01 | | |
| TRAV6 | TRAV6*02, TRAV6*01, | AGC CAA AAG ATA GAA CAG | 134 |
| TRAV7 | TRAV7*01, TRAV20*03, | GAA RAC CAG GTG RMG CAS | 135 |
| | TRAV20*04, TRAV20*02, | | |
| | TRAV20*01 | | |
| TRAV8 | TRAV8-5*01, TRAV8-3*03, | RCC CAG TCD GTG ASC CAG | 136 |
| | TRAV8-3*01, TRAV8-3*02, | | |
| | TRAV8-7*01, TRAV8-1*01, | | |
| | TRAV8-1*02, TRAV8-1*02, | | |
| | TRAV8-6*01, TRAV8-6*02, | | |
| | TRAV8-2*01, TRAV8-2*02, | | |
| | TRAV8-4*03, TRAV8-4*05, | | |
| | TRAV8-4*04, TRAV8-4*01, | | |
| | TRAV8 4*02 | | |
| TRAV9 | TRAV9-1*01, TRAV9-2*04, | GGA RAT TCA GTG RYC CAG | 137 |
| | TRAV9-2*01, TRAV9-2*03, | | |
| | TRAV9-2*02 | | |
| TRAV10 | TRAV10*01, TRAV41*01 | AAA AAY SAA GTG GAG CAG | 138 |
| TRAV11 | TRAV11*01, TRAV15*01 | CTM CAT AYW CTG GAG YAG | 139 |
| TRAV12 | TRAV12-1*01, TRAV12-1*02, | CRG AAG GAG GTG GAG CAG | 140 |
| | TRAV12-2*01, TRAV12-2*02, | | |
| | TRAV12-3*01, TRAV12-3*02 | | |
| TRAV14 | TRAV33*01, TRAV19*01, | GCY CAG AAR RTA ACY CAA | 141 |
| | TRAV14/DV4*02, | | |
| | TRAV14/DV4*01, | | |
| | TRAV14/DV4*03 | | |
| TRAV16 | TRAV38-2/DV8*01, TRAV38- | GCY CAG ASA GTC ACT CAG | 142 |
| | 1*04, TRAV38-1*03, TRAV38- | | |
| | 1*01, TRAV38-1*02, | | |
| | TRAV16*01 | | |
| TRAV17 | TRAV17*01 | AGT CAA CAG GGA GAA GAG | 143 |
| TRAV18 | TRAV18*01 | GGA GAC TCG GTT ACC CAG | 144 |
| TRAV21 | TRAV21*01, TRAV21*02 | AAA CAG GAG GTG ACR CAG | 145 |
| TRAV22 | TRAV22*01 | GGA ATA CAA GTG GAG CAG | 146 |
| TRAV23 | TRAV23/DV6*02, | CAG GTG AAA CAA | 147 |
| | TRAV23/DV6*01, TRAV23/DV6*03 | | |
| TRAV24 | TRAV24*01, TRAV24*02, | RWR CTG AAM GTG GAA CAA | 148 |
| | TRAV39*01 | | |
| TRAV25 | TRAV25*01 | GGA CAA CAG GTA ATG CAA | 149 |
| TRAV27 | TRAV27*02, TRAV27*01, | ACC CAG CTG GAG CAG | 150 |
| | TRAV27*03 | | |
| TRAV28 | TRAV28*01 | AAA GTG GAG CAG AGT CCT | 151 |
| TRAV29 | TRAV29/DV5*03, | GAC CAG CAA GTT AAG CAA | 152 |
| | TRAV29/DV5*02, TRAV29/DV5*01 | | |
| TRAV30 | TRAV30*04, TRAV30*01, | CAA CCA GTG CAG AGT | 153 |
| | TRAV30*03, TRAV30*02, | | |
| TRAV31 | TRAV31*01 | CAG AGG GTC ATT CAA TCC | 154 |
| TRAV32 | TRAV32*01 | AAG GAT GTG ATA CAG AGT | 155 |
| TRAV34 | TRAV34*01 | AGC CAA GAA CTG GAG CAG | 156 |
| TRAV35 | TRAV35*01, TRAV35*02 | GGT CAA CAG CTG AAT CAG | 157 |
| TRAV36 | TRAV36/DV7*02, | GAA GAC AAG GTG GTA CAA | 158 |
| | TRAV36/DV7*01, | | |
| | TRAV36/DV7*03, TRAV36/DV7*04 | | |
| TRAV37 | TRAV37*01 | CAA CTG CCA GTG GAA CAG | 159 |
| TRAV40 | TRAV40*01 | AGC AAT TCA GTC AAG CAG | 160 |

The scRNA libraries are sequenced on an Illumina NextSeq or HiSeq 4000 to a minimum sequencing depth of 25,000 reads per cell using read lengths of 26 bp read 1, 8 bp i7 index, 98 bp read 2. The single-cell TCR libraries are sequenced on an Illumina MiSeq or HiSeq 4000 to a minimum sequencing depth of 5,000 reads per cell using read lengths of 150 bp read 1, 8 bp i7 index, 150 bp read 2.

The scRNA-seq reads are aligned to the GRCh38 reference genome and quantified using cellranger count (10× Genomics, version 2.1.0). Filtered gene-barcode matrices that contained only barcodes with unique molecular identifier (UMI) counts that passed the threshold for cell detection are used for further analysis.

TCR reads are aligned to the GRCh38 reference genome and consensus TCR annotation is performed using cellranger vdj (10× Genomics, version 2.1.0). TCR libraries are sequenced to a minimum depth of 5,000 reads per cell, with a final average of 15,341 reads per cell.

T cells that recognize tumor antigens may proliferate to generate discernible clonal subpopulations defined by an identical T cell receptor (TCR) sequence. To identify potentially expanded T cell clones, we use ribonucleic acid sequencing (RNA-seq) reads that map to the TCR to classify single T cells by their isoforms of the V and J segments of the alpha and beta

TABLE Q
The following are examples of sequenced TCR alpha:

| SEQ | CDR3 (aa | SEQ | |
|---|---|---|---|
| ID | sequence) | ID | TCR (full aa sequence) |
| 161 | CAVGGSGGGADGLTF | 495 | MLLELIPLLGIHFVLRTARAQSVTQPDIHITVSEGASLELRCNYSYGATP |
| | | | YLFWYVQSPGQGLQLLLKYFSGDTLVQGIKGFEAEFKRSQSSFNLRKPSV |
| | | | HWSDAAEYFCAVGGSGGGADGLIFGKGTHLIIQPYIQKPDPAVYQLRDSK |
| | | | SSDKSVCLETDFDSQTNVSQSKDSDVYITDKTVLDMRSMDFKSNSAVAWS |
| | | | NKSDFACANAFNNSIIPEDTFFPSPESSCDVKLVEKSFETDTNLNFQNLS |
| | | | VIGFRILLLKVAGENLLMTLRLWSS |
| 162 | CAVGINARLMF | 496 | MMKSLRVLLVILWLQLSWVWSQQKEVEQNSGPLSVPEGAIASLNCTYSDR |
| | | | GSQSFFWYRQYSGKSPELIMFIYSNGDKEDGRETAQLNKASQYVSLLIRD |
| | | | SQPSDSATYLCAVGINARLMFGDGTQLVVKPNIQKPDPAVYQLRDSKSSD |
| | | | KSVCLFTDEDSQTNVSQSKDSDVYITDKTVLDMRSMDFKSNSAVAWSNKS |
| | | | DFACANAFNNSIIPEDTFFPSPESSCDVKLVEKSFETDINLNFQNLSVIG |
| | | | FRILLLKVAGFNLLMTLRLWSS |
| 163 | CAGPMKTSYDKVIF | 497 | MLLEHLLIILWMQLTWVSGQQLNQSPQSMFIQEGEDVSMNCTSSSIFNTW |
| | | | LWYKQDPGEGPVLLIALYKAGELTSNGRLTAQFGITRKDSFLNISASIPS |
| | | | DVGIYFCAGPMKTSYDKVIFGPGTSLSVIPNIQKPDPAVYQLRDSKSSDK |
| | | | SVCLFTDFDSQTNVSQSKDSDVYITDKTVLDMRSMDFKSNSAVAWSNKSD |
| | | | FACANAFNNSIIPEDTFFPSPESSCDVKLVEKSFETDINLNFQNLSVIGF |
| | | | RILLLKVAGFNLLMTLRLWSS |
| 164 | CAVRDLGAGGGNKLTF | 498 | MASAPISMLAMLFILSGLRAQSVAQPEDQVNVAEGNPLTVKCTYSVSGNP |
| | | | YLFWYVQYPNRGLQFLLKYITGDNLVKGSYGFEAEFNKSQTSFHLKKPSA |
| | | | LVSDSALYFCAVRDLGAGGGNKLTFGTGTQLKVELNIQNPDPAVYQLRDS |
| | | | KSSDKSVCLFTDFDSQTNVSQSKDSDVYITDKTVLDMRSMDFKSNSAVAW |
| | | | SNKSDEACANAENNSIIPEDTEFPSPESSCDVKLVEKSFETDINLNEQNL |
| | | | SVIGFRILLLKVAGFNLLMTLRLWSS |
| 165 | CAASKPGNQFYF | 499 | MTSIRAVFIFLWLQLDLVNGENVEQHPSTLSVQEGDSAVIKCTYSDSASN |
| | | | YFPWYKQELGKRPQLIIDIRSNVGEKKDQRIAVTLNKTAKHFSLHITETQ |
| | | | PEDSAVYFCAASKPGNQFYFGTGTSLTVIPNIQNPDPAVYQLRDSKSSDK |
| | | | SVCLFTDFDSQINVSQSKDSDVYITDKTVLDMRSMDFKSNSAVAWSNKSD |
| | | | FACANAFNNSIIPEDTFFPSPESSCDVKLVEKSFETDINLNFQNLSVIGF |
| | | | RILLLKVAGFNLLMTLRLWSS |
| 166 | CVVNRLHSYNYGQNFV | 50C | MMISLRVLLVILWLQLSWVWSQRKEVEQDPGPFNVPEGATVAFNCTYSNS |
| | F | | ASQSFFWYRQDCRKEPKLLMSVYSSGNEDGRETAQLNRASQYISLLIRDS |
| | | | KLSDSATYLCVVNRLHSYNYGQNFVFGPGTRLSVLPYIQKPDPAVYQLRD |
| | | | SKSSDKSVCLFTDFDSQTNVSQSKDSDVYITDKTVLDMRSMDFKSNSAVA |
| | | | WSNKSDFACANAFNNSIIPEDTFFPSPESSCDVKLVEKSFETDINLNFQN |
| | | | LSVIGFRILLLKVAGFNLLMTLRLWSS |
| 167 | CAVRGTDSWGKLQF | 50: | MEKMLECAFIVLWLQLGWLSGEDQVTQSPEALRLQEGESSSLNCSYTVSG |
| | | | LRGLFWYRQDPGKGPEFLFTLYSAGEEKEKERLKATLTKKESFLHITAPK |
| | | | PEDSATYLCAVRGTDSWGKLQFGAGTQVVVTPDIQNPDPAVYQLRDSKSS |
| | | | DKSVCLFTDFDSQTNVSQSKDSDVYITDKTVLDMRSMDFKSNSAVAWSNK |
| | | | SDFACANAENNSIIPEDTFFPSPESSCDVKLVEKSFETDTNLNFQNLSVI |
| | | | GFRILLLKVAGFNLLMTLRLWSS |

TABLE R
The following are examples of sequenced TCR beta:

| SEQ | CDR3 (aa | SEQ | |
|---|---|---|---|
| ID | sequence) | ID | TCR (full aa sequence) |
| 168 | CASSAVGNTIYF | 502 | MTIRLLCYVGFYFLGAGLMEADIYQTPRYLVIGTGKKITLECSQTMGHDK |
| | | | MYWYQQDPGMELHLIHYSYGVNSTEKGDLSSESTVSRIRTEHFPLTLESA |
| | | | RPSHTSQYLCASSAVGNTIYFGEGSWLTVVEDLNKVFPPEVAVFEPSEAE |
| | | | ISHTQKATLVCLATGFFPDHVELSWWVNGKEVHSGVSTDPQPLKEQPALN |
| | | | DSRYCLSSRLRVSATFWQNPRNHFRCQVQFYGLSENDEWTQDRAKPVTQI |
| | | | VSAEAWGRADCGFTSVSYQQGVLSATILYEILLGKATLYAVLVSALVLMA |
| | | | MVKRKDF |
| 169 | CVVSEGDSGGFKTIF | 503 | MGSRLLCWVLLCLLGAGPVKAGVTQTPRYLIKTRGQQVTLSCSPISGHRS |
| | | | VSWYQQTPGQGLQFLFEYFSETQRNKGNFPGRFSGRQFSNSRSEMNVSTL |
| | | | ELGDSALYLCASSPLAGGISDTQYFGPGTRLTVLEDLKNVFPPKVAVFEP |
| | | | SEAEISHTQKATLVCLATGFYPDHVELSWWVNGKEVHSGVSTDPQPLKEQ |
| | | | PALNDSRYCLSSRLRVSATFWQNPRNHFRCQVQFYGLSENDEWTQDRAKP |
| | | | VTQIVSAEAWGRADCGFTSESYQQGVLSATILYEILLGKATLYAVLVSAL |
| | | | VLMAMVKRKDSRG |
| 170 | CASSPLAGGISDIQY | 504 | MGSRLLCWVLLCLLGAGPVKAGVTQTPRYLIKTRGQQVTLSCSPISGHRS |
| | F | | VSWYQQTPGQGLQFLFEYFSETQRNKGNFPGRFSGRQFSNSRSEMNVSTL |
| | | | ELGDSALYLCASSPLAGGISDTQYFGPGTRLTVLEDLKNVFPPKVAVFEP |
| | | | SEAEISHTQKATLVCLATGFYPDHVELSWWVNGKEVHSGVSTDPQPLKEQ |
| | | | PALNDSRYCLSSRLRVSATFWQNPRNHFRCQVQFYGLSENDEWTQDRAKP |
| | | | VTQIVSAEAWGRADCGFTSESYQQGVLSATILYEILLGKATLYAVLVSAL |
| | | | VLMAMVKRKDSRG |
| 171 | CASSSANYGYTF | 505 | MGSWTLCCVSLCILVAKHTDAGVIQSPRHEVTEMGQEVTLRCKPISGHDY |
| | | | LFWYRQTMMRGLELLIYFNNNVPIDDSGMPEDRFSAKMPNASFSTLKIQP |
| | | | SEPRDSAVYFCASSSANYGYTEGSGTRLTVVEDLNKVFPPEVAVFEPSEA |
| | | | EISHTQKATLVCLATGFFPDHVELSWWVNGKEVHSGVSTDPQPLKEQPAL |
| | | | NDSRYCLSSRLRVSATFWQNPRNHFRCQVQFYGLSENDEWTQDRAKPVTQ |
| | | | IVSAEAWGRADCGFTSVSYQQGVLSATILYEILLGKATLYAVLVSALVLM |
| | | | AMVKRKDF |
| 172 | CASSTLVRETQYF | 506 | MGPGLLCWALLCLLGAGSVETGVTQSPTHLIKTRGQQVTLRCSSQSGHNT |
| | | | VSWYQQALGQGPQFIFQYYREEENGRGNFPPRFSGLQFPNYSSELNVNAL |
| | | | ELDDSALYLCASSILVRETQYFGPGTRLLVLEDLKNVFPPKVAVFEPSEA |
| | | | EISHTQKATLVCLATGFYPDHVELSWWVNGKEVHSGVSTDPQPLKEQPAL |
| | | | NDSRYCLSSRLRVSATFWQNPRNHFRCQVQFYGLSENDEWTQDRAKPVTQ |
| | | | IVSAEAWGRADCGFTSESYQQGVLSATILYEILLGKATLYAVLVSALVLM |
| | | | AMVKRKDSRG |
| 173 | CSAREYQGVKSPLHF | 507 | MEAVVTTLPREGGVRPSRKMLLLLLLLGPGSGLGAVVSQHPSRVICKSGT |
| | | | SVKIECRSLDFQATTMFWYRQFPKQSLMLMATSNEGSKATYEQGVEKDKF |
| | | | LINHASLTLSTLTVTSAHPEDSSFYICSAREYQGVKSPLHFGNGTRLIVT |
| | | | EDLNKVFPPEVAVFEPSEAEISHTQKATLVCLATGFFPDHVELSWWVNGK |
| | | | EVHSGVSTDPQPLKEQPALNDSRYCLSSRLRVSATFWQNPRNHERCQVQF |
| | | | YGLSENDEWTQDRAKPVTQIVSAEAWGRADCGETSVSYQQGVLSATILYE |
| | | | ILLGKATLYAVLVSALVLMAMVKRKDF |
| 174 | CASSFKMNTEAFF | 508 | MGPRLLFWALLCLLGIGPVEAGVTQSPTHLIKTRGQQVTLRCSPISGHTS |
| | | | VYWYQQALGLGLQFLLWYDEGEERNRGNFPPRFSGRQFPNYSSELNVNAL |
| | | | ELEDSALYLCASSFKMNTEAFFGQGTRLTVVEDLNKVFPPEVAVFEPSEA |
| | | | EISHTQKATLVCLATGFFPDHVELSWWVNGKEVHSGVSTDPQPLKEQPAL |
| | | | NDSRYCLSSRLRVSATFWQNPRNHFRCQVQFYGLSENDEWTQDRAKPVTQ |
| | | | IVSAEAWGRADCGFTSVSYQQGVLSATILYEILLGKATLYAVLVSALVLM |
| | | | AMVKRKDF |

Examples of lentiviral or retroviral plasmids.

The TCR is inserted in a retroviral or lentiviral vector, including pELNS (lentivirus), pRRL (lentiviral), pCR2.1 (lentiviral), pMSGV (retroviral), pMSCV (murine stem cell virus, retroviral) pMIG II (retroviral), LENTIVECTOR® or LENTIMAX™. LENTIMAX™ US patent number: US 2019/0134091 A1, which is hereby incorporated by reference.

The following are two examples of commercially-available plasmids with their corresponding DNA sequences.

| Plasmid | |
|---|---|
| ID | Backbone (DNA sequence) |
| pMSGV | AATGAAAGACCCCACCTGTAGGTTTGGCAAGCTAGCTTAAGTAACGCCATTTTGCAAGGCATGGAAAATA |
| Seq id | CATAACTGAGAATAGAGAAGTTCAGATCAAGGTTAGGAACAGAGAGACAGCAGAATATGGGCCAAACAGG |
| no: | ATATCTGTGGTAAGCAGTTCCTGCCCCGGCTCAGGGCCAAGAACAGATGGTCCCCAGATGCGGTCCCGCC |
| 175 | CTCAGCAGTTTCTAGAGAACCATCAGATGTTTCCAGGGTGCCCCAAGGACCTGAAAATGACCCTGTGCCT |
| | TATTTCAACTAACCAATCACTTCGCTTCTCCCTTCTGTTCCCCCCCTTCTCCTCCCCGAGCTCAATAAAA |
| | GAGCCCACAACCCCTCACTCGGCGCGCCAGTCCTCCGATAGACTGCGTCGCCCGGGTACCCGTATTCCCA |
| | ATAAAGCCTCTTGCTGTCTGCATCCGAATCGTGGACTCGCTGATCCTTGGGAGGGTCTCCTCAGATTGAT |
| | TGACTGCCCACCTCGGGGGTCTTTCATTTGGAGGTTCCACCGAGATTTGGAGACCCCTGCCTAGGGACCA |
| | CCGACCCCCCCGCCGGGAGGTAAGCTGGCCAGCGGTCGTTTCGTGTCTGTCTCTGTCTTTGTGCGTGTTT |
| | GTGCCGGCATCTAATGTTTGCGCCTGCGTCTGTACTAGTTAGCTAACTAGCTCTGTATCTGGCGGACCCG |
| | TGGTGGAACTGACGAGTTCGGAACACCCGGCCGCAACCCTGGGAGACGTCCCAGGGACTTCGGGGGCCGT |
| | TTTTGTGGCCCGACCTGAGTCCAAAAATCCCGATCGTTTTGGACTCTTTGGTGCACCCCCCTTAGAGGAG |
| | GGATATGTGGTTCTGGTAGGAGACGAGAACCTAAAACAGTTCCCGCCTCCGTCTGAATTTTTGCTTTCGG |
| | TTTGGGACCGAAGCCGCGCCGCGCGTCTTGTCTGCTGCAGCATCGTTCTGTGTTGTCTCTGTCTGACTGT |
| | GTTTCTGTATTTGTCTGAGAATATGGGCCCGGGCTAGCCTGTTACCACTCCCTTAAGTTTGACCTTAGGT |
| | CACTGGAAAGATGTCGAGCGGATCGCTCACAACCAGTCGGTAGATGTCAAGAAGAGACGTTGGGTTACCT |
| | TCTGCTCTGCAGAATGGCCAACCTTTAACGTCGGATGGCCGCGAGACGGCACCTTTAACCGAGACCTCAT |
| | CACCCAGGTTAAGATCAAGGTCTTTTCACCTGGCCCGCATGGACACCCAGACCAGGTCCCCTACATCGTG |
| | ACCTGGGAAGCCTTGGCCTTTGACCCCCCTCCCTGGGTCAAGCCCTTTGTACACCCTAAGCCTCCGCCTC |
| | CTCTTCCTCCATCCGCCCCGTCTCTCCCCCTTGAACCTCCTCGTTCGACCCCGCCTCGATCCTCCCTTTA |
| | TCCAGCCCTCACTCCTTCTCTAGGCGCCCCCATATGGCCATATGAGATCTTATATGGGGCACCCCCGCCC |
| | CTTGTAAACTTCCCTGACCCTGACATGACAAGAGTTACTAACAGCCCCTCTCTCCAAGCTCACTTACAGG |
| | CTCTCTACTTAGTCCAGCACGAAGTCTGGAGACCTCTGGCGGCAGCCTACCAAGAACAACTGGACCGACC |
| | GGTGGTACCTCACCCTTACCGAGTCGGCGACACAGTGTGGGTCCGCCGACACCAGACTAAGAACCTAGAA |
| | CCTCGCTGGAAAGGACCTTACACAGTCCTGCTGACCACCCCCACCGCCCTCAAAGTAGACGGCATCGCAG |
| | CTTGGATACACGCCGCCCACGTGAAGGCTGCCGACCCCGGGGGTGGACCATCCTCTAGACCCTGCAGGTT |
| | AATTAAGCCACCAAGTCCTCTTTTTTTGAAGACTTCAGAACCCTGATCCTGCCGTGTACCAGCTGCGGGA |
| | CAGCAAGAGCAGCGACAAGAGCGTGTGCCTGTTCACCGACTTCGACAGCCAGACCAACGTGTCCCAGAGC |
| | AAGGACAGCGACGTGTACATCACCGACAAGACCGTGCTGGACATGCGGAGCATGGACTTCAAGAGCAACA |
| | GCGCCGTGGCCTGGTCCAACAAGAGCGATTTCGCCTGCGCCAACGCCTTCAACAACAGCATTATCCCCGA |
| | GGACACATTCTTCCCAAGCCCCGAGAGCAGCTGCGACGTGAAGCTGGTGGAAAAGAGCTTCGAGACAGAC |
| | ACCAACCTGAACTTCCAGAACCTGAGCGTGATCGGCTTCCGGATCCTGCTGCTGAAGGTGGCCGGCTTCA |
| | ACCTGCTGATGACCCTGAGACTGTGGTCCAGCCGGGCCAAGAGATCTGGCAGCGGCGCCACCAATTTCAG |
| | CCTGCTGAAACAGGCCGGCGACGTGGAAGAGAACCCTAAGTCTTCTTTTTGAAGACTTGATCTGAACAAG |
| | GTGTTCCCCCCAGAGGTGGCCGTGTTCGAGCCTTCTGAGGCCGAGATCAGCCACACCCAGAAAGCCACCC |
| | TCGTGTGCCTGGCCACCGGCTTTTTCCCCGACCACGTGGAACTGTCTTGGTGGGTCAACGGCAAAGAGGT |
| | CCACACCCCCCTCTCCACCCATCCCCACCCTCTCAAAGAACACCCCCCCCTCAACCACAGCCCCTACTGC |
| | CTGAGCAGCAGACTGAGAGTGTCCGCCACCTTCTGGCAGAACCCCCGGAACCACTTCAGATGCCAGGTGC |
| | AGTTCTACGGCCTGAGCGAGAACGACGAGTGGACCCAGGACAGAGCCAAGCCCGTGACCCAGATCGTGTC |
| | TGCCGAAGCCTGGGGCAGAGCCGATTGCGGCTTTACCAGCGTGTCCTATCAGCAGGGCGTGCTGAGCGCC |
| | ACCATCCTGTACGAGATCCTGCTGGGCAAGGCCACCCTGTACGCCGTGCTGGTGTCTGCCCTGGTGCTGA |
| | TGGCCATGGTCAAGCGGAAGGACTTCTGAATTTGTAGATCGACGGATCCCTCGAGCTCAAGCTTCGAATT |
| | CTGCAGTCGACGGTACCGCGGGCCCGGGATCCGATAAAATAAAAGATTTTATTTAGTCTCCAGAAAAAGG |
| | GGGGAATGAAAGACCCCACCTGTAGGTTTGGCAAGCTAGCTTAAGTAACGCCATTTTGCAAGGCATGGAA |
| | AATACATAACTGAGAATAGAGAAGTTCAGATCAAGGTTAGGAACAGAGAGACAGCAGAATATGGGCCAAA |
| | CAGGATATCTGTGGTAAGCAGTTCCTGCCCCGGCTCAGGGCCAAGAACAGATGGTCCCCAGATGCGGTCC |
| | CGCCCTCAGCAGTTTCTAGAGAACCATCAGATGTTTCCAGGGTGCCCCAAGGACCTGAAATGACCCTGTG |
| | CCTTATTTGAACTAACCAATCAGTTCGCTTCTCGCTTCTGTTCGCGCGCTTCTGCTCCCCGAGCTCAATA |
| | AAAGAGCCCACAACCCCCCACTCGGCGCGCCAGTCCTCCGATAGACTGCGTCGCCCGGGTACCCGTGTAT |
| | CCAATAAACCCTCTTGCAGTTGCATCCGACTTGTGGTCTCGCTGTTCCTTGGGAGGGTCTCCTCTGAGTG |
| | ATTGACTACCCGTCAGCGGGGGTCTTTCATGGGTAACAGTTTCTTGAAGTTGGAGAACAACATTCTGAGG |
| | GTAGGAGTCGAATATTAAGTAATCCTGACTCAATTAGCCACTGTTTTGAATCCACATACTCCAATACTCC |
| | TGAAATCCATCGATGGAGTTCATTATGGACAGCGCAGAAAGAGCTGGGGAGAATTGTGAAATTGTTATCC |
| | GCTCACAATTCCACACAACATACGAGCCGGAAGCATAAAGTGTAAAGCCTGGGGTGCCTAATGAGTGAGC |
| | TAACTCACATTAATTGCGTTGCGCTCACTGCCCGCTTTCCAGTCGGGAAACCTGTCGTGCCAGCTGCATT |
| | AATGAATCGGCCAACGCGCGGGGAGAGGCGGTTTGCGTATTGGGCGCTCTTCCGCTTCCTCGCTCACTGA |
| | CTCGCTGCGCTCGGTCGCTCGGCTGCGGCGAGCGGTATCAGCTCACTCAAAGGCGGTAATACGGTTATCC |
| | ACAGAATCAGGGGATAACGCAGGAAAGAACATGTGAGCAAAAGGCCAGCAAAAGGCCAGGAACCGTAAAA |
| | ACCCCCCCTTCCTGCCCTTTTTCCATACGCTCCCCCCCCCTCACCACCATCACAAAAATCCACCCTCAAC |
| | TCAGAGGTGGCGAAACCCGACAGGACTATAAAGATACCAGGCGTTTCCCCCTGGAAGCTCCCTCGTGCGC |
| | TCTCCTGTTCCGACCCTGCCGCTTACCGGATACCTGTCCGCCTTTCTCCCTTCGGGAAGCGTGGCGCTTT |
| | CTCATAGCTCACGCTGTAGGTATCTCAGTTCGGTGTAGGTCGTTCGCTCCAAGCTGGGCTGTGTGCACGA |
| | ACCCCCCGTTCAGCCCGACCGCTGCGCCTTATCCGGTAACTATCGTCTTGAGTCCAACCCGGTAAGACAC |
| | GACTTATCGCCACTGGCAGCAGCCACTGGTAACAGGATTAGCAGAGCGAGGTATGTAGGCGGTGCTACAG |
| | AGTTCTTGAAGTGGTGGCCTAACTACGGCTACACTAGAAGGACAGTATTTGGTATCTGCGCTCTGCTGAA |
| | GCCAGTTACCTTCGGAAAAAGAGTTGGTAGCTCTTGATCCGGCAAACAAACCACCGCTGGTAGCGGTGGT |
| | TTTTTTGTTTGCAAGCAGCAGATTACGCGCAGAAAAAAAGGATCTCAAGAAGATCCTTTGATCTTTTCTA |
| | CGGGGTCTGACGCTCAGCGGAACGAAAACTCACGTTAAGGGATTTTGGTCATGAGATTATCAAAAAGGAT |
| | CTTCACCTAGATCCTTTTAAATTAAAAATGAAGTTTTAAATCAATCTAAAGTATATATGAGTAAACTTGG |
| | TCTGACAGTTACCAATGCTTAATCAGTGAGGCACCTATCTCAGCGATCTGTCTATTTCGTTCATCCATAG |
| | TTGCCTGACTCCCCGTCGTGTAGATAACTACGATACGGGAGGGCTTACCATCTGGCCCCAGTGCTGCAAT |
| | GATACCGCGAGACCCACGCTCACCGGCTCCAGATTTATCAGCAATAAACCAGCCAGCCGGAAGGGCCGAG |
| | CGCAGAAGTGGTCCTGCAACTTTATCCGCCTCCATCCAGTCTATTAATTGTTGCCGGGAAGCTAGAGTAA |
| | GTAGTTCGCCAGTTAATAGTTTGCGCAACGTTGTTGCCATTGCTACAGGCATCGTGGTGTCACGCTCGTC |
| | GTTTGGTATGGCTTCATTCAGCTCCGGTTCCCAACGATCAAGGCGAGTTACATGATCCCCCATGTTGTGC |
| | AAAAAACCCCTTAGCTCCTTCCGTCCTCCCATCCTTCTCACAACTAAGTTGGCCCCAGTGTTATCACTCA |
| | TGGTTATGGCAGCACTGCATAATTCTCTTACTGTCATGCCATCCGTAAGATGCTTTTCTGTGACTGGTGA |
| | GTACTCAACCAAGTCATCCTGAGAATAGTGTATGCGGCGACCGAGTTGCTCTTGCCCGGCGTCAATACGG |
| | GATAATACCGCGCCACAZAGCAGAACTTTAAAAGTGCTCATCATTGGAAAACGTTCTTCGGGGCGAAAAC |
| | TCTCAAGGATCTTACCGCTGTTGAGATCCAGTTCGATGTAACCCACTCGTGCACCCAACTGATCTTCAGC |
| | ATCTTTTACTTTCACCAGCGTTTCTGGGTGAGCAAAAACAGGAAGGCAAAATGCCGCAAAAAAGGGAATA |
| | AGGGCGACACGGAAATGCTGAATACTCATACTCTTCCTTTTTCAATATTATTGAAGCATTTATCAGGGTT |
| | ATTGTCTCATGAGCGGAZACATATTTGAATGTATTTAGAAAAATAAACAAATAGGGGTTCCGCGCACATT |
| | TCCCCGAAAAGTGCCACCTGACGTCTAAGAAACCATTATTATCATGACATTAACCTATAAAAATAGGCGT |
| | ATCACGAGGCCCTTTCGCCTCGCGCGTTTCGGTGATGACGGTGAAAACCTCTGACACATGCAGCTCCCGG |
| | AGACGGTCACAGCTTGTCTGTAAGCGGATGCCGGGAGCAGACAAGCCCGTCAGGGCGCGTCAGCGGGTGT |
| | TGGCGGGTGTCGGGGCTGGCTTAACTATGCGGCATCAGAGCAGATTGTACTGAGAGTGCACCATATGCGG |
| | TGTGAAATACCGCACAGATGCGTAAGGAGAAAATACCGCATCAGGCGCCATTCGCCATTCAGGCTGCGCA |
| | ACTGTTGGGAAGGGCGATCGGTGCGGGCCTCTTCGCTATTACGCCAGCTGGCGAAAGGGGGATGTGCTGC |
| | AAGGCGATTAAGTTGGGCAACGCCAGGGTTTTCCCAGTCACGACGTTGTAAAACGACGGCCAGTGCCACG |
| | CTCTCCCTTATGCGACTCCTGCATTAGGAAGCAGCCCAGTAGTAGGTTGAGGCCGTTGAGCACCGCCGCC |
| | GCAAGGAATGGTGCATGCAAGGAGATGGCGCCCAACAGTCCCCCGGCCACGGGGCCTGCCACCATACCCA |
| | CGCCGAAACAAGCGCTCATGAGCCCGAAGTGGCGAGCCCGATCTTCCCCATCGGTGATGTCGGCGATATA |
| | GGCGCCAGCAACCGCACCTGTGGCGCCGGTGATGCCGGCCACGATGCGTCCGGCGTAGAGGCGATTTAAA |
| | GACAGGATATCAGTGGTCCAGGCTCTAGTTTTGACTCAACAATATCACCAGCTGAAGCCTATAGAGTACG |
| | AGCCATAGATAAAATAAAAGATTTTATTTAGTCTCCAGAAAAAGGGGGG |
| PMSCV | AATGAAAGACCCCACCTGTAGGTTTGGCAAGCTAGCTTAAGTAACGCCATTTEGCAAGGCATGGAAAATA |
| Seq id | CATAACTGAGAATAGAGAAGTTCAGATCAAGGTTAGGAACAGAGAGACAGCAGAATATGGGCCAAACAGG |
| no: | ATATCTGTGGTAAGCAGTTCCTGCCCCGGCTCAGGGCCAAGAACAGATGGTCCCCAGATGCGGTCCCGCC |
| 176 | CTCAGCAGTTTCTAGAGAACCATCAGATGTTTCCAGGGTGCCCCAAGGACCTGAAATGACCCTGTGCCTT |
| | ATTTGAACTAACCAATCAGTTCGCTTCTCGCTTCTGTTCGCGCGCTTCTGCTCCCCGAGCTCAATAAAAG |
| | AGCCCACAACCCCTCACTCGGCGCGCCAGTCCTCCGATAGACTGCGTCGCCCGGGTACCCGTATTCCCAA |
| | TAAAGCCTCTTGCTGTTEGCATCCGAATCGTGGACTCGCTGATCCTTGGGAGGGTCTCCTCAGATTGATT |
| | GACTGCCCACCTCGGGGGTCTTTCATTTGGAGGTTCCACCGAGATTTGGAGACCCCTGCCCAGGGACCAC |
| | CGACCCCCCCGCCGGGAGGTAAGCTGGCCAGCGGTCGTTTCGTGTCTGTCTCTGTCTTTGTGCGTGTTTG |
| | TGCCGGCATCTAATGTTTGCGCCTGCGTCTGTACTAGTTAGCTAACTAGCTCTGTATCTGGCGGACCCGT |
| | GGTGGAACTGACGAGTTCTGAACACCCGGCCGCAACCCTGGGAGACGTCCCAGGGACTTTGGGGGCCGTT |
| | TTTGTGGCCCGACCTGAGGAAGGGAGTCGATGTGGAATCCGACCCCGTCAGGATATGTGGTTCTGGTAGG |
| | AGACGAGAACCTAAAACAGTTCCCGCCTCCGTCTGAATTTTTGCTTTCGGTTTGGAACCGAAGCCGCGCG |
| | TCTTGTCTGCTGCAGCGCTGCAGCATCGTTCTGTGTTGTCTCTGTCTGACTGTGTTTCTGTATTTGTCTG |
| | AAAATTAGGGCCAGACTGTTACCACTCCCTTAAGTTTGACCTTAGGTCACTGGAAAGATGTCGAGCGGAT |
| | CGCTCACAACCAGTCGGTAGATGTCAAGAAGAGACGTTGGGTTACCTTCTGCTCTGCAGAATGGCCAACC |
| | TTTAACGTCGGATGGCCGCGAGACGGCACCTTTAACCGAGACCTCATCACCCAGGTTAAGATCAAGGTCT |
| | TTTCACCTGCCCCCCATCCACACCCACACCAGGTCCCCTACATCGTCACCTCCCAACCCTTCGCTTTTCA |
| | CCCCCCTCCCTGGGTCAAGCCCTTTGTACACCCTAAGCCTCCGCCTCCTCTTCCTCCATCCGCCCCGTCT |
| | CTCCCCCTTGAACCTCCECGTTCGACCCCGCCTCGATCCTCCCTTTATCCAGCCCTCACTCCTTCTCTAG |
| | GCGCCGGAATTCAGATCETACGTAGCTAGCGGATCCCAATTGCTCGAGCCTGCAGGCATGCAAGCTTCAG |
| | GTAGCCGGCTAACGTTAACAACCGGTACCTCTAGAACTATAGCTAGCATGCGCAAATTTAAAGCGCTGAT |
| | ATCGATAAAATAAAAGASTTTATTTAGTCTCCAGAAAAAGGGGGGAATGAAAGACCCCACCTGTAGGTTT |
| | GGCAAGCTAGCTTAAGTAACGCCATTTTGCAAGGCATGGAAAATACATAACTGAGAATAGAGAAGTTCAG |
| | ATCAAGGTTAGGAACAGAGAGACAGCAGAATATGGGCCAAACAGGATATCTGTGGTAAGCAGTTCCTGCC |
| | CCGGCTCAGGGCCAAGAACAGATGGTCCCCAGATGCGGTCCCGCCCTCAGCAGTTTCTAGAGAACCATCA |
| | GATGTTTCCAGGGTGCCCCAAGGACCTGAAATGACCCTGTGCCTTATTTGAACTAACCAATCAGTTCGCT |
| | TCTCGCTTCTGTTCGCGCGCTTCTGCTCCCCGAGCTCAATAAAAGAGCCCACAACCCCTCACTCGGCGCG |
| | CCAGTCCTCCGATAGACTGCGTCGCCCGGGTACCCGTGTATCCAATAAACCCTCTTGCAGTTGCATCCGA |
| | CTTGTGGTCTCGCTGTTCCTTGGGAGGGTCTCCTCTGAGTGATTGACTACCCGTCAGCGGGGGTCTTTCA |
| | TGGGTAACAGTTTCTTGAAGTTGGAGAACAACATTCTGAGGGTAGGAGTCGAATATTAAGTAATCCTGAC |
| | TCAATTAGCCACTGTTTTGAATCCACATACTCCAATACTCCTGAAATAGTTCATTATGGACAGCGCAGAA |
| | AGAGCTGGGGAGAATTGEGAAATTGTTATCCGCTCACAATTCCACACAACATACGAGCCGGAAGCATAAA |
| | GTGTAAAGCCTGGGGTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAGAGGCGGTTTGCGTA |
| | TTGGGCGCTCTTCCGCTTCCTCGCTCACTGACTCGCTGCGCTCGGTCGTTCGGCTGCGGCGAGCGGTATC |
| | AGCTCACTCAAAGGCGGEAATACGGTTATCCACAGAATCAGGGGATAACGCAGGAAAGAACATGTGAGCA |
| | AAAGGCCAGCAAAAGGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGACTATAAAGATACCA |
| | CCCCTTTCCCCCTCGAACCTCCCTCCTGCCCTCTCCTCTTCCCACCCTGCCCCTTACCCGATACCTGTCC |
| | GCCTTTCTCCCTTCGGGAAGCGTGGCGCTTTCTCATAGCTCACGCTGTAGGTATCTCAGTTCGGTGTAGG |
| | TCGTTCGCTCCAAGCTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCACTGGTAACAGGATT |
| | AGCAGAGCGAGGTATGTAGGCGGTGCTACAGAGTTCTTGAAGTGGTGGCCTAACTACGGCTACACTAGAA |
| | GAACAGTATTTGGTATCTGCGCTCTGCTGAAGCCAGTTACCTTCGGAAAAAGAGTTGGTAGCTCTTGATC |
| | CGGCAAACAAACCACCGCTGGTAGCGGTGGTTTTTTTGTTTGCAAGCAGCAGATTACGCGCAGAAAAAAA |
| | GGATCTCAAGAAGATCCETTGATCTTTTCTACGGGGTCTGACGCTCAGTGGAACGAAAACTCACGTTAAG |
| | GGATTTTGGTCATGAGATTATCAAAAAGGATCTTCACCTAGATCCTTTTAAATTAAAAATGAAGTTTTAA |
| | ATCAATCTAAAGTATATATGAGTAAACTTGGTCTGACAGTTACCAATGCTTAATCAGTGAGGCACCTATC |
| | TCAGCGATCTGTCTATTECGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCGGCTCCAGATTTATC |
| | AGCAATAAACCAGCCAGCCGGAAGGGCCGAGCGCAGAAGTGGTCCTGCAACTTTATCCGCCTCCATCCAG |
| | TCTATTAATTGTTGCCGGGAAGCTAGAGTAAGTAGTTCGCCAGTTAATAGTTTGCGCAACGTTGTTGCCA |
| | TTGCTACAGGCATCGTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTCCTCCGATCGTTGTC |
| | AGAAGTAAGTTGGCCGCAGTGTTATCACTCATGGTTATGGCAGCACTGCATAATTCTCTTACTGTCATGC |
| | CATCCCTAAGATCCTTTTCTCTGACTCCTGAGTACTCAACCAACTCATTCTGAGAATAGTGTATGCGGCC |
| | ACCGAGTTGCTCTTGCCCGGCGTCAATACGGGATAATACCGCGCCACATAGCAGAACTTTAAAAGTGCTC |
| | ATCATTGGAAAACGTTCETCGGGGCGAAAACTCTCAAGGATCTTACCGCTGTTGAGATCCAGTTCGATGT |
| | AACCCACTCGTGCACCCAACTGATCTTCAGCATCTTTTACTTTCACCAGCGTTTCTGGGTGAGCAAAAAC |
| | AGGAAGGCAAAATGCCGCAAAAAAGGGAATAAGGGCGACACGGAAATGTTGAATACTCATACTCTTCCTT |
| | TTTCAATATTATTGAAGCATTTATCAGGGTTATTGTCTCATGAGCGGATACATATTTGAATGTATTTAGA |
| | AAAATAAACAAATAGGGGTTCCGCGCACATTTCCCCGAAAAGTGCCACCTGACGTCTAAGAAACCATTAT |
| | TATCATGACATTAACCTATAAAAATAGGCGTATCACGAGGCCCTTTCGTCTCGCGCGTTTCGGTGATGAC |
| | GGTGAAAACCTCTGACACATGCAGCTCCCGGAGACGGTCACAGCTTGTCTGTAAGCGGATGCCGGGAGCA |
| | GACAAGCCCGTCAGGGCGCGTCAGCGGGTGTTGGCGGGTGTCGGGGCTGGCTTAACTATGCGGCATCAGA |
| | GCAGATTGTACTGAGAGEGCACCATATGCGGTGTGAAATACCGCACAGATGCGTAAGGAGAAAATACCGC |
| | ATCAGGCGCCATTCGCCATTCAGGCTGCGCAACTGTTGGGAAGGGCGATCGGTGCGGGCCTCTTCGCTAT |
| | TACGCCAGCTGGCGAAAGGGGGATGTGCTGCAAGGCGATTAAGTTGGGTAACGCCAGGGTTTTCCCAGTC |
| | ACGACGTTGTAAAACGACGGCGCAAGGAATGGTGCATGCAAGGAGATGGCGCCCAACAGTCCCCCGGCCA |
| | CGGGGCCTGCCACCATACCCACGCCGAAACAAGCGCTCATGAGCCCGAAGTGGCGAGCCCGATCTTCCCC |
| | ATCGGTGATGTCGGCGAZATAGGCGCCAGCAACCGCACCTGTGGCGCCGGTGATGCCGGCCACGATGCGT |
| | CCGGCGTAGAGGCGATTAGTCCAATTTGTTAAAGACAGGATATCAGTGGTCCAGGCTCTAGTTTTGACTC |
| | AACAATATCACCAGCTGAAGCCTATAGAGTACGAGCCATACATAAAATAAAAGATTTTATTTAGTCTCCA |
| | GAAAAAGGGGGG |

TCR-transfected cells will be cocultured with patient's own antigen presenting cells (APC), or artificial APCs (e.g. HLA-matched K562). Antigen-specific T cells will be identified by CD69 upregulation 18 hours following coculture. The expected affinity between the peptide-HLA complex and the TCR is less than about 500 nM.

### In Vitro Cytotoxicity

To test cytotoxicity in vitro, antigen-specific TCR T cells are activated in a 96 well round-bottom plate with anti CD3/CD28 beads (ThermoFisher Scientific) and supplemented with AIM V/5% human AB serum and 20 ng/ml IL-2 for 36 h. Then, 2-fold serial dilutions of activated T cells are plated in 96 well round-bottom plates starting at 1×105 cells per well in AIM V/5% human AB serum. HLA-matched luciferase-expressing K562 cells are co-cultured with T cells for 18 h at different ratios (e.g., 1:1, 1:3, 1:10 effector: target ratio), either transduced to express the neoantigen of interest or an irrelevant neoantigen as a control. Alternatively, HLA-matched luciferase-expressing K562 cells are pulsed with relevant (e.g. YLGSGIHSGA (SEQ ID NO: 1) in the example of CTNNB1 mutation D32G, TTAPPLSGK (SEQ ID NO: 16) in the example of CTNNB1 mutation S45P) or irrelevant neoantigen epitopes. Luciferase assay is performed with Bright-Glo™ Luciferase Assay System (Promega) following the manufacturer's protocol to correlate luciferase expression with T cell cytotoxicity. Briefly, cells in 96-well plates are equilibrated at room temperature for 5 minutes. Then, a volume of Bright-Glo™ reagent equal to the volume of culture medium is added and mixed. After 2 minutes of cell lysis, the luminescence can be measured in a luminometer.

Alternatively, in vitro cytotoxicity can be assessed by flow cytometry. Here, after 18 hours of co-culture (antigen-specific TCR T cells: HLA-matched K562) cells plated in 96-well plates are spun down at 1500 RPM, 5 minutes, at room temperature. Then, cells are washed in 200 uL of PBS, centrifuged again (same settings). Cells are resuspended in 1 mL of FACS buffer (PBS, 0.5-1% BSA), transferred to FACS tubes (5 mL, conical) and centrifuged at 1500 RPM, 5 minutes, at room temperature. Next, the pellet is resuspended in 100 uL of a master mix including the fluorescent-conjugated antibodies against CD45, Annexin V, and 7-AAD and incubated for 30 minutes at 4° C. in the dark. Upon incubation is completed, cells are washed (1500 RPM, 5 minutes, at room temperature) and resuspended in 300 uL of FACS buffer for further analysis in the flow cytometer. The CD45 negative, Annexin V/7-AAD double-positive cells correspond to apoptotic HLA-matched K562. Thus, the more apoptotic cells observed, the more T cell cytotoxicity (Perales-Puchalt, Mol Ther. 2019).

### In Vivo Cytotoxicity

To measure cytotoxicity in vivo, HLA-matched human tumor cell lines of the relevant tumor type are transduced with the relevant neoantigen, an irrelevant neoantigen or the wild-type epitope. Tumor cell lines are grown in vitro under sterile conditions in RPMI 10%-FBS culture media supplemented with 1% penicillin/streptomycin at 37° C., 5%-CO2. Next, tumor cells (1×106 in 100 uL PBS) are injected subcutaneously in the back of immunodeficient NOD-SCID-Gamma (NSG) mouse and tumor volume is monitored three times per week with a digital caliper. TCR-transduced T cells are cultured in vitro under sterile conditions in RPMI 10%-FBS culture media supplemented with 1% penicillin/streptomycin at 37° C., 5%-CO2. When tumors reach a volume of approximately 100 mm3, the TCR-transduced T cells are injected (2×106 in 100 uL PBS) intraperitoneally in the mice. Then, tumor burden is monitored three times per week with a digital caliper until the endpoint (2000 mm3 tumor volume) is reached. Mice adoptively transferred with the corresponding neoantigen-specific CD8+ T cells can control tumor growth. Instead, mock-treated mice show control-level tumor growth.

When performing the above-steps, we can remove or omit one or more cytotoxicity experiments so that T cells expressing antigen-specific TCRs are isolated after only a single cytoxicity experiment.

### Example 2—Cancer Epitopes that are not Neoantigens

Following vaccination against 20-40 epitopes from individualized neoantigens from a sample, PBMC are collected from the patient. Then, a cocktail of cytokines (IL-2+IL-4+IL-7) are utilized in a 10-day time course to activate human T cells ex vivo (Karanikas et al. J Immunol 2003; 171:4898-4904; Roudko et al. Cell 2020; 183:1634-1649). The protocol is as follows: on day 0, PBMC are thawed and rest overnight in complete RPMI 10%-human serum media. On day 1, cells are counted and 250,000 PBMC are plated in 0.2 mL of media in 96 well plate. The antigenic peptides are added to a final concentration of 10 μg/mL. Then, the cytokines are added to the media using the following concentrations: IL-2 (20 IU/mL), IL4 (10 ng/ml), and IL-7 (10 ng/ml). Cells are left untouched in the incubator until the next time point at 37° C., 5%-CO2. On days 3 and 6, PBMC are spun down at room temperature, 1500 RPM, for 5 min. Then half of the media (0.1 mL) is carefully removed and replaced with fresh RPMI10%-human serum containing cytokines for final concentrations of: IL-2 (20 IU/mL), IL4 (10 ng/ml), and IL-7 (10 ng/ml). Cells are left untouched in the incubator until the next time point at 37° C., 5%-CO2. On day 9, cells are spun down at room temperature, 1500 RPM, for 5 min, and the media is completely replaced with fresh, cytokine-free RPMI10%-human serum media. Cells are left untouched in the incubator until the next time point at 37° C., 5%-CO2. On day 10, cells are stimulated with the corresponding peptides (10 ug/mL), and returned to the incubator at 37° C., 5%-CO2. 18 to 24 hours later, cells are stained and sorted based on CD137 and/or CD69 status.

The following are three examples of full-length cancer proteins and their HLA class I-binding epitopes (9- or 10-mers) that are not neoantigens. Additional 15-mer sequences listed can be utilized to experimentally determine the most adequate TCR.

MAGEA-10 (MAGE family member A10):
FASTA:
>sp|P43363|MAGAA_HUMAN Melanoma-associated antigen 10 GN=MAGEA10

Full length protein sequence
(SEQ ID NO: 177)
MPRAPKRQRCMPEEDLQSQSETQGLEGAQAPLAVEEDASSSTSTSSSFP
SSFPSSSSSSSSSCYPLIPSTPEEVSADDETPNPPQSAQIACSSPSVVA
SLPLDQSDEGSSSQKEESPSTLQVLPDSESLPRSEIDEKVTDLVQFLLF
KYQMKEPITKAEILESVIRNYEDHFPLLFSEASECMLLVFGIDVKEVDP
TGHSFVLVTSLGLTYDGMLSDVQSMPKTGILILILSIVFIEGYCTPEEV
IWEALNMMGLYDGMEHLIYGEPRKLLTQDWVQENYLEYRQVPGSDPARY
EFLWGPRAHAEIRKMSLLKFLAKVNGSDPRSFPLWYEEALKDEEERAQD
RIATTDDTTAMASASSSATGSFSYPE.

| Name | Sequence | Length | SEQ ID |
|---|---|---|---|
| MAGE-A10-TAA1 | EEVIWEALNMMGLYDGMEHLIYGEPRKLLTQ | 31 | 178 |
| MAGE-A10-TAA1-1 | GLYDGMEHL | 9 | 179 |
| MAGE-A10-TAA1-2 | EEVIWEALNMMGLYD | 15 | 180 |
| MAGE-A10-TAA1-3 | ALNMMGLYDGMEHLI | 15 | 181 |
| MAGE-A10-TAA1-4 | LYDGMEHLIYGEPRK | 15 | 182 |
| MAGE-A10-TAA2 | GPRAHAEIRKMSLLKFLAKVNGSDPRSFPLW | 31 | 183 |
| MAGE-A10-TAA2-1 | SLLKFLAKV | 9 | 184 |
| MAGE-A10-TAA2-2 | GPRAHAEIRKMSLLK | 15 | 185 |
| MAGE-A10-TAA2-3 | EIRKMSLLKFLAKVN | 15 | 186 |
| MAGE-A10-TAA2-4 | LLKFLAKVNGSDPRS | 15 | 187 |
| MAGE-A10-TAA3 | SESLPRSEIDEKVTDLVQFLLFKYQMKEPIT | 31 | 188 |
| MAGE-A10-TAA3-1 | KVTDLVQFL | 9 | 189 |
| MAGE-A10-TAA3-2 | SESLPRSEIDEKVTD | 15 | 190 |
| MAGE-A10-TAA3-3 | SEIDEKVTDLVQFLL | 15 | 191 |
| MAGE-A10-TAA3-4 | VTDLVQFLLFKYQMK | 15 | 192 |
| MAGE-A10-TAA4 | QVPGSDPARYEFLWGPRAHAEIRKMSLLKFL | 31 | 193 |
| MAGE-A10-TAA4-1 | FLWGPRAHA | 9 | 194 |
| MAGE-A10-TAA4-2 | QVPGSDPARYEFLWG | 15 | 195 |
| MAGE-A10-TAA4-3 | PARYEFLWGPRAHAE | 15 | 196 |
| MAGE-A10-TAA4-4 | LWGPRAHAEIRKMSL | 15 | 197 |
| MAGE-A10-TAA5 | VLVTSLGLTYDGMLSDVQSMPKTGILILILS | 31 | 198 |
| MAGE-A10-TAA5-1 | GMLSDVQSM | 9 | 199 |
| MAGE-A10-TAA5-2 | VLVTSLGLTYDGMLS | 15 | 200 |
| MAGE-A10-TAA5-3 | GLTYDGMLSDVQSMP | 15 | 201 |
| MAGE-A10-TAA5-4 | MLSDVQSMPKTGILI | 15 | 202 |
| MAGE-A10-TAA6 | VTDLVQFLLFKYQMKEPITKAEILESVIRNY | 31 | 203 |
| MAGE-A10-TAA6-1 | YQMKEPITKA | 10 | 204 |
| MAGE-A10-TAA6-2 | VTDLVQFLLFKYQMK | 15 | 205 |
| MAGE-A10-TAA6-3 | FLLFKYQMKEPITKA | 15 | 206 |
| MAGE-A10-TAA6-4 | QMKEPITKAEILESV | 15 | 207 |
| MAGE-A10-TAA7 | CMLLVFGIDVKEVDPTGHSFVLVTSLGLTYD | 31 | 208 |
| MAGE-A10-TAA7-1 | EVDPTGHSF | 9 | 209 |
| MAGE-A10-TAA7-2 | CMLLVFGIDVKEVDP | 15 | 210 |
| MAGE-A10-TAA7-3 | GIDVKEVDPTGHSFV | 15 | 211 |
| MAGE-A10-TAA7-4 | VDPTGHSFVLVISLG | 15 | 212 |
| MAGE-A10-TAA8 | KYQMKEPITKAEILESVIRNYEDHFPLLFSE | 32 | 213 |
| MAGE-A10-TAA8-1 | EILESVIRNY | 10 | 214 |
| MAGE-A10-TAA8-2 | KYQMKEPITKAEILE | 15 | 215 |
| MAGE-A10-TAA8-3 | PITKAEILESVIRNY | 15 | 216 |
| MAGE-A10-TAA8-4 | ILESVIRNYEDHFPL | 15 | 217 |
| MAGE-A10-TAA8-5 | RNYEDHFPLLFSE | 14 | 218 |
| MAGE-A10-TAA9 | IVFIEGYCTPEEVIWEALNMMGLYDGMEHLI | 31 | 219 |
| MAGE-A10-TAA9-1 | EVIWEALNM | 9 | 220 |
| MAGE-A10-TAA9-2 | IVFIEGYCTPEEVIW | 15 | 221 |
| MAGE-A10-TAA9-3 | YCTPEEVIWEALNMM | 15 | 222 |
| MAGE-A10-TAA9-4 | VIWEALNMMGLYDGM | 15 | 223 |
| MAGE-A10-TAA10 | YGEPRKLLTQDWVQENYLEYRQVPGSDPARY | 31 | 224 |
| MAGE-A10-TAA10-1 | WVQENYLEY | 9 | 225 |
| MAGE-A10-TAA10-2 | YGEPRKLLTQDWVQE | 15 | 226 |
| MAGE-A10-TAA10-3 | LLTQDWVQENYLEYR | 15 | 227 |
| MAGE-A10-TAA10-4 | VQENYLEYRQVPGSD | 15 | 228 |
| MAGE-A10-TAA11 | RNYEDHFPLLFSEASECMLLVFGIDVKEVDP | 31 | 229 |
| MAGE-A10-TAA11-1 | SEASECMLL | 9 | 230 |
| MAGE-A10-TAA11-2 | RNYEDHFPLLFSEAS | 15 | 231 |
| MAGE-A10-TAA11-3 | FPLLFSEASECMLLV | 15 | 232 |
| MAGE-A10-TAA11-4 | EASECMLLVFGIDVK | 15 | 233 |
| MAGE-A10-TAA12 | ESPSTLQVLPDSESLPRSEIDEKVTDLVQFL | 31 | 234 |
| MAGE-A10-TAA12-1 | SESLPRSEI | 9 | 235 |
| MAGE-A10-TAA12-2 | ESPSTLQVLPDSESL | 15 | 236 |
| MAGE-A10-TAA12-3 | QVLPDSESLPRSEID | 15 | 237 |
| MAGE-A10-TAA12-4 | ESLPRSEIDEKVTDL | 15 | 238 |
| MAGE-A10-TAA13 | DQSDEGSSSQKEESPSTLQVLPDSESLPRSE | 31 | 239 |
| MAGE-A10-TAA13-1 | EESPSTLQVL | 10 | 240 |
| MAGE-A10-TAA13-2 | DQSDEGSSSQKEESP | 15 | 241 |
| MAGE-A10-TAA13-3 | SSSQKEESPSTLQVL | 15 | 242 |
| MAGE-A10-TAA13-4 | ESPSTLQVLPDSESL | 15 | 243 |
| MAGE-A10-TAA14 | PNPPQSAQIACSSPSVVASLPLDQSDEGSSS | 31 | 244 |
| MAGE-A10-TAA14-1 | SSPSVVASL | 9 | 245 |
| MAGE-A10-TAA14-2 | PNPPQSAQIACSSPS | 15 | 246 |
| MAGE-A10-TAA14-3 | AQIACSSPSVVASLP | 15 | 247 |
| MAGE-A10-TAA14-4 | SPSVVASLPLDQSDE | 15 | 248 |
| MAGE-A10-TAA15 | AVEEDASSSTSTSSSFPSSFPSSSSSSSSSC | 31 | 249 |
| MAGE-A10-TAA15-1 | TSSSFPSSF | 9 | 250 |
| MAGE-A10-TAA15-2 | AVEEDASSSTSTSSS | 15 | 251 |
| MAGE-A10-TAA15-3 | SSSTSTSSSFPSSFP | 15 | 252 |
| MAGE-A10-TAA15-4 | SSSFPSSFPSSSSSS | 15 | 253 |

PMEL (Melanocyte protein):
FASTA:
>sp|P40967|PMEL_HUMAN Melanocyte protein PMEL GN=PMEL

Full length protein sequence (SEQ ID NO: 254):
MDLVLKRCLLHLAVIGALLAVGATKVPRNQDWLGVSRQLRTKAWNRQLY
PEWTEAQRLDCWRGGQVSLKVSNDGPTLIGANASFSIALNFPGSQKVLP
DGQVIWVNNTIINGSQVWGGQPVYPQETDDACIFPDGGPCPSGSWSQKR
SFVYVWKTWGQYWQVLGGPVSGLSIGTGRAMLGTHTMEVTVYHRRGSRS
YVPLAHSSSAFTITDQVPFSVSVSQLRALDGGNKHFLRNQPLTFALQLH
DPSGYLAEADLSYTWDFGDSSGTLISRALVVTHTYLEPGPVTAQVVLQA
AIPLTSCGSSPVPGTTDGHRPTAEAPNTTAGQVPTTEVVGTTPGQAPTA
EPSGTTSVQVPTTEVISTAPVQMPTAESTGMTPEKVPVSEVMGTTLAEM
STPEATGMTPAEVSIVVLSGTTAAQVTTTEWVETTARELPIPEPEGPDA
SSIMSTESITGSLGPLLDGTATLRLVKRQVPLDCVLYRYGSFSVTLDIV
QGIESAEILQAVPSGEGDAFELTVSCQGGLPKEACMEISSPGCQPPAQR
LCQPVLPSPACQLVLHQILKGGSGTYCLNVSLADTNSLAVVSTQLIMPG
QEAGLGQVPLIVGILLVLMAVVLASLIYRRRLMKQDFSVPQLPHSSSHW
LRLPRIFCSCPIGENSPLLSGQQV

TABLE T

| Name (SEQ ID NO) | Sequence | Length | SEQ ID |
|---|---|---|---|
| PMEL-TAA1 (256) | LISRALVVTHTYLEPGPVTAQVVLQAAIPLI | 31 | 255 |
| PMEL-TAA1-1 | YLEPGPVTA | 9 | 256 |
| PMEL-TAA1-2 | LISRALVVTHTYLEP | 15 | 257 |
| PMEL-TAA1-3 | VVTHTYLEPGPVTAQ | 15 | 258 |
| PMEL-TAA1-4 | LEPGPVTAQVVLQAA | 15 | 259 |
| PMEL-TAA2 | AVVLASLIYRRRLMKQDFSVPQLPHSSSHWL | 31 | 260 |
| PMEL-TAA2-1 | RLMKQDFSV | 9 | 261 |
| PMEL-TAA2-2 | AVVLASLIYRRRLMK | 15 | 262 |
| PMEL-TAA2-3 | LIYRRRLMKQDFSVP | 15 | 263 |
| PMEL-TAA2-4 | LMKQDFSVPQLPHSS | 15 | 264 |
| PMEL-TAA3 | TLIGANASFSIALNFPGSQKVLPDGQVIWVN | 31 | 265 |
| PMEL-TAA3-1 | ALNFPGSQKV | 10 | 266 |
| PMEL-TAA3-2 | TLIGANASFSIALNF | 15 | 267 |
| PMEL-TAA3-3 | ASFSIALNFPGSQKV | 15 | 268 |
| PMEL-TAA3-4 | LNFPGSQKVLPDGQV | 15 | 269 |
| PMEL-TAA4 | VSGLSIGTGRAMLGTHTMEVTVYHRRGSRSY | 31 | 270 |
| PMEL-TAA4-1 | MLGTHTMEV | 9 | 271 |
| PMEL-TAA4-2 | VSGLSIGTGRAMLGT | 15 | 272 |
| PMEL-TAA4-3 | GTGRAMLGTHTMEVT | 15 | 273 |
| PMEL-TAA4-4 | LGTHTMEVTVYHRRG | 15 | 274 |
| PMEL-TAA5 | WSQKRSFVYVWKTWGQYWQVLGGPVSGLSIG | 31 | 275 |
| PMEL-TAA5-1 | KTWGQYWQV | 9 | 276 |
| PMEL-TAA5-2 | WSQKRSFVYVWKTWG | 15 | 277 |
| PMEL-TAA5-3 | FVYVWKTWGQYWQVL | 15 | 278 |
| PMEL-TAA5-4 | TWGQYWQVLGGPVSG | 15 | 279 |
| PMEL-TAA6 | IMSTESITGSLGPLLDGTATLRLVKRQVPLD | 31 | 280 |
| PMEL-TAA6-1 | GPLLDGTATL | 10 | 281 |
| PMEL-TAA6-2 | IMSTESITGSLGPLL | 15 | 282 |
| PMEL-TAA6-3 | ITGSLGPLLDGTATL | 15 | 283 |
| PMEL-TAA6-4 | PLLDGTATLRLVKRQ | 15 | 284 |
| PMEL-TAA7 | DLVLKRCLLHLAVIGALLAVGATKVPRNQDW | 31 | 285 |
| PMEL-TAA7-1 | AVIGALLAV | 9 | 286 |
| PMEL-TAA7-2 | DLVLKRCLLHLAVIG | 15 | 287 |
| PMEL-TAA7-3 | CLLHLAVIGALLAVG | 15 | 288 |
| PMEL-TAA7-4 | VIGALLAVGATKVPR | 15 | 289 |
| PMEL-TAA8 | YCLNVSLADTNSLAVVSTQLIMPGQEAGLGQ | 31 | 290 |
| PMEL-TAA8-1 | SLAVVSTQL | 9 | 291 |
| PMEL-TAA8-2 | YCLNVSLADINSLAV | 15 | 292 |
| PMEL-TAA8-3 | LADTNSLAVVSTQLI | 15 | 293 |
| PMEL-TAA8-4 | LAVVSTQLIMPGQEA | 15 | 294 |
| PMEL-TAA9 | VPLAHSSSAFTITDQVPFSVSVSQLRALDGG | 31 | 295 |
| PMEL-TAA9-1 | ITDQVPFSV | 9 | 296 |
| PMEL-TAA9-2 | VPLAHSSSAFTITDQ | 15 | 297 |
| PMEL-TAA9-3 | SSAFTITDQVPFSVS | 15 | 298 |
| PMEL-TAA9-4 (300) | TDQVPFSVSVSQLRA | 15 | 299 |
| PMEL-TAA10 | SRQLRTKAWNRQLYPEWTEAQRLDCWRGGQV | 31 | 300 |
| PMEL-TAA10-1 | QLYPEWTEA | 9 | 301 |
| PMEL-TAA10-2 | SRQLRTKAWNRQLYP | 15 | 302 |
| PMEL-TAA10-3 | KAWNRQLYPEWTEAQ | 15 | 303 |
| PMEL-TAA10-4 | LYPEWTEAQRLDCWR | 15 | 304 |
| PMEL-TAA11 | VPFSVSVSQLRALDGGNKHFLRNQPLTFALQ | 31 | 305 |
| PMEL-TAA11-1 | ALDGGNKHFL | 10 | 306 |
| PMEL-TAA11-2 | VPFSVSVSQLRALDG | 15 | 307 |
| PMEL-TAA11-3 | VSQLRALDGGNKHFL | 15 | 308 |
| PMEL-TAA11-4 | LDGGNKHFLRNQPLT | 15 | 309 |
| PMEL-TAA12 | STQLIMPGQEAGLGQVPLIVGILLVLMAVVL | 31 | 310 |
| PMEL-TAA12-1 | GLGQVPLIV | 9 | 311 |
| PMEL-TAA12-2 | STQLIMPGQEAGLGQ | 15 | 312 |
| PMEL-TAA12-3 | PGQEAGLGQVPLIVG | 15 | 313 |
| PMEL-TAA12-4 | LGQVPLIVGILLVLM | 15 | 314 |
| PMEL-TAA13 | TGMTPEKVPVSEVMGTTLAEMSTPEATGMTP | 31 | 315 |
| PMEL-TAA13-1 | EVMGTTLAEM | 10 | 316 |
| PMEL-TAA13-2 | TGMTPEKVPVSEVMG | 15 | 317 |
| PMEL-TAA13-3 | KVPVSEVMGTTLAEM | 15 | 318 |
| PMEL-TAA13-4 | VMGTTLAEMSTPEAT | 15 | 319 |
| PMEL-TAA14 | SGTTSVQVPTTEVISTAPVQMPTAESTGMTP | 31 | 320 |
| PMEL-TAA14-1 | EVISTAPVQM | 10 | 321 |
| PMEL-TAA14-2 | SGTTSVQVPTTEVIS | 15 | 322 |
| PMEL-TAA14-3 | QVPTTEVISTAPVQM | 15 | 323 |
| PMEL-TAA14-4 | VISTAPVQMPTAEST | 15 | 324 |
| PMEL-TAA15 | IWVNNTIINGSQVWGGQPVYPQETDDACIFP | 31 | 325 |
| PMEL-TAA15-1 | QVWGGQPVY | 9 | 326 |
| PMEL-TAA15-2 | IWVNNTIINGSQVWG | 15 | 327 |
| PMEL-TAA15-3 | IINGSQVWGGQPVYP | 15 | 328 |
| PMEL-TAA15-4 (330) | VWGGQPVYPQETDDA | 15 | 329 |

Surviving (Human Baculoviral IAP repeat-containing protein 5) (SEQ ID NO: 330):
FASTA:
>sp|O15392|BIRC5_HUMAN Baculoviral IAP repeat-containing protein 5 GN=BIRC5

Full length protein sequence:
MGAPTLPPAWQPFLKDHRISTFKNWPFLEGCACTPERMAEAGFIHCPTE
NEPDLAQCFFCFKELEGWEPDDDPIEEHKKHSSGCAFLSVKKQFEELTL
GEFLKLDRERAKNKIAKETNNKKKEFEETAKKVRRAIEQLAAMD

| Name | Sequence | Length | SEQ ID |
|---|---|---|---|
| BIRC5-TAA1 | AFLSVKKQFEELTLGEFLKLDRERAKNKIAK | 31 | 331 |
| BIRC5-TAA1-1 | LTLGEFLKL | 9 | 332 |
| BIRC5-TAA1-2 | AFLSVKKQFEELTLG | 15 | 333 |
| BIRC5-TAA1-3 | KQFEELTLGEFLKLD | 15 | 334 |
| BIRC5-TAA1-4 | TLGEFLKLDRERAKN | 15 | 335 |
| BIRC5-TAA2 | MGAPTLPPAWQPFLKDHRISTFKNWPFLEG | 30 | 336 |
| BIRC5-TAA2-1 | TLPPAWQPFL | 10 | 337 |
| BIRC5-TAA2-2 | MGAPTLPPAWQPFLK | 15 | 338 |
| BIRC5-TAA2-3 | PPAWQPFLKDHRIST | 15 | 339 |
| BIRC5-TAA2-4 | FLKDHRISTFKNWPF | 15 | 340 |
| BIRC5-TAA3 | GAPTLPPAWQPFLKDHRISTFKNWPFLEGCA | 31 | 341 |
| BIRC5-TAA3-1 | FLKDHRIST | 9 | 342 |
| BIRC5-TAA3-2 | GAPTLPPAWQPFLKD | 15 | 343 |
| BIRC5-TAA3-3 | PAWQPFLKDHRISTF | 15 | 344 |
| BIRC5-TAA3-4 | LKDHRISTFKNWPFL | 15 | 345 |
| BIRC5-TAA4 | AWQPFLKDHRISTFKNWPFLEGCACTPERMA | 31 | 346 |
| BIRC5-TAA4-1 | STFKNWPFL | 9 | 347 |
| BIRC5-TAA4-2 | AWQPFLKDHRISTFK | 15 | 348 |
| BIRC5-TAA4-3 | KDHRISTFKNWPFLE | 15 | 349 |
| BIRC5-TAA4-4 | TFKNWPFLEGCACTP | 15 | 350 |
| BIRC5-TAA5 | FLEGCACTPERMAEAGFIHCPTENEPDLAQC | 31 | 351 |
| BIRC5-TAA5-1 | RMAEAGFIHC | 10 | 352 |
| BIRC5-TAA5-2 | FLEGCACTPERMAEA | 15 | 353 |
| BIRC5-TAA5-3 | CTPERMAEAGFIHCP | 15 | 354 |
| BIRC5-TAA5-4 | AEAGFIHCPTENEPD | 15 | 355 |

After PBMC isolation from subject blood draw, 4-1BB+ and/or CD69+CD8+ and CD4+ T cells will be sorted. If a particular clone responds to an epitope and comprises a high frequency of a particular marker expression, one can isolate that clone by way of CD3+, or CD4+, or CD8+ cells flow cytometry; magnetic beads specific for CD3+ or CD4+ or CD8+ cells; or any solid state column comprising antibodies specific for CD3+ or CD4+ or CD8+ cells. 000405 Prepare a single-cell suspension of T-Cells in DMSO or 1×PBS.

TCR sequencing; Methods:

The single cell (sc) RNA-seq and scTCR-seq libraries are prepared using the 10× Single Cell Immune Profiling Solution Kit. In brief, PBMC are washed once with PBS containing 0.04% bovine serum albumin (BSA) and resuspended in PBS containing 0.04% BSA to a final concentration of 100-800 cells per μl as determined by hemacytometer. Cells are captured in droplets at a targeted cell recovery of 500-7,000 cells, resulting in estimated multiplet rates of 0.4-5.4%. Following reverse transcription and cell barcoding in droplets, emulsions are broken, and cDNA purified using Dynabeads MyOne SILANE followed by PCR amplification (98° C. for 45 s; 13-18 cycles of 98° C. for 20 s, 67° C. for 30 s, 72° C. for 1 min; 72° C. for 1 min). Amplified cDNA is then used for both 5′ gene expression library construction and TCR enrichment. For gene expression library construction, 2.4-50 ng of amplified cDNA is fragmented and end-repaired, double-sided size-selected with SPRIselect beads, PCR-amplified with sample indexing primers (98° C. for 45 s; 14-16 cycles of 98° C. for 20 s, 54° C. for 30 s, 72° C. for 20 s; 72° C. for 1 min), and double-sided size-selected with SPRIselect beads. For TCR library construction, TCR transcripts are enriched from 2 μl of amplified cDNA by PCR (primer sets 1 and 2:98° C. for 45 s; 10 cycles of 98° C. for 20 s, 67° C. for 30 s, 72° C. for 1 min; 72° C. for 1 min). Following TCR enrichment, 5-50 ng of enriched PCR product is fragmented and end-repaired, size-selected with SPRIselect beads, PCR-amplified with sample-indexing primers (98° C. for 45 s; 9 cycles of 98° C. for 20 s, 54° C. for 30 s, 72° C. for 20 s; 72° C. for 1 min), and size-selected with SPRIselect beads.

Alternatively, TCR library can be performed utilizing primers for TCR V gene amplification focusing on alpha and beta families as described in Ch′ng, et al. (Eur J Immunol. 2019). Briefly, the forward primers correspond to the first amino acid-coding nucleotides of the variable mRNA (table below). The reverse primer is located at the beginning of the constant region of the TCR (α family TRAC_Rv: 5′-CC GCT CGA GAC AGG GTT CTG GAT ATT-3′ (SEQ ID NO: 365), β family TRBC2_Rv: 5′-TTT TCC TTT TGC GGC CGC GAA CAC GTT TTT CAG GTC-3′ (SEQ ID NO: 366)). The forward primers for TCR (α and β families) library construction is found in Table P of Example 1 above.

The scRNA libraries are sequenced on an Illumina NextSeq or HiSeq 4000 to a minimum sequencing depth of 25,000 reads per cell using read lengths of 26 bp read 1, 8 bp i7 index, 98 bp read 2. The single-cell TCR libraries are sequenced on an Illumina MiSeq or HiSeq 4000 to a minimum sequencing depth of 5,000 reads per cell using read lengths of 150 bp read 1, 8 bp i7 index, 150 bp read 2.

The scRNA-seq reads are aligned to the GRCh38 reference genome and quantified using cellranger count (10× Genomics, version 2.1.0). Filtered gene-barcode matrices that contained only barcodes with unique molecular identifier (UMI) counts that passed the threshold for cell detection are used for further analysis.

TCR reads are aligned to the GRCh38 reference genome and consensus TCR annotation is performed using cellranger vdj (10× Genomics, version 2.1.0). TCR libraries are sequenced to a minimum depth of 5,000 reads per cell, with a final average of 15,341 reads per cell.

T cells that recognize tumor antigens may proliferate to generate discernible clonal subpopulations defined by an identical T cell receptor (TCR) sequence. To identify potentially expanded T cell clones, we use ribonucleic acid sequencing (RNA-seq) reads that map to the TCR to classify single T cells by their isoforms of the V and J segments of the alpha and beta

The following are examples of sequenced TCR alpha (Table X):

| SEQ | CDR3 (aa | SEQ | |
|---|---|---|---|
| ID | sequence) | ID | TCR (full aa sequence) |
| 367 | CAVGGSGGGADGLTF | 368 | MLLELIPLLGIHFVLRTARAQSVTQPDIHITVSEGASLELRCN |
| | | | YSYGATPYLFWYVQSPGQGLQLLLKYFSGDTLVQGIKGFEAEF |
| | | | KRSQSSFNLRKPSVHWSDAAEYFCAVGGSGGGADGLTFGKGTH |
| | | | LIIQPYIQKPDPAVYQLRDSKSSDKSVCLFTDFDSQTNVSQSK |
| | | | DSDVYITDKTVLDMRSMDFKSNSAVAWSNKSDFACANAFNNSI |
| | | | IPEDTFFPSPESSCDVKLVEKSFETDINLNFQNLSVIGFRILL |
| | | | LKVAGFNLLMTLRLWSS |
| 369 | CAVGINARLMF | 370 | MMKSLRVLLVILWLQLSWVWSQQKEVEQNSGPLSVPEGAIASL |
| | | | NCTYSDRGSQSFFWYRQYSGKSPELIMFIYSNGDKEDGRFTAQ |
| | | | LNKASQYVSLLIRDSQPSDSATYLCAVGINARLMFGDGTQLVV |
| | | | KPNIQKPDPAVYQLRDSKSSDKSVCLFTDFDSQTNVSQSKDSD |
| | | | VYITDKTVLDMRSMDEKSNSAVAWSNKSDEACANAENNSLIPE |
| | | | DTFFPSPESSCDVKLVEKSFETDINLNFQNLSVIGFRILLLKV |
| | | | AGFNLLMTLRLWSS |
| 371 | CAGPMKTSYDKVIF | 372 | MLLEHLLIILWMQLTWVSGQQLNQSPQSMFIQEGEDVSMNCTS |
| | | | SSIFNTWLWYKQDPGEGPVLLIALYKAGELTSNGRLTAQFGIT |
| | | | RKDSFLNISASIPSDVGIYFCAGPMKTSYDKVIFGPGTSLSVI |
| | | | PNIQKPDPAVYQLRDSKSSDKSVCLFTDFDSQTNVSQSKDSDV |
| | | | YITDKTVLDMRSMDFKSNSAVAWSNKSDFACANAFNNSIIPED |
| | | | TFFPSPESSCDVKLVEKSFETDTNLNFQNLSVIGFRILLLKVA |
| | | | CFNLLMTLRLWSS |
| 373 | CAVRDLGAGGGNKLTF | 374 | MASAPISMLAMLFTLSGLRAQSVAQPEDQVNVAEGNPLTVKCT |
| | | | YSVSGNPYLFWYVQYPNRGLQFLLKYITGDNLVKGSYGFEAEF |
| | | | NKSQTSFHLKKPSALVSDSALYFCAVRDLGAGGGNKLTFGTGT |
| | | | QLKVELNIQNPDPAVYQLRDSKSSDKSVCLFTDFDSQTNVSQS |
| | | | KDSDVYITDKTVLDMRSMDFKSNSAVAWSNKSDFACANAFNNS |
| | | | IIPEDTFFPSPESSCDVKLVEKSFETDINLNFQNLSVIGFRIL |
| | | | LLKVAGFNLLMTLRLWSS |
| 375 | CAASKPGNQFYF | 376 | MTSIRAVFIFLWLQLDLVNGENVEQHPSELSVQEGDSAVIKCT |
| | | | YSDSASNYFPWYKQELGKRPQLIIDIRSNVGEKKDQRIAVTLN |
| | | | KTAKHFSLHITETQPEDSAVYFCAASKPGNQFYFGTGTSLTVI |
| | | | PNIQNPDPAVYQLRDSKSSDKSVCLFTDFDSQTNVSQSKDSDV |
| | | | YITDKTVLDMRSMDFKSNSAVAWSNKSDFACANAFNNSIIPED |
| | | | TFFPSPESSCDVKLVEKSFETDINLNFQNLSVIGFRILLLKVA |
| | | | CFNLLMTLRLWSS |
| 377 | CVVNRLHSYNYGQNFV | 378 | MMISLRVLLVILWLQLSWVWSQRKEVEQDPGPFNVPEGATVAF |
| | F | | NCTYSNSASQSFFWYRQDCRKEPKLLMSVYSSGNEDGRFTAQL |
| | | | NRASQYISLLIRDSKLSDSATYLCVVNRLHSYNYGQNFVFGPG |
| | | | TRLSVLPYIQKPDPAVYQLRDSKSSDKSVCLFTDFDSQTNVSQ |
| | | | SKDSDVYITDKTVLDMRSMDFKSNSAVAWSNKSDFACANAFNN |
| | | | SIIPEDTFFPSPESSCDVKLVEKSFETDENLNFQNLSVIGFRI |
| | | | LLLKVAGFNLLMELRLWSS |
| 379 | CAVRCTDSWCKLQF | 380 | MEKMLECAFIVLWLQLCWLSCEDQVTQSPEALRLQECESSSLN |
| | | | CSYTVSGLRGLFWYRQDPGKGPEFLFTLYSAGEEKEKERLKAT |
| | | | LTKKESFLHITAPKPEDSATYLCAVRGTDSWGKLQFGAGTQVV |
| | | | VTPDIQNPDPAVYQLRDSKSSDKSVCLFTDFDSQTNVSQSKDS |
| | | | DVYITDKTVLDMRSMDFKSNSAVAWSNKSDFACANAFNNSIIP |
| | | | EDTFFPSPESSCDVKLVEKSFETDINLNFQNLSVIGFRILLLK |
| | | | VAGFNLLMTLRLWSS |

The following are examples of sequenced TCR beta (Table Y):

| SEQ | CDR3 (aa | SEQ | |
|---|---|---|---|
| ID | sequence) | ID | TCR (full aa sequence) |
| 381 | CASSAVGNTIYF | 382 | MTIRLLCYVGFYFLGAGLMEADIYQTPRYLVIGTGKKITLECS |
| | | | QTMGHDKMYWYQQDPGMELHLIHYSYGVNSTEKGDLSSESTVS |
| | | | RIRTEHFPLTLESARPSHTSQYLCASSAVGNTIYFGEGSWLIV |
| | | | VEDLNKVFPPEVAVFEPSEAEISHTQKATLVCLATGFFPDHVE |
| | | | LSWWVNGKEVHSGVSTDPQPLKEQPALNDSRYCLSSRLRVSAT |
| | | | FWQNPRNHFRCQVQFYGLSENDEWTQDRAKPVTQIVSAEAWGR |
| | | | ADCGFTSVSYQQGVLSATILYEILLGKATLYAVLVSALVLMAM |
| | | | VKRKDF |
| 383 | CVVSEGDSGGFKTIF | 384 | MGSRLLCWVLLCLLGAGPVKAGVTQTPRYLIKTRGQQVTLSCS |
| | | | PISGHRSVSWYQQTPGQGLQFLFEYFSETQRNKGNFPGRFSGR |
| | | | QFSNSRSEMNVSTLELGDSALYLCASSPLAGGISDTQYFGPGT |
| | | | RLTVLEDLKNVFPPKVAVFEPSEAEISHTQKATLVCLATGFYP |
| | | | DHVELSWWVNGKEVHSGVSTDPQPLKEQPALNDSRYCLSSRLR |
| | | | VSATFWQNPRNHFRCQVQFYGLSENDEWTQDRAKPVTQIVSAE |
| | | | AWGRADCGFTSESYQQGVLSATILYEILLGKATLYAVLVSALV |
| | | | LMAMVKRKDSRG |
| 385 | CASSPLAGGISDTQYF | 386 | MGSRLLCWVLLCLLGAGPVKAGVTQTPRYLIKTRGQQVILSCS |
| | | | PISGHRSVSWYQQTPGQGLQFLFEYFSETQRNKGNFPGRFSGR |
| | | | QFSNSRSEMNVSTLELGDSALYLCASSPLAGGISDTQYFGPGT |
| | | | RLTVLEDLKNVFPPKVAVFEPSEAEISHTQKATLVCLATGFYP |
| | | | DHVELSWWVNGKEVHSGVSTDPQPLKEQPALNDSRYCLSSRLR |
| | | | VSATFWQNPRNHFRCQVQFYGLSENDEWTQDRAKPVTQIVSAE |
| | | | AWGRADCGFTSESYQQGVLSATILYEILLGKATLYAVLVSALV |
| | | | LMAMVKRKDSRG |
| 387 | CASSSANYGYTF | 388 | MGSWTLCCVSLCTLVAKHTDAGVIQSPRHEVTEMGQEVTLRCK |
| | | | PISGHDYLFWYRQTMMRGLELLIYFNNNVPIDDSGMPEDRFSA |
| | | | KMPNASESTLKIQPSEPRDSAVYFCASSSANYGYTEGSGIRLT |
| | | | VVEDLNKVFPPEVAVFEPSEAEISHTQKATLVCLATGFFPDHV |
| | | | ELSWWVNGKEVHSGVSTDPQPLKEQPALNDSRYCLSSRLRVSA |
| | | | TFWQNPRNHFRCQVQFYGLSENDEWTQDRAKPVTQIVSAEAWG |
| | | | RADCGFTSVSYQQGVLSATILYEILLGKATLYAVLVSALVLMA |
| | | | MVKRKDF |
| 389 | CASSTLVRETQYF | 390 | MGPGLLCWALLCLLGAGSVETGVTQSPTHLIKTRGQQVTLRCS |
| | | | SQSGHNTVSWYQQALGQGPQFIFQYYREEENGRGNFPPRFSGL |
| | | | QFPNYSSELNVNALELDDSALYLCASSTLVRETQYFGPGTRLL |
| | | | VLEDLKNVFPPKVAVFEPSEAEISHTQKATLVCLATGFYPDHV |
| | | | ELSWWVNGKEVHSGVSTDPQPLKEQPALNDSRYCLSSRLRVSA |
| | | | TFWQNPRNHFRCQVQFYGLSENDEWTQDRAKPVTQIVSAEAWG |
| | | | RADCGFTSESYQQGVLSATILYEILLGKATLYAVLVSALVLMA |
| | | | MVKRKDSRG |
| 391 | CSAREYQGVKSPLHF | 392 | MEAVVTTLPREGGVRPSRKMLLLLLLLGPGSGLGAVVSQHPSR |
| | | | VICKSGTSVKIECRSLDFQATTMFWYRQFPKQSLMLMATSNEG |
| | | | SKATYEQGVEKDKFLINHASLILSTLTVESAHPEDSSFYICSA |
| | | | REYQGVKSPLHFGNGTRLTVTEDLNKVEPPEVAVFEPSEAEIS |
| | | | HTQKATLVCLATGFFPDHVELSWWVNGKEVHSGVSTDPQPLKE |
| | | | QPALNDSRYCLSSRLRVSATFWQNPRNHFRCQVQFYGLSENDE |
| | | | WTQDRAKPVTQIVSAEAWGRADCGFTSVSYQQGVLSATILYEI |
| | | | LLGKATLYAVLVSALVLMAMVKRKDF |
| 393 | CASSFKMNTEAFF | 394 | MGPRLLFWALLCLLGIGPVEAGVTQSPTHLIKTRGQQVTLRCS |
| | | | PISGHTSVYWYQQALGLGLQFLLWYDEGEERNRGNFPPRFSGR |
| | | | QFPNYSSELNVNALELEDSALYLCASSFKMNTEAFFGQGIRLT |
| | | | VVEDLNKVEPPEVAVFEPSEAEISHTQKATLVCLATGFFPDHV |
| | | | ELSWWVNGKEVHSGVSTDPQPLKEQPALNDSRYCLSSRLRVSA |
| | | | TFWQNPRNHFRCQVQFYGLSENDEWTQDRAKPVTQIVSAEAWG |
| | | | RADCGFTSVSYQQGVLSATILYEILLGKATLYAVLVSALVLMA |
| | | | MVKRKDF |

Examples of lentiviral or retroviral plasmids.

The TCR is inserted in a retroviral or lentiviral vector, including pELNS (lentivirus), pRRL (lentiviral), pCR2.1 (lentiviral), pMSGV (retroviral), pMSCV (murine stem cell virus, retroviral) pMIG II (retroviral), LENTIVECTOR® or LENTIMAX™. LENTIMAX™ US patent number: US 2019/0134091 A1, which is hereby incorporated by reference.

Two examples of commercially-available plasmids are pMSGC and pMSCV. pMSGV can be found above as SEQ ID NO 175. pMSCV can be found above as SEQ ID NO 176

TCR-transfected cells will be cocultured with patient's own antigen presenting cells (APC), or artificial APCs (e.g. HLA-matched K562). Antigen-specific T cells will be identified by CD69 upregulation 18 hours following coculture. The expected affinity between the peptide-HLA complex and the TCR is less than about 500 nM.

### In Vitro Cytotoxicity

To test cytotoxicity in vitro, antigen-specific TCR T cells are activated in a 96 well round-bottom plate with anti CD3/CD28 beads (ThermoFisher Scientific) and supplemented with AIM V/5% human AB serum and 20 ng/ml IL-2 for 36 h. Then, 2-fold serial dilutions of activated T cells are plated in 96 well round-bottom plates starting at 1×105 cells per well in AIM V/5% human AB serum. HLA-matched luciferase-expressing K562 cells are co-cultured with T cells for 18 h at different ratios (e.g., 1:1, 1:3, 1:10 effector: target ratio), either transduced to express the neoantigen of interest or an irrelevant neoantigen as a control. Alternatively, HLA-matched luciferase-expressing K562 cells are pulsed with relevant (e.g. YLGSGIHSGA [SEQ ID NO: 1] in the example of CTNNB1 mutation D32G, TTAPPLSGK [SEQ ID NO: 16] in the example of CTNNB1 mutation S45P) or irrelevant neoantigen epitopes. Luciferase assay is performed with Bright-Glo™ Luciferase Assay System (Promega) following the manufacturer's protocol to correlate luciferase expression with T cell cytotoxicity. Briefly, cells in 96-well plates are equilibrated at room temperature for 5 minutes. Then, a volume of Bright-Glo™ reagent equal to the volume of culture medium is added and mixed. After 2 minutes of cell lysis, the luminescence can be measured in a luminometer.

Alternatively, in vitro cytotoxicity can be assessed by flow cytometry. Here, after 18 hours of co-culture (antigen-specific TCR T cells: HLA-matched K562) cells plated in 96-well plates are spun down at 1500 RPM, 5 minutes, at room temperature. Then, cells are washed in 200 uL of PBS, centrifuged again (same settings). Cells are resuspended in 1 mL of FACS buffer (PBS, 0.5-1% BSA), transferred to FACS tubes (5 mL, conical) and centrifuged at 1500 RPM, 5 minutes, at room temperature. Next, the pellet is resuspended in 100 uL of a master mix including the fluorescent-conjugated antibodies against CD45, Annexin V, and 7-AAD and incubated for 30 minutes at 4° C. in the dark. Upon incubation is completed, cells are washed (1500 RPM, 5 minutes, at room temperature) and resuspended in 300 uL of FACS buffer for further analysis in the flow cytometer. The CD45 negative, Annexin V/7-AAD double-positive cells correspond to apoptotic HLA-matched K562. Thus, the more apoptotic cells observed, the more T cell cytotoxicity (Perales-Puchalt, Mol Ther. 2019).

### In Vivo Cytotoxicity

To measure cytotoxicity in vivo, HLA-matched human tumor cell lines of the relevant tumor type are transduced with the relevant neoantigen, an irrelevant neoantigen or the wild-type epitope. Tumor cell lines are grown in vitro under sterile conditions in RPMI 10%-FBS culture media supplemented with 1% penicillin/streptomycin at 37° C., 5%-CO2. Next, tumor cells (1×106 in 100 uL PBS) are injected subcutaneously in the back of immunodeficient NOD-SCID-Gamma (NSG) mouse and tumor volume is monitored three times per week with a digital caliper. TCR-transduced T cells are cultured in vitro under sterile conditions in RPMI 10%-FBS culture media supplemented with 1% penicillin/streptomycin at 37° C., 5%-CO2. When tumors reach a volume of approximately 100 mm3, the TCR-transduced T cells are injected (2×106 in 100 uL PBS) intraperitoneally in the mice. Then, tumor burden is monitored three times per week with a digital caliper until the endpoint (2000 mm3 tumor volume) is reached. Mice adoptively transferred with the corresponding neoantigen-specific CD8+ T cells can control tumor growth. Instead, mock-treated mice show control-level tumor growth.

### Example 3

Following vaccination against 20-40 epitopes from individualized neoantigens from a sample, PBMC are collected from the patient. Cells are stimulated with the corresponding peptides (10 ug/mL), and returned to the incubator at 37° C., 5%-CO2. 18 to 24 hours later, cells are stained and sorted based on CD137 and/or CD69 status.

Examples of neoantigens: Full-length and fragments 1-15.

### Neoantigens

Catenin beta-1 (CTNB1):
FASTA: >sp|P35222|CTNB1_HUMAN Catenin beta-1 OS=Homo sapiens

OX=9606 GN=CTNNB1 PE=1 SV=1 (Beta catenin wild type)

Full-length protein sequence (SEQ ID NO: 362):
MATQADLMELDMAMEPDRKAAVSHWQQQSYLDSGIHSGATTTAPSLSGKGNPEEEDVDTSQVLYEWEQGF
SQSFTQEQVADIDGQYAMTRAQRVRAAMFPETLDEGMQIPSTQFDAAHPTNVQRLAEPSQMLKHAVVNLI
NYQDDAELATRAIPELTKLLNDEDQVVVNKAAVMVHQLSKKEASRHAIMRSPQMVSAIVRIMQNINDVET
ARCTAGTLHNLSHHREGLLAIFKSGGIPALVKMLGSPVDSVLFYAITTLHNLLLHQEGAKMAVRLAGGLQ
KMVALLNKTNVKFLAITTDCLQILAYGNQESKLIILASGGPQALVNIMRTYTYEKLLWITSRVLKVLSVC
SSNKPAIVEAGGMQALGLHLTDPSQRLVQNCLWTLRNLSDAATKQEGMEGLLGTLVQLLGSDDINVVTCA
AGILSNLTCNNYKNKMMVCQVGGIEALVRTVLRAGDREDITEPAICALRHLTSRHQEAEMAQNAVRLHYG
LPVVVKLLHPPSHWPLIKATVGLIRNLALCPANHAPLREQGAIPRLVQLLVRAHQDTQRRTSMGGTQQQF
VEGVRMEEIVEGCTGALHILARDVHNRIVIRGLNTIPLFVQLLYSPIENIQRVAAGVLCELAQDKEAAEA
IEAEGATAPLTELLHSRNEGVATYAAAVLFRMSEDKPQDYKKRLSVELTSSLFRTEPMAWNETADLGLDI
GAQGEPLGYRQDDPSYRSFHSGGYGQDALGMDPMMEHEMGGHHPGADYPVDGLPDLGHAQDLMDGLPPGD
SNQLAWFDTDL

Example of Mutated Catenin beta-1 (CTNB1_D32G) number 1
FASTA: >sp|P35222|CTNB1_D32G (mutated beta catenin and potential vaccine 000431 sequences)

Full-length protein sequence (SEQ ID NO: 363):
MATQADLMELDMAMEPDRKAAVSHWQQQSYLGSGIHSGATTTAPSLSGKGNPEEEDVDTSQVLY
EWEQGFSQSFTQEQVADIDGQYAMTRAQRVRAAMFPETLDEGMQIPSTQFDAAHPTNVQRLAEP
SQMLKHAVVNLINYQDDAELATRAIPELTKLLNDEDQVVVNKAAVMVHQLSKKEASRHAIMRSP
QMVSAIVRIMQNTNDVETARCTAGTLHNLSHHREGLLAIFKSGGIPALVKMLGSPVDSVLFYAI
TTLHNLLLHQEGAKMAVRLAGGLQKMVALLNKTNVKFLAITTDCLQILAYGNQESKLIILASGG
PQALVNIMRTYTYEKLLWTTSRVLKVLSVCSSNKPAIVEAGGMQALGLHLTDPSQRLVQNCLWT
LRNLSDAATKQEGMEGLLGTLVQLLGSDDINVVTCAAGILSNLTCNNYKNKMMVCQVGGIEALV
RTVLRAGDREDITEPAICALRHLTSRHQEAEMAQNAVRLHYGLPVVVKLLHPPSHWPLIKATVG
LIRNLALCPANHAPLREQGAIPRLVQLLVRAHQDTQRRTSMGGTQQQFVEGVRMEEIVEGCTGA
LHILARDVHNRIVIRGLNTIPLFVQLLYSPIENIQRVAAGVLCELAQDKEAAEAIEAEGATAPL
TELLHSRNEGVATYAAAVLERMSEDKPQDYKKRLSVELTSSLERTEPMAWNETADLGLDIGAQG
EPLGYRQDDPSYRSFHSGGYGQDALGMDPMMEHEMGGHHPGADYPVDGLPDLGHAQDLMDGLPP
GDSNQLAWFDTDL

Example of fragments from mutated Catenin beta-1 (CTNB1_D32G) number 1 that can be used in a vaccine are found in Table N, above.

Example of mutated Catenin beta-1 (CTNB1_S45P) number 2
FASTA: >sp|P35222|CTNB1_S45P (mutated beta catenin and potential vaccine sequences)

Full-length protein sequence (SEQ ID NO: 364):
MATQADLMELDMAMEPDRKAAVSHWQQQSYLDSGIHSGATTTAPPLSGKGNPEEEDVDTSQVLYEWEQGF
SQSFTQEQVADIDGQYAMTRAQRVRAAMFPETLDEGMQIPSTQFDAAHPTNVQRLAEPSQMLKHAVVNLI
NYQDDAELATRAIPELTKLLNDEDQVVVNKAAVMVHQLSKKEASRHAIMRSPQMVSAIVRTMQNTNDVET
ARCTAGTLHNLSHHREGLLAIFKSGGIPALVKMLGSPVDSVLFYAITTLHNLLLHQEGAKMAVRLAGGLQ
KMVALLNKTNVKFLAITTDCLQILAYGNQESKLIILASGGPQALVNIMRTYTYEKLLWITSRVLKVLSVC
SSNKPAIVEAGGMQALGLHLTDPSQRLVQNCLWTLRNLSDAATKQEGMEGLLGTLVQLLGSDDINVVTCA
AGILSNLTCNNYKNKMMVCQVGGIEALVRTVLRAGDREDITEPAICALRHLISRHQEAEMAQNAVRLHYG
LPVVVKLLHPPSHWPLIKATVGLIRNLALCPANHAPLREQGAIPRLVQLLVRAHQDTQRRTSMGGTQQQF
VEGVRMEEIVEGCTGALHILARDVHNRIVIRGLNTIPLFVQLLYSPIENIQRVAAGVLCELAQDKEAAEA
IEAEGATAPLTELLHSRNEGVATYAAAVLFRMSEDKPQDYKKRLSVELTSSLFRTEPMAWNETADLGLDI
GAQGEPLGYRQDDPSYRSFHSGGYGQDALGMDPMMEHEMGGHHPGADYPVDGLPDLGHAQDLMDGLPPGD
SNQLAWFDTDL

Examples of fragments from mutated Catenin beta-1 (CTNB1_S45P) number 2 that can be used in a vaccine can be found in Table M, above.

Table O, above, includes tumor-associated antigens (short fragments/epitopes) utilized to design neoantigen DNA vaccines.

After PBMC isolation from subject blood draw, 4-1BB+ and/or CD69+CD8+ and CD4+ T cells will be sorted. If a particular clone responds to an epitope and comprises a high frequency of a particular marker expression, one can isolate that clone by way of CD3+, or CD4+, or CD8+ cells flow cytometry; magnetic beads specific for CD3+ or CD4+ or CD8+ cells; or any solid state column comprising antibodies specific for CD3+ or CD4+ or CD8+ cells.

Prepare a single-cell suspension of T-Cells in DMSO or 1×PBS.

### TCR Sequencing; Methods:

The single cell (sc) RNA-seq and scTCR-seq libraries are prepared using the 10× Single Cell Immune Profiling Solution Kit. In brief, PBMC are washed once with PBS containing 0.04% bovine serum albumin (BSA) and resuspended in PBS containing 0.04% BSA to a final concentration of 100-800 cells per μl as determined by hemacytometer. Cells are captured in droplets at a targeted cell recovery of 500-7,000 cells, resulting in estimated multiplet rates of 0.4-5.4%. Following reverse transcription and cell barcoding in droplets, emulsions are broken, and cDNA purified using Dynabeads MyOne SILANE followed by PCR amplification (98° C. for 45 s; 13-18 cycles of 98° C. for 20 s, 67° C. for 30 s, 72° C. for 1 min; 72° C. for 1 min). Amplified cDNA is then used for both 5′ gene expression library construction and TCR enrichment. For gene expression library construction, 2.4-50 ng of amplified cDNA is fragmented and end-repaired, double-sided size-selected with SPRIselect beads, PCR-amplified with sample indexing primers (98° C. for 45 s; 14-16 cycles of 98° C. for 20 s, 54° C. for 30 s, 72° C. for 20 s; 72° C. for 1 min), and double-sided size-selected with SPRIselect beads. For TCR library construction, TCR transcripts are enriched from 2 μl of amplified cDNA by PCR (primer sets 1 and 2:98° C. for 45 s; 10 cycles of 98° C. for 20 s, 67° C. for 30 s, 72° C. for 1 min; 72° C. for 1 min). Following TCR enrichment, 5-50 ng of enriched PCR product is fragmented and end-repaired, size-selected with SPRIselect beads, PCR-amplified with sample-indexing primers (98° C. for 45 s; 9 cycles of 98° C. for 20 s, 54° C. for 30 s, 72° C. for 20 s; 72° C. for 1 min), and size-selected with SPRIselect beads.

Alternatively, TCR library can be performed utilizing primers for TCR V gene amplification focusing on alpha and beta families as described in Ch′ng, et al. (Eur J Immunol. 2019). Briefly, the forward primers correspond to the first amino acid-coding nucleotides of the variable mRNA (table below). The reverse primer is located at the beginning of the constant region of the TCR (α family TRAC_Rv: 5′-CC GCT CGA GAC AGG GTT CTG GAT ATT-3′ (SEQ ID NO: 365), β family TRBC2_Rv: 5′-TTT TCC TTT TGC GGC CGC GAA CAC GTT TTT CAG GTC-3′ (SEQ ID NO: 366)). The forward primers for TCR (α and β families) library construction is found in Table P of Example 1 above.

The scRNA libraries are sequenced on an Illumina NextSeq or HiSeq 4000 to a minimum sequencing depth of 25,000 reads per cell using read lengths of 26 bp read 1, 8 bp i7 index, 98 bp read 2. The single-cell TCR libraries are sequenced on an Illumina MiSeq or HiSeq 4000 to a minimum sequencing depth of 5,000 reads per cell using read lengths of 150 bp read 1, 8 bp i7 index, 150 bp read 2.

The scRNA-seq reads are aligned to the GRCh38 reference genome and quantified using cellranger count (10× Genomics, version 2.1.0). Filtered gene-barcode matrices that contained only barcodes with unique molecular identifier (UMI) counts that passed the threshold for cell detection are used for further analysis.

TCR reads are aligned to the GRCh38 reference genome and consensus TCR annotation is performed using cellranger vdj (10× Genomics, version 2.1.0). TCR libraries are sequenced to a minimum depth of 5,000 reads per cell, with a final average of 15,341 reads per cell.

T cells that recognize tumor antigens may proliferate to generate discernible clonal subpopulations defined by an identical T cell receptor (TCR) sequence. To identify potentially expanded T cell clones, we use ribonucleic acid sequencing (RNA-seq) reads that map to the TCR to classify single T cells by their isoforms of the V and J segments of the alpha and beta

Examples of sequenced TCR alpha can be found in Table X, above. Examples of sequenced TCR beta can be found in Table Y, above.

Examples of lentiviral or retroviral plasmids.

The TCR is inserted in a retroviral or lentiviral vector, including pELNS (lentivirus), pRRL (lentiviral), pCR2.1 (lentiviral), pMSGV (retroviral), pMSCV (murine stem cell virus, retroviral) pMIG II (retroviral), LENTIVECTOR® or LENTIMAX™. LENTIMAX™ US patent number: US 2019/0134091 A1, which is hereby incorporated by reference.

Two examples of commercially-available plasmids are pMSGC and pMSCV. pMSGV can be found above as SEQ ID NO 175. pMSCV can be found above as SEQ ID NO 176

TCR-transfected cells will be cocultured with patient's own antigen presenting cells (APC), or artificial APCs (e.g. HLA-matched K562). Antigen-specific T cells will be identified by CD69 upregulation 18 hours following coculture. The expected affinity between the peptide-HLA complex and the TCR is less than about 500 nM.

### In Vitro Cytotoxicity

To test cytotoxicity in vitro, antigen-specific TCR T cells are activated in a 96 well round-bottom plate with anti CD3/CD28 beads (ThermoFisher Scientific) and supplemented with AIM V/5% human AB serum and 20 ng/ml IL-2 for 36 h. Then, 2-fold serial dilutions of activated T cells are plated in 96 well round-bottom plates starting at 1×105 cells per well in AIM V/5% human AB serum. HLA-matched luciferase-expressing K562 cells are co-cultured with T cells for 18 h at different ratios (e.g., 1:1, 1:3, 1:10 effector: target ratio), either transduced to express the neoantigen of interest or an irrelevant neoantigen as a control. Alternatively, HLA-matched luciferase-expressing K562 cells are pulsed with relevant (e.g. YLGSGIHSGA (SEQ ID NO: 1) in the example of CTNNB1 mutation D32G, TTAPPLSGK (SEQ ID NO: 16) in the example of CTNNB1 mutation S45P) or irrelevant neoantigen epitopes. Luciferase assay is performed with Bright-Glo™ Luciferase Assay System (Promega) following the manufacturer's protocol to correlate luciferase expression with T cell cytotoxicity. Briefly, cells in 96-well plates are equilibrated at room temperature for 5 minutes. Then, a volume of Bright-Glo™ reagent equal to the volume of culture medium is added and mixed. After 2 minutes of cell lysis, the luminescence can be measured in a luminometer.

Alternatively, in vitro cytotoxicity can be assessed by flow cytometry. Here, after 18 hours of co-culture (antigen-specific TCR T cells: HLA-matched K562) cells plated in 96-well plates are spun down at 1500 RPM, 5 minutes, at room temperature. Then, cells are washed in 200 μL of PBS, centrifuged again (same settings). Cells are resuspended in 1 mL of FACS buffer (PBS, 0.5-1% BSA), transferred to FACS tubes (5 mL, conical) and centrifuged at 1500 RPM, 5 minutes, at room temperature. Next, the pellet is resuspended in 100 μL of a master mix including the fluorescent-conjugated antibodies against CD45, Annexin V, and 7-AAD and incubated for 30 minutes at 4° C. in the dark. Upon incubation is completed, cells are washed (1500 RPM, 5 minutes, at room temperature) and resuspended in 300 μL of FACS buffer for further analysis in the flow cytometer. The CD45 negative, Annexin V/7-AAD double-positive cells correspond to apoptotic HLA-matched K562. Thus, the more apoptotic cells observed, the more T cell cytotoxicity (Perales-Puchalt, Mol Ther. 2019).

### In Vivo Cytotoxicity

To measure cytotoxicity in vivo, HLA-matched human tumor cell lines of the relevant tumor type are transduced with the relevant neoantigen, an irrelevant neoantigen or the wild-type epitope. Tumor cell lines are grown in vitro under sterile conditions in RPMI 10%-FBS culture media supplemented with 1% penicillin/streptomycin at 37° C., 5%-CO2. Next, tumor cells (1×106 in 100 uL PBS) are injected subcutaneously in the back of immunodeficient NOD-SCID-Gamma (NSG) mouse and tumor volume is monitored three times per week with a digital caliper. TCR-transduced T cells are cultured in vitro under sterile conditions in RPMI 10%-FBS culture media supplemented with 1% penicillin/streptomycin at 37° C., 5%-CO2. When tumors reach a volume of approximately 100 mm3, the TCR-transduced T cells are injected (2×106 in 100 uL PBS) intraperitoneally in the mice. Then, tumor burden is monitored three times per week with a digital caliper until the endpoint (2000 mm3 tumor volume) is reached. Mice adoptively transferred with the corresponding neoantigen-specific CD8+ T cells can control tumor growth. Instead, mock-treated mice show control-level tumor growth.

### Example 4

### Background

Tumor neoantigens are epitopes derived from tumor-specific mutations that can be incorporated in personalized vaccines to prime T cell responses. DNA vaccines delivered with electroporation have recently shown strong CD8 and CD4 T cell responses in clinical trials. In preclinical studies, DNA-encoded neoantigen vaccines have shown induction of CD8 T cells against 50% of predicted high affinity epitopes with the ability to impact tumor growth.

### Methods

Paired blood and tumor biopsy samples were collected from a patient with hepatocellular carcinoma before and after treatment with GNOS-PV02 (DNA neoantigen targeted vaccine)+plasmid IL-12+pembrolizumab. Treatment resulted in a partial response with a decrease in tumor size of 44% by RECIST (168 mm to 94 mm). TCRbeta sequencing was performed on all 4 samples and single cell TCR and transcriptome sequencing was performed from T cells isolated from the post-treatment blood sample. Newly identified TCRs in blood and tumor after vaccination were inserted into an expression vector and used to generate engineered TCR T cells. Engineered TCR T cells were tested against the neoantigens included in the vaccine and their responses characterized by flow cytometry.

### Results

67,893 new clones were identified in PBMC after vaccination, 3 of which comprised between 0.1 to 1% of the total T cell clones. Moreover, 5126 new clones were identified in the tumor post vaccination, out of these, 3878 (75.68%) were not found within the patient's pre vaccination PBMCs and 556 (10.86%) were identified within the pre vaccination PBMC pool. Importantly, of the newly identified T cells infiltrating the tumor post vaccination, high frequency TCR clones were observed, of which 44 and 7 clones were above 0.1% and 1%, respectively. The majority of the newly identified T cell clones were CD8 T cells (68.75%) with an activated phenotype. Importantly, the 6 most expanded clones in blood were identified to be activated CD8+CD69+ T cells (81.82%). Engineered TCR T cells generated encoding the TCRs of these newly identified CD8 T cells showed activation when exposed to the tumor neoantigens encoded in the neoantigen DNA vaccine GNOS-PV02.

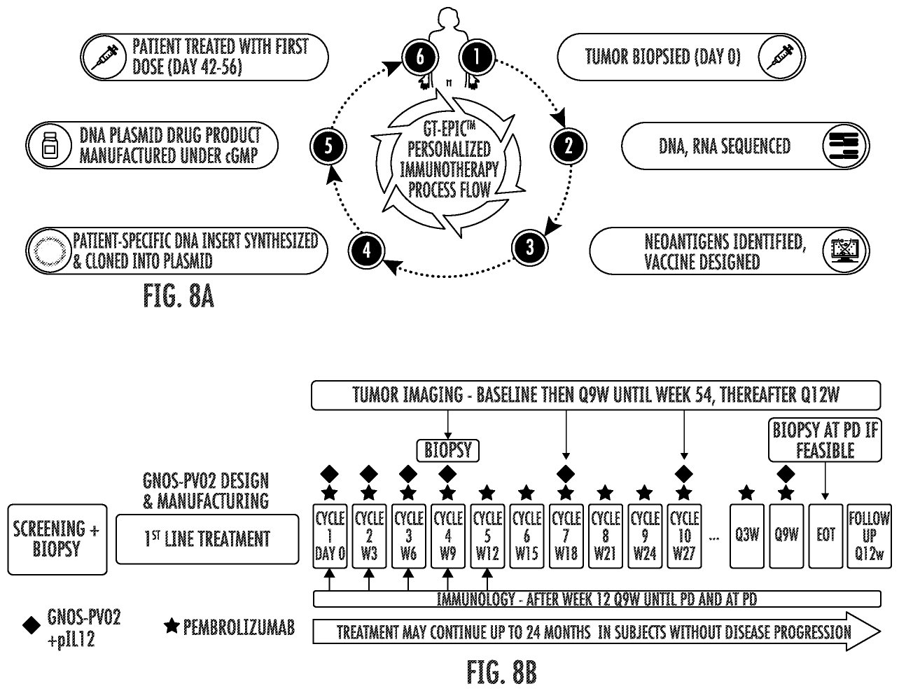

Personalized vaccines can be manufactured in 6-8 weeks allowing concurrent start with anti-PD1. FIG. 8 shows and example of the steps involved in a personalized vaccine.

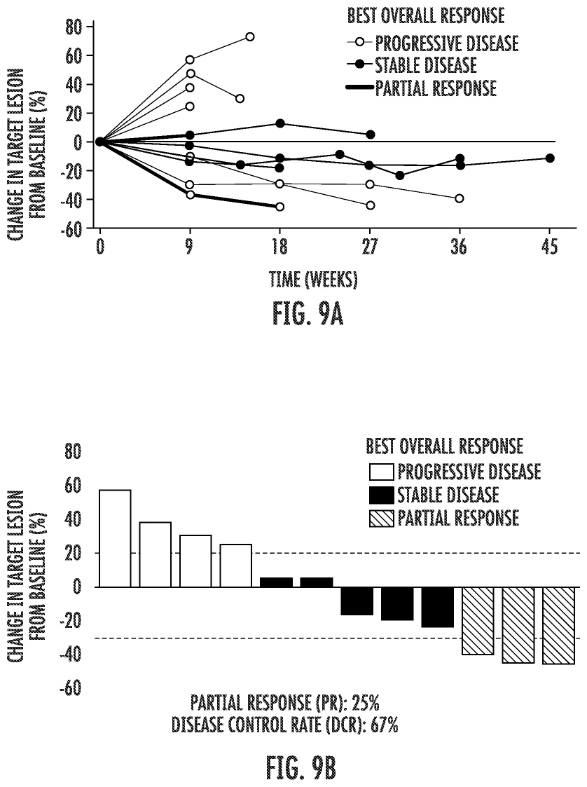

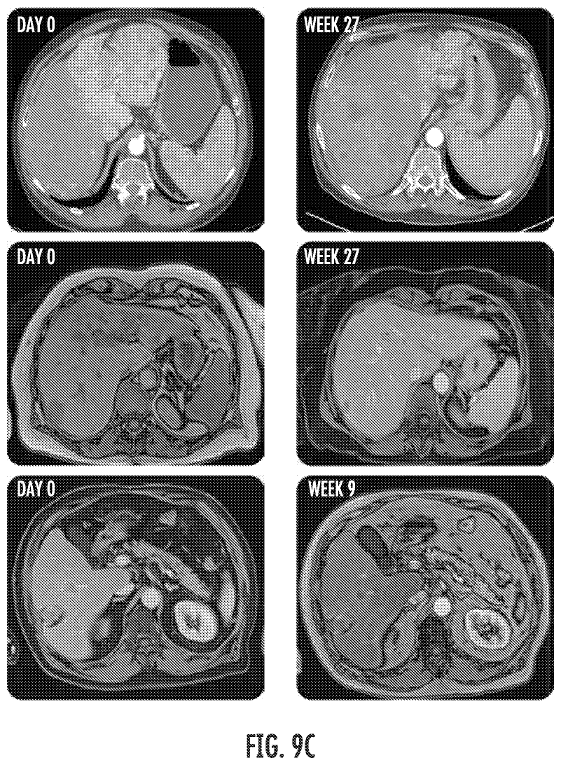

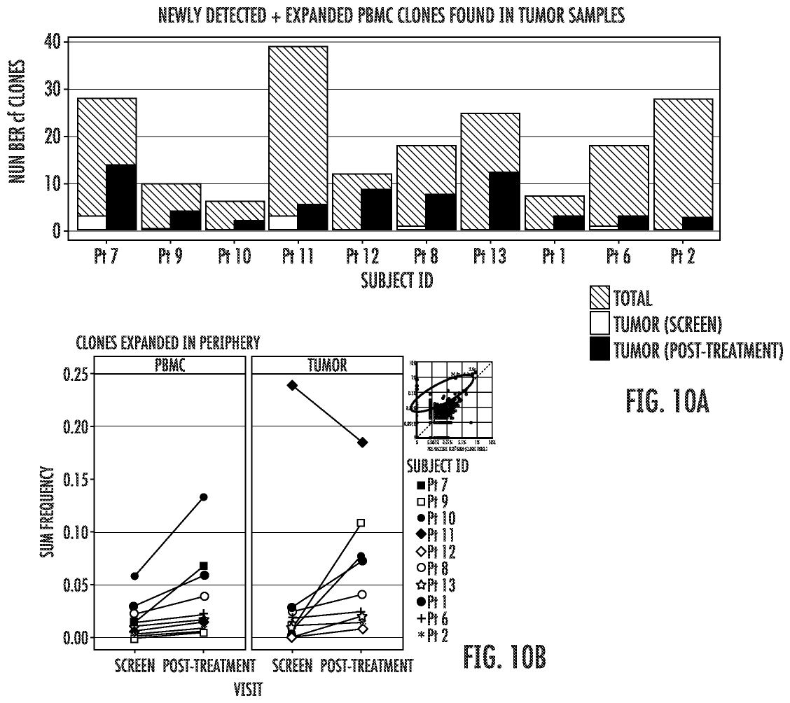

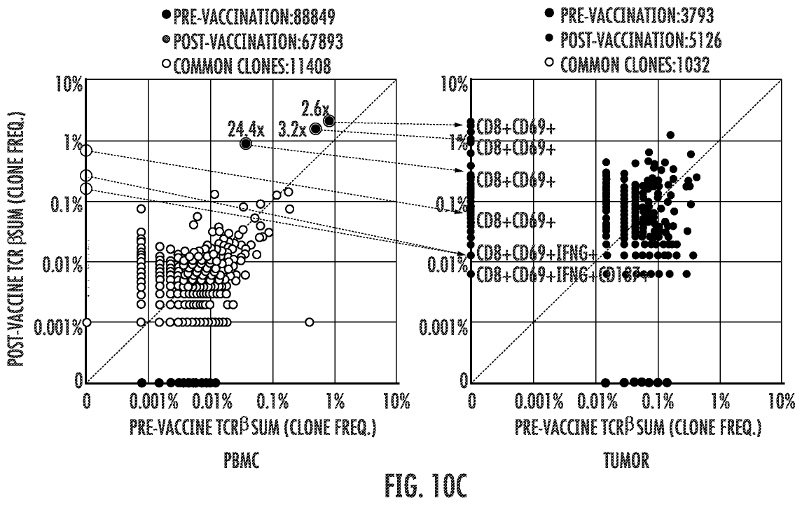

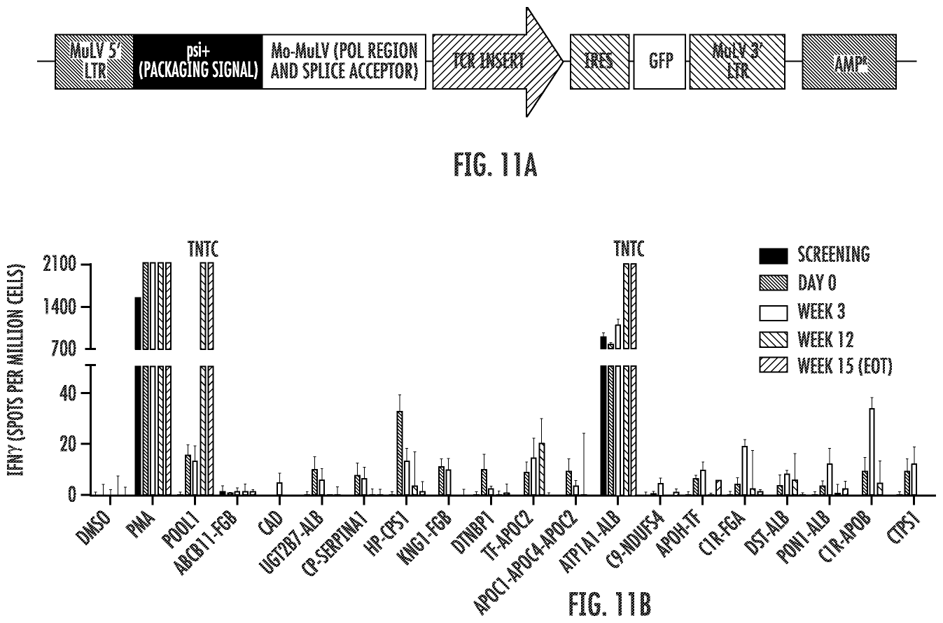

FIG. 9 shows a combination of GNOS-PV02 and anti-PD1 resulted in a 25% ORR in the first 12 patients. GNOS-PV02 results in expansion of new T cells that traffic to the tumor (see FIG. 10). As seen in FIG. 10A, post-treatment tumor samples contain significantly more of these clones than screen tumor samples (p=0.006, paired Wilcox test). FIG. 10B shows post-vaccination expansion of new T cell clones in the PBMC and their infiltration into the tumor in 9 out of 10 subjects. FIG. 10C shows that the most abundant clones show an active phenotype (CD8+CD69+) as assessed by TCRβ and RNA sequencing. Approx. 75% of new TIL clones were undetectable in blood prior to vaccination.

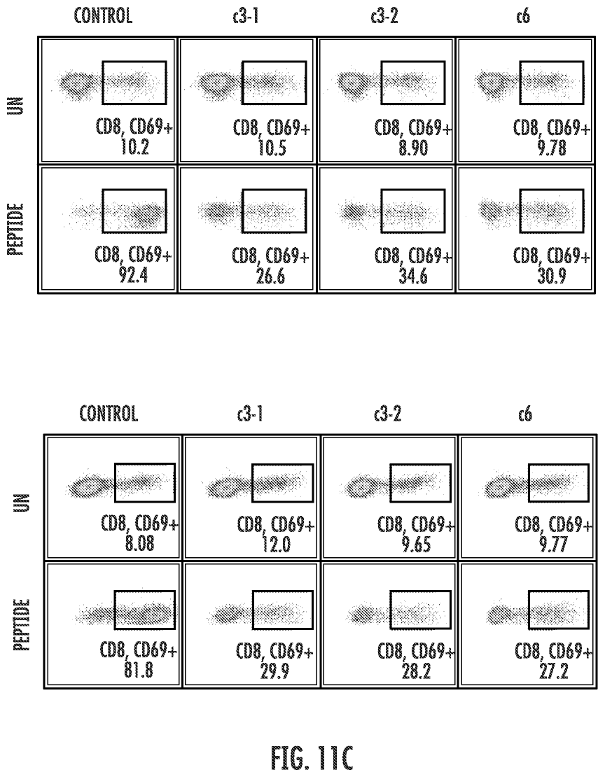

FIG. 11 shows that GNOS-PV02 generates neoantigen-specific, CD8+ and CD4+ anti-tumor responses. Table Z shows the most frequent TCRs identified by TCRβ and RNA sequencing in subjects Pt 8 and Pt 7 on week 9 post-vaccination.

### Example 5: Clinical Activity of PTCV and Pembrolizumab

A single-arm, open-label, multi-center phase 1/2 study of a personalized therapeutic cancer vaccine (PTCV) in combination with pembrolizumab (α PD-1 inhibitor) in patients with advanced HCC was conducted. The PTCV consisted of a DNA plasmid encoding up to 40 neoantigens (GNOS-PV02) identified through sequencing of an individual patient's tumor DNA and RNA as well as their germline DNA, which is designed to enhance immune processing and epitope presentation. GNOS-PV02 is co-formulated with a second DNA plasmid encoding IL-12 as a vaccine adjuvant (pIL12) and administered via intradermal injection followed by in vivo electroporation.

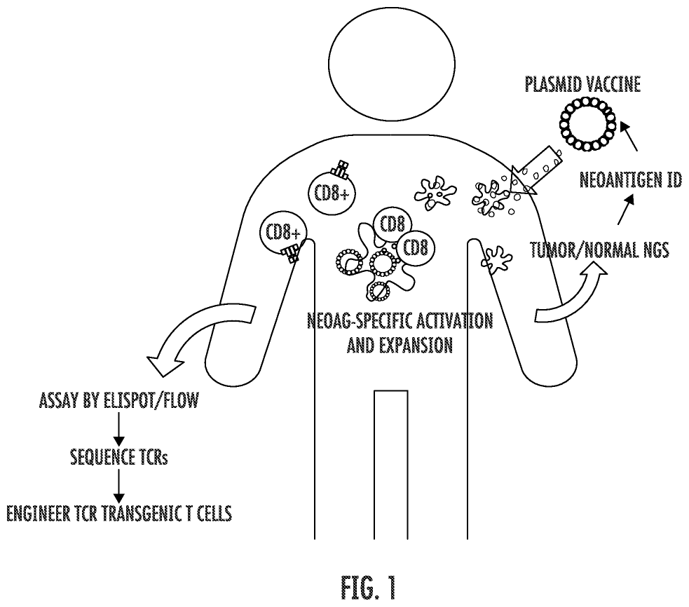

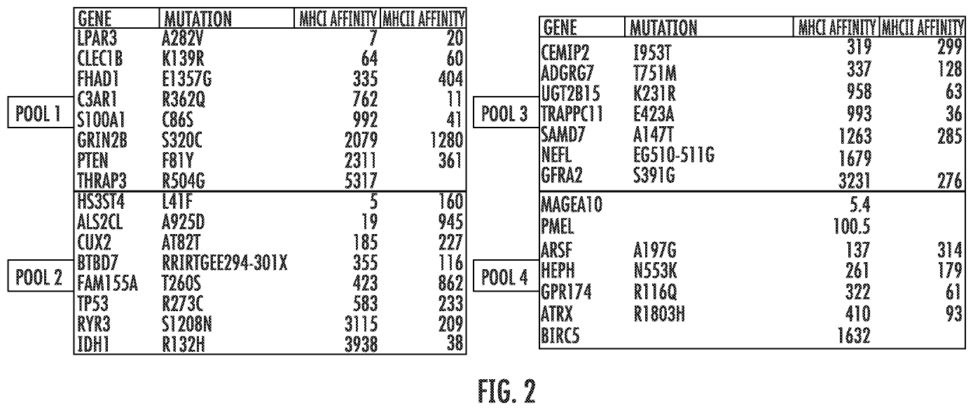

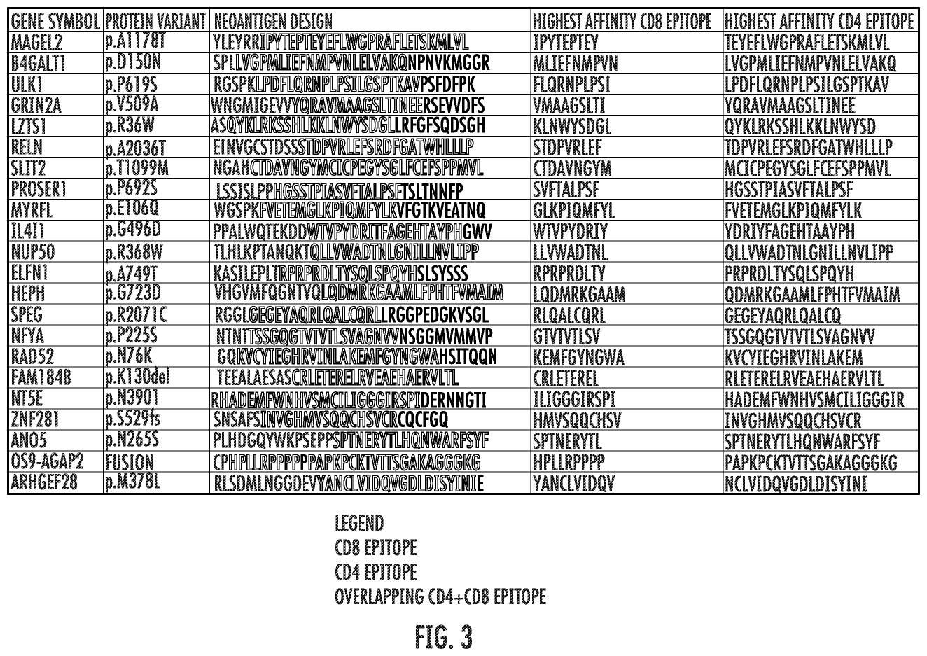

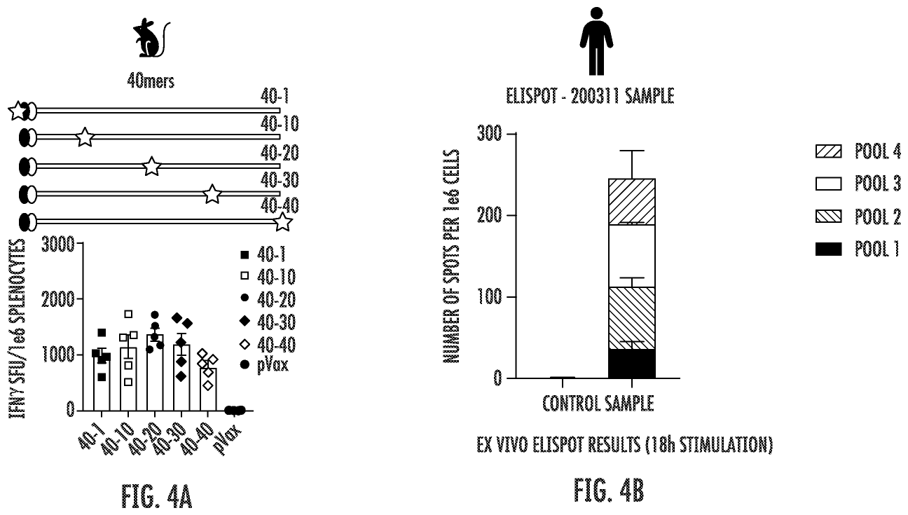

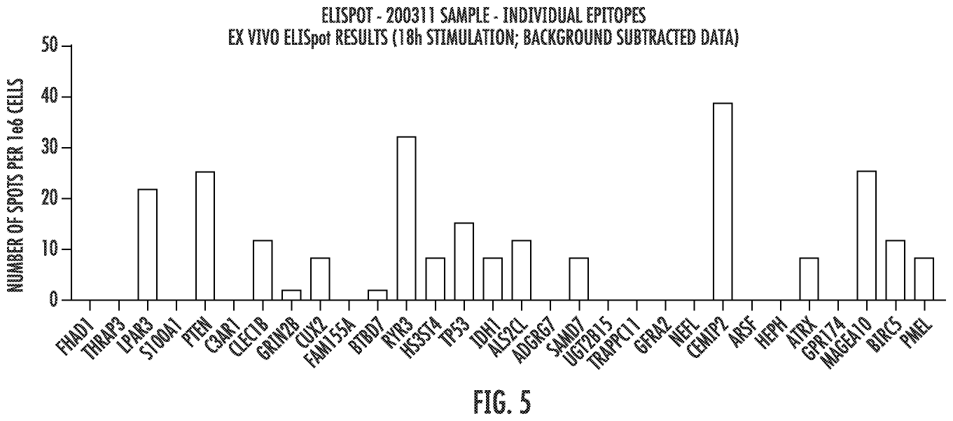

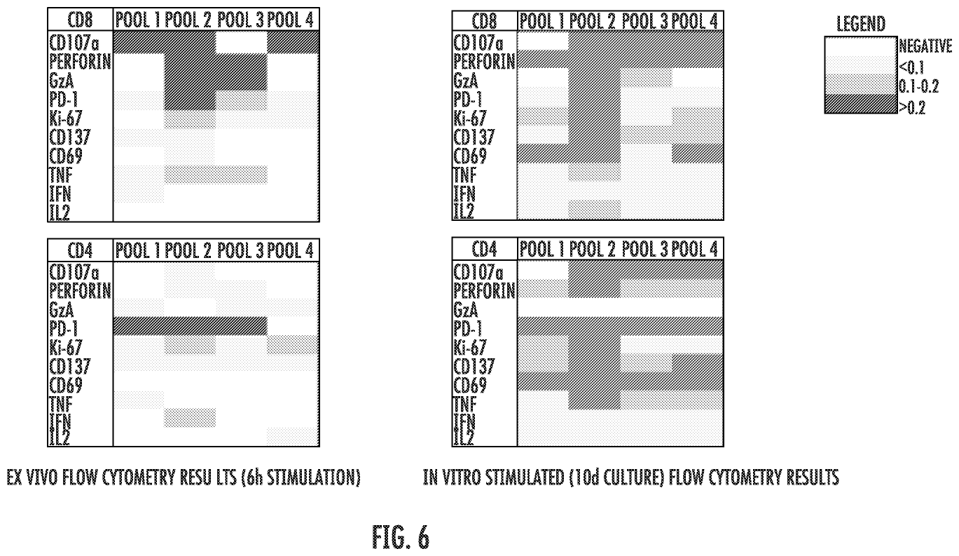

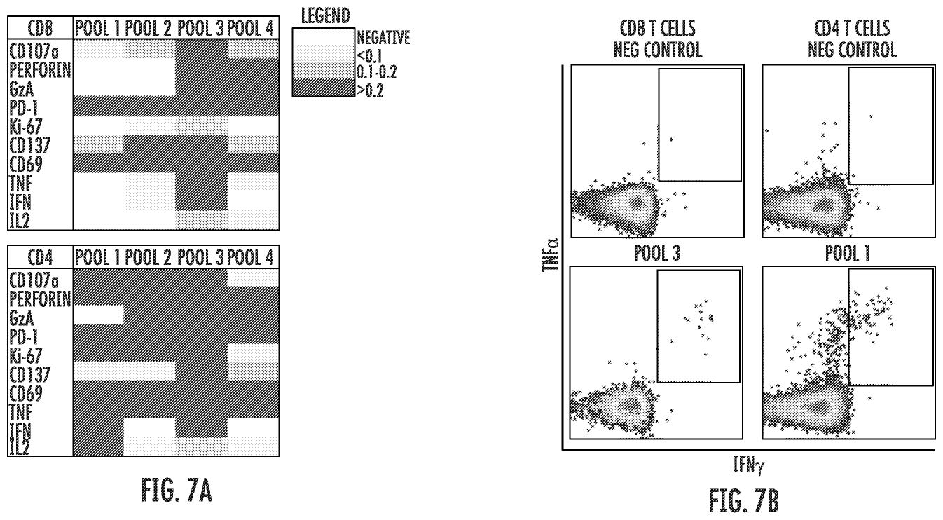

At the time of data analysis, 29/31 patients (out of a targeted enrollment of 36) had received at least one on-treatment restaging scan and were evaluable for response by RECIST 1.1. By investigator review, the objective response rate (ORR) (confirmed+ unconfirmed, mITT) per RECIST 1.1 was 32.3% (10/31) with 9.7% (3/31) of patients achieving a complete response (CR) and 22.6% (7/31) of patients achieving a partial response (PR). The disease control rate (DCR) was 54.8% (17/31) (FIG. 1b-d). One patient discontinued therapy due to an unrelated SAE after the first dose of therapy and was deemed unevaluable but was included in the mITT analysis. Among the patients who were evaluable for response, the ORR and DCR per RECIST 1.1 were 34.5% (10/29) and 58.6% (17/29), respectively. Initial response assessment was at 9 weeks, and among patients who had a response, the median time to response was 9 weeks (range 9-46 weeks). One patient with initially unresectable HCC achieved secondary resectability after five PTCV doses and discontinued therapy to pursue a potentially curative resection; this patient has not developed HCC recurrence as of the data cutoff. At data cutoff, the median duration of response (mDOR) had not been reached. OS and PFS were calculated from the first day of study treatment to death or progression or to the date of the last follow-up. The median PFS (mPFS) was 4.2 months and the median OS (mOS) was 19.9 months. Clinical results were generally consistent across major demographic and etiologic disease subgroups, including virally associated and unassociated HCC. The observed ORR of 32.3% at this interim 31-patient timepoint is numerically higher than the historical comparator rate for anti-PD1 monotherapy. The totality of the biomarker data suggests that the clinical benefit observed in GT-30 patients is unlikely to be explained by a response to anti-PD1 immunotherapy alone.

### Example 6: Vaccination Elicits Neoantigen-Specific Responses

Twenty-two patients with available peripheral blood mononuclear cell (PBMC) samples were evaluated for the presence of vaccine-induced neoantigen-specific responses prior to and following treatment using the ex vivo interferon (IFN)-γ enzyme-linked immune absorbent spot (ELISpot) assay (overnight peptide stimulation; no cytokines added). A T cell response to a specific epitope at a given time point was considered positive if it met each of three criteria to assure with 95% confidence that the response could be attributed to the specific peptide. The epitope-specific response (a) had to be at least 2 standard deviations above the corresponding unstimulated control sample (background); (b) had to be at least 2-fold above the corresponding unstimulated control sample (background); and (c) had to be at least 5 spot forming units (SFU). These criteria were used to evaluate both pre-treatment samples (for pre-existing neoepitope responses) and on-treatment samples.

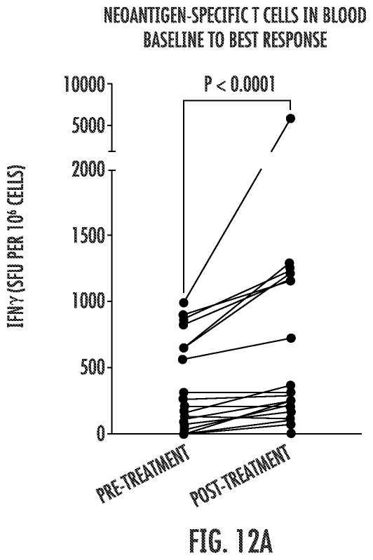

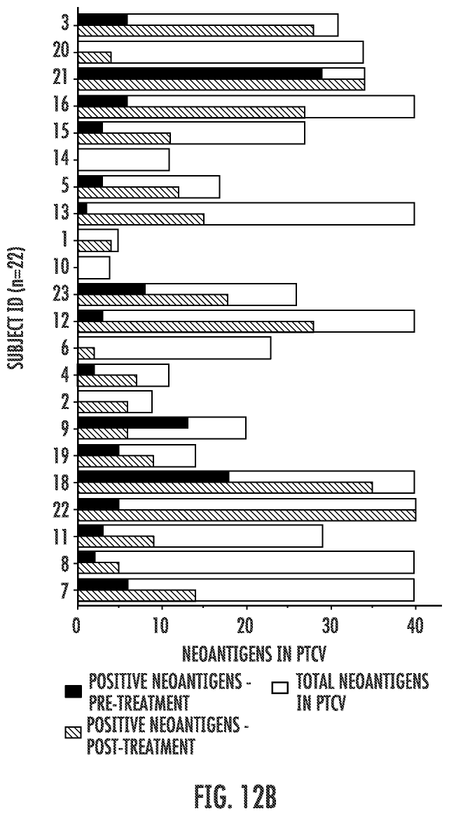

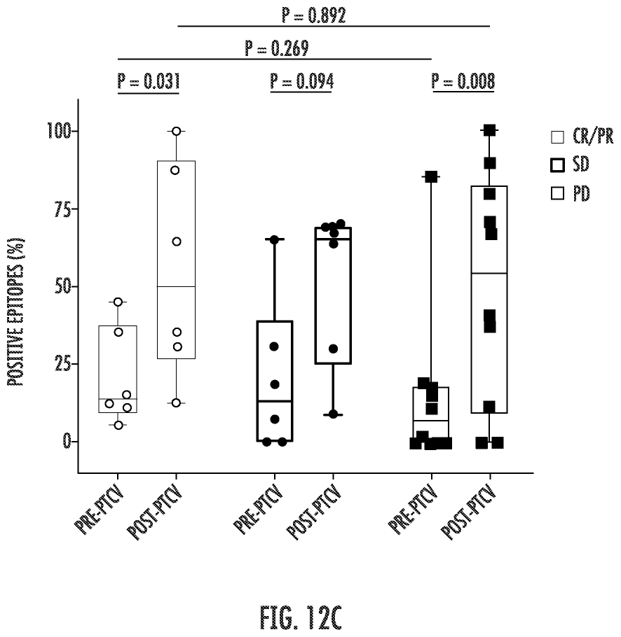

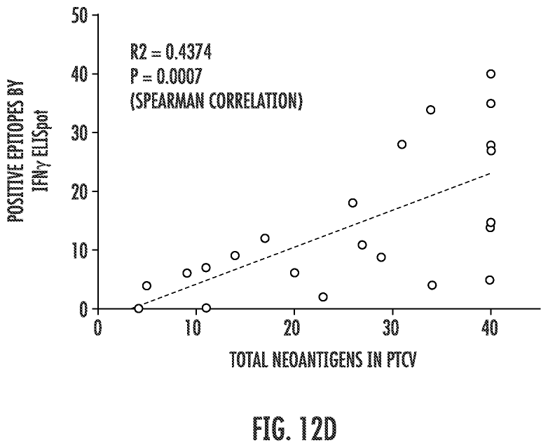

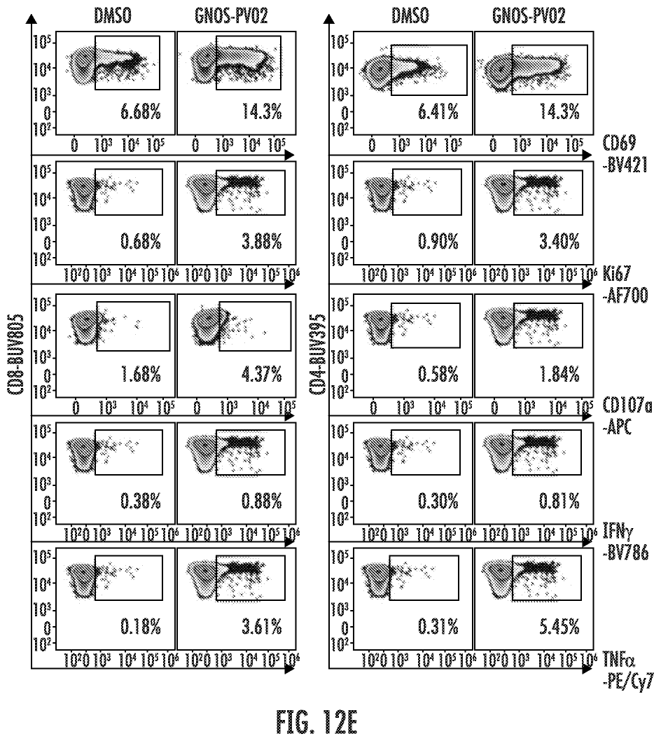

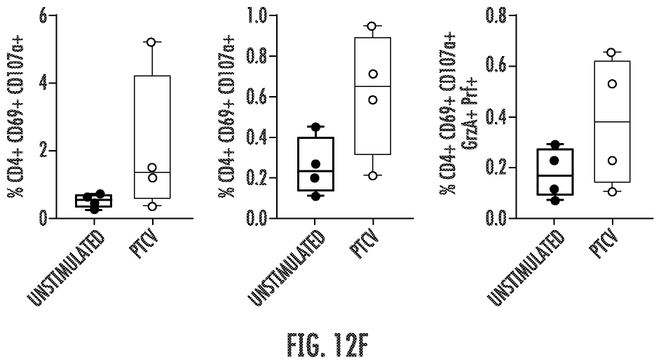

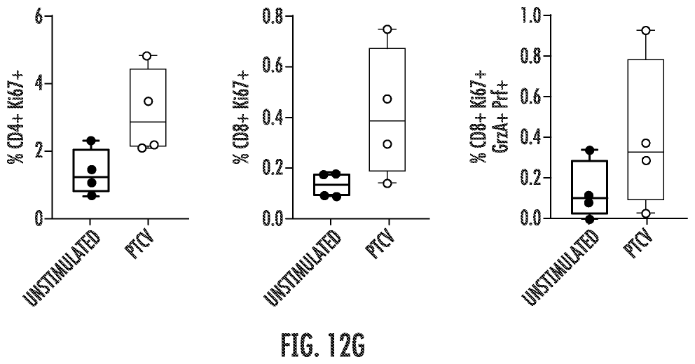

Vaccine neoepitope reactivity and T cell expansion relative to baseline levels were observed as early as week 3. In almost all patients, treatment was associated with an increase in the magnitude of cumulative PTCV neoantigen specific T cell responses (p<0.0001) (FIG. 12A) as well as the number of responding neoantigens (FIG. 12B). In 19/22 patients (86.4%), the number of vaccine encoded neoantigens with T cell reactivity was higher post-treatment than pre-treatment (FIG. 12β). Two patients with progressive disease, treated with PTCVs encoding 4 and 11 neoepitopes respectively, did not yield detectable ELISpot responses either pre- or post-treatment; and 1 patient (20 neoantigens; stable disease) had a reduced number of reactive epitopes detected post-treatment relative to their pre-treatment baseline. Individual epitope analyses across the cohort revealed PTCV encoded neoantigen specific T cell responses to a median of 11.8% (range 0.0%-85.3%) versus 64.0% (range 0.0%-100.0%) epitopes at pre-versus post-treatment, respectively. PTCV immunization resulted in a significant increase of positive epitopes in both clinically responding and non-responding patients (FIG. 12C).

Furthermore, there was a positive correlation between the total number of neoantigens included in the PTCV and the number of positive neoantigen responses by ELISpot assay (p=0.0007, Spearman correlation coefficient) (FIG. 12D). Immune responses were observed against neoepitopes with predicted HLA class I high binding affinity (kd<500 nM) as well as predicted medium and low binding affinity (kd 500-2000 nM). The increase in numbers of PTCV-encoded reactive neoantigens post vaccination is not consistent with anti-PD1 therapy but is consistent with the vaccine mechanism of action.

Neoantigen-specific responses were confirmed in a subset of 4 responding patients (1 CR, 3 PR) through intracellular cell staining of peripheral blood mononuclear cells (PBMCs) stimulated with patient-specific neoepitope pools in vitro. Upon neoantigen stimulation, both CD4+ and CD8+ T cell populations presented an increased activation profile as determined by the individual expression of the CD69+, Ki67+, CD107a+, IFNγ+, and TNFα+ markers (FIG. 12E). Boolean gating confirmed an increasing trend of active (CD69+CD107a+) (FIG. 12F) and proliferative (Ki67+) (FIG. 12G) polyfunctional CD4 and CD8 T cells with cytolytic capabilities (GrzA+Prf+) post-stimulation. Taken together, these data indicated that vaccination was capable of eliciting polyfunctional neoantigen-specific CD4+ and CD8+ T cell responses.

### Example 7: Vaccine-Enriched T Cell Clone Expansion and Tumor Infiltration

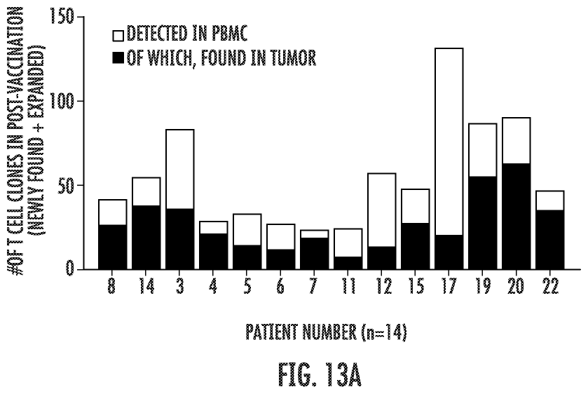

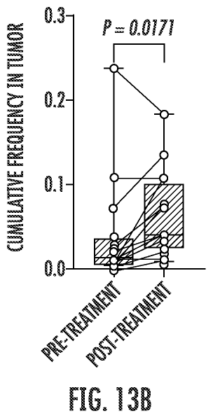

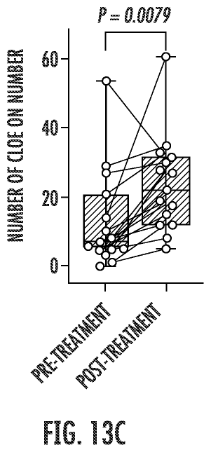

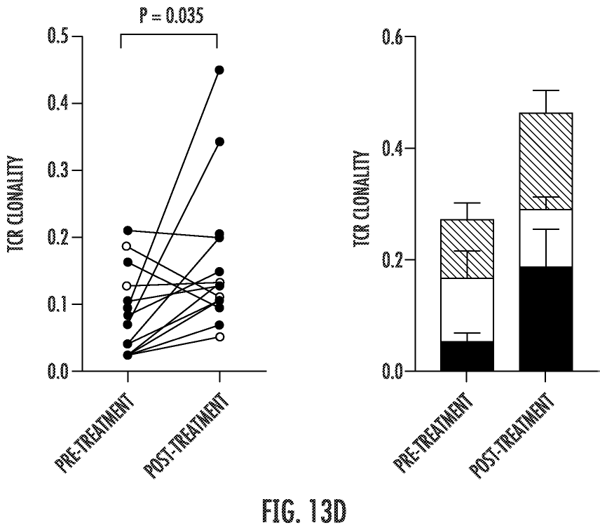

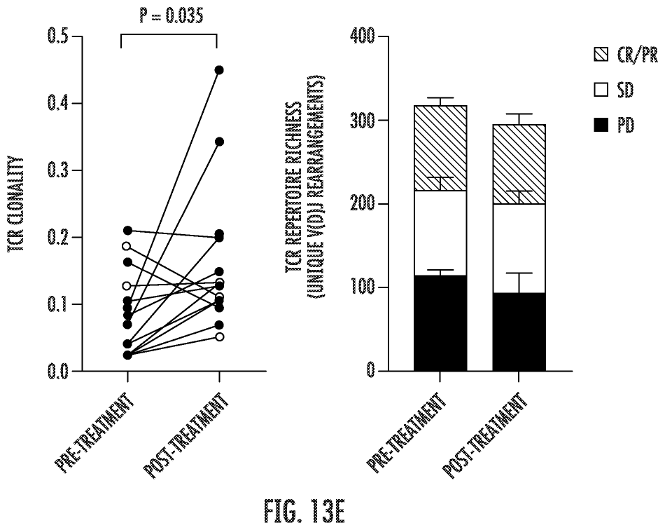

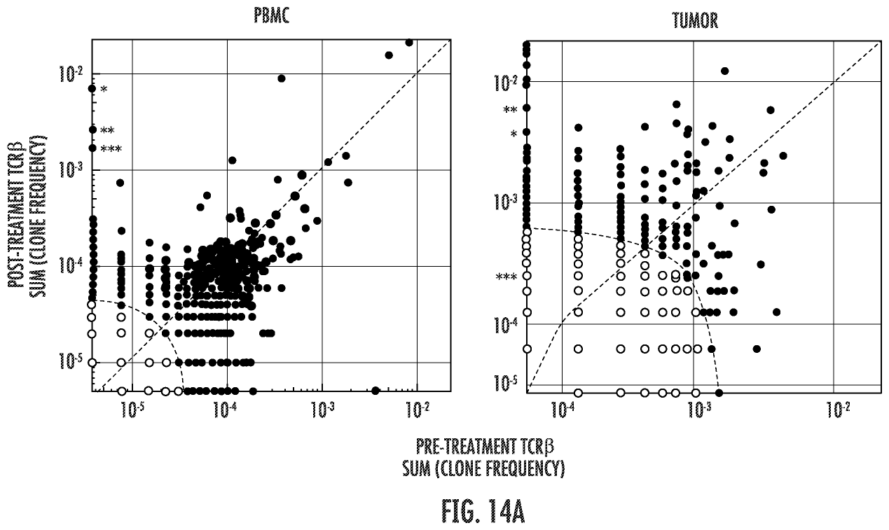

To evaluate T cell clonal dynamics with therapy, CDR3 regions of human TCRβ chains were sequenced from paired pre-treatment and post-treatment (weeks 9-12) PBMCs and tumor biopsies in 14 patients with available paired tumor biopsies. Although anti-PD-1 therapy is not known to modulate the diversity of tumor-reactive T cell clones, the inventors hypothesized that the addition of PTCV to anti-PD-1 therapy would lead to both an increase in abundance and broadening of the circulating HCC-reactive T cell clonal repertoire which would subsequently traffic to the tumor microenvironment. Consistent with this hypothesis, we observed significant T cell clonal expansion in 14/14 (100%) of subjects in both peripheral blood and tumor using a differential abundance statistical framework. The median number of new or expanded T cell clones in the periphery was 47 (range 24-132), of which a median of 21 (range 6-71) T cell clones were also new or expanded in the post-treatment tumor (FIG. 13A). Across all patients, the median increase in cumulative clonal frequencies of the significantly expanded clones was 1.94% (Range 0.35%-8.70%). An increase was observed in both abundance and number of expanded T cell clones within the tumor post-treatment that were also identified in the peripheral blood post-treatment (FIGS. 13B and C). Importantly, we observed higher frequencies and numbers of T cell clones newly present in the tumor post-vaccination. Additionally, we found a significantly increased TCR clonality in the tumor (p=0.035) (FIG. 13D) but no significant change in TCR repertoire richness in the tumor (p=0.216) (FIG. 13E). These data suggest that therapy with PTCV results in expansion of T cells in the periphery as well as in the tumor.

TABLE Z
TCR sequences

| | | | |
|---|---|---|---|
| 2905_c3-1 | CAVRDKGSARQLTF (SEQ | CSARDLYRNTEAFF (SEQ ID | 1.59E−02 |
| | ID NO: 509) | NO: 510) | |
| 2905_c3-2 | CAVSAGNNNARLMF | CSARDLYRNTEAFF (SEQ ID | 1.59E−02 |
| | (SEQ ID NO: 511) | NO: 512) | |
| 2905_c6 | CALDSSASKIIF (SEQ ID | CASSRQGQEQYF (SEQ ID | 3.72E−02 |
| | NO: 513) | NO: 514) | |
| 2101_c01 | CAEKGAYGQNFVF (SEQ ID | CASSSDALAGGIHRKLFF | 2.11E−02 |
| | NO: 515) | (SEQ ID NO: 516) | |
| 2101_c02 | CAFNAGNNRKLIW (SEQ ID | CASSSFRYNQPQHF (SEQ ID | 2.25E−04 |
| | NO: 517) | NO: 518) | |
| 2101_c03 | CAGVFEARLMF (SEQ ID | CASSLLQGTTAFF (SEQ ID | 1.86E−04 |
| | NO: 519) | NO: 520) | |
| 2101_c04 | CAVEDQGGKLIF (SEQ ID | CASSQDFAGSEQYF (SEQ ID | 1.37E−04 |
| | NO: 521) | NO: 522) | |
| 2101_c05 | CAVNDPPVDSSYKLIF (SEQ | CASSFADTQYF (SEQ ID | 1.27E−04 |
| | ID NO: 523) | NO: 524) | |
| 2101_c06 | CAVNDPPVDSSYKLIF (SEQ | CASSFADTQYF (SEQ ID | 1.27E−04 |
| | ID NO: 525) | NO: 526) | |
| 2101_c07 | CALAGGSYIPTF (SEQ ID | CASADSYEQYF (SEQ ID | 1.17E−04 |
| | NO: 527) | NO: 528) | |
| 2101_c08 | CAFGDSWGKFQF (SEQ ID | CASSIDQTGITEAFF (SEQ ID | 1.08E−04 |
| | NO: 529) | NO: 530) | |
| 2101_c09 | CAAIRSGSARQLTF (SEQ ID | CASSQDLGVAYNEQFF (SEQ | 1.08E−04 |
| | NO: 531) | ID NO: 532) | |
| 2101_c10 | CAYSGSARQLTF (SEQ ID | CASSFGGNTETQYF (SEQ ID | 1.08E−04 |
| | NO: 533) | NO: 534) | |
| 2101_c11 | CGTEAIGGTYKYIF (SEQ ID | CASSFGGNTETQYF (SEQ ID | 1.08E−04 |
| | NO: 535) | NO: 536) | |
| 2101_c12 | CIDSGGSNYKLTF (SEQ ID | CASSPAGPAQYF (SEQ ID | 1.57E−02 |
| | NO: 537) | NO: 538) | |
| 2101_c13 | CAGRLSSGGSYIPTF (SEQ | CASSAIGTPSGEQFF (SEQ ID | 9.01E−03 |
| | ID NO: 539) | NO: 540) | |
| 2101_c14 | CAVGSDGAGNMLTF (SEQ | CASTRDESYNSPLHF (SEQ | 6.94E−03 |
| | ID NO: 541) | ID NO: 542) | |
| 2101_c15 | CAVGGRSNAGGTSYGKLTF | CASTRDESYNSPLHF (SEQ | 6.94E−03 |
| | (SEQ ID NO: 543) | ID NO: 544) | |
| 2101_c16 | CAASVGGAGANNLFF (SEQ | CASSVDIYNEQFF (SEQ ID | 2.64E−03 |
| | ID NO: 545) | NO: 546) | |
| 2101_c17 | CAMREPYNFNKFYF (SEQ | CATHEGWDTGELFF (SEQ | 1.66E−03 |
| | ID NO: 547) | ID NO: 548) | |
| 2101_c18 | CAAGGTSYGKLTF (SEQ ID | CATTSGSPAGELFF (SEQ ID | 1.42E−03 |
| | NO: 549) | NO: 550) | |
| 2101_c19 | CAAETRNNNARLMF (SEQ | CAGRLAGASGELFF (SEQ ID | 1.27E−03 |
| | ID NO: 551) | NO: 552) | |
| 2101_c20 | CAFLLSDGQKLLF (SEQ ID | CAGRLAGASGELFF (SEQ ID | 1.27E−03 |
| | NO: 553) | NO: 554) | |
| 2101_c21 | CAAAGYSSASKIIF (SEQ ID | CASSLGPPGQTEAFF (SEQ | 7.43E−04 |
| | NO: 555) | ID NO: 556) | |
| 2101_c22 | CAMSAPWDTGNQFYF | CASSHGQGKDGELFF (SEQ | 7.43E−04 |
| | (SEQ ID NO: 557) | ID NO: 558) | |
| 2101_c23 | CAAEGNTDKLIF (SEQ ID | CASSQDPAGRWTEAFF | 7.34E−04 |
| | NO: 559) | (SEQ ID NO: 560) | |
| 2101_c24 | CTTSGTYKYIF (SEQ ID | CASSPRQGPGELFF (SEQ ID | 1.76E−04 |
| | NO: 561) | NO: 562) | |
| 2101_c25 | CAVEEISGGYQKVTF | (SEQ CASSENRGRADTQYF (SEQ | 2.93E−04 |
| | ID NO: 563) | ID NO: 564) | |
| 2101_c26 | CAVEEISGGYQKVTF | (SEQ CASSENRGRADTQYF (SEQ | 2.93E−04 |
| | ID NO: 565) | ID NO: 566) | |
| 2101_c27 | CAVEEISGGYQKVTF (SEQ | CASSENRGRADTQYF (SEQ | 2.93E−04 |
| | ID NO: 567) | ID NO: 568) | |
| 2101_c28 | CAESIGDYKLSF (SEQ ID | CASSQELVGPNTGELFF | 9.78E−05 |
| | NO: 569) | (SEQ ID NO: 570) | |
| 2101_c29 | CAATSMDSNYQLIW (SEQ | CASSIEQGVAQYF (SEQ ID | 1.37E−04 |
| | ID NO: 571) | NO: 572) | |
| 2101_c30 | CAFTYSGGGADGLTF (SEQ | CATSREGATFYNEQFF (SEQ | 5.48E−04 |
| | ID NO: 573) | ID NO: 574) | |
| 2101_c31 | CIVRVAGFLYSGGGADGLTF | CASSVGEFTGTDTQYF (SEQ | 8.80E−05 |
| | (SEQ ID NO: 575) | ID NO: 576) | |
| 2101_c32 | CVVSRGDYKLSF (SEQ ID | CAINPDSNQPQHF (SEQ ID | 3.13E−04 |
| | NO: 577) | NO: 578) | |
| 2101_c33 | CAVGNARLMF (SEQ ID | CAINPDSNQPQHF (SEQ ID | 3.13E−04 |
| | NO: 579) | NO: 580) | |
| 2101_c34 | CAVSPMTGFQKLVF (SEQ | CASSELEGDGSGNTIYF | 8.80E−05 |
| | ID NO: 581) | (SEQ ID NO: 582) | |
| 2101_c35 | CAASGTSYGKLTF (SEQ ID | CASSQGGSGTLFHEQFF | 4.01E−04 |
| | NO: 583) | (SEQ ID NO: 584) | |
| 2101_c36 | CAVPHGATNKLIF (SEQ ID | CASKGVVYEQYF (SEQ ID | 2.25E−04 |
| | NO: 585) | NO: 586) | |
| 2101_c37 | CAEIPFSGGYNKLIF (SEQ | CASSGRTGGGYEQYF (SEQ | 2.15E−04 |
| | ID NO: 587) | ID NO: 588) | |

### Example 8: Post-Vaccination Expanded TCR Clones Identified in the Tumor are Reactive to PTCV Encoded Antigens

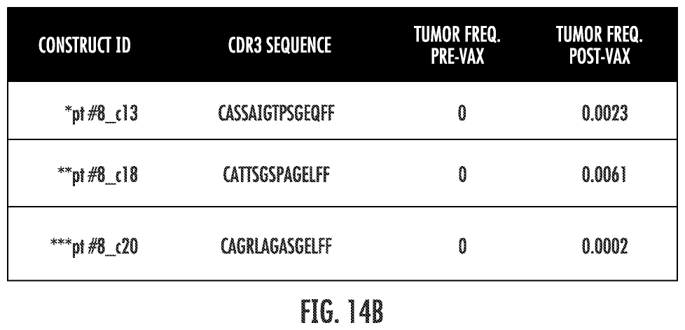

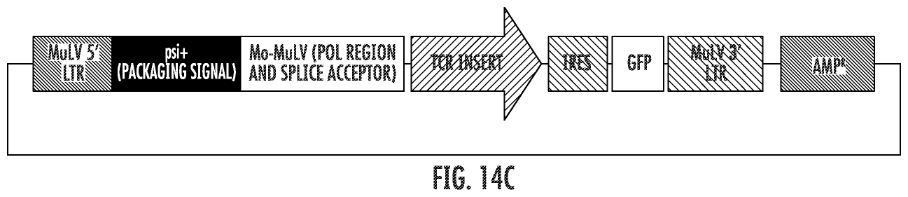

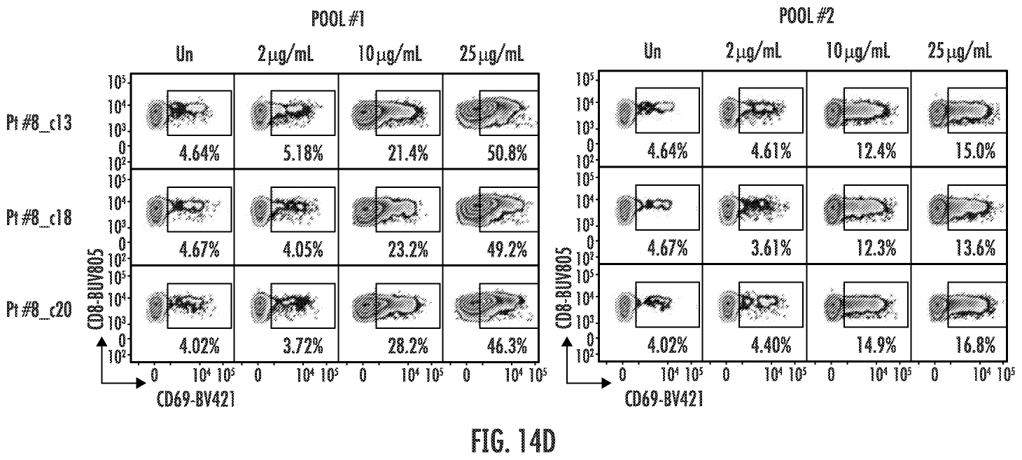

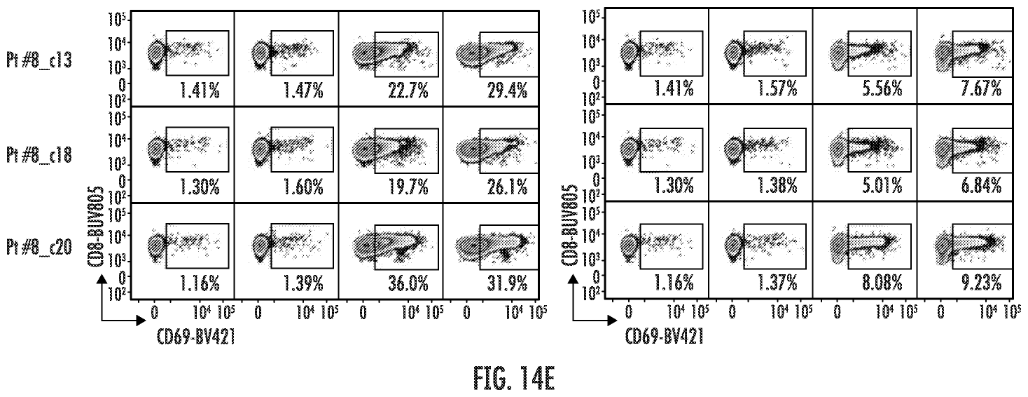

Lastly, the neoantigen-specific activity of tumor-infiltrating T cells in two representative patients was evaluated. The first patient had 42 significantly expanded clones in the periphery, of which 27 were found in the tumor sample post-treatment (FIG. 14A). Three TCR sequences from T cell clones newly present in the tumor post-vaccination were selected and cloned into the pMXs-IRES-GFP retroviral plasmid vector for further studies (FIGS. 14 B and C). To characterize the neoantigen-specific cellular response driven by the treatment with GNOS-PV02, TCR-engineered T cells from patient-derived PBMCs were stimulated with patient-specific neoantigen pools that were included in the patient-specific PTCV. There was a dose-dependent CD8+ (FIG. 14D) and CD4+ (FIG. 14E) T cell activation (CD69+) associated with pool #1, which included the most reactive epitopes measured by ELISpot. In the second representative patient, we were able to further map the new T cells/TCRs to a specific vaccine-encoded epitope. From IFNγ ELISpot analysis we first identified a strongly immunogenic epitope (ATP1A1-ALB) encoded in the patient's personalized vaccine. Patient-derived PBMCs were subjected to IVS for T cell enrichment and expansion and then stimulated with ATP1A1-ALB peptides. We found both CD4+ and CD8+ T cells with specific polyfunctional responses (CD69+, Ki67+, CD137+, IFNγ+, IL2+) against ATP1A1-ALB Moreover, high-frequency TCRs were identified by TCR/RNAseq (33 clones expanded in the periphery, of which 15 clones were found in the tumor) and engineered as previously described. Engineered TCRs were stimulated with a pool of epitopes containing all the neoantigens in the patient's PTCV. Similar to the first patient, we observed CD4+ and CD8+ T cell specificity against the patient-specific neoantigens. Together, these data validate the post-vaccination infiltration and increase in the frequency of T cells in the tumor with specificity to vaccine-encoded neoantigens.

### CONCLUSIONS

GT-EPIC™ personalized vaccines containing up to about 40 neoantigens can be designed, manufactured, and administered successfully in as short as 6 weeks allowing concurrent start with anti-PD1 in 2nd line HCC. GNOS-PV02+INO-9012 in combination with pembrolizumab achieved an ORR of 25% in the first 12 patients of the clinical trial (3 PR) and a DCR of 67%. Patients treated with GNOS-PV02+INO-9012 in combination with pembrolizumab had new T cell clones in blood following vaccination, with new clones comprising up to 1% of the peripheral T cell repertoire. Engineered TCRs identified from high-frequency T cell clones in tumor post-treatment, respond to peptides encoded in the vaccine. GNOS-PV02+INO-9012 present an unremarkable safety profile with no treatment-related SAEs.

GNOS-PV02, a neoantigen DNA vaccine, in combination with plasmid encoding IL-12 and pembrolizumab resulted in expansion of newly identified T cells, primarily activated CD8, which trafficked to the tumor. These new tumor-infiltrating T cells showed TCR specificity against tumor neoantigens encoded in GNOS-PV02 and may account for the observed objective decrease in tumor size.

The present study provides new evidence that a neoantigen-specific vaccine in combination with pembrolizumab can be effective in immunotherapy-resistant tumor types, even in later line settings. One notable difference between the vaccine platform utilized in the present study and other neoantigen-vaccine platforms is that other vaccine platforms have generally utilized algorithms based on predicted MHC Class I binding affinity to target a more limited selection of neoantigens. Here it is shown that vaccines that encode for a larger repertoire of tumor derived neoantigens may lead to the priming of a broader set of immune responses, increasing the likelihood of effective tumor control and also leading to better coverage of tumor heterogeneity and oligoclonality thus reducing the likelihood of tumor escape from vaccine induced immune pressure.

1.-56. (canceled)

57. A method of preventing metastases of a cancer comprising one or a plurality of antigens in a subject, the method comprising:
(a) administering to the subject one or a plurality of nucleic acid sequences encoding the one or plurality of neoantigens;
(b) allowing clonal T cells primed against the one or plurality of neoantigens in the subject to expand;
(c) isolating the clonal T cells from the subject;
(d) identifying one or a plurality of nucleotide sequences encoding a subset of TCRs that are highly immunogenic in response to the one or plurality of neoantigens in the subject; and
(e) administering a therapeutically effective amount of T cells comprising a nucleic acid molecule encoding one or a plurality of the subset of TCRs to the subject in need thereof.

58. The method of claim 57, wherein step (d) comprises performing an assay measuring one or a combination of: (i) the avidity or affinity of cells expressing the TCRs to bind cells in vitro; and (ii) the percentage of CD8+ and/or CD4+ on cells expressing the TCRs.

59. The method of claim 57, further comprising identifying the one or plurality of neoantigens from a tissue sample removed from the subject.

60. The method of claim 57, wherein the method is free of an in vitro expansion of PBMC and/or tumor infiltrating lymphocytes.

61. The method of claim 57, wherein a total number of the clonal T cells primed against the one or plurality of neoantigens in the subject comprise from about 25% to about 50% CD8+ reactivity to the one or plurality of neoantigens.

62. The method of claim 57, wherein step (a) comprises administering a nucleic acid molecule comprising the one or plurality of nucleotide sequences encoding the one or plurality of neoantigens.

63. The method of claim 57, wherein the cancer is HCC.

64. The method of claim 57 further comprising administering to the subject a checkpoint inhibitor.

65. A method of treating cancer expressing one or a plurality of antigens in a subject in need thereof, the method comprising:
(a) administering one or a plurality of nucleic acid sequences encoding the one or plurality of antigens to the subject in need thereof; and
(b) administering a therapeutically effective amount of T cells comprising one or a plurality of nucleic acid sequences encoding one or a plurality of T cell receptors (TCRs) or functional fragments thereof from the subject that are highly immunogenic in response to the one or plurality of neoantigens to the subject.

66. The method of claim 65, wherein the method is free of an in vitro expansion of PBMC and/or tumor infiltrating lymphocytes.

67. The method of claim 65 further comprising allowing the subject to elicit an immune response against the one or plurality of neoantigens.

68. The method of claim 65 further comprising sequencing the one or plurality of nucleic acid sequences encoding the one or plurality of TCRs or functional fragments thereof from T cells isolated from the subject after step (a) but prior to step (b).

69. The method of claim 65, wherein, after step (a), allowing a time period sufficient for the subject to expand a clonal T cell population primed against the one or plurality of neoantigens, wherein the clonal T cell population comprises from about 25% to about 50% CD8+ reactivity to the one or plurality of neoantigens.

70. The method of claim 65, wherein step (a) comprises administering a nucleic acid molecule comprising the one or plurality of nucleic acid sequence encoding the one or plurality of neoantigens.

71. The method of claim 65, wherein the cancer is HCC.

72. A method of treating cancer in a subject in need thereof, the cancer expressing one or a plurality of neoantigens in a subject in need thereof, the method comprising:
(a) administering one or a plurality of nucleic acid sequences encoding the one or plurality of neoantigens to the subject; and
(b) administering a therapeutically effective amount of a checkpoint inhibitor to the subject.

73. The method of claim 72, wherein step (a) comprises administering a DNA plasmid encoding from about 10 to about 55 neoantigens.

74. The method of claim 72, wherein the checkpoint inhibitor is a PD-1 inhibitor.

75. A method of treating cancer comprising one or a plurality of neoantigens in a subject in need thereof, the method comprising:
(a) administering to the subject in need thereof one or a plurality of nucleic acid sequences encoding the one or plurality of neoantigens;
(b) allowing clonal T cells primed against the one or plurality of antigens in the subject to expand;
(c) isolating the clonal T cells from the subject;
(d) identifying one or a plurality of nucleotide sequences encoding a subset of T cell receptors (TCRs) that are highly immunogenic in response to the one or plurality of antigens in the subject; and
(e) administering a therapeutically effective amount of T cells comprising a nucleic acid molecule encoding one or a plurality of the subset of TCRs to the subject in need thereof.

76. The method of claim 75, wherein the clonal T cells are isolated by drawing a blood sample from the subject and sorting the peripheral blood mononuclear cells (PBMCs) from the sample according to receptor expression on the PBMC surface.

77. The method of claim 75, wherein step (d) comprises performing an assay measuring one or a combination of: (i) the avidity or affinity of cells expressing the TCRs to bind cells in vitro; and (ii) the percentage of CD8+ and/or CD4+ on cells expressing the TCRs.

78. The method of claim 75 further comprising sequencing the one or plurality of nucleotide sequences encoding the subset of TCRs that are highly immunogenic from the T cells expressing the TCRs.

79. The method of claim 75 further comprising identifying the one or plurality of antigens from a tissue sample removed from the subject.

80. A method of manufacturing a population of T cells expressing one or a plurality of TCRs, or functional fragments thereof, that recognize one or a plurality of neoantigens, the method comprising:
(a) administering one or a plurality of nucleic acid sequences encoding the one or plurality of neoantigens to a subject comprising one or a plurality of cells expressing the one or plurality of neoantigens; and
(b) isolating clonally derived T cells expressing the one or plurality of TCRs or functional fragments thereof from the subject.
